SEQUENCE LISTING (59 sequences)

SEQ ID NO: 1 - protein, 12 aa, Homo sapiens
  spacer (IgG4hinge)
ESKYGPPCPPCP

SEQ ID NO: 2 - DNA, 36 bp, Homo sapiens
  spacer (IgG4hinge)
gaatctaagtacggaccgccctgccccccttgccct

SEQ ID NO: 3 - protein, 119 aa, Homo sapiens
  Hinge-CH3 spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 229 aa, Homo sapiens
  Hinge-CH2-CH3 spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 282 aa, Homo sapiens
  IgD-hinge-Fc
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 6 - protein, 24 aa, Artificial Sequence
  T2A
LEGGGEGRGSLLTCGDVEENPGPR

SEQ ID NO: 7 - protein, 357 aa, Artificial Sequence
  tEGFR
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 8 - protein, 27 aa, Homo sapiens
  CD28 (amino acids 153-179 of P10747)
FWVLVVVGGVLACYSLLVTVAFIIFWV

SEQ ID NO: 9 - protein, 66 aa, Homo sapiens
  CD28 (amino acids 114-179 of P10747)
IEVMYPPPYLDNEKSNGTIIHVKGKHLCPSPLFPGPSKPFWVLVVVGGVLACYSLLVTVAFIIFWV

SEQ ID NO: 10 - protein, 41 aa, Homo sapiens
  CD28 (amino acids 180-220 of P10747)
RSKRSRLLHSDYMNMTPRRPGPTRKHYQPYAPPRDFAAYRS

SEQ ID NO: 11 - protein, 41 aa, Homo sapiens
  CD28 (LL to GG)
RSKRSRGGHSDYMNMTPRRPGPTRKHYQPYAPPRDFAAYRS

SEQ ID NO: 12 - protein, 42 aa, Homo sapiens
  4-1BB (amino acids 214-255 of Q07011.1)
KRGRKKLLYIFKQPFMRPVQTTQEEDGCSCRFPEEEEGGCEL

SEQ ID NO: 13 - protein, 112 aa, Homo sapiens
  CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 112 aa, Homo sapiens
  CD3 zeta
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 112 aa, Homo sapiens
  CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 335 aa, Artificial Sequence
  tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 17 - protein, 18 aa, Artificial Sequence
  T2A
EGRGSLLTCGDVEENPGP

SEQ ID NO: 18 - protein, 22 aa, Artificial Sequence
  P2A
GSGATNFSLLKQAGDVEENPGP

SEQ ID NO: 19 - protein, 19 aa, Artificial Sequence
  P2A
ATNFSLLKQAGDVEENPGP

SEQ ID NO: 20 - protein, 20 aa, Artificial Sequence
  E2A
QCTNYALLKLAGDVESNPGP

SEQ ID NO: 21 - protein, 22 aa, Artificial Sequence
  F2A
VKQTLNFDLLKLAGDVESNPGP

SEQ ID NO: 22 - protein, 10 aa, Artificial Sequence
  Linker
  REPEAT (5)...(9): SGGGG is repeated 5 time
PGGGSGGGGP

SEQ ID NO: 23 - protein, 17 aa, Artificial Sequence
  Linker
GSADDAKKDAAKKDGKS

SEQ ID NO: 24 - DNA, 66 bp, Artificial Sequence
  GMCSFR alpha chain signal sequence
atgcttctcctggtgacaagccttctgctctgtgagttaccacacccagcattcctcctgatccca

SEQ ID NO: 25 - protein, 22 aa, Artificial Sequence
  GMCSFR alpha chain signal sequence
MLLLVTSLLLCELPHPAFLLIP

SEQ ID NO: 26 - protein, 18 aa, Artificial Sequence
  CD8 alpha signal peptide
MALPVTALLLPLALLLHA

SEQ ID NO: 27 - protein, 15 aa, Artificial Sequence
  Hinge
EPKSCDKTHTCPPCP

SEQ ID NO: 28 - protein, 12 aa, Artificial Sequence
  Hinge
ERKCCVECPPCP

SEQ ID NO: 29 - protein, 61 aa, Artificial Sequence
  Hinge
ELKTPLGDTHTCPRCPEPKSCDTPPPCPRCPEPKSCDTPPPCPRCPEPKSCDTPPPCPRCP

SEQ ID NO: 30 - protein, 12 aa, Artificial Sequence
  Hinge
ESKYGPPCPSCP

SEQ ID NO: 31 - protein, 12 aa, Artificial Sequence
  Hinge
ESKYGPPCPPCP

SEQ ID NO: 32 - protein, 9 aa, Artificial Sequence
  Hinge
YGPPCPPCP

SEQ ID NO: 33 - protein, 10 aa, Artificial Sequence
  Hinge
KYGPPCPPCP

SEQ ID NO: 34 - protein, 14 aa, Artificial Sequence
  Hinge
EVVVKYGPPCPPCP

SEQ ID NO: 35 - protein, 11 aa, Artificial Sequence
  CDR L1
RASQDISKYLN

SEQ ID NO: 36 - protein, 7 aa, Artificial Sequence
  CDR L2
SRLHSGV

SEQ ID NO: 37 - protein, 9 aa, Artificial Sequence
  CDR L3
GNTLPYTFG

SEQ ID NO: 38 - protein, 5 aa, Artificial Sequence
  CDR H1
DYGVS

SEQ ID NO: 39 - protein, 16 aa, Artificial Sequence
  CDR H2
VIWGSETTYYNSALKS

SEQ ID NO: 40 - protein, 7 aa, Artificial Sequence
  CDR H3
YAMDYWG

SEQ ID NO: 41 - protein, 120 aa, Artificial Sequence
  VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 42 - protein, 107 aa, Artificial Sequence
  VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 43 - protein, 245 aa, Artificial Sequence
  scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 44 - protein, 11 aa, Artificial Sequence
  CDR L1
KASQNVGTNVA

SEQ ID NO: 45 - protein, 7 aa, Artificial Sequence
  CDR L2
SATYRNS

SEQ ID NO: 46 - protein, 9 aa, Artificial Sequence
  CDR L3
QQYNRYPYT

SEQ ID NO: 47 - protein, 5 aa, Artificial Sequence
  CDR H1
SYWMN

SEQ ID NO: 48 - protein, 17 aa, Artificial Sequence
  CDR H2
QIYPGDGDTNYNGKFKG

SEQ ID NO: 49 - protein, 13 aa, Artificial Sequence
  CDR H3
KTISSVVDFYFDY

SEQ ID NO: 50 - protein, 122 aa, Artificial Sequence
  VH
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 51 - protein, 108 aa, Artificial Sequence
  VL
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 52 - protein, 15 aa, Artificial Sequence
  Linker
GGGGSGGGGSGGGGS

SEQ ID NO: 53 - protein, 245 aa, Artificial Sequence
  scFv
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 54 - protein, 12 aa, Artificial Sequence
  HC-CDR3
HYYYGGSYAMDY

SEQ ID NO: 55 - protein, 7 aa, Artificial Sequence
  LC-CDR2
HTSRLHS

SEQ ID NO: 56 - protein, 9 aa, Artificial Sequence
  LC-CDR3
QQGNTLPYT

SEQ ID NO: 57 - DNA, 735 bp, Artificial Sequence
  Sequence encoding scFv
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 58 - protein, 5 aa, Artificial Sequence
  Hinge
  VARIANT (1)...(1): Xaa is glycine, cysteine or arginine
  VARIANT (4)...(4): Xaa is cysteine or threonine
XPPXP

SEQ ID NO: 59 - protein, 18 aa, Artificial Sequence
  Linker
GSTSGSGKPGSGEGSTKG

(19) United States
(12) Patent Application Publication
(10) Pub. No.: US 2022/0249637 A1
(43) Pub. Date: Aug. 11, 2022

(54) COMBINATION THERAPY OF A CELL-MEDIATED CYTOTOXIC THERAPY AND AN INHIBITOR OF A PROSURVIVAL BCL2 FAMILY PROTEIN

(71) Applicant: Juno Therapeutics, Inc., Seattle, WA (US)

(72) Inventors: Michael PORTS, Seattle, WA (US); Evan Paul THOMAS, Seattle, WA (US); Rupesh AMIN, Seattle, WA (US); Jason DUBOVSKY, Seattle, WA (US); Ronald DUBOWY, Seattle, WA (US); Archana BRAHMANDAM, Seattle, WA (US)

(73) Assignee: Juno Therapeutics, Inc., Seattle, WA (US)

(21) Appl. No.: 17/617,808

(22) PCT Filed: Jun. 11, 2020
(86) PCT No.: PCT/US2020/037333

§ 371 Date: Dec. 9, 2021

Related U.S. Application Data
(60) Provisional application No. 62/890,594, filed on Aug. 22, 2019.
(60) Provisional application No. 62/860,748, filed on Jun. 12, 2019.

(51) Int. Cl. A61K 39/00; A61K 31/635; C07K 16/28; C07K 14/725; C07K 14/705; A61K 31/7076; A61K 31/675; A61P 35/02; A61P 35/00

(52) U.S. Cl. CPC A61K 39/001112; A61K 31/635; C07K 16/2803; C07K 14/7051; C07K 14/70578; C07K 14/70521; A61K 2039/804; A61K 31/675; A61P 35/02; A61P 35/00; A61K 2039/5156; A61K 2039/545; A61K 31/7076

(57) ABSTRACT

Provided are methods, uses, and articles of manufacture of combination therapies involving immunotherapies and cell therapies, such as adoptive cell therapy, e.g. a T cell therapy, and the use of an inhibitor of a prosurvival BCL2 family protein, e.g. a BCL2 inhibitor, for treating subjects having or suspected of having a cancer, and related methods, uses, and articles of manufacture. The T cell therapy includes cells that express recombinant receptors such as chimeric antigen receptors (CARs).

### CROSS-REFERENCE TO RELATED APPLICATIONS

This application claims priority to U.S. provisional applications 62/860,748, filed Jun. 12, 2019, entitled “COMBINATION THERAPY OF A CELL-MEDIATED CYTOTOXIC THERAPY AND AN INHIBITOR OF A PROSURVIVAL BCL2 FAMILY PROTEIN” and 62/890,594, filed Aug. 22, 2019, entitled “COMBINATION THERAPY OF A CELL-MEDIATED CYTOTOXIC THERAPY AND AN INHIBITOR OF A PROSURVIVAL BCL2 FAMILY PROTEIN,” the contents of which are incorporated by reference in their entirety for all purposes.

### INCORPORATION BY REFERENCE OF SEQUENCE LISTING

The present application is being filed along with a Sequence Listing in electronic format. The Sequence Listing is provided as a file entitled 735042021340SeqList.TXT, created Jun. 8, 2020, which is 35,272 bytes in size. The information in the electronic format of the Sequence Listing is incorporated by reference in its entirety.

### FIELD

The present disclosure relates in some aspects to methods and uses of combination therapies involving a therapy such as an immunotherapy or a cell therapy, e.g., a T cell therapy, and the use of an inhibitor of a prosurvival BCL2 family protein, e.g., a BCL2 inhibitor, for treating subjects with cancers such as leukemias and lymphomas, and related methods, uses, and articles of manufacture. The T cell therapy includes cells that express recombinant receptors such as chimeric antigen receptors (CARs).

### BACKGROUND

Various strategies are available for immunotherapy and cell therapy for treating cancers, for example, adoptive cell therapies, including those involving the administration of cells expressing chimeric receptors specific for a disease or disorder of interest, such as chimeric antigen receptors (CARs) and/or other recombinant antigen receptors, as well as other adoptive immune cell and adoptive T cell therapies. Subsets of cancers are resistant to or develop resistance to such therapies. Improved methods are therefore needed, for example, to overcome this resistance and increase the efficacy of such methods. Provided are methods and uses that meet such needs.

### SUMMARY

Provided herein are methods and uses involving combination therapies for treating subjects having or suspected of having a cancer, such as a chronic lymphocytic leukemia (CLL), a non-Hodgkin lymphoma (NHL), or a subtype thereof. In some embodiments, the cancer is a small lymphocytic lymphoma (SLL). The methods and other embodiments generally relate to combinations involving administering to the subject a therapy, which is an immunotherapy or a cell therapy, and an inhibitor of a prosurvival BCL2 family protein, such as a BCL2 inhibitor. In some aspects, the provided methods and uses involve the administration of a T cell therapy such as CAR-expressing T cells comprising an antigen-binding domain that binds to an antigen expressed on B cells.

Provided herein is a method of treating cancer including administering a cytotoxic therapy to a subject having a cancer, wherein the cytotoxic therapy is an immunotherapy or a cell therapy and specifically binds to an antigen associated with, expressed by, or present on cells of the cancer; and administering to the subject an inhibitor of a prosurvival BCL2 family protein in a dosing regimen sufficient to achieve a steady state concentration (Css) of the inhibitor at a time between at or about 1 day and at or about 14 days after initiation of administration of the cytotoxic therapy and/or at a time before peak levels of the cytotoxic therapy is detectable in the blood of the subject following administration of the cytotoxic therapy.

Provided herein is a method of treating cancer including administering to a subject having a cancer an inhibitor of a prosurvival BCL2 family protein in a dosing regimen sufficient to achieve a steady state concentration (Css) of the inhibitor at a time between at or about 1 day and at or about 14 days after initiation of administration of a cytotoxic therapy and/or at a time before peak levels of the cytotoxic therapy is detectable in the blood of the subject following administration of the cytotoxic therapy, wherein the cytotoxic therapy is an immunotherapy or a cell therapy and specifically binds to an antigen associated with, expressed by, or present on cells of the cancer.

Provided herein is a method of treating cancer including administering a cytotoxic therapy to a subject having a cancer, wherein the cytotoxic therapy is an immunotherapy or a cell therapy and specifically binds to an antigen associated with, expressed by, or present on cells of the cancer; and administering to the subject an inhibitor of a prosurvival BCL2 family protein in a dosing regimen including initiation of administration of the inhibitor at a time between at or about 7 days prior to and at or about 14 days after initiation of administration of the cytotoxic therapy.

Provided herein is a method of treating cancer including administering to a subject having a cancer an inhibitor of a prosurvival BCL2 family protein, wherein the inhibitor is administered in a dosing regimen including initiation of administration of the inhibitor at a time between at or about 7 days prior to and at or about 14 days after initiation of administration of a cytotoxic therapy, wherein the cytotoxic therapy is an immunotherapy or a cell therapy and specifically binds to an antigen associated with, expressed by, or present on cells of the cancer.

Provided herein is a method of treating a cancer in a subject including administering a cytotoxic therapy to a subject having a cancer, wherein the cytotoxic therapy is an immunotherapy or a cell therapy and specifically binds to an antigen associated with, expressed by, or present on cells of the cancer, wherein the subject is administered or is to be administered an inhibitor of a prosurvival BCL2 family protein for a period of time in a dosing regimen including initiation of administration of the inhibitor at a time between at or about 7 days prior to and at or about 14 days after initiation of administration of the cytotoxic therapy. In some embodiments, the dosing regimen of the inhibitor includes initiation of administration of the inhibitor at a time between at or about 7 days prior to and at or about 14 days after initiation of administration of the cytotoxic therapy. In some embodiments, the dosing regimen of the inhibitor includes initiation of administration of the inhibitor at a time between at or about 3 days prior to and at or about 14 days after initiation of administration of the cytotoxic therapy. In some embodiments, the dosing regimen of the inhibitor includes initiation of administration of the inhibitor at a time between at or about 1 day prior to and at or about 8 days after initiation of administration of the cytotoxic therapy.

In some of any of the embodiments, the dosing regimen of the inhibitor includes initiation of administration of the inhibitor after administration of the cytotoxic therapy. In some embodiments, the dosing regimen includes initiation of the administration of the inhibitor between about 1 day prior to and about 8 days after initiation of administration of the cytotoxic therapy. Also provided herein is a composition including a cytotoxic therapy for use in the manufacture of a medicament for treatment of a subject having a cancer, wherein the cytotoxic therapy is an immunotherapy or a cell therapy and specifically binds to an antigen associated with, expressed by, or present on cells of the cancer; and the composition is for use in combination with an inhibitor of a prosurvival BCL2 family protein in a dosing regimen comprising initiation of administration of the inhibitor between at or about 1 day prior to and at or about 8 days after initiation of administration of the cytotoxic therapy. Provided herein is a composition including an inhibitor of a prosurvival BCL2 family protein for use in the manufacture of a medicament for treatment of a subject having a cancer, wherein the composition is for use in combination with a cytotoxic therapy that is an immunotherapy or a cell therapy and specifically binds to an antigen associated with, expressed by, or present on cells of the cancer; and the composition is for use in a dosing regimen comprising initiation of administration of the composition between at or about 1 day prior to and at or about 8 days after initiation of administration of the cytotoxic therapy. In some embodiments, the cytotoxic therapy is a recombinant receptor-expressing cell therapy. In some embodiments, the inhibitor is for use in a dosing regimen comprising initiation of administration of the composition within about 7 days after initiation of administration of the cytotoxic therapy. In some embodiments, the inhibitor is venetoclax. In some embodiments, the inhibitor is for use in a dosing regimen comprising initiation of administration of the composition at about 7 days after initiation of administration of the cytotoxic therapy. In some embodiments, the inhibitor is venetoclax.

Also provided herein is a kit including (i) an inhibitor of a prosurvival BCL2 family protein and/or a cytotoxic therapy that is an immunotherapy or a cell therapy and specifically binds to an antigen associated with, expressed by, or present on cells of a cancer; and (ii) instructions for using the kit to treat a subject having the cancer, wherein the instructions specify: the inhibitor is for use in combination with the cytotoxic therapy; and the inhibitor is to be administered between at or about 1 day prior to and at or about 8 days after initiation of administration of the cytotoxic therapy. In some embodiments, the cytotoxic therapy is a cell therapy comprising T cells expressing a recombinant receptor. In some embodiments, the instructions specify the inhibitor is to be administered within about 7 days after initiation of administration of the cytotoxic therapy. In some embodiments, the instructions specify the inhibitor is to be administered at about 7 days after initiation of administration of the cytotoxic therapy. In some embodiments, the inhibitor is venetoclax.

In some embodiments, the dosing regimen of the inhibitor includes initiation of administration of the inhibitor within 7 days after initiation of administration of the cytotoxic therapy. In some embodiments, the dosing regimen of the inhibitor includes initiation of administration of the inhibitor within 3 days after initiation of administration of the cytotoxic therapy. In some embodiments, the dosing regimen of the inhibitor includes initiation of administration of the inhibitor at or after activation-induced cell death (AICD) of the cells of the cell therapy has peaked following initiation of administration of the cell therapy. In some embodiments, the dosing regimen of the inhibitor includes initiation of administration of the inhibitor at the time activation-induced cell death (AICD) of the cells of the cell therapy has peaked following initiation of administration of the cell therapy. In some embodiments, the dosing regimen of the inhibitor includes initiation of administration of the inhibitor after activation-induced cell death (AICD) of the cells of the cell therapy has peaked following initiation of administration of the cell therapy.

In some embodiments, at least one dose of the inhibitor in the dosing regimen is administered concurrently with the cytotoxic therapy and/or on the same day as the cytotoxic therapy. In some embodiments, the dosing regimen of the inhibitor is sufficient to achieve a steady state concentration (Css) of the inhibitor at a time between at or about 1 day and at or about 14 days after initiation of administration of the cytotoxic therapy and/or at a time before peak levels of the cytotoxic therapy is detectable in the blood of the subject following administration of the cytotoxic therapy.

In some embodiments, the subject is not administered or has not received administration of rituximab and/or ibrutinib within 7 days prior to the initiation of administration of the cytotoxic therapy.

In some embodiments, the cytotoxic therapy is capable of or results in cell-mediated cytotoxicity of one of more of cells of the cancer. In some embodiments, the cytotoxic therapy is capable of or mediates perforin- and/or granzyme-mediated apoptosis of one or more cells of the cancer.

In some embodiments, the cytotoxic therapy is an immunotherapy. In some embodiments, the immunotherapy is a T cell engaging therapy capable of stimulating activity of T cells. In some embodiments, the cytotoxic therapy is a bispecific T cell engager (BiTE) therapy.

In some embodiments, the cytotoxic therapy is a cell therapy. In some embodiments, the cell therapy includes cells that are autologous to the subject. In some embodiments, the cell therapy is selected from among the group consisting of a tumor infiltrating lymphocytic (TIL) therapy, an endogenous T cell therapy, a natural kill (NK) cell therapy, a transgenic TCR therapy, and a recombinant-receptor expressing cell therapy, which optionally is a chimeric antigen receptor (CAR)-expressing cell therapy. In some embodiments, the cytotoxic therapy is a recombinant receptor-expressing cell therapy. In some embodiments, the cytotoxic therapy is a chimeric antigen receptor (CAR)-expressing cell therapy.

In some embodiments, the cell therapy includes a dose of cells expressing a recombinant receptor that specifically binds to the antigen. In some embodiments, administration of the cell therapy includes administration of from or from about 1×10⁵ to 5×10⁸ total cells of the cell therapy, recombinant receptor-expressing cells, total T cells, or total peripheral blood mononuclear cells (PBMCs); from or from about 1×10⁵ to 2×10⁸ total cells of the cell therapy, recombinant receptor-expressing cells, total T cells, or total peripheral blood mononuclear cells (PBMCs); from or from about 1×10⁶ to 1×10⁸ total cells of the cell therapy, recombinant receptor-expressing cells, total T cells, or total peripheral blood mononuclear cells (PBMCs); or 1×10⁶ to 5×10⁷ total cells of the cell therapy, recombinant receptor-expressing cells, total T cells, or total peripheral blood mononuclear cells (PBMCs).

In some embodiments, the administration of the cytotoxic therapy includes administration of between at or about 1×10⁵ and at or about 5×10⁸ total recombinant receptor-expressing T cells or total T cells, between at or about 1×10⁵ and at or about 1×10⁸ total recombinant receptor-expressing T cells or total T cells, between at or about 5×10⁵ and at or about 1×10⁷ total recombinant receptor-expressing T cells or total T cells, or between at or about 1×10⁶ and at or about 1×10⁷ total recombinant receptor-expressing T cells or total T cells, each inclusive. In some embodiments, the administration of the cytotoxic therapy includes administration of between at or about 1×10⁵ and at or about 5×10⁸ total recombinant receptor-expressing T cells or total T cells. In some embodiments, the administration of the cytotoxic therapy includes administration of between at or about 1×10⁵ and at or about 1×10⁸ total recombinant receptor-expressing T cells or total T cells. In some embodiments, the administration of the cytotoxic therapy includes administration of between at or about 5×10⁵ and at or about 1×10⁷ total recombinant receptor-expressing T cells or total T cells. In some embodiments, the administration of the cytotoxic therapy includes administration of between at or about 1×10⁶ and at or about 1×10⁷ total recombinant receptor-expressing T cells or total T cells. In some embodiments, the dose of cells includes or is enriched in T cells. In some embodiments, the T cells are CD4+ T cells, CD8+ T cells, or CD4+ and CD8+ T cells. In some embodiments, the T cells are CD4+ T cells. In some embodiments, the T cells are CD8+ T cells. In some embodiments, the T cells are CD4+ T cells and CD8+ T cells. In some embodiments, administration of the dose of cells includes administering CD4+ and CD8+ T cells, each of the CD4+ and the CD8+ T cells, individually, including a receptor, optionally a CAR, that specifically binds to the antigen, wherein the administration includes administering a plurality of separate compositions, the plurality of separate compositions including a first composition including or enriched in the CD8+ T cells and a second composition including or enriched in the CD4+ T cells.

In some embodiments, the dose of CD4+ and CD8+ T cells includes a defined ratio of CD4+ cells expressing a recombinant receptor to CD8+ cells expressing a recombinant receptor and/or of CD4+ cells to CD8+ cells, that is or is approximately 1:1 or is between approximately 1:3 and approximately 3:1; and/or the CD4+ T cells including the receptor in the one of the first and second compositions and the CD8+ T cells including the receptor in the other of the first and second compositions are present at a defined ratio that is or is approximately 1:1 or is between approximately 1:3 and approximately 3:1; and/or the CD4+ T cells including the receptor and the CD8+ T cells including the receptor administered in the first and second compositions are present at a defined ratio, which ratio is or is approximately 1:1 or is between approximately 1:3 and approximately 3:1. In some embodiments, the ratio is between approximately 1:3 and approximately 3:1. In some embodiments, the ratio is or is approximately 1:1. In some embodiments, the dose of cells includes or is enriched in natural killer (NK) cells. In some embodiments, the dose of cells includes iPS-derived cells.

In some embodiments, the recombinant receptor is a T cell receptor (TCR) or a functional non-T cell receptor. In some embodiments, the recombinant receptor is a chimeric antigen receptor (CAR).

In some embodiments, the cell therapy includes administration of from or from about 1×10⁵ to 5×10⁸ total CAR-expressing T cells, 1×10⁶ to 2.5×10⁸ total CAR-expressing T cells, 5×10⁶ to 1×10⁸ total CAR-expressing T cells, 1×10⁷ to 2.5×10⁸ total CAR-expressing T cells, 5×10⁷ to 1×10⁸ total CAR-expressing T cells, each inclusive. In some embodiments, the cell therapy includes administration of at least or at least about 1×10⁵ CAR-expressing cells, at least or at least about 2.5×10⁵ CAR-expressing cells, at least or at least about 5×10⁵ CAR-expressing cells, at least or at least about 1×10⁶ CAR-expressing cells, at least or at least about 2.5×10⁶ CAR-expressing cells, at least or at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 2.5×10⁸ CAR-expressing cells, or at least or at least about 5×10⁸ CAR-expressing cells. In some embodiments, the cell therapy includes administration of at or about 5×10⁷ total CAR-expressing T cells. In some embodiments, the cell therapy includes administration of at or about 1×10⁸ CAR-expressing cells.

In some embodiments, the CAR includes an extracellular antigen-recognition domain that specifically binds to the antigen and an intracellular signaling domain including an ITAM. In some embodiments, the antigen is a tumor antigen or is expressed on cells of the cancer. In some embodiments, the antigen is selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha(IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1). In some embodiments, the antigen is associated with a B cell malignancy, optionally wherein the antigen is expressed on human B cells. In some embodiments, the B cell antigen is CD20, CD19, CD22, ROR1, CD45, CD21, CD5, CD33, Igkappa, Iglambda, CD79a, CD79b or CD30. In some embodiments, the antigen is CD19.

In some embodiments, the intracellular signaling domain includes an intracellular domain of a CD3-zeta (CD3) chain. In some embodiments, the intracellular signaling region further includes a costimulatory signaling region. In some embodiments, the costimulatory signaling region includes a signaling domain of CD28 or 4-1BB, optionally human CD28 or human 4-1BB. In some embodiments, the costimulatory domain is or includes a signaling domain of CD28. In some embodiments, the costimulatory domain is or includes a signaling domain of 4-1BB. In some embodiments, the costimulatory signaling region comprises a signaling domain of 4-1BB. In some embodiments, the costimulatory signaling region comprises a signaling domain of CD28. In some embodiments, the CAR comprises an extracellular antigen binding domain that binds to the antigen, a transmembrane domain, and an intracellular signaling region comprising an intracellular signaling domain of a CD3-zeta (CD3) chain and a costimulatory signaling domain.

In some embodiments, the cell therapy includes autologous cells from the subject. In some embodiments, the method includes collecting a biological sample from the subject including the autologous cells prior to initiation of administration of the inhibitor. In some embodiments, the biological sample from the subject is or includes a whole blood sample, a buffy coat sample, a peripheral blood mononuclear cells (PBMC) sample, an unfractionated T cell sample, a lymphocyte sample, a white blood cell sample, an apheresis product, or a leukapheresis product. In some embodiments, the biological sample from the subject is or includes a peripheral blood mononuclear cells (PBMC) sample. In some embodiments, the biological sample from the subject is or includes an apheresis product. In some embodiments, the biological sample from the subject is or includes a leukapheresis product.

In some embodiments, at the time of initiation of administration of the cytotoxic therapy, the subject exhibits (i) measurable disease, optionally lymph nodes of greater than 1.5 centimeters in greatest transverse diameter (GTD), or evaluable or measurable disease in peripheral blood (PB), bone marrow (BM), liver, or spleen. In some embodiments, at the time of initiation of administration of the bridging therapy, the subject exhibits (i) measurable disease, optionally lymph nodes of greater than 1.5 centimeters in greatest transverse diameter (GTD), or evaluable or measurable disease in peripheral blood (PB), bone marrow (BM), liver, or spleen. In some embodiments, at the time of initiation of administration of the cytotoxic therapy, the subject exhibits minimum residual disease (MRD) in peripheral blood of greater than or equal to 10⁻⁴. In some embodiments, at the time of initiation of administration of the bridging therapy, the subject exhibits minimum residual disease (MRD) in peripheral blood of greater than or equal to 10⁻⁴.

In some embodiments, at the time of initiation of administration of the cytotoxic therapy, the subject exhibits (i) measurable disease, optionally lymph nodes of greater than 1.5 centimeters in greatest transverse diameter (GTD), or evaluable or measurable disease in peripheral blood (PB), bone marrow (BM), liver, or spleen; and (ii) minimum residual disease (MRD) in peripheral blood of greater than or equal to 10⁻⁴. In some embodiments, at the time of initiation of administration of the bridging therapy, the subject exhibits (i) measurable disease, optionally lymph nodes of greater than 1.5 centimeters in greatest transverse diameter (GTD), or evaluable or measurable disease in peripheral blood (PB), bone marrow (BM), liver, or spleen; and (ii) minimum residual disease (MRD) in peripheral blood of greater than or equal to 10⁻⁴.

In some embodiments, the lymphodepleting therapy is completed between 2 and 7 days before the initiation of administration of the cytotoxic therapy. In some embodiments, the lymphodepleting therapy includes the administration of fludarabine and/or cyclophosphamide. In some embodiments, the lymphodepleting therapy includes the administration of fludarabine. In some embodiments, the lymphodepleting therapy includes the administration of cyclophosphamide. In some embodiments, the lymphodepleting therapy includes the administration of fludarabine and cyclophosphamide. In some embodiments, the lymphodepleting therapy includes administration of cyclophosphamide at about 200-400 mg/m², optionally at or about 300 mg/m², inclusive, and/or fludarabine at about 20-40 mg/m², optionally 30 mg/m², daily for 2-4 days, optionally for 3 days, or the lymphodepleting therapy includes administration of cyclophosphamide at about 500 mg/m². In some embodiments, the lymphodepleting therapy includes administration of cyclophosphamide at or about 300 mg/m² and fludarabine at about 30 mg/m² daily for 3 days; and/or the lymphodepleting therapy includes administration of cyclophosphamide at or about 500 mg/m² and fludarabine at about 30 mg/m² daily for 3 days. In some embodiments, the lymphodepleting therapy includes administration of cyclophosphamide at or about 300 mg/m² and fludarabine at about 30 mg/m² daily for 3 days. In some embodiments, the lymphodepleting therapy includes administration of cyclophosphamide at or about 500 mg/m² and fludarabine at about 30 mg/m² daily for 3 days.

In some embodiments, the initiation of administration of the inhibitor is within at or about 3 days prior to initiation of administration of the cytotoxic therapy. In some embodiments, the initiation of administration of the inhibitor is within at or about 2 days prior to initiation of administration of the cytotoxic therapy. In some embodiments, the initiation of administration of the inhibitor is within at or about 1 day prior to initiation of administration of the cytotoxic therapy. In some embodiments, the initiation of administration of the inhibitor is concurrent with or on the same day as initiation of administration of the inhibitor. In some embodiments, the initiation of administration of the inhibitor is no more than 2 days after initiation of administration of the cytotoxic therapy, optionally wherein the initiation of administration of the inhibitor is within 1 day after the initiation of administration of the cytotoxic therapy. In some embodiments, the initiation of administration of the inhibitor is within about 7 days after initiation of administration of the cytotoxic therapy. In some embodiments, the initiation of administration of the inhibitor is between about 1 day after and about 7 days after initiation of administration of the cytotoxic therapy. In some embodiments, the initiation of administration of the inhibitor is about 2 days after initiation of administration of the cytotoxic therapy. In some embodiments, the initiation of administration of the inhibitor is about 3 days after initiation of administration of the cytotoxic therapy. In some embodiments, the initiation of administration of the inhibitor is about 4 days after initiation of administration of the cytotoxic therapy. In some embodiments, the initiation of administration of the inhibitor is about 5 days after initiation of administration of the cytotoxic therapy. In some embodiments, the initiation of administration of the inhibitor is about 6 days after initiation of administration of the cytotoxic therapy. In some embodiments, the initiation of administration of the inhibitor is about 7 days after initiation of administration of the cytotoxic therapy. In some embodiments, the initiation of administration of the inhibitor is about 8 days after initiation of administration of the cytotoxic therapy.

In some embodiments, the dosing regimen of the inhibitor includes a subtherapeutic amount of the inhibitor; the dosing regimen of the inhibitor is not sufficient to reduce tumor burden in the subject or reduces tumor burden by less than 10% when administered as a monotherapy in the absence of the combined administration with the cytotoxic therapy; and/or the dosing regimen of the inhibitor does not result in a complete or partial response in a group of similarly treated subjects, or results in such a response in no more than 10% of such subjects, when administered as a monotherapy in the absence of the combined administration with the cytotoxic therapy. In some embodiments, the dosing regimen of the inhibitor includes a subtherapeutic amount of the inhibitor. In some embodiments, the dosing regimen of the inhibitor is not sufficient to reduce tumor burden in the subject or reduces tumor burden by less than 10% when administered as a monotherapy in the absence of the combined administration with the cytotoxic therapy. In some embodiments, the dosing regimen of the inhibitor does not result in a complete or partial response in a group of similarly treated subjects, or results in such a response in no more than 10% of such subjects, when administered as a monotherapy in the absence of the combined administration with the cytotoxic therapy.

In some embodiments, the dosing regimen of the inhibitor includes once daily dosing. In some embodiments, the once daily dose is an amount of the inhibitor of between at or about 20 mg and at or about 800 mg, between at or about 20 mg and at or 400 mg, between at or about 20 mg and at or about 350 mg, between at or about 20 mg and at or about 300 mg, between at or about 20 mg and at or about 250 mg, between at or about 20 mg and at or about 200 mg, between at or about 20 mg and at or about 150 mg, between at or about 20 mg and at or about 100 mg, between at or about 20 mg and at or about 50 mg, between at or about 20 mg and at or about 40 mg, between at or about 40 mg and at or about 800 mg, between at or about 40 mg and at or 400 mg, between at or about 40 mg and at or about 350 mg, between at or about 40 mg and at or about 300 mg, between at or about 40 mg and at or about 250 mg, between at or about 40 mg and at or about 200 mg, between at or about 40 mg and at or about 150 mg, between at or about 40 mg and at or about 100 mg, between at or about 40 mg and at or about 50 mg, between at or about 50 mg and at or about 800 mg, between at or about 50 mg and at or about 400 mg, between at or about 50 mg and at or about 350 mg, between at or about 50 mg and at or about 300 mg, between at or about 50 mg and at or about 250 mg, between at or about 50 mg and at or about 200 mg, between at or about 50 mg and at or about 150 mg, between at or about 50 mg and at or about 100 mg, between at or about 100 mg and at or about 800 mg, between at or about 100 mg and at or about 400 mg, between at or about 100 mg and at or about 350 mg, between at or about 100 mg and at or about 300 mg, between at or about 100 mg and at or about 250 mg, between at or about 100 mg and at or about 200 mg, between at or about 100 mg and at or about 150 mg, between at or about 150 mg and at or about 800 mg, between at or about 150 mg and at or about 400 mg, between at or about 150 mg and at or about 350 mg, between at or about 150 mg and at or about 300 mg, between at or about 150 mg and at or about 250 mg, between at or about 150 mg and at or about 200 mg, between at or about 200 mg and at or about 800 mg, between at or about 200 mg and at or about 400 mg, between at or about 200 mg and at or about 350 mg, between at or about 200 mg and at or about 300 mg, between at or about 200 mg and at or about 250 mg, between at or about 250 mg and at or about 300 mg, between at or about 300 mg and at or about 350 mg, between at or about 300 mg and at or about 400 mg, between at or about 300 mg and at or about 800 mg, between at or about 350 mg and at or about 400 mg, and between at or about 350 mg and at or about 800 mg, each inclusive. In some embodiments, the once daily dose is an amount of the inhibitor of between at or about 20 mg and at or about 100 mg, inclusive.

In some embodiments, the once daily dose is between at or about 20 mg and at or 400 mg, inclusive. In some embodiments, the once daily dose is an amount of the inhibitor of between at or about 40 mg and at or about 200 mg, inclusive. In some embodiments, the once daily dose is an amount of the inhibitor of between at or about 40 mg and at or about 400 mg, inclusive.

In some embodiments, the once daily dose is an amount of the inhibitor of between at or about 40 mg and at or about 100 mg, inclusive. In some embodiments, the once daily dose is an amount of the inhibitor of between at or about 50 mg and at or about 100 mg, inclusive. In some embodiments, the once daily dose is an amount of the inhibitor of at or about 20 mg. In some embodiments, the once daily dose is an amount of the inhibitor of at or about 40 mg. In some embodiments, the once daily dose is an amount of the inhibitor of at or about 50 mg. In some embodiments, the once daily dose is an amount of the inhibitor of at or about 100 mg. In some embodiments, the once daily dose is at or about 200 mg. In some embodiments, the once daily dose is at or about 300 mg. In some embodiments, the once daily dose is at or about 400 mg. In some embodiments, the dosing regimen of the inhibitor comprises once daily dosing, and the once daily dose amount is 200 mg for at least about 11 weeks. In some embodiments, the dosing regimen of the inhibitor comprises once daily dosing, and the once daily dose amount is 200 mg given until at least about 12 weeks after initiation of administration of a cytotoxic therapy.

In some embodiments, the inhibitor is administered in a dose-ramp up schedule prior to administration of the dosing regimen of the inhibitor. In some embodiments, the prior or previous treatment with the inhibitor is a dose-ramp up schedule that includes administration of increasing amounts of the inhibitor. In some embodiments, the increasing amount of the inhibitor is up to the amount of the once daily dose. In some embodiments, the prior or previous treatment with the inhibitor is administered at a time between the collecting of the cells, e.g. T cells, such as autologous cells from the subject, and prior to administering a lymphodepleting therapy to the subject, as a bridging therapy prior to the administration of the cytotoxic therapy. In some embodiments, the collecting of the cells, e.g. T cells, such as autologous cells, is by apheresis or leukapheresis. In some embodiments, the previous treatment of the inhibitor is administered in a dose-ramp up schedule, wherein the dose-ramp up schedule includes administration of escalating doses of the inhibitor. In some embodiments, the inhibitor is administered to the subject in escalating doses until a maximum dose of 100 mg daily is reached. In some embodiments, the escalating doses include a first dose that is at about 20 mg per day, a second dose that is at about 50 mg per day, and a third dose that is at about 100 mg per day. In some embodiments, the inhibitor is administered to the subject in escalating doses until a maximum dose of 200 mg daily is reached. In some embodiments, the escalating doses include a first dose that is at about 20 mg per day, a second dose that is at about 50 mg per day, a third dose that is at about 100 mg per day, and a fourth dose that is at or about 200 mg per day. In some embodiments, the inhibitor is administered to the subject in escalating doses until a maximum dose of 400 mg daily is reached. In some embodiments, the escalating doses include a first dose that is at about 20 mg per day, a second dose that is at about 50 mg per day, a third dose that is at about 100 mg per day, a fourth dose that is at or about 200 mg per day, and a fifth dose that is at or about 400 mg. In some embodiments, the last escalating dose of the dose-ramp up schedule is administered once daily for a week or until the end of the bridging therapy. In some embodiments, each escalating doses (e.g. first, second, third) are administered once daily for a week and the last escalating dose is administered once daily for a week or until the end of the bridging therapy. In some embodiments, the previous treatment with the inhibitor is ceased at least 1 day prior to administration of the lymphodepleting therapy.

In some embodiments, the method includes, prior to administration of the cytotoxic therapy, administering a lymphodepleting agent or therapy to the subject. In some embodiments, the subject has been previously treated with an inhibitor of a prosurvival Bcl-2 family protein, optionally wherein the subject has been previously treated with venetoclax. In some embodiments, the previous treatment with the inhibitor is administered at a time between the collecting of the autologous cells and prior to a lymphodepleting therapy. In some embodiments, the previous treatment with the inhibitor is ceased for at least at or about 3 days or for at least at or about 4 days prior to administration of a lymphodepleting therapy and/or for at least an amount of time until the concentration of the inhibitor in the subject's bloodstream is reduced by about three or about four half-lives and/or for at least an amount of time until the inhibitor is eliminated from the bloodstream of the subject.

In some embodiments, the inhibitor inhibits one or more prosurvival BCL2 family protein selected from among the group consisting of BCL2, BCLXL, BCLW, BCLB, MCL1, and combinations thereof. In some embodiments, the inhibitor inhibits one or more prosurvival BCL2 family protein selected from among the group consisting of BCL2, BCLXL, BCLW, BCLB, BFL1, MCL1, and combinations thereof. In some embodiments, the one or more prosurvival BCL2 family protein is BCL2, BCLXL, and/or BCLW. In some embodiments, the one or more prosurvival BCL2 family protein is BCL2, BCLXL, BCLW, and/or BFL1. In some embodiments, the one or more prosurvival BCL2 family protein is BCL2. In some embodiments, the inhibitor inhibits the one or more prosurvival BCL2 family protein with a half-maximal inhibitory concentration (IC50) of less than or less than about 1000 nM, 900 nM, 800 nM, 600 nM, 500 nM, 400 nM, 300 nM, 200 nM, 100 nM, 50 nM or less than or less than about 10 nM.

In some embodiments, the inhibitor is selected from among venetoclax, navitoclax, ABT737, maritoclax, obatoclax, and clitocine. In some embodiments, the inhibitor is navitoclax. In some embodiments, the inhibitor is venetoclax.

In some embodiments, the cancer is a hematological malignancy. In some embodiments, the cancer is a B cell malignancy. In some embodiments, the cancer is a myeloma, leukemia or lymphoma. In some embodiments, the cancer is a myeloma. In some embodiments, the cancer is a leukemia. In some embodiments, the cancer is a lymphoma. In some embodiments, the cancer is an acute lymphoblastic leukemia (ALL), adult ALL, a chronic lymphocytic leukemia (CLL), a small lymphocytic lymphoma (SLL), a non-Hodgkin lymphoma (NHL), or a large B cell lymphoma. In some embodiments, the cancer is a chronic lymphocytic leukemia (CLL). In some embodiments, the cancer is a non-Hodgkin lymphoma (NHL). In some embodiments, the NHL is a diffuse large B-cell lymphoma (DLBCL). In some embodiments, the cancer is a small lymphocytic lymphoma (SLL). In some embodiments, the cancer is primary mediastinal B-cell lymphoma (PMBCL) or a follicular lymphoma (FL), optionally follicular lymphoma grade 3B (FL3B). In some embodiments, the cancer is primary mediastinal B-cell lymphoma (PMBCL). In some embodiments, the cancer is a follicular lymphoma (FL). In some embodiments, the cancer is a follicular lymphoma grade 3B (FL3B).

In some embodiments, the subject has relapsed following remission after treatment with, or become refractory to, failed and/or was intolerant to treatment with the one or more prior therapies for treating the cancer. In some embodiments, the cancer is resistant to treatment with the cytotoxic therapy alone. In some embodiments, the cancer is resistant to treatment with the inhibitor of the prosurvival BCL2 family protein alone. In some embodiments, the cancer exhibits overexpression or aberrant expression of a prosurvival BCL2 family protein. In some embodiments, the cancer exhibits overexpression of a prosurvival BCL2 family protein that is targeted by the inhibitor. In some embodiments, the cancer exhibits overexpression or aberrant expression of BCL2. In some embodiments, the cancer exhibits overexpression or aberrant expression of BCLXL. In some embodiments, the cancer exhibits overexpression or aberrant expression of MCL1. In some embodiments, the cancer exhibits overexpression or aberrant expression of BFL1.

In some embodiments, the dosing regimen of the inhibitor includes administration of the inhibitor, optionally once daily, for up to 6 months after the initiation of the administration of the cytotoxic therapy. In some embodiments, the dosing regimen of the inhibitor includes administration of the inhibitor once daily, for up to about 6 months after the initiation of the administration of the cytotoxic therapy. In some embodiments, the dosing regimen of the inhibitor includes administration of the inhibitor, optionally once daily, for a period of time of at least 3 months after the initiation of the administration of the cytotoxic therapy. In some embodiments, the dosing regimen of inhibitor includes administration of the inhibitor, optionally once daily, for up to 3 months after the initiation of the administration of the cytotoxic therapy. In some embodiments, the dosing regimen of inhibitor includes administration of the inhibitor once daily, for at least about 3 months after the initiation of the administration of the cytotoxic therapy. In some embodiments, the dosing regimen of the inhibitor includes administration of the inhibitor once daily for up to about 12 months after the initiation of the administration of the cytotoxic therapy. In some embodiments, the dosing regimen of the inhibitor includes administration of the inhibitor once daily for up to about 24 months after the initiation of the administration of the cytotoxic therapy. In some embodiments, administration of the inhibitor in the dosing regimen is discontinued if the subject exhibits clinical remission. In some embodiments, administration of the inhibitor in the dosing regimen is discontinued at the end of the period of time if the subject exhibits clinical remission. In some embodiments, the period of time is 3 months. In some embodiments, the method further comprises continued administration of the dosing regimen of the inhibitor if at the end of the period of time, the subject does not exhibit a clinical remission. In some embodiments, the method further comprises continued administration of the dosing regimen of the inhibitor if at the end of the period of time, the subect exhibits a partial response (PR). In some embodiments, the method further comprises continued administration of the dosing regimen of the inhibitor if at the end of the period of time, the subect exhibits stable disease (SD). In some embodiments, the method further comprises continued administration of the dosing regimen of the inhibitor if at the end of the period of time, the subect exhibits has minimal residual disease (MRD) greater than or equal to 10⁴. In some embodiments, the period of time is 3 months.

In some embodiments, the method increases the cytotoxic activity of the cytotoxic therapy compared to a method that does not involve the administration of the inhibitor; and/or the method increases cytolytic killing, optionally via perforin- and/or granzyme-mediated apoptosis, of one or more of the cancer cells compared to a method that does not involve the administration of the inhibitor.

In some embodiments, at least 35%, at least 40% or at least 50% of subjects treated according to the method achieve a complete response (CR) that is durable, or is durable in at least 60, 70, 80, 90, or 95% of subjects achieving the CR, for at or greater than 6 months or at or greater than 9 months; and/or wherein at least 60, 70, 80, 90, or 95% of subjects achieving a CR by six months remain in response, remain in CR, and/or survive or survive without progression, for greater at or greater than 3 months and/or at or greater than 6 months and/or at greater than nine months; and/or at least 50%, at least 60% or at least 70% of the subjects treated according to the method achieve objective response (OR) optionally wherein the OR is durable, or is durable in at least 60, 70, 80, 90, or 95% of subjects achieving the OR, for at or greater than 6 months or at or greater than 9 months; and/or wherein at least 60, 70, 80, 90, or 95% of subjects achieving an OR by six months remain in response or surviving for greater at or greater than 3 months and/or at or greater than 6 months.

In some embodiments, the subject is a human.

Provided herein is a method of treatment with a cytotoxic therapy, the method including (a) assessing the level or amount of one or more prosurvival gene in a biological sample from a subject having or suspected of having a cancer; (b) selecting the subject for treatment with a cytotoxic therapy if the level or amount of the one or more prosurvival gene is below a gene reference value; and (c) administering to the selected patient the cytotoxic therapy, wherein the cytotoxic therapy is an immunotherapy or a cell therapy and binds an antigen associated with, expressed by, or present on cells of the cancer. In some embodiments, the cytotoxic therapy is a recombinant receptor-expressing cell therapy. In some embodiments, an inhibitor of a prosurvival gene is not administered to the subject at or after initiation of the administration of the cytotoxic therapy. In some embodiments, an inhibitor of a prosurvival gene is not administered to the subject within 7 days, 14 days or 28 days after the administration of the cytotoxic therapy

Provided herein is a method of selecting a subject having a cancer for treatment with an inhibitor of a prosurvival BCL2 family protein, the method including (a) assessing the level or amount of one or more prosurvival gene in a biological sample from the subject, wherein: (i) the level or amount of the one or more prosurvival gene is the level or amount of a protein and/or a polynucleotide encoded by the one or more prosurvival gene; (ii) the subject is to receive administration of a cytotoxic therapy that is an immunotherapy or a cell therapy and specifically binds to an antigen associated with, expressed by, or present on cells of the cancer; and (iii) the biological sample is obtained from the subject prior to the administration of the cytotoxic therapy; and (b) selecting the subject having the cancer for treatment with an inhibitor of a prosurvival BCL2 family protein if the level or amount of the one or more prosurvival gene is above a gene reference value, where the inhibitor is administered in a dosing regimen including initiation of administration of the inhibitor between at or about 1 day prior to and at or about 8 days after initiation of administration of the cytotoxic therapy. In some embodiments, the cytotoxic therapy is a recombinant-receptor expressing cell therapy. In some embodiments, the dosing regimen of the inhibitor comprises initiation of administration of the inhibitor within about 7 days after initiation of administration of the cytotoxic therapy. In some embodiments, the dosing regimen of the inhibitor comprises initiation of administration of the inhibitor about 7 days after initiation of administration of the cytotoxic therapy. In some embodiments, the inhibitor is venetoclax. In some embodiments, if the subject is not selected for treatment with the inhibitor, the method comprises administering only the cytotoxic therapy to the subject. In some embodiments, if the subject is selected for treatment with the inhibitor, the method further comprises administering the inhibitor and the cytotoxic therapy to the subject.

Provided herein is a method of identifying a subject having a cancer that is predicted to be resistant to treatment with a cytotoxic therapy, the method including (a) assessing the level or amount of one or more prosurvival gene in a biological sample from the subject, wherein: (i) the level or amount of the one or more prosurvival gene is the level or amount of a protein and/or a polynucleotide encoded by the one or more prosurvival gene; (ii) the subject is a candidate for administration of a dose of a cytotoxic therapy that is an immunotherapy or a cell therapy and specifically binds to an antigen associated with, expressed by, or present on cells of the cancer; and (iii) the biological sample is obtained from the subject prior to the administration of the cytotoxic therapy; and (b) identifying the subject as having a cancer that is predicted to be resistant to treatment with the cytotoxic therapy if the level or amount of the one or more prosurvival gene is above a gene reference value.

In some embodiments, the cytotoxic therapy is a recombinant-receptor expressing cell therapy. In some embodiments, if the subject is identified as having a cancer that is predicted to be resistant to treatment with the cytotoxic therapy, the method further includes administering an alternative treatment to the identified subject, wherein the alternative treatment is selected from among the following: a combination treatment including the cytotoxic therapy and an additional agent that modulates or increases the activity of the cytotoxic therapy; an increased dose of the cytotoxic therapy; and/or a chemotherapeutic agent. In some embodiments, the alternative treatment is a combination treatment including the cytotoxic therapy and an additional agent that modulates or increases the activity of the T cell therapy, optionally wherein the additional agent is an immune checkpoint inhibitor, a modulator of a metabolic pathway, an adenosine receptor antagonist, a kinase inhibitor, an anti-TGFbeta antibody or an anti-TGFbetaR antibody, a cytokine, and/or a prosurvival BCL2 family protein inhibitor. In some embodiments, the alternative treatment is a combination treatment including the cytotoxic therapy and a prosurvival BCL2 family protein inhibitor. In some embodiments, the alternative treatment includes an increased dose of the cytotoxic therapy compared to a dose of the cytotoxic therapy given to a subject identified as having a cancer that is not predicted to be resistant to treatment with the cytotoxic therapy. In some embodiments, the increased dose of the cytotoxic therapy comprises an increased number of cells of the cytotoxic therapy compared to a dose of the cytotoxic therapy given to a subject identified as having a cancer that is not predicted to be resistant to treatment with the cytotoxic therapy. In some embodiments, the alternative treatment is a chemotherapeutic agent. In some embodiments, the chemotherapeutic agent is cyclophosphamide, doxorubicin, prednisone, vincristine, fludarabine, bendamustine, and/or rituximab. In some embodiments, if the subject is identified as having a cancer that is not predicted to be resistant to treatment with the cytotoxic therapy, the method includes administering only the cytotoxic therapy to the subject. In any of the provided embodiments, the method further includes administering to the identified subject a prosurvival BCL2 family protein inhibitor. In some embodiments, the gene reference value is within 25%, within 20%, within 15%, within 10%, or within 5% of an average level or amount of the one or more gene in (a) a population of subjects not having the cancer or (b) a population of subjects having the cancer and administered the therapy, who went on to exhibit a partial response (PR) or complete response (CR) following administration of the therapy. In some embodiments, the population of subjects having the cancer went on to exhibit the PR or CR at least 1 month, 2 months, 3 months, 4 months, 5 months, 6 months, 7 months, 8 months, 9 months, or more following administration of the therapy. In some embodiments, the level or amount of the one or more prosurvival gene is assessed in the biological sample before a lymphodepleting therapy is administered to the subject, optionally within 7 days before, 6 days before, 5 days before, 4 days before, 3 days before, 2 days before, 1 day before, 16 hours before, 12 hours before, 6 hours before, 2 hours before, or 1 hour before the lymphodepleting therapy is administered to the subject

Provided herein is a method of determining responsiveness of a subject having a cancer to a cytotoxic therapy, the method including (a) assessing the level or amount of expression of one or more prosurvival gene in a biological sample from the subject having a cancer, wherein: (i) the level or amount of the one or more prosurvival gene is the level or amount of a protein and/or a polynucleotide encoded by the one or more prosurvival gene; (ii) the biological sample is obtained from the subject at a first time prior to the subject being administered the a cytotoxic therapy, wherein the cytotoxic therapy is an immunotherapy or a cell therapy and binds an antigen associated with, expressed by, or present on cells of the cancer; and (iii) the subject is to receive treatment with the cytotoxic therapy; (b) assessing the level or amount of expression of the one or more prosurvival gene in a biological sample from the subject at a second time after administration of the cytotoxic therapy to the subject, wherein: (i) the level or amount of the one or more prosurvival gene is the level or amount of a protein and/or a polynucleotide encoded by the one or more prosurvival gene; (ii) the biological sample is obtained at a second time after the administration of the cytotoxic therapy to the subject; and (iii) the subject has been administered the cytotoxic therapy prior to the assessing in (b); and (c) determining that the subject is responsive to the therapy if the level or amount of the one or more prosurvival gene at the second time is lower than the level or amount of the one or more prosurvival gene at the first time.

In some embodiments, wherein the one or more pro-survival gene is selected from the group consisting of a MYC family gene, p53, and enhancer of zeste homolog 2 (EZH2). In some embodiments, the one or more pro-survival gene is a MYC family gene. In some embodiments, the one or more pro-survival gene is p53. In some embodiments, the one or more pro-survival gene is EZH2.

In any of the embodiments provided herein, the inhibitor is administered in combination with the cytotoxic therapy in accord with any of the provided combination methods herein that includes administering a cytotoxic therapy and an inhibitor of a prosurvival BCL2 family protein.

Also provided herein is a method of treating cancer, including (1) administering a cytotoxic therapy to a subject having a cancer, wherein the cytotoxic therapy is a T cell therapy including or enriched in T cells and specifically binds to an antigen associated with, expressed by, or present on cells of the cancer; and (2) administering to the subject an inhibitor of a prosurvival BCL2 family protein in a dosing regimen including initiation of administration of the inhibitor at a time between at or about 1 day prior and at or about 8 days after initiation of administration of the cytotoxic therapy.

Also provided herein is a method of treating cancer, the method including administering to a subject having a cancer an inhibitor of a prosurvival BCL2 family protein, wherein the inhibitor is administered in a dosing regimen including initiation of administration of the inhibitor at a time between at or about 1 day prior to and at or about 8 days after initiation of administration of a cytotoxic therapy, wherein the cytotoxic therapy a T cell therapy including or enriched in T cells and specifically binds to an antigen associated with, expressed by, or present on cells of the cancer.

Also provided herein is a method of treating a cancer in a subject, the method including administering a cytotoxic therapy to a subject having a cancer, wherein the cytotoxic therapy is a T cell therapy including or enriched in T cells and specifically binds to an antigen associated with, expressed by, or present on cells of the cancer, wherein the subject is administered or is to be administered an inhibitor of a prosurvival BCL2 family protein for a period of time in a dosing regimen including initiation of administration of the inhibitor at a time between at or about 1 day prior to and at or about 8 days after initiation of administration of the cytotoxic therapy.

In some embodiments, the dosing regimen of the inhibitor comprises initiation of administration of the inhibitor after administration of the cytotoxic therapy. In some embodiments, the dosing regimen of the inhibitor comprises initiation of administration of the inhibitor within 7 days after initiation of administration of the cytotoxic therapy. In some embodiments, the dosing regimen of the inhibitor comprises initiation of administration of the inhibitor within 3 days after initiation of administration of the cytotoxic therapy. In some embodiments, the initiation of administration of the inhibitor is no more than 2 days after initiation of administration of the cytotoxic therapy. In some embodiments, initiation of administration of the inhibitor is within 1 day after the initiation of administration of the cytotoxic therapy. In some embodiments, the dosing regimen of the inhibitor comprises initiation of administration of the inhibitor at or after activation-induced cell death (AICD) of the cells of the cell therapy has peaked following initiation of administration of the cell therapy. In some embodiments, at least one dose of the inhibitor in the dosing regimen is administered concurrently with the cytotoxic therapy and/or on the same day as the cytotoxic therapy.

In some embodiments, the subject is not administered or has not received administration of rituximab and/or ibrutinib within 7 days prior to the initiation of administration of the cytotoxic therapy.

In some embodiments, the cytotoxic therapy is capable of or results in cell-mediated cytotoxicity of one of more of cells of the cancer. In some embodiments, the cytotoxic therapy is capable of or mediates perforin- and/or granzyme-mediated apoptosis of one or more cells of the cancer.

In some embodiments, the cell therapy comprises cells that are autologous to the subject. In some embodiments, the cell therapy is selected from among the group consisting of a tumor infiltrating lymphocytic (TIL) therapy, a transgenic TCR therapy, and a chimeric antigen receptor (CAR)-expressing cell therapy. In some embodiments, the cell therapy comprises a dose of cells expressing a recombinant receptor that specifically binds to the antigen.

In some embodiments, the administration of the cytotoxic therapy comprises administration of between at or about 1×10⁵ and at or about 5×10⁸ total recombinant receptor-expressing T cells or total T cells, between at or about 1×10⁵ and at or about 1×10⁸ total recombinant receptor-expressing T cells or total T cells, between at or about 5×10⁵ and at or about 1×10⁷ total recombinant receptor-expressing T cells or total T cells, or between at or about 1×10⁶ and at or about 1×10⁷ total recombinant receptor-expressing T cells or total T cells, each inclusive.

In some embodiments, the cell therapy comprises or is enriched in CD3+, CD4+, CD8+, or CD4+ and CD8+ T cells. In some embodiments, the cell therapy comprises or is enriched in CD4+ and CD8+ T cells. In some embodiments, the CD4+ and CD8+ T cells of the cell therapy comprises a defined ratio of CD4+ recombinant receptor-expressing T cells to CD8+ recombinant receptor-expressing T cells that is or is approximately 1:1 or is between approximately 1:3 and approximately 3:1. In some embodiments, the cell therapy comprises administering CD4+ and CD8+ T cells, wherein T cells of each dose comprises a recombinant receptor that specifically binds to the antigen, wherein the administration comprises administering a plurality of separate compositions, the plurality of separate compositions including a first composition including or enriched in the CD8+ T cells and a second composition including or enriched in the CD4+ T cells. In some embodiments, the CD4+ T cells including the recombinant receptor in the one of the first and second compositions and the CD8+ T cells including the recombinant receptor in the other of the first and second compositions are present at a defined ratio that is or is approximately 1:1 or is between approximately 1:3 and approximately 3:1. In some embodiments, the CD4+ T cells including the recombinant receptor and the CD8+ T cells including the recombinant receptor administered in the first and second compositions are present at a defined ratio, which ratio is or is approximately 1:1 or is between approximately 1:3 and approximately 3:1. In some embodiments, the recombinant recetor is a CAR,

In some embodiments, the recombinant receptor is a T cell receptor (TCR) or a functional non-T cell receptor. In some embodiments, the recombinant receptor is a chimeric antigen receptor (CAR).

In some embodiments, the cell therapy comprises administration of from or from about 1×10⁵ to 5×10⁸ total CAR-expressing T cells, 1×10⁶ to 2.5×10⁸ total CAR-expressing T cells, 5×10⁶ to 1×10⁸ total CAR-expressing T cells, 1×10⁷ to 2.5×10⁸ total CAR-expressing T cells, 5×10⁷ to 1×10⁸ total CAR-expressing T cells, each inclusive. In some embodiments, the cell therapy comprises administration of at or about 1×10⁸ CAR-expressing cells.

In some embodiments, the antigen is a tumor antigen. In some embodiments, the antigen is selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha(IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1). In some embodiments, the antigen is associated with a B cell malignancy. In some embodiments, the antigen is expressed on human B cells. In some embodiments, wherein the antigen is CD20, CD19, CD22, ROR1, CD45, CD21, CD5, CD33, Igkappa, Iglambda, CD79a, CD79b or CD30. In some embodiments, the antigen is CD19.

In some embodiments, the CAR comprises an extracellular antigen binding domain that binds to the antigen, a transmembrane domain, and an intracellular signaling region including an intracellular signaling domain of a CD3-zeta (CD3) chain and a costimulatory signaling domain. In some embodiments, the costimulatory signaling region comprises a signaling domain of 4-1BB. In some embodiments, the costimulatory signaling region comprises a signaling domain of CD28.

In some embodiments, the method comprises, prior to administration of the cytotoxic therapy, administering a lymphodepleting agent or therapy to the subject. In some embodiments, the lymphodepleting therapy is completed between 2 and 7 days before the initiation of administration of the cytotoxic therapy. In some embodiments, the lymphodepleting therapy comprises the administration of fludarabine and/or cyclophosphamide. In some embodiments, the lymphodepleting therapy comprises administration of cyclophosphamide at about 200-400 mg/m². In some embodiments, the lymphodepleting therapy comprises administration of cyclophosphamide at or about 300 mg/m², inclusive, and/or fludarabine at about 20-40 mg/m², optionally 30 mg/m², daily for 2-4 days; or wherein the lymphodepleting therapy comprises administration of cyclophosphamide at about 500 mg/m². In some embodiments, the fludarabine is administered for 3 days. In some embodiments, the lymphodepleting therapy comprises administration of cyclophosphamide at or about 300 mg/m² and fludarabine at about 30 mg/m² daily for 3 days. In some embodiments, the lymphodepleting therapy comprises administration of cyclophosphamide at or about 500 mg/m² and fludarabine at about 30 mg/m² daily for 3 days.

In some embodiments, the dosing regimen of the inhibitor comprises a subtherapeutic amount of the inhibitor. In some embodiments, the dosing regimen of the inhibitor is not sufficient to reduce tumor burden in the subject or reduces tumor burden by less than 10% when administered as a monotherapy in the absence of the combined administration with the cytotoxic therapy. In some embodiments, the dosing regimen of the inhibitor does not result in a complete or partial response in a group of similarly treated subjects, or results in such a response in no more than 10% of such subjects, when administered as a monotherapy in the absence of the combined administration with the cytotoxic therapy.

In some embodiments, the dosing regimen of the inhibitor comprises once daily dosing. In some embodiments, the once daily dose is between at or about 20 mg and at or 400 mg, inclusive. In some embodiments, the once daily dose is between at or about 20 mg and at or about 200 mg, inclusive. In some embodiments, the once daily dose is an amount of the inhibitor of between at or about 50 mg and at or about 100 mg, inclusive. In some embodiments, the once daily dose is at or about 50 mg. In some embodiments, the once daily dose is at or about 100 mg. In some embodiments, the once daily dose is at or about 200 mg. In some embodiments, the once daily dose is at or about 400 mg.

In some embodiments, prior to administration of the dosing regimen of the inhibitor, the subject has been previously treated with an inhibitor of a prosurvival Bcl-2 family protein. In some embodiments, the inhibitor is venetoclax. In some embodiments, the previous treatment with the inhibitor is administered at a time between the collecting of the autologous cells from the subject and prior to administering a lymphodepleting therapy to the subject, as a bridging therapy prior to the administration of the cytotoxic therapy. In some embodiments, the collecting is by apheresis or leukapheresis. In some embodiments, the previous treatment of the inhibitor is administered in a dose-ramp up schedule, wherein the dose-ramp up schedule comprises administration of escalating doses of the inhibitor. In some embodiments, the inhibitor is administered to the subject in escalating doses until a maximum dose of 100 mg daily is reached. In some embodiments, the escalating doses comprise a first dose that is at about 20 mg per day, a second dose that is at about 50 mg per day, and a third dose that is at about 100 mg per day. In some embodiments, each escalating dose is administered once daily for a week and/or the last escalating dose is administered once daily for a week or until the end of the bridging therapy. In some embodiments, the previous treatment with the inhibitor is ceased at least 1 day prior to administration of the lymphodepleting therapy.

In some embodiments, the previous treatment with the inhibitor is ceased: for at least at or about 3 days or for at least at or about 4 days prior to administration of the lymphodepleting therapy; for at least an amount of time until the concentration of the inhibitor in the subject's bloodstream is reduced by about three half-lives or about four half-lives; or for at least an amount of time until the inhibitor is eliminated from the bloodstream of the subject.

In some embodiments, the inhibitor inhibits one or more prosurvival BCL2 family protein selected from among the group consisting of BCL2, BCLXL, BCLW, BCLB, MCL1, and combinations thereof. In some embodiments, the one or more prosurvival BCL2 family protein is BCL2, BCLXL, and/or BCLW. In some embodiments, the inhibitor is selected from among the group consisting of venetoclax, navitoclax, ABT737, maritoclax, obatoclax, and clitocine. In some embodiments, the inhibitor is venetoclax.

In some embodiments, the cancer is a hematological malignancy. In some embodiments, the cancer is a B cell malignancy. In some embodiments, the cancer is a myeloma, leukemia or lymphoma. In some embodiments, the cancer is an acute lymphoblastic leukemia (ALL), adult ALL, chronic lymphoblastic leukemia (CLL), a small lymphocytic lymphoma (SLL), non-Hodgkin lymphoma (NHL), a large B cell lymphoma. In some embodiments, the cancer is a chronic lymphocytic leukemia (CLL). In some embodiments, the cancer is a small lymphocytic lymphoma (SLL). In some embodiments, the cancer is a non-Hodgkin lymphoma (NHL). In some embodiments, the NHL is a diffuse large B cell lymphoma.

In some embodiments, the subject has relapsed following remission after treatment with, or become refractory to, failed and/or was intolerant to treatment with the one or more prior therapies for treating the cancer. In some embodiments, the cancer exhibits overexpression of a prosurvival BCL2 family protein that is targeted by the inhibitor.

In some embodiments, the dosing regimen of the inhibitor comprises administration of the inhibitor, once daily for a period of time of at least 3 months after the initiation of the administration of the cytotoxic therapy. In some embodiments, the method further comprises continued administration of the dosing regimen of the inhibitor if at the end of the period of time, the subject does not exhibit a clinical remission, such as if the subject exhibits a partial response (PR) or stable disease (SD), or has minimal residual disease (MRD) greater than or equal to 10⁻⁴. In some embodiments, administration of the inhibitor in the dosing regimen is discontinued at the end of the period of time if the subject exhibits clinical remission. In some embodiments, the period of time is 3 months.

In some embodiments, the method increases the cytotoxic activity of the cytotoxic therapy compared to a method that does not involve the administration of the inhibitor. In some embodiments, the method increases cytolytic killing. In some embodiments, the cytolytic killing is increased via perforin- and/or granzyme-mediated apoptosis, of one or more of the cancer cells compared to a method that does not involve the administration of the inhibitor.

In some embodiments, at least 35%, at least 40% or at least 50% of subjects treated according to the method achieve a complete response (CR) that is durable, or is durable in at least 60, 70, 80, 90, or 95% of subjects achieving the CR, for at or greater than 6 months or at or greater than 9 months. In some embodiments, at least 60, 70, 80, 90, or 95% of subjects achieving a CR by six months remain in response, remain in CR, and/or survive or survive without progression, for greater at or greater than 3 months and/or at or greater than 6 months and/or at greater than nine months. In some embodiments, at least 50%, at least 60% or at least 70% of the subjects treated according to the method achieve objective response (OR). In some embodiments, the OR is durable, or is durable in at least 60, 70, 80, 90, or 95% of subjects achieving the OR, for at or greater than 6 months or at or greater than 9 months. In some embodiments, at least 60, 70, 80, 90, or 95% of subjects achieving an OR by six months remain in response or surviving for greater at or greater than 3 months and/or at or greater than 6 months.

In some embodiments, the subject is a human.

Provided herein is a method of treatment with a cytotoxic therapy, including: (a) assessing the level or amount of one or more prosurvival gene in a biological sample from a subject having or suspected of having a cancer, wherein the level or amount of the one or more prosurvival gene is the level or amount of a protein or a polynucleotide encoded by the one or more prosurvival gene; (b) selecting the subject for treatment with a cytotoxic therapy if the level or amount of the one or more prosurvival gene is below a gene reference value, wherein the cytotoxic therapy is a T cell therapy including or enriched in T cells and specifically binds to an antigen associated with, expressed by, or present on cells of the cancer; and (c) administering to the selected patient the cytotoxic therapy.

In some embodiments, an inhibitor of a prosurvival gene is not administered to the subject at or after initiation of the administration of the cytotoxic therapy. In some embodiments, the inhibitor is not administered within 7 days, 14 days or 28 days after the administration of the cytotoxic therapy.

Provided herein is a method of selecting a subject having a cancer for administering an inhibitor of a prosurvival BCL2 family protein, including: (a) assessing the level or amount of one or more prosurvival gene in a biological sample from a subject having or suspected of having a cancer, wherein the level or amount of the one or more prosurvival gene is the level or amount of a protein or a polynucleotide encoded by the one or more prosurvival gene, wherein the subject is to receive administration of a cytotoxic therapy, that is a T cell therapy including or enriched in T cells and that specifically binds to an antigen associated with, expressed by, or present on cells of the cancer, and wherein the biological sample is obtained from the subject prior to the administration of the cytotoxic therapy; and (b) selecting the subject for treatment with an inhibitor of a prosurvival BCL2 family protein if the level or amount of the one or more prosurvival gene is above a gene reference value.

In some embodiments, the method further includes administering to the selected subject the inhibitor in combination with the cytotoxic therapy.

Provided herein is a method of identifying a subject having a cancer that is predicted to be resistant to treatment with a cytotoxic therapy, including: (a) assessing the level or amount of one or more prosurvival gene in a biological sample from the subject, wherein the level or amount of the one or more prosurvival gene is the level or amount of a protein and a polynucleotide encoded by the one or more prosurvival gene, wherein the subject is a candidate for administration of a dose of a cytotoxic therapy, wherein the cytotoxic therapy is a T cell therapy including or enriched in T cells and that specifically binds to an antigen associated with, expressed by, or present on cells of the cancer, and wherein the biological sample is obtained from the subject prior to the administration of the cytotoxic therapy; and (b) identifying the subject as having a cancer that is predicted to be resistant to treatment with the cytotoxic therapy if the level or amount of the one or more prosurvival gene is above a gene reference value.

In some embodiments, the subject is identified as having a cancer that is predicted to be resistant to treatment with the cytotoxic therapy, further including administering an alternative treatment to the identified subject, wherein the alternative treatment is selected from among the following: a combination treatment including the cytotoxic therapy and an additional agent that modulates or increases the activity of the cytotoxic therapy; an increased dose of the cytotoxic therapy; and/or a chemotherapeutic agent. In some embodiments, the alternative treatment is a combination treatment including the cytotoxic therapy and an additional agent that modulates or increases the activity of the T cell therapy. In some embodiments, the additional agent is an immune checkpoint inhibitor, a modulator of a metabolic pathway, an adenosine receptor antagonist, a kinase inhibitor, an anti-TGFβ antibody or an anti-TGFβR antibody, a cytokine, or a prosurvival BCL2 family protein inhibitor. In some embodiments, the alternative treatment is a combination treatment including the cytotoxic therapy and a prosurvival BCL2 family protein inhibitor. In some embodiments, the method further includes administering to the selected subject the inhibitor in combination with the cytotoxic therapy.

In some embodiments, the gene reference value is within 25%, within 20%, within 15%, within 10%, or within 5% of an average level or amount of the one or more prosurvuval gene in (a) a population of subjects not having the cancer or (b) a population of subjects having the cancer and administered the cytotoxic therapy, who went on to exhibit a partial response (PR) or complete response (CR) following administration of the therapy.

In some embodiments, the population of subjects having the cancer went on to exhibit the PR or CR at least 1 month, 2 months, 3 months, 4 months, 5 months, 6 months, 7 months, 8 months, 9 months, or more following administration of the cytotoxic therapy. In some embodiments, the level or amount of the one or more prosurvival genes is assessed in the biological sample before a lymphodepleting therapy is administered to the subject, optionally within 7 days before, 6 days before, 5 days before, 4 days before, 3 days before, 2 days before, 1 day before, 16 hours before, 12 hours before, 6 hours before, 2 hours before, or 1 hour before the lymphodepleting therapy is administered to the subject.

Provided herein is a method of determining responsiveness of a subject having a cancer to a cytotoxic therapy, wherein the cytotoxic therapy is a T cell therapy including or enriched in T cells and that specifically binds to an antigen associated with, expressed by, or present on cells of the cancer, the method including: (a) assessing the level or amount of expression of one or more prosurvival gene in a biological sample from the subject, wherein the level or amount of the one or more prosurvival gene is the level or amount of a protein or a polynucleotide encoded by the one or more prosurvival gene, wherein the biological sample is obtained from the subject at a first time prior to the subject being administered the cytotoxic therapy, and wherein the subject is to receive treatment with the cytotoxic therapy; (b) assessing the level or amount of expression of the one or more prosurvival gene in a biological sample from the subject at a second time after administration of the cytotoxic therapy to the subject, wherein the level or amount of the one or more prosurvival gene is the level or amount of a protein and/or a polynucleotide encoded by the one or more prosurvival gene, wherein the biological sample is obtained at a second time after the administration of the cytotoxic therapy to the subject, and wherein the subject has been administered the cytotoxic therapy prior to the assessing in (b); and (c) determining that the subject is responsive to the therapy if the level or amount of the one or more prosurvival gene at the second time is lower than the level or amount of the one or more prosurvival gene at the first time.

In some embodiments, the method further includes prior to the assessing in (b), administering to the subject the cytotoxic therapy. In some embodiments, the biological sample is obtained from the subject at a time before a lymphodepleting therapy is administered to the subject. In some embodiments, the biological sample is obtained within 7 days before, 6 days before, 5 days before, 4 days before, 3 days before, 2 days before, 1 day before, 16 hours before, 12 hours before, 6 hours before, 2 hours before, or 1 hour before the lymphodepleting therapy is administered to the subject.

In some embodiments, the one or more pro-survival gene is selected from among the following: a myc family gene, p53, and enhancer of zeste homolog 2 (EZH2). In some embodiments, the one or more pro-survival gene is or comprises a myc family gene. In some embodiments, a myc family gene comprises one or more of c-myc, l-myc, and n-myc. In some embodiments, the one or more pro-survival gene is or comprises p53. In some embodiments, the one or more pro-survival gene is or comprises EZH2.

In some embodiments, the cytotoxic therapy comprises cells that are autologous to the subject. In some embodiments, the cytotoxic therapy is selected from among the group consisting of a tumor infiltrating lymphocytic (TIL) therapy, a transgenic TCR therapy, and a chimeric antigen receptor (CAR)-expressing cell therapy. In some embodiments, the cytotoxic therapy comprises a dose of cells expressing a recombinant receptor that specifically binds to the antigen.

In some embodiments, the cytotoxic therapy comprises or is enriched in CD3+, CD4+, CD8+, or CD4+ and CD8+ T cells. In some embodiments, the cytotoxic therapy comprises or is enriched in CD4+ and CD8+ T cells. In some embodiments, the CD4+ and CD8+ T cells of the cytotoxic therapy comprises a defined ratio of CD4+ recombinant receptor-expressing T cells to CD8+ CAR-expressing T cells and/or of CD4+ recombinant-expressing T cells to CD8+ CAR-expressing T cells, that is or is approximately 1:1 or is between approximately 1:3 and approximately 3:1.

In some embodiments, the recombinant receptor is a T cell receptor (TCR) or a functional non-T cell receptor. In some embodiments, the recombinant receptor is a chimeric antigen receptor (CAR). In some embodiments, the CAR comprises an extracellular antigen binding domain that binds to the antigen, a transmembrane domain, and an intracellular signaling region including an intracellular signaling domain of a CD3-zeta (CD3) chain and a costimulatory signaling domain. In some embodiments, the costimulatory signaling region comprises a signaling domain of 4-1BB. In some embodiments, the costimulatory region comprises a signaling domain of CD28.

In some embodiments, the inhibitor is administered in combination with the cytotoxic therapy in accord with any of the provided methods.

In some embodiments, the inhibitor of a prosurvival gene is a BCL2 family protein inhibitor, wherein the inhibitor inhibits one or more prosurvival BCL2 family protein selected from among the group consisting of BCL2, BCLXL, BCLW, BCLB, MCL1, and combinations thereof. In some embodiments, the one or more prosurvival BCL2 family protein is BCL2, BCLXL, and/or BCLW.

In some embodiments, the inhibitor is selected from among the group consisting of venetoclax, navitoclax, ABT737, maritoclax, obatoclax, and clitocine. In some embodiments, the inhibitor is venetoclax.

In some embodiments, the cancer is a hematological malignancy. In some embodiments, the cancer is a B cell malignancy. In some embodiments, the cancer is a myeloma, leukemia or lymphoma. In some embodiments, the cancer is an acute lymphoblastic leukemia (ALL), adult ALL, chronic lymphoblastic leukemia (CLL), a small lymphocytic lymphoma (SLL), non-Hodgkin lymphoma (NHL), a large B cell lymphoma. In some embodiments, the cancer is a chronic lymphocytic leukemia (CLL). In some embodiments, the cancer is a small lymphocytic lymphoma (SLL). In some embodiments, the cancer is a non-Hodgkin lymphoma (NHL). In some embodiments, the NHL is a diffuse large B-cell lymphoma (DLBCL).vin some embodiments, the subject has relapsed following remission after treatment with, or become refractory to, failed and/or was intolerant to treatment with the one or more prior therapies for treating the cancer. In some embodiments, the biological sample is a tumor biopsy. In some embodiments, the sample is a lymph node biopsy. In some embodiments, the subject is a human.

### BRIEF DESCRIPTION OF THE DRAWINGS

FIG. 1A shows the cell viability of human CD19-expressing lymphoma and leukemia target cell lines co-cultured with anti-CD19 CAR T cells at increasing ratios of effector cells to target cells (E:T).

FIG. 1B shows the size of spheroids generated from the non-Hodgkin lymphoma (NHL) RL cell line over time, co-cultured with CD19-targeting CAR T cells at E:T ratios of 0.25:1, 0.5:1 and 1:1.

FIG. 2 shows the fold change in cell count of human CD19-expressing leukemia and lymphoma target cell lines co-cultured for 120 hours with CD19-targeting CAR T cells at an E:T ratio of 2.5:1.

FIG. 3 shows the cell viability of anti-CD19 CAR-expressing T cells treated for 96 hours with an exemplary BCL2 inhibitor.

FIG. 4A shows the cell count of human CD19-expressing lymphoma cell lines cultured alone, with CD19-targeting CAR T cells at an E:T ratio of 2.5:1, with an exemplary BCL2 inhibitor, or with both.

FIG. 4B shows the cell count of CD19-expressing Granta-519 lymphoma target cells cultured alone, with anti-CD19 CART cells at a suboptimal E:T ratio of 1:1, with an exemplary BCL2 inhibitor, or both.

FIG. 5A shows the cell count of RL cells cultured alone, with CD19-targeting CAR T cells at an E:T ratio of 1:1, with an exemplary BCL2 inhibitor, or with both.

FIG. 5B shows the tumor volume of RL spheroids cultured alone, with CD19-targeting CAR T cells at an E:T ratio of 1:1, with an exemplary BCL2 inhibitor, or with both.

FIG. 5C shows the tumor volume of RL spheroids cultured alone or with CD19-targeting CAR T cells for 9 days, in the presence of increasing concentrations of an exemplary BCL2 inhibitor.

FIG. 6A shows the number of RL cells cultured for 8 days alone or with anti-CD19 CAR T cells (E:T ratio of 1:1) that had been previously subjected to chronic stimulation by incubation with anti-idiotypic antibody-coated beads, in the presence or absence of an exemplary BCL2 inhibitor.

FIG. 6B shows the tumor volume of RL spheroids cultured for 8 days alone or with anti-CD19 CAR T cells (E:T ratio of 1:1) that had been previously subjected to chronic stimulation by incubation with anti-idiotypic antibody-coated beads, in the presence or absence of an exemplary BCL2 inhibitor.

FIG. 7 shows gene expression data of tumor biopsies from 36 DLBCL patients enrolled in a clinical trial for a CD19-targeting CAR T cell therapy. A hypothetical threshold was set assuming, at 3 months after admistration of the cell therapy, 30% of subjects (11/36) would not respond and 70% of subjects (25/36) would respond. Actual responses are shown, designated as complete response (CR), partial response (PR), progressive disease (PD), or status unavailable (Not Available).

FIG. 8A shows the expression, indicated by mean fluorescence intensity (MFI), of BCL2 by anti-CD19 CAR-expressing T cell compositions generated from three healthy human donors, compared to a fluorescence minus one (FMO) control.

FIG. 8B shows the percent of CAR+caspase 3+ cells among CD19 CAR T cell compositions generated from three healthy human donors (each dot represents an individual donor), following chronic stimulation by incubation with anti-idiotypic antibody-coated beads and exposure to increasing concentrations of an exemplary BCL2 inhibitor.

FIGS. 8C and 8D show cell viability and expansion kinetics of CD19-targeting CAR T cells, respectively, following chronic stimulation by incubation with anti-idiotypic antibody-coated beads and exposure to an exemplary BCL2 inhibitor.

FIG. 9 shows JeKo-1 mantle cell lymphoma (MCL) target cells cultured alone, with CD19-targeting CAR T cells, with an exemplary BCL2 inhibitor, or with both.

FIG. 10 shows the IC50 of an exemplary BCL2 inhibitor against anti-CD19 CAR T cells in culture with 5%, 10%, or 20% serum.

FIGS. 11A and 11B show the tumor burden and body weight, respectively, of NOD scid gamma (NSG) mice injected with JeKo-1 MCL cells at Day −7 and treated daily with an exemplary BCL2 inhibitor from Day 0 to Day 21.

FIGS. 12A-12C show tumor burden (FIG. 12A) and survival in NSG mice injected with JeKo-1 MCL cells at Day −7 and treated with an exemplary BCL2 inhibitor daily from Day 0 to Day 21 (FIG. 12B), anti-CD19 CAR T cells on Day −1 and Day 0 (FIG. 12C), or both (FIG. 12C).

FIG. 13 shows the number of CD4+CAR+ and CD8+CAR+ cells in the blood of NSG mice injected with JeKo-1 MCL cells and treated with CD19-targeting CAR T cells, in the presence or absence of an exemplary BCL2 inhibitor, on Days 7, 13, and 19.

FIGS. 14A and 14B show the number of CD3+CAR+ T cells and RL target cells, respectively, following a 6-day co-culture. Various concentrations of an exemplary BCL2 inhibitor were provided in the co-culture at its initiation, or 24, 48, or 72 hours after the initiation of co-culture.

### DETAILED DESCRIPTION

Provided herein are combination therapies for treating a subject having a cancer involving administration of an immunotherapy or a cell therapy (e.g. T cell therapy, such as CAR-T cell) for treating a cancer and an inhibitor of a prosurvival BCL2 family protein, such as a BCL2 inhibitor. In some embodiments, the immunotherapy or cell therapy includes any such therapy that specifically binds to an antigen associated with, expressed by, or present on cells of the cancer. In particular embodiments, the immunotherapy or cell therapy is or involves a therapy that results in cytolytic effector-mediated killing of cancer cells, such as by target cell apoptosis (hereinafter “cytotoxic therapy”). Among the provided embodiments are combination therapy involving administration of an immunotherapy involving T cell function or activity, such as a T-cell engaging therapy or a T cell therapy (e.g., CAR-expressing T cells), and administration of an inhibitor of a prosurvival BCL2 family protein, such as a BCL2 inhibitor. In some embodiments, the cytotoxic therapy is a T cell therapy, such as CAR-T cells. In particular embodiments, the provided combination therapies and methods improve responses to the immunotherapy or cell therapy by activity of the inhibitor to increase susceptibility of the cancer cells, such as tumor cells, to apoptosis thereby rendering the cells more sensitize to cytolytic effector-mediated killing.

Also provided are combinations and articles of manufacture, such as kits, that contain a composition comprising the therapy and/or a composition comprising a prosurvival BCL2 family protein inhibitor, such as a BCL2 inhibitor, e.g., venetoclax or navitoclax, and uses of such compositions and combinations to treat or prevent cancers, such as a B cell malignancy.

Cell therapies, such as T cell-based therapies, for example, adoptive T cell therapies (including those involving the administration of cells expressing chimeric receptors specific for a cancer of interest, such as chimeric antigen receptors (CARs) and/or other recombinant antigen receptors, as well as other adoptive immune cell and adoptive T cell therapies) can be effective in the treatment of diseases and disorders such as a B cell malignancies. The engineered expression of recombinant receptors, such as chimeric antigen receptors (CARs), on the surface of T cells enables the redirection of T cell specificity. In clinical studies, CAR-T cells, for example anti-CD19 CAR-T cells, have produced durable, complete responses in both leukemia and lymphoma patients (Porter et al. (2015) Sci Transl Med., 7:303ra139; Kochenderfer (2015) J. Clin. Oncol., 33: 540-9; Lee et al. (2015) Lancet, 385:517-28; Maude et al. (2014) N Engl J Med, 371:1507-17).

In certain contexts, available approaches to adoptive cell therapy may not always be entirely satisfactory. In some contexts, optimal efficacy can depend on the ability of the administered cells to recognize and bind to a target, e.g., target antigen, and to exert various effector functions, including cytotoxic killing of cancer cells and secretion of various factors such as cytokines. In some cases, however, certain cancer cells exhibit resistance to certain therapies, such as immunotherapies and cell therapies. In particular, results herein demonstrate that certain cancers are resistant to CAR T cell-mediated killing while others are more sensitive.

In some aspects, the provided methods, combinations and uses provide for or achieve improved or more durable responses or efficacy as compared to alternative methods, such as alternative methods involving only the administration of the immunotherapy or cell therapy but not in combination with an inhibitor of a prosurvival BCL2 family protein (e.g. venetoclax). In some embodiments, the methods are advantageous by virtue of administering an inhibitor of a prosurvival BCL2 family protein (e.g. venetoclax) shortly before (e.g. within 7 days) or concurrently with administration of an immunotherapy or a cell therapy (e.g. T cell therapy, such as CAR-T cell), thereby sensitizing the tumor and/or making the tumor less resistant to, or more susceptible to, treatment with the immunotherapy or cell therapy. In some embodiments of the provided methods, the cytotoxic therapy is a cell therapy (e.g. T cell therapy, such as CAR-T cell) and it is further found that the advantageous effect of sensitizing the tumor and/or making the tumor less resistant to, or more susceptible to, treatment with the cell therapy can be achieved by initiating administration of the inhibitor of a prosurvival BCL2 family protein (e.g. venetoclax) in a window of time even after initiation of administration of the cell therapy to minimize or avoid a detrimental effect of the inhibitor on cells of the cell therapy. In particular, observations herein demonstrate that an inhibitor of a prosurvival BCL2 family protein (e.g. venetoclax), particularly if given at too high of a dosage, can exacerbate activation induced cell death (AICD) of cells of the cell therapy. However, delaying administration of the inhibitor of a prosurvival BCL2 family protein (e.g. venetoclax) until a time after AICD is at or has reached it peak or been reduced after having peaked can avoid detrimental effects on the cells of the cell therapy while substantially improving, e.g. synergistically increasing, T cell-mediated killing of the tumor by cells of the cell therapy (e.g. CAR-T cells).

The provided methods are based on observations that certain cancers that are resistant to CAR T cell-mediated killing exhibit high expression of the prosurvival tumor suppressor p53 and other genes involved in prosurvival cell cycle pathways. It is found herein that the presence of an inhibitor of a prosurvival BCL2 family protein, such as a BCL2 inhibitor, e.g. venetoclax, improves T cell-mediated killing of cancer cells by a T cell therapy (e.g. CAR T cells), particularly among cancers that exhibit resistance following exposure to the T cell therapy (e.g. CAR T cells) alone. Such results were observed with low doses of the inhibitor that did not exhibit any activity on the tumor when administered alone. These results evidence that administration of inhibitors of a prosurvival BCL2 family protein, including at subtherapeutic doses, may improve responses to certain effector-mediated immunotherapies, such as T cell engagers or T cell therapies.

It is additionally found herein that the presence of an inhibitor of a prosurvival BCL2 family protein, such as a BCL2 inhibitor, e.g. venetoclax, increases the susceptibility of cancer cells to T cell-mediated killing by a T cell therapy (e.g. CAR T cells), such as among cancers that exhibit resistance T cell therapy-mediated cell death (e.g. CAR T cells) alone, even when initiation of administration of the inhibitor is after or subsequent to administration of the T cell therapy (e.g. CAR T cells). Thus, in some cases, the cytotoic effects of a BCL2 inhibitor, e.g. venetoclax, and a T cell therapy (e.g. CAR T cells) may be synergistic to result in cell death of cells otherwise resistant to treatment with the T cell therapy alone. In some cases, administration of the inhibitor, e.g. venetoclax, may sensitize otherwise resistant cancer cells to CAR T cell-mediated killing when it is administered within or at about 7 days after administration of the cell therapy. For example, as demonstrated in Example 9, in the absence of venetoclax, tumor cells were relatively resistant to cell death mediated by CAR T cells. Although a therapeutic dose of venetoclax given concurrently or at 24 hours reduced CAR-T cell numbers and activity, a therapeutic dose of venetoclax administered 48 or 72 hours after initiation of co-culture of tumor cells and CAR T cells was able to sensitize the tumor cells to CAR T cell-mediated killing, without deleterious effects on the CAR T cells being observed. These results surprisingly indicated that there is a window of time after initiation of the cell therapy (e.g. CAR-T cells) at which deleterious effects on cells of the cell therapy by an inhibitor of a prosurvival BCL2 family protein, e.g. venetoclax, even at therapeutic doses, can be avoided or minimized while maintaining the ability of the inhibitor to increase susceptibility of the tumor to the T cell therapy. These results indicate that administration of inhibitors of a prosurvival BCL2 family protein, e.g. venetoclax, such as within or at about 7 days following administration of a T cell therapy, including at therapeutic doses, may improve responses to T cell therapies without exerting significant deleterious effects on T cells.

Venetoclax (ABT-199) is a small molecule inhibitor (SMI) that blocks the activity of the B-cell lymphoma 2 (BCL2) protein. Venetoclax is approved for use in chronic lymphocytic leukemia (CLL), small lymphocytic leukemia (SLL), and in combination with azacitidine, decitabine, or low-dose cytarabine for newly diagnosed acute myeloid leukemia (AML) in adult who are at least 75 years of age, or who have comorbidities that preclude use of intensive induction chemotherapy. Navitoclax (ABT-263), another prosurvival BCL2 inhibitor, is a SMI that blocks the activity of the BCL2, B-cell lymphoma extra-large (BCLXL), and BCL2-like protein 2 (BCLW) proteins. Other BCL2 inhibitors include, but are not limited to ABT-737, AT-101/GDC-0199 (Gossypol), apogossypol, TW-37, G3139 (Genasense), GX15-070 (obatoclax), sabutoclax, HA14-1, antimycin A, BH3I-1, YC137, maritoclax, clitocine, UMI-77, WEHI-539, and 544563.

Members of the BCL2 family include both pro-apoptotic and anti-apoptotic proteins, and it is the balance of signaling between these two groups that can determine whether a cell is more sensitized or more resistant to apoptosis. In some cases, the overexpression of one or more prosurvival BCL2 family proteins (e.g. BCL2) can result in increased anti-apoptotic signaling and resistance to cell death. For example, in some cases, overexpression of BCL2 may be caused by the (14;18)(q32;q21) translocation. In some cases, overexpression of BCL2 may be caused by amplification of the gene encoding the BCL2 protein. Resistance to cell death may, in some cases, occur when the signaling of prosurvival (anti-apoptotic) BCL2 proteins (e.g. BCL2, BCLXL, BCLB, BCLW, BFL1, MCL1) outweighs the signaling of pro-apoptotic BCL2 proteins (e.g. BAX, BAK, BIG, BIM, NOXA, PUMA), such as when one or more prosurvival BCL2 proteins are overexpressed. In certain aspects, overexpression of one or more prosurvival BCL2 family proteins can support and increase cancer cell survival. In some aspects, increased expression of prosurvival BCL2 family proteins blocks pro-apoptotic BCL2 family proteins and inhibits a cancer cell's intrinsic (mitochondrial) apoptotic pathway.

Among the provided embodiments, the methods involve combination therapy of a therapy that targets or is directed to killing of cells of a cancer, e.g. a cytotoxic therapy, such as a CAR T cell therapy, an an inhibitor of a BCL2 family protein. In some aspects, the inhibitor is inhibits activity of a BCL2 family protein that is a prosurvival (antiapoptotic) BCL2 family protein such as BCL2, B-cell lymphoma extra-large (BCLXL), BCL2 related protein A1 (BFL1), BCL2-like protein 2 (BCLW), BCL2-like protein 10 (BCLB), induced myeloid leukemia cell differentiation protein (MCL1), or combinations thereof. In some aspects, the cancer is one in which the prosurvival BCL2 family protein is overexpressed. In some aspects, the inhibitor does not inhibit or reduce the activity of a BCL2 family protein that is a proapoptopic BCL2 family protein such as BCL2 associated X (BAX), BCL2 antagonist/killer 1 (BAK), DIVA, BCLXS, BCL2 interacting killer (BIK), BCL2-like protein 11 (BIM), BCL2 associated agonist of cell death (BAD), or combinations thereof. In some aspects, the cancer is one in which the proapoptopic BCL2 family protein is underexpressed or its activity is inhibited.

In some aspects, overexpression of a prosurvival BCL2 family protein is implicated in a number of cancers, including bladder cancer, brain cancer, breast cancer, cervical cancer, colorectal cancer, endometrial cancer, head and neck cancers, lung cancer, ovarian cancer, pancreatic cancer, renal cancer, skin cancer, and hematologic malignancies, such as leukemias and lymphomas (see e.g., WO 2005/049593; WO2005/024636). In some cases, overexpression or aberrant expression of one or more prosurvival BCL2 family proteins is a mechanism underlying leukemias, lymphomas, and solid tumors, whereby overexpression dampens pro-apoptotic signaling to promote the survival of cancer cells (see e.g., Campbell, K. J. and Tait, S. W. G. (2018) Open Biol., 8:180002). In many cases, existing methods of employing prosurvival BCL2 family protein inhibitors, such as BCL2 inhibitors, e.g. venetoclax, involve use to the inhibitors as therapeutics for treating a variety of cancers. For example, venetoclax is indicated for treatment of certain cancers, such as B cell lymphomas, with a dose of 400 to 800 milligrams per day after a ramp-up period and for a duration of time that can extend for months to years.

However, reports indicate that certain inhibitors of prosurvival BCL2 family proteins may have deleterious effects, including direct cytotoxicity, on T cells (Karlsson et al. (2013) Cancer Gene Ther., 20:386-93). Further BCL2 is known to be involved in and regulate the survival of T cells (Wilson et al. (2010) Lancet Oncol., 11:70261-8). A study investigating the effect of navitoclax, an inhibitor of BCL2, BCLXL, and BCLW, on T cells found that exposure to navitoclax induced a relative and absolute reduction of CD3+CD4+ and CD3+CD8+ T cells in the peripheral blood of mice (Cippa et al., (2012) Cell Death and Disease, 3:e299). In a Phase I clinical trial of navitoclax ((NCT00406809), it was reported that patients in the trial showed a relatively rapid and substantial decrease in T cells (an average decrease of 241 CD3+ cells/μL) after 14 days of drug exposure to navitoclax at a dose of 200 milligrams/day or more, which was maintained at the time of the patients' final visits occurring on average 89 days after drug exposure (Wilson et al. (2010) Lancet Oncol., 11:70261-8).

These reports indicate that the combination of a T cell therapy and a BCL2 family inhibitor may not be a viable therapeutic strategy, particularly at therapeutic doses of the prosurvival BCL2 family protein inhibitor. In particular, such observations indicate that the combination of a prosurvival BCL2 family protein inhibitor and CAR T cell therapy would result in a loss of CAR T cells. As the proliferation and persistence of CAR T cells in vivo represent an ongoing challenge in the field of T cell therapies, the addition of an agent that is cytotoxic to T cells, such as an inhibitor of a prosurvival BCL2 family protein, would be not be favored as it would diminish efficacy of a CAR T cell therapy by reducing proliferation and persistence of the CAR-expressing T cells.

In addition, CAR-expressing T cells therapy may undergo activation-induced cell death (AICD). Specifically, reports indicate that CAR T cells may be prone to upregulatedion of expression of Fas, FasL, DRS, and TRAIL, such as upon excessive T cell stimulation, thereby resulting in programmed cell death. (Tschumi et al., J. Immunother. Cancer (2018) 71:6). To this end, combinatorial treatment with a BCL2 inhibitor (e.g. venetoclax), which blocks anti-apoptotic signaling, could exacerbate the programmed cell death observed in CAR T cells. Despite this, the observations herein indicate that the combination of a therapy, e.g. cytotoxic therapy, including a T cell therapy such as a CAR-T cell therapy is advantageous, even at therapeutic doses of the inhibitor capable of exerting deleterious effects on CAR T cells. When the inhibitor (e.g. venetoclax) is administered at a time when activation-induced cell death of the CAR T cells has peaked (e.g. 2-7 days after administration of the CAR T cells), not only were deleterious effects on CAR T cells not observed, but the CAR T cells demonstrated potent antitumor effects (see Example 9). In some aspects, the methods are advantageous by virtue of administering a BCL2 inhibitor at a therapeutic dose subsequent to administration of a T cell therapy (e.g. within or at about 7 days after administration of the T cell therapy), such as a dose in which the inhibitor would be expected to exert deleterious effects on T cells, including T cell viability. In some aspects, the provided methods and uses provide for or achieve improved or more durable responses or efficacy as compared to certain alternative methods.

Further, the observations herein indicate that the combination of a therapy, e.g. cytotoxic therapy, including a T cell therapy such as a CAR-T cell therapy is advantageous, even at subtherapeutic or lower doses of the inhibitor. The results herein also show that certain doses of the inhibitor do not impact viability of T cells. In some aspects, the methods are advantageous by virtue of administering a BCL2 inhibitor at a subtherapeutic dose, such as a dose in which the inhibitor alone would not be expected to or would not reduce tumor burden in the subject. In some aspects, the provided methods and uses provide for or achieve improved or more durable responses or efficacy as compared to certain alternative methods. In some aspects, the provided methods enhance or modulate the cytotoxic activity, such as via perforin- and/or granzyme-mediated apoptosis, toward one or more cells of a cancer of T cells against cancer cells, such as associated with administration of a T cell engaging therapy or a T cell therapy (e.g. CAR-expressing T cells),In some embodiments, observations herein indicate that a prosurvival BCL2 family protein inhibitor, such as a BCL2 inhibitor, e.g. venetoclax, may improve CAR T cell-mediated cytotoxicity activation at therapeutic and/or subtherapeutic doses. The provided findings indicate that combination therapy of the inhibitor in methods involving T cells, such as involving administration of adoptive T cell therapy, achieves improved function of the T cell therapy. In some embodiments, combination of the cell therapy (e.g., administration of engineered T cells) with the prosurvival BCL2 family protein inhibitor, e.g., BCL2 inhibitor and/or MCL1 inhibitor, improves or enhances one or more functions and/or effects of the T cell therapy, such as cytotoxicity and/or therapeutic outcomes, e.g., ability to kill or reduce the burden of tumor or other disease or target cell.

In some aspects, such effects are observed despite that the tumor or disease or target cell itself is insensitive, resistant and/or otherwise not sufficiently responsive to the therapy, e.g. cytotoxic therapy, such as immunotherapy including T cell engaging therapy or T cell therapy (e.g. CAR T cells), or to the dose of the inhibitor when each is administered alone. In some embodiments, the cancer is insensitive to or has become resistant to treatment with a therapy for treating the cancer that is directed to or targets killing of the cancer, e.g. cytotoxic therapy, such as an immunoterhapy, including a T cell engaging therapy or a T cell therapy (e.g. CAR T cell therapy). In some embodiments, the cancer is insensitive to or has become resistant to such therapies by virtue of the cells of the cancer suppressing pro-apoptotic signaling. For example, in some embodiments, the cancer is insensitive to or has become resistant to CAR T cells targeting the cancer-associated antigen, e.g. CD19. In some embodiments, the provided combination therapy achieves synergistic effects and activity compared to a therapy involving only administration of the therapy, e.g. cytotoxic therapy, or of the prosurvival BCL2 family protein inhibitor given at the same dosing regimen, e.g. dose and frequency.

In some embodiments, the provided methods, uses and combination therapies include administration of a prosurvival BCL2 family protein inhibitor, in combination with a therapy for treating the cancer that is directed to or targets killing of the cancer, e.g. cytotoxic therapy, such as an immunotherapy, including a T cell engaging therapy or a T cell therapy (e.g. CAR T cell therapy) in a subject that has already been administered the inhibitor or another prosurvival BCL2 family protein inhibitor. In some embodiments, the combination therapy, methods and uses include continued administration of the prosurvival BCL2 family protein inhibitor, e.g., venetoclax, in combination with a T cell therapy (e.g. CAR+ T cells) in a subject that has previously received administration of the inhibitor, e.g., venetoclax, but in the absence of (or not in combination with) a therapy for treating the cancer that is directed to or targets killing of the cancer, e.g. cytotoxic therapy, such as an immunotherapy, including a T cell engaging therapy or a T cell therapy (e.g. CAR T cell therapy). In some embodiments, the combination therapy methods include or involve administration of a lower dose of the prosurvival BCL2 family protein inhibitor, e.g., venetoclax, than the previous treatment.

In some embodiments, the methods and combinations result in improvements in T cell-mediated cytotoxicity against cancer cells. In some embodiments, the methods and combinations result in improvements in T cell-mediated cytotoxicity against cancer cells, optionally by increasing perforin- and/or granzyme-mediated apoptosis. Such improvements in some aspects result without compromising, or without substantially compromising, one or more other desired properties of functionality, e.g., of CAR-T cell functionality, proliferation, and/or persistence. In some embodiments, the combination with the inhibitor, while improving the cytotoxicity of the T cells, does not reduce the ability of the cells to become activated, secrete one or more desired cytokines, expand and/or persist, e.g., as measured in an in vitro assay as compared to such cells cultured under conditions otherwise the same but in the absence of the inhibitor.

In some embodiments, the provided embodiments involve initiating the administration of a prosurvival BCL2 family protein inhibitor, such as a BCL2 inhibitor, e.g., venetoclax, prior to or concurrently with or just after administration of the therapy for treating the cancer that is directed to or targets killing of the cancer, e.g. cytotoxic therapy, such as an immunotherapy, including a T cell engaging therapy or a T cell therapy (e.g. CAR T cell therapy) in a dosing regimen that is continued until after the administration of the therapy. In some embodiments, the inhibitor is administered at a time and in a dosing regimen to achieve a steady state concentration (Css) of the inhibitor at a time between at or about 1 day and at or about 14 days after initiation of administration of the cytotoxic therapy and/or at a time before peak levels of the cytotoxic therapy is detectable in the blood of the subject following administration of the cytotoxic therapy. In some embodiments, the initiation of administration of the inhibitor is at a time between at or about 7 days prior to and at or about 14 days after initiation of administration of the cytotoxic therapy, at or about 3 days prior to and at or about 14 days after initiation of administration of the cytotoxic therapy, or at or about 1 days prior to and at or about 8 days after initiation of administration of the cytotoxic therapy. In particular embodiments, administration of the inhibitor in the provided combination therapy methods is initiatied between at or about day 1 prior to and at or about day 1 after initation of administration of the cytotoxic therapy, such as at or about a day prior, on the same day as or a day after initaiton of administration of the cytoxic therapy.

In some embodiments, the provided embodiments involve initiating the administration of a prosurvival BCL2 family protein inhibitor, such as a BCL2 inhibitor, e.g., venetoclax, after administration of a cytotoxic therapy (e.g. CAR T cell therapy) in a dosing regimen. In some embodiments, the initiation of the administration of a prosurvival BCL2 family protein inhibitor, such as a BCL2 inhibitor, e.g., venetoclax, is after administration of the cytotoxic therapy, such as between about 1 day after and about 7 days after administration of the therapy for treating the cancer. In some embodiments, the initiation not the administration of a prosurvival BCL2 family protein inhibitor, such as a BCL2 inhibitor, e.g., venetoclax, is not until activation-induced cell death (AICD) of the cells of the cytotoxic therapy has peaked. In some embodiments, administration of a prosurvival BCL2 family protein inhibitor, such as a BCL2 inhibitor, e.g., venetoclax, is not until between about 1 day and about 7 days, between about 2 days and about 6 days, or between about 3 days and about 5 days, after administration of the cytotoxic therapy. In some embodiments, the initiation of administration of the inhibitor is at a time between at or about 1 day after and at or about 7 days after initiation of administration of the cytotoxic therapy. In some embodiments, the initiation of administration of the inhibitor is at a time between at or about 2 days after and at or about 6 days after initiation of administration of the cytotoxic therapy. In some embodiments, the initiation of administration of the inhibitor is at a time between at or about 3 days after and at or about 5 days after initiation of administration of the cytotoxic therapy. In some embodiments, the initiation of administration of the inhibitor is at a time that is at least or at least about 1 day after initiation of administration of the cytotoxic therapy. In some embodiments, the initiation of administration of the inhibitor is at a time at or about 2 days after initiation of administration of the cytotoxic therapy. In some embodiments, the initiation of administration of the inhibitor is at a time at or about 3 days after initiation of administration of the cytotoxic therapy. In some embodiments, the initiation of administration of the inhibitor is at a time at or about 4 days after initiation of administration of the cytotoxic therapy. In some embodiments, the initiation of administration of the inhibitor is at a time at or about 5 days after initiation of administration of the cytotoxic therapy. In some embodiments, the initiation of administration of the inhibitor is at a time at or about 6 days after initiation of administration of the cytotoxic therapy. In some embodiments, the initiation of administration of the inhibitor is at a time at or about 7 days after initiation of administration of the cytotoxic therapy.

In some cases, the combination therapy involves administration of the inhibitor up to or or about 1 month, 2 months, 3 months, 4 months, 5 months, 6 months, 7 months, 8 months, 9 months, 10 months, 11 months, 12 or more after the subject as received administration of the therapy, e.g. cytotoxic therapy. In some aspects, a prosurvival BCL2 family protein inhibitor, e.g., venetoclax, is administered for no more than three months after administration of the therapy for treating the cancer that is directed to or targets killing of the cancer, e.g. cytotoxic therapy, such as an immunotherapy, including a T cell engaging therapy or a T cell therapy (e.g. CAR T cell therapy). In some embodiments, inhibitor is administered in combination with a T cell therapy (e.g. CAR T cells) and administration of the inhibitor is continued in a dosing regimen that includes continued administration of the inhibitor until a time after which the cells of the T cell therapy have reached peak levels in the subject and/or are persisting in the subject. In some aspects, advantages of the provided embodiments also include the ability to modulate the dosing or administration of the inhibitor, e.g., venetoclax, or removing or discontinuing the administration of the inhibitor, e.g., venetoclax, depending on the tolerability in the subject. In some aspects, the provided embodiments, e.g., involving combination treatment with a prosurvival BCL2 family protein inhibitor, e.g., venetoclax, can help reduce tumor burden and/or mitigate cancer cell resistance to certain cytotoxic therapies, such as cell therapies, e.g. CAR T cell therapy.

In some embodiments, the provided methods can potentiate CAR-T cell therapy, which, in some aspects, can improve outcomes for treatment of subjects that have a cancer that is resistant or refractory to other therapies, is an aggressive or high-risk cancer, and/or that is or is likely to exhibit a relatively lower response rate to a CAR-T cell therapy when administered without the inhibitor. In some aspects, administering a prosurvival BCL2 family protein inhibitor, e.g., venetoclax, according to the provided methods could increase the activity of CAR-expressing cells for treating a cancer, e.g. B cell malignancy such as CLL or NHL, e.g. DLBCL or SLL, by increasing T cell cytotoxicity by reducing the resistance of cancer cells to the CAR T cell therapy, optionally by increasing perforin- and/or granzyme-mediated apoptosis of the cancer cells. In some aspects, anti-tumor activity of administered CAR+ T cells against human lymphoma cell is improved.

### Combination Therapy

Provided herein are methods for combination therapy for treating a disease or condition, such as a proliferative disease (e.g. cancer) that include administering to a subject a combination therapy of 1) an inhibitor of a prosurvival BCL2 family protein (e.g. venetoclax) and 2) an cytotoxic therapy, such as an immunotherapy or a cell therapy, e.g. a T cell therapy (e.g. CAR-expressing T cells). In some embodiments, a prosurvival BCL2 family protein is a BCL2 family protein that has the ability to promote survival and/or to mitigate pro-apoptotic signaling.

In some embodiments, the cytotoxic therapy is an immunotherapy or cell therapy, such as an adoptive immune cell therapy comprising T cells (e.g. CAR-expressing T cells) that specifically recognizes and/or binds to an antigen associated with, expressed by or present on cells of the cancer. Also provided are combinations and articles of manufacture, such as kits, that contain a composition comprising the T cell therapy and/or a composition comprising the inhibitor of a prosurvival BCL2 family proteins, and uses of such compositions and combinations to treat or prevent conditions or diseases such as cancers, including hematologic malignancies.

In some embodiments, methods can include administration of the inhibitor (e.g. venetoclax) prior to, simultaneously with, during, during the course of (including once and/or periodically during the course of), and/or subsequently to, the administration (e.g., initiation of administration) of the cytotoxic therapy, wherein the therapy specifically binds to an antigen associated with, expressed by or present on cells of the cancer, and wherein the inhibitor is administered in a dosing regimen sufficient to achieve a steady state concentration of the inhibitor within about 7 days prior to and about 14 days after initiation of administration of therapy and/or wherein the inhibitor is administered at a time before a peak level of the therapy is detectable in the blood of the subject following administration of the therapy. In some embodiments, steady state concentration refers to a time at which, or during, the concentration of an agent, such as a compound, remains stable or consistent in a subject with continued treatment or dosing. In some embodiments, a steady state concentration may include or be represented by a the concentration of an agent or compound plateauing with continued treatment or dosing of a subject. In some embodiments, the steady state concentration may describe a state where the amount of agent or compound administered on a dosing occasion is equivalent to the amount of the agent or compound leaving a subject's body between each dose, i.e. that rate in equals the rate out.

In some embodiments, methods can include administration of the inhibitor (e.g. venetoclax) prior to, simultaneously with, during, during the course of (including once and/or periodically during the course of), and/or subsequently to, the administration (e.g., initiation of administration) of the cytotoxic therapy, wherein the therapy specifically binds to an antigen associated with, expressed by or present on cells of the cancer, and wherein the inhibitor is administered in a dosing regimen sufficient to achieve a steady state concentration of the inhibitor within about 7 days prior to and about 14 days after initiation of administration of therapy.

In some embodiments, methods can include administration of the inhibitor (e.g. venetoclax) prior to, simultaneously with, during, during the course of (including once and/or periodically during the course of), and/or subsequently to, the administration (e.g., initiation of administration) of the cytotoxic therapy, wherein the therapy specifically binds to an antigen associated with, expressed by or present on cells of the cancer, and wherein the inhibitor is administered at a time before a peak level of the therapy is detectable in the blood of the subject following administration of the therapy.

In some embodiments, the inhibitor (e.g. venetoclax) has reached a steady state concentration (Css) when it has been administered for a duration of about four half-lives of the inhibitor. In some embodiments, “half-life” refers to the amount of time it takes for an initial concentration of an agent or compound in the plasma/blood of a subject or an initial total amount of an agent or compound in the body of a subject to be reduced by one half. In some embodiments, the inhibitor has reached a steady state concentration when it has been administered daily for about 2 days, about 3 days, about 4 days, or about 5 days. In some embodiments, the inhibitor has reached a steady state concentration when it has been administered daily for about 3 days. In some embodiments, a steady state concentration has been reached when the concentration of the inhibitor in the plasma of the subject or the total amount of the inhibitor in the subject's body is relatively stable with continued dosing.

In some embodiments, the inhibitor (e.g. venetoclax) is administered to the subject as a single agent therapy. In some embodiments, the inhibitor is administered to the subject as a single agent therapy (e.g. monotherapy) in combination with a cytotoxic therapy. In some embodiments administration as a monotherapy consists of a single type of treatment alone, to treat a disease or condition, except where otherwise provided. In some embodiments, an inhibitor of a prosurvival BCL2 family protein is provided as a monotherapy, such that no other treatment is provided to treat a disease or condition. In some embodiments, an inhibitor of a prosurvival BCL2 family protein is provided as a monotherapy with an immunotherapy or cell therapy, such that no other treatment is provided to treat a disease or condition beyond provision of (1) the inhibitor and (2) the immunotherapy or the cell therapy.

In some embodiments, the subject is not administered or has not received administration of rituximab and/or ibrutinib simultaneously with, during, or during the course of administration of the cytotoxic therapy. In some embodiments, the subject is not administered or has not received administration of rituximab and/or ibrutinib within about 7 days of initiation of administration of the cytotoxic therapy.

In some embodiments, the cytotoxic therapy is adoptive cell therapy. In some embodiments, the cell therapy is or comprises a tumor infiltrating lymphocytic (TIL) therapy, a transgenic TCR therapy or a recombinant-receptor expressing cell therapy (optionally T cell therapy), which optionally is a chimeric antigen receptor (CAR)-expressing cell therapy. In some embodiments, the therapy targets CD19 or is a B cell targeted therapy. In some embodiments, the cells and dosage regimens for administering the cells can include any as described in the following subsection B under “Administration of an Immunotherapy or Cell Therapy.”

In some embodiments, the cytotoxic therapy is capable of mediating and/or inducing a cell's intrinsic, mitochondrial-mediated apoptotic pathway. In some embodiments, the cytotoxic therapy is capable of mediating and/or inducing a cell's perforin- and or granzyme-mediated apoptotic pathway. In some embodiments, the cancer cells are resistant to the instrinsic apoptotic pathways. In some embodiments, the inhibitor sensitizes cells to apoptosis via the intrinsic apoptotic pathways. In some embodiments, the inhibitor sensitizes cells to apoptosis, as mediated or induced by the cytotoxic therapy. In some ways, the inhibitor lowers the apoptotic resistance of cells to the cytotoxic therapy.

In some embodiments, the cytotoxic therapy is an adoptive cell therapy (e.g. a T cell therapy). In some embodiments, the adoptive cell therapy comprises cells that are autologous to the subject. In some embodiments, the cells that are autologous to the subject are engineered to express a chimeric antigen receptor (CAR). In some embodiments, CAR-expressing autologous T cells are provided to the subject.

In some embodiments, the cytotoxic therapy, such as a T cell therapy (e.g. CAR-expressing T cells) or a T cell-engaging therapy, and inhibitor are provided as pharmaceutical compositions for administration to the subject. In some embodiments, the pharmaceutical compositions contain therapeutically effective amounts of one or both of the agents for combination therapy, e.g., T cells for adoptive cell therapy and an inhibitor as described. In some embodiments, the pharmaceutical compositions contain subtherapeutically effective amounts of one or both of the agents for combination therapy, e.g., T cells for adoptive cell therapy and an inhibitor as described. In some embodiments, the agents are formulated for administration in separate pharmaceutical compositions. In some embodiments, any of the pharmaceutical compositions provided herein can be formulated in dosage forms appropriate for each route of administration.

In some embodiments, the combination therapy, which includes administering the cytotoxic therapy (e.g. T cell therapy, including engineered cells, such as CAR-T cell therapy) and the inhibitor, is administered to a subject or patient having a cancer or at risk for cancer. In some aspects, the methods treat, e.g., ameliorate one or more symptom of, the disease or condition, such as by lessening tumor burden in a cancer expressing an antigen recognized by the immunotherapy or cell therapy, e.g. recognized by an engineered T cell.

In some embodiments, the disease or condition that is treated can be any in which expression of an antigen is associated with and/or involved in the etiology of a disease condition or disorder such as a cancer, e.g. causes, exacerbates or otherwise is involved in such disease, condition, or disorder. Exemplary diseases and conditions can include diseases or conditions associated with malignancy or transformation of cells (e.g. cancer). Exemplary antigens, which include antigens associated with various diseases and conditions that can be treated, include any of antigens described herein. In particular embodiments, the recombinant receptor expressed on engineered cells of a combination therapy, including a chimeric antigen receptor or transgenic TCR, specifically binds to an antigen associated with the cancer.

In some embodiments, the antigen associated with the disease or disorder such as cancer is selected from the group consisting of ROR1, B cell maturation antigen (BCMA), tEGFR, Her2, L1-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, EPHa2, ErbB2, 3, or 4, erbB dimers, EGFR vIII, FBP, FCRL5, FCRH5, fetal acethycholine e receptor, GD2, GD3, G Protein Coupled Receptor 5D (GPCR5D), HMW-MAA, IL-22R-alpha, IL-13R-alpha2, kdr, kappa light chain, Lewis Y, L1-cell adhesion molecule, (L1-CAM), Melanoma-associated antigen (MAGE)-A1, MAGE-A3, MAGE-A6, Preferentially expressed antigen of melanoma (PRAME), survivin, EGP2, EGP40, TAG72, B7-H6, IL-13 receptor a2 (IL-13Ra2), CA9, GD3, HMW-MAA, CD171, G250/CAIX, HLA-AI MAGE A1, HLA-A2 NY-ESO-1, PSCA, folate receptor-a, CD44v6, CD44v7/8, avb6 integrin, 8H9, NCAM, VEGF receptors, 5T4, Foetal AchR, NKG2D ligands, CD44v6, dual antigen, and an antigen associated with a universal tag, a cancer-testes antigen, mesothelin, MUC1, MUC16, PSCA, NKG2D Ligands, NY-ESO-1, MART-1, gp100, G Protein Coupled Receptor 5D (GPCR5D), oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, CCL-1, CD138, and a pathogen-specific antigen. In some embodiments, the antigen is associated with or is a universal tag.

In some embodiments, the disease or condition is a cancer or proliferative disease. In some embodiments, the cancer or proliferative disease is a tumor, such as a solid tumor, lymphoma, leukemia, blood tumor, metastatic tumor, or other cancer or tumor type. In some embodiments, the cancer is a hematologic malignancy. In some embodiments, the cancer is a B cell malignancy. In some embodiments, the cancer is a leukemia or a lymphoma. In some embodiments, the cancer is a leukemia. In some embodiments, the cancer is a leukemia such as a chronic lymphocytic leukemia (CLL), which can include small lymphocytic lymphoma (SLL). In some embodiments, the cancer is small lymphocytic lymphoma (SLL). In some embodiments, the cancer is chronic lymphocytic leukemia (CLL). In some embodiments, the cancer is a leukemia such as a acute lymphoblastic leukemia (ALL). In some embodiments, the cancer is a leukemia, such as acute myelogenous leukemia (AML). In some embodiments, the cancer is a leukemia, such as chronic myelogenous leukemia (CML). In some embodiments, the cancer is a myelodysplastic syndrome (MDS). In some embodiments, the cancer is multiple myeloma. In some embodiments, the cancer is a lymphoma. In some embodiments, the cancer is a lymphoma, such as a non-Hodgkin lymphoma (NHL). In some embodiments, the NHL is a subtype of NHL, such as diffuse large B-cell lymphoma (DLBCL). In some embodiments, the NHL is a subtype of NHL, such as SLL.

In some embodiments, the Eastern Cooperative Oncology Group (ECOG) performance status indicator can be used to assess or select subjects for treatment, e.g., subjects who have had poor performance from prior therapies (see, e.g., Oken et al. (1982) Am J Clin Oncol. 5:649-655). The ECOG Scale of Performance Status describes a patient's level of functioning in terms of their ability to care for themselves, daily activity, and physical ability (e.g., walking, working, etc.). In some embodiments, an ECOG performance status of 0 indicates that a subject can perform normal activity. In some aspects, subjects with an ECOG performance status of 1 exhibit some restriction in physical activity but the subject is fully ambulatory. In some aspects, patients with an ECOG performance status of 2 is more than 50% ambulatory. In some cases, the subject with an ECOG performance status of 2 may also be capable of selfcare; see e.g., Sørensen et al., (1993) Br J Cancer 67(4) 773-775. The criteria reflective of the ECOG performance status are described in Table 1 below:

TABLE 1
ECOG Performance Status Criteria

| Grade | ECOG performance status |
|---|---|
| 0 | Fully active, able to carry on all pre-disease performance without |
| | restriction |
| 1 | Restricted in physically strenuous activity but ambulatory and |
| | able to carry out work of a light or sedentary nature, e.g., light |
| | house work, office work |
| 2 | Ambulatory and capable of all selfcare but unable to carry out |
| | any work activities; up and about more than 50% of waking |
| | hours |
| 3 | Capable of only limited selfcare; confined to bed or chair more |
| | than 50% of waking hours |
| 4 | Completely disabled; cannot carry on any selfcare; totally |
| | confined to bed or chair |
| 5 | Dead |

Antigens targeted by the receptors (e.g. CAR) in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30. In some embodiments, the antigen is CD19.

In particular, among provided embodiments are methods of treating subjects with CLL or SLL. In some embodiments of the provided methods, the subjects have a high risk CLL or SLL. In some embodiments of the provided methods, the subjects have a high risk CLL. In some embodiments of the provided methods, the subjects have a high risk SLL. In some embodiments, the subjects are a heavily pretreated population of subjects with high-risk CLL (or SLL), all of whom have received one or more prior therapies including ibrutinib.

In some embodiments, subjects with CLL include those with CLL diagnosis with indication of treatment based on the International Workshop on Chronic Lymphocytic Leukemia (iwCLL) guidelines and clinical measurable disease (bone marrow involvement by >30% lymphocytes, peripheral blood lymphocytosis >5×10⁹/L, and/or measurable lymph nodes and/or hepatic or splenomegaly. In some embodiments, subjects with SLL include those with SLL diagnosis is based on lymphadenopathy and/or splenomegaly and <5×10⁹ CD19+ CD5+ clonal B lymphocytes/L [<5000/μL] in the peripheral blood at diagnosis with measurable disease defined as at least one lesion >1.5 cm in the greatest transverse diameter, and that is biopsy-proven SLL.

In some cases, existing treatment strategies for high risk and very high risk subjects may include fludarabine, cyclophosphamide, and rituximab (FCR), Bruton's tyrosine kinase (BTK) inhibitors (e.g. ibrutinib), and/or allogeneic stem cell transplantation. (Puiggros et al., BioMed Research International, Volume 2014 (2014), Article ID 435983). Many of the existing therapies include oral-targeted drugs, which have, for some patients with CLL, improved treatment outcomes. Nonetheless, some patients prove intolerant or resistant to therapy and/or fail to achieve complete response with undetectable MRD (uMRD). In some aspects, subjects who have progressive disease after treatment with available therapies have poor outcomes. For instance, in some aspects, subjects treated for CLL exhibit poor long-term outcomes. For example, in some cases, refractory (R/R) high-risk CLL subjects exhibit poor survival after ibrutinib discontinuation (Jain et al. (2015) Blood 125(13):2062-2067). There is a need for improved methods of treating CLL, and in some aspects, for those appropriate for treating high and/or very high-risk CLL and/or subjects having relapsed or become refractory to multiple prior therapies.

Chronic lymphocytic leukemia (CLL) is a generally a variable disease. Some subjects with CLL may survive without treatment while others may require immediate intervention. In some cases, subjects with CLL may be classified into groups that may inform disease prognosis and/or recommended treatment strategy. In some cases, these groups may be “low risk,” “intermediate risk,” “high risk,” and/or “very high risk” and patients may be classified as such depending on a number of factors including, but not limited to, genetic abnormalities and/or morphological or physical characteristics. In some embodiments, subjects treated in accord with the method are classified or identified based on the risk of CLL. In some embodiments, the subject is one that has high risk CLL.

In some embodiments, the provided methods and uses provide for or achieve improved or more durable responses or efficacy as compared to certain alternative methods, such as in particular groups of subjects treated, such as in patients with a leukemia, such as CLL or SLL, including those with high-risk disease. In some embodiments, the methods are advantageous by virtue of administering T cell therapy, such as a composition including cells for adoptive cell therapy, e.g., such as a CAR-expressing T cells, e.g. anti-CD19 CAR+ T cells, and an inhibitor of a prosurvival BCL2 protein (e.g. venetoclax). In some embodiments, the methods also include, prior to the T cell therapy, a lymphodepleting therapy, e.g. such as cyclophosphamide, fludarabine, or combinations thereof.

In some embodiments, the treated subjects include subjects that have relapsed following initial remission on ibrutinib or who are refractory or intolerant to treatment with ibrutinib. In particular embodiments, the treated subjects include subjects that have relapsed following remission or are refractory or intolerant to one or more further prior therapy in addition to ibrutinib, such as 1, 2, 3, 4, 5 or more prior therapies. In some embodiments, the subjects have relapsed or are refractory to both a prior treatment of ibrutinib and venetoclax. In some embodiments, subjects that are refractory to such treatment have progressed following one or more prior therapy. In some embodiments, subjects treated, including those treated with one or more prior therapies (e.g. ibrutinib and/or venetoclax) include those with a high-risk cytogenetics, including TP53 mutation, complex karyotype (i.e. at least three chromosomal alterations) and dell7(p).

In some embodiments of any of the provided methods, the subject has CLL or is suspected of having CLL; or the subject is identified or selected as having CLL. In some embodiments of any of the provided methods, the subject has CLL or is suspected of having CLL. In some embodiments of any of the provided methods, the subject is identified or selected as having CLL. In some embodiments, the CLL is relapsed or refractory CLL. In particular, CLL is generally considered to be incurable and patients often eventually relapse or become refractory to available therapies (Dighiero and Hamblin (2008) The Lancet, 371:1017-1029).

In some embodiments, the subject has SLL or is suspected of having SLL; or the subject is identified or selected as having SLL. In some embodiments, the subject has SLL or is suspected of having SLL. In some embodiments, the subject is identified or selected as having SLL. In some embodiments, the SLL is a relapsed or refractory SLL.

In some embodiments, prior to the administration of the dose of engineered T cells, the subject has been treated with one or more prior therapies for the CLL or SLL, other than the therapy, e.g. dose of cells expressing CAR, or a lymphodepleting therapy. In some embodiments, prior to the administration of the dose of engineered T cells, the subject has been treated with one or more prior therapies for the CLL, other than the therapy, e.g. dose of cells expressing CAR, or a lymphodepleting therapy. In some embodiments, prior to the administration of the dose of engineered T cells, the subject has been treated with one or more prior therapies for the SLL, other than the therapy, e.g. dose of cells expressing CAR, or a lymphodepleting therapy. In some embodiments, the one or more prior therapy comprises at least two prior therapies, optionally three, four, five, six, seven, eight, nine or more.

In some embodiments, at or immediately prior to the time of the administration of the dose of cells, the subject has relapsed following remission after treatment with, or become refractory to, failed and/or was intolerant to treatment with the one or more prior therapies for the CLL. In some embodiments, the subject has relapsed following remission after treatment with, or become refractory to, failed and/or was intolerant to treatment with two or more prior therapies. In some embodiments, at or immediately prior to the time of the administration of the dose of cells, the subject has relapsed following remission after treatment with, or become refractory to, failed and/or was intolerant to treatment with three or more prior therapies. In some embodiments, the prior therapies are selected from a kinase inhibitor, optionally an inhibitor of Bruton's tyrosine kinase (BTK), optionally ibrutinib; venetoclax; a combination therapy comprising fludarabine and rituximab; radiation therapy; and hematopoietic stem cell transplantation (HSCT). In some embodiments, the prior therapies comprise ibrutinib and/or venetoclax. In some embodiments, the prior therapies comprise ibrutinib and venetoclax. In some embodiments, the prior therapy comprises ibrutinib. In some embodiments, the prior therapy comprises venetoclax.

In some embodiments, the subject has relapsed following remission after treatment with, become refractory to failed treatment with and/or is intolerant to ibrutinib and/or venetoclax. In some embodiments, the subject has relapsed following remission after treatment with, become refractory to failed treatment with and/or is intolerant to ibrutinib. In some embodiments, the subject has relapsed following remission after treatment with, become refractory to failed treatment with and/or is intolerant to venetoclax. In some embodiments, the subject has relapsed following remission after treatment with, become refractory to, failed treatment with and/or is intolerant to ibrutinib and venetoclax.

In some embodiments a subject has been previously treated with an inhibitor of a prosurvival Bcl-2 family protein (e.g. venetoclax), prior to the provided methods is not intolerant to venetoclax. In some embodiments a subject has been treated with an inhibitor of a prosurvival Bcl-2 family protein (e.g. venetoclax) within 6 months prior to the provided methods, e.g. prior to receiving a lymphodepleting therapy or prior to receiving administration of a cytotoxic therapy. In some embodiments a subject has been treated with an inhibitor of a prosurvival Bcl-2 family protein (e.g. venetoclax) within 6 months prior to the provided methods and does not have progressive disease (PD). In some embodiments, the subject did not exhibit progressive disease during the treatment with the inhibitor. In some embodiments a subject has been treated with an inhibitor of a prosurvival Bcl-2 family protein (e.g. venetoclax) within 6 months prior to the provided methods and did not exhibit progressive disease during the treatment with the inhibitor.

In some embodiments, the subject is not intolerant to the inhibitor (e.g. venetoclax) at a time immediately following collection of autologous cells from the subject and/or immediately before administration of a lymphodepleting therapy to the subject. In some embodiments, the subject is not intolerant to the inhibitor (e.g. venetoclax) at the time of initiation of administration of the cytotoxic therapy. In some embodiments, the subject does not have a mutation in BCL2 at a time immediately following collection of autologous cells from the subject and/or immediately before administration of a lymphodepleting therapy to the subject. In some embodiments, the subject does not have a mutation in BCL2 at the time of initiation of administration of the cytotoxic therapy.

In some embodiments, the subject exhibits measurable disease or evaluable or measurable disease in peripheral blood (PB), bone marrow (BM), liver, or spleen at a time immediately following collection of autologous cells from the subject and/or immediately before administration of a lymphodepleting therapy to the subject. In some embodiments, at the time of initiation of the cytotoxic therapy, the subject exhibits measurable disease or evaluable or measurable disease in peripheral blood (PB), bone marrow (BM), liver, or spleen. In some embodiments, the measurable disease is or includes lymph nodes of greater than 1.0 centimeters in greatest transverse diameter (GTD). In some embodiments, the measurable disease is or includes lymph nodes of greater than 1.5 centimeters in greatest transverse diameter (GTD). In some embodiments, the measurable disease is or includes lymph nodes of greater than 2.0 centimeters in greatest transverse diameter (GTD).

In some embodiments, the subject exhibits minimum residual disease (MRD) in peripheral blood of greater than or equal to 10⁻⁴ at a time immediately following collection of autologous cells from the subject and/or immediately before administration of a lymphodepleting therapy to the subject. In some embodiments, at the time of initiation of the cytotoxic therapy, the subject exhibits minimum residual disease (MRD) in peripheral blood of greater than or equal to 10⁻⁴.

In some embodiments, the subject exhibits measurable disease or evaluable or measurable disease in peripheral blood (PB), bone marrow (BM), liver, or spleen and minimum residual disease (MRD) in peripheral blood of greater than or equal to 10⁻⁴, at a time immediately following collection of autologous cells from the subject and/or immediately before administration of a lymphodepleting therapy to the subject. In some embodiments, at the time of initiation of administration of the cytotoxic therapy, the subject exhibits measurable disease or evaluable or measurable disease in peripheral blood (PB), bone marrow (BM), liver, or spleen and minimum residual disease (MRD) in peripheral blood of greater than or equal to 10⁻⁴. In some embodiments, the measurable disease is or includes lymph nodes of greater than 1.0 centimeters in greatest transverse diameter (GTD). In some embodiments, the measurable disease is or includes lymph nodes of greater than 1.5 centimeters in greatest transverse diameter (GTD). In some embodiments, the measurable disease is or includes lymph nodes of greater than 2.0 centimeters in greatest transverse diameter (GTD).

In some embodiments, at or prior to the administration of the dose of cells: the subject is or has been identified as having one or more cytogenetic abnormalities, optionally associated with high-risk CLL, optionally selected from among: complex karyotype or cytogenetic abnormalities, del 17p, unmutated IGVH gene, and TP53 mutation; the subject is or has been identified as having high-risk CLL. In some embodiments, at or prior to initiation of administration of the cytotoxic therapy, the subject is or has been identified as having one or more cytogenetic abnormalities. In some embodiments, the one or more cytogenetic abnormalities are associated with high-risk CLL. In some embodiments, the one or more cytogenetic abnormalities selected from among complex karyotype or cytogenetic abnormalities, del 17p, unmutated IGVH gene, and TP53 mutation. In some embodiments, at or prior to initiation of administration of the cytotoxic therapy, the subject is or has been identified as having high-risk CLL.

In some embodiments, the subject is or has been identified as having an ECOG status of 0 or 1; and/or the subject does not have an ECOG status of >1. In some embodiments, at or immediately prior to the administration of the dose of engineered cells or the lymphodepleting therapy the subject does not have a Richter's transformation of the CLL or SLL.

In some embodiments, the methods involve treating a subject having a lymphoma or a leukemia, or a B cell malignancy, such as a large B cell lymphoma or a non-Hodgkin lymphoma (NHL).

In some embodiments, the provided methods involve treating a specific group or subset of subjects, e.g., subjects identified as having high-risk disease, e.g., high-risk NHL or a high-risk large B cell lymphoma. In some aspects, the methods treat subjects having a form of aggressive and/or poor prognosis B-cell non-Hodgkin lymphoma (NHL), such as NHL that has relapsed or is refractory (R/R) to standard therapy and/or has a poor prognosis.

In some embodiments, the subject has a B cell malignancy, such as a B cell lymphoma and/or a non-Hodgkin lymphoma (NHL). In some embodiments, the subject has a B cell malignancy, such as a large B cell lymphoma, e.g., a relapsed/refractory (R/R) large B cell lymphoma. In some embodiments, the subject has a large B cell lymphoma, such as a diffuse large B-cell lymphoma (DLBCL) (e.g., a DLBCL not otherwise specified (NOS; de novo or transformed from indolent) or other DLBCL). In some embodiments, the subject has a primary mediastinal B-cell lymphoma (PMBCL) or a follicular lymphoma, such as a follicular lymphoma grade 3B (FL3B). In some aspects, the B cell lymphoma is or includes diffuse large B-cell lymphoma (DLBCL), follicular lymphoma or PBMCL. In some aspects, the subject has a DLBCL that is a DLBCL, not otherwise specified (NOS). In some embodiments, the lymphoma, such as the DLBCL, is de novo. In some embodiments, the lymphoma, such as the DLBCL, is transformed from another indolent lymphoma. In some embodiments the lymphoma, such as the DLBCL, is transformed from a follicular lymphoma (tFL).

In some embodiments, the methods involve treating a subject that has an Eastern Cooperative Oncology Group Performance Status (ECOG) of 0-1 or 0-2. In some embodiments, subjects have Eastern Cooperative Oncology Group (ECOG) scores of between 0 and 2. In some embodiments, subjects are not excluded based on an ECOG score of 2. In some embodiments, the methods treat a poor-prognosis population or of DLBCL patients or subject thereof that generally responds poorly to therapies or particular reference therapies, such as one having one or more, such as two or three, chromosomal translocations (such as so-called “double-hit” or “triple-hit” lymphoma; having translocations MYC/8q24 loci, usually in combination with the t(14; 18) (q32; q21) bcl-2 gene or/and BCL6/3q27 chromosomal translocation; see, e.g., Xu et al. (2013) Int J Clin Exp Pathol. 6(4): 788-794), and/or one having relapsed, such as relapsed within 12 months, following administration of an autologous stem cell transplant (ASCT), and/or one having been deemed chemorefractory.

In some embodiments, the subject has been previously treated with a therapy or a therapeutic agent targeting the disease or condition, e.g., a large B cell lymphoma or an NHL, prior to administration of the therapy, e.g. cells expressing the recombinant receptor. In some embodiments, the subject has been previously treated with a hematopoietic stem cell transplantation (HSCT), e.g., allogeneic HSCT or autogeneic HSCT. In some embodiments, the subject has had poor prognosis after treatment with standard therapy and/or has failed one or more lines of previous therapy. In some embodiments, the subject has been treated or has previously received at least or at least about or about 1, 2, 3, or 4 other therapies for treating the disease or disorder, such as a large B cell lymphoma or NHL, other than a lymphodepleting therapy and/or the therapy, e.g. dose of cells expressing the antigen receptor. In some embodiments, the subject has been treated or has previously received a therapy that includes anthracycline, a CD20 targeted agent, and/or ibrutinib.

In some embodiments, the subject has been previously treated with chemotherapy or radiation therapy. In some aspects, the subject is refractory or non-responsive to the other therapy or therapeutic agent. In some embodiments, the subject has persistent or relapsed disease, e.g., following treatment with another therapy or therapeutic intervention, including chemotherapy or radiation.

In some embodiments, the subject is one that is eligible for a transplant, such as is eligible for a hematopoietic stem cell transplantation (HSCT), e.g., allogeneic HSCT. In some such embodiments, the subject has not previously received a transplant, despite being eligible, prior to administration of the therapy, such as cell therapy containing engineered cells (e.g. CAR-T cells) or a composition containing the cells to the subject as provided herein. In some such embodiments, the subject has previously received an allogeneic stem cell transplantation (SCT). In some such embodiments, the subject has not previously received an allogeneic stem cell transplantation (SCT). In some embodiments, subjects are not excluded based on prior allogeneic stem cell transplantation (SCT).

In some embodiments, the subject is one that is not eligible for a transplant, such as is not eligible for a hematopoietic stem cell transplantation (HSCT), e.g., allogeneic HSCT.

In some embodiments, the subject has a lymphoma that is associated with or involves central nervous system (CNS) involvement. In some embodiments, the subject has a lymphoma that is associated with or involves central nervous system (CNS) involvement, and the subject has been previously treated with an anticonvulsant, such as levetiracetam. In some embodiments, subjects are not excluded based on secondary central nervous system (CNS) involvement.

In some embodiments, subjects are not required to have a minimum absolute lymphocyte count (ALC) for apheresis.

In some embodiments, the methods include administration of cells to a subject selected or identified as having a high-risk large B cell lymphoma or a high-risk NHL. In some embodiments, the subject exhibits one or more cytogenetic abnormalities, such as associated with the B cell malignancy, such as a high-risk B cell lymphoma or a high-risk NHL. In some embodiments, the subject is selected or identified based on having a disease or condition characterized or determined to be aggressive NHL, diffuse large B cell lymphoma (DLBCL), primary mediastinal large B cell lymphoma (PMBCL), T cell/histocyte-rich large B cell lymphoma (TCHRBCL), Burkitt's lymphoma (BL), mantle cell lymphoma (MCL), and/or follicular lymphoma (FL). In particular embodiments, the subject to be treated using the methods provided herein include subjects with an aggressive large B cell lymphoma or an aggressive NHL, in particular, with diffuse large B-cell lymphoma (DLBCL), not otherwise specified (NOS; de novo or transformed from indolent), primary mediastinal B-cell lymphoma (PMBCL) or follicular lymphoma grade 3B (FL3B). In particular embodiments, the subject to be treated using the methods provided herein include subjects with DLBCL that is transformed from a follicular lymphoma (FL), or another indolent lymphoma. In some embodiments, the subject has DLBCL transformed from marginal zone lymphoma (MZL) or chronic lymphocytic leukemia (CLL) (e.g., Richter's). In some embodiments, a subject with transformation from CLL can exhibit Richter's syndrome (RS), defined as the transformation of CLL into an aggressive lymphoma, most commonly diffuse large B-cell lymphoma (DLBCL) (see, e.g., Parikh et al. Blood 2014 123:1647-1657). In some embodiments, the subject has mantle cell lymphoma (MCL). In some embodiments, the subjects has mantle cell lymphoma (MCL) that has failed (relapsed/refractory, R/R) after ≥1 prior lines of therapy. In some embodiments, the subject has confirmed cyclin D1 expressing MCL with R/R disease.

In some embodiments, the subject has poor performance status. In some aspects, the population to be treated includes subjects having an Eastern Cooperative Oncology Group Performance Status (ECOG) that is anywhere from 0-2. In other aspects of any of the embodiments, the subjects to be treated included ECOG 0-1 or do not include ECOG 2 subjects. In some aspects of any of the embodiments, the subjects to be treated have failed two or more prior therapies. In some embodiments, the subject does not have DLBCL transformed from marginal zone lymphoma (MZL) or chronic lymphocytic leukemia (CLL) (e.g., Richter's). In some embodiments, the subject has features that correlate with poor overall survival. In some embodiments, the subject has never achieved a complete response (CR), never received autologous stem cell transplant (ASCT), is refractory to 1 or more second line therapy, has primary refractory disease, and/or has an ECOG performance score of 2 or an ECOG score of between 0 and 1.

In some embodiments, the subject to be treated includes a group of subjects with diffuse large B-cell lymphoma (DLBCL), de novo or transformed from indolent lymphoma (not otherwise specified, NOS), primary mediastinal large b-cell lymphoma (PMBCL), and follicular lymphoma grade 3b (FL3B) after failure of 2 lines of therapy, and ECOG score of 0-2, and the subject may optionally have previously been treated with allogeneic stem cell transplantation (SCT). In some embodiments, the subject to be treated includes a group of subjects with diffuse large B-cell lymphoma (DLBCL), de novo or transformed from indolent lymphoma (not otherwise specified, NOS), primary mediastinal large b-cell lymphoma (PMBCL), and follicular lymphoma grade 3b (FL3B) after failure of 2 lines of therapy, and ECOG score of 0-2. In some embodiments, the subject may optionally have previously been treated with allogeneic stem cell transplantation (SCT). In some embodiments, the subject is not selected for treatment or excluded from treatment, if the subject has a poor performance status (e.g. ECOG 2) and/or has DLBCL transformed from marginal zone lymphomas (MZL) or chronic lymphocytic leukemia (CLL, Richter's). Thus, in some embodiments, the subject is selected for treatment if the subject has diffuse large B-cell lymphoma (DLBCL), de novo or transformed from indolent lymphoma (NOS), primary mediastinal large b-cell lymphoma (PMBCL), and follicular lymphoma grade 3b (FL3B) after failure of 2 lines of therapy, and ECOG score of 0 or 1, and the subject may optionally have previously been treated with allogeneic stem cell transplantation (SCT) but does not have DLBCL transformed from marginal zone lymphomas (MZL) or chronic lymphocytic leukemia (CLL, Richter's).

In some embodiments, the cancer is characterized by overexpression or aberrant expression of one or more prosurvival BCL2 family proteins. In some embodiments, the cancer is characterized by overexpression or aberrant expression of BCL2. In some cases, overexpression of BCL2 may be caused by the (14;18)(q32;q21) translocation. In some cases, overexpression of BCL2 may be caused by amplification of the gene encoding the BCL2 protein. In some embodiments, the cancer is characterized by overexpression or aberrant expression of BCLXL, MCL1, and/or BFL1. In some embodiments, the cancer is characterized by a mutation in one or more genes encoding for a prosurvival BCL2 family protein. In some embodiments, the cancer is characterized by a mutation in the gene encoding the BCL2 protein. In some cases, the mutation is (14;18)(q32;q21) translocation. In some embodiments, the cancer is resistant to treatment with an immunotherapy or cell therapy. In some embodiments, the cancer is resistant to treatment with a cell therapy, such as a CAR-expressing T cell therapy. In some embodiments, the cancer is resistant to treatment with a CD19-targeting CAR T cell therapy. In some embodiments, the inhibitor sensitizes a cancer to treatment with an immunotherapy or cell therapy. In some embodiments, the inhibitor sensitizes a cancer to treatment with a cell therapy, such as a CAR-expressing T cell therapy. In some embodiments, the inhibitor sensitizes a cancer to treatment with a CD19-targeting CAR T cell therapy.

In some embodiments the cancer or proliferative disease is a non-hematologic cancer. In some embodiments, the cancer is a solid cancer or tumor. In some embodiments, the cancer is a bladder cancer. In some embodiments, the cancer is a brain cancer. In some embodiments, the cancer is a breast cancer. In some embodiments, the cancer is a cervical cancer, In some embodiments, the cancer is a colorectal cancer. In some embodiments, the cancer is a endometrial cancer. In some embodiments, the cancer is a cancer of the head and/or neck. In some embodiments, the cancer is a lung cancer. In some embodiments, the cancer is an ovarian cancer. In some embodiments, the cancer is a pancreatic cancer. In some embodiments, the cancer is a renal cancer. In some embodiments, the cancer is a skin cancer.

In some embodiments, the combination therapy provided herein is carried out in a subject that has been previously treated with an inhibitor of a prosurvival BCL2 family protein, e.g. a BCL2 inhibitor, such as venetoclax, but in the absence of administration of a T cell therapy (e.g. CAR+ T cells) or T cell-engaging therapy. In some cases, after such previous treatment the subject is refractory to and/or develops resistance to, has relapsed following remission, has not achieved a CR after receiving such previous treatment for at least 6 months and/or exhibits an aggressive disease and/or high-risk features of the cancer. Thus, it is understood that the provided combination therapy can be carried out in a subject that has previously received administration of an inhibitor of a prosurvival BCL2 family protein, e.g. a BCL2 inhibitor, such as venetoclax. Reference to timing of administration of an inhibitor in the present disclosure refers to timing of its administration relative to the immunotherapy or immunotherapeutic agent, e.g. T cell therapy (e.g. CAR+ T cells) or T cell-engaging therapy, in accord with the provided combination therapy methods and does not exclude the possibility that the subject has additionally previously been administered an inhibitor of a prosurvival BCL2 family protein, e.g. a BCL2 inhibitor, such as venetoclax.

For the prevention or treatment of disease, the appropriate dosage of inhibitor of a prosurvival BCL2 family protein and/or immunotherapy, such as a T cell therapy (e.g. CAR-expressing T cells) or a T cell-engaging therapy, may depend on the type of disease to be treated, the particular inhibitor, cells and/or recombinant receptors expressed on the cells, the severity and course of the disease, route of administration, whether the inhibitor and/or the immunotherapy, e.g., T cell therapy, are administered for preventive or therapeutic purposes, previous therapy, frequency of administration, the subject's clinical history and response to the cells, and the discretion of the attending physician. The compositions and cells are in some embodiments suitably administered to the subject at one time or over a series of treatments. Exemplary dosage regimens and schedules for the provided combination therapy are described.

In some embodiments, the immunotherapy, e.g. T cell therapy, and the inhibitor of a prosurvival BCL2 family protein are administered as part of a further combination treatment, which can be administered simultaneously with or sequentially to, in any order, another therapeutic intervention. In some contexts, the immunotherapy, e.g. engineered T cells, such as CAR-expressing T cells, are co-administered with another therapy sufficiently close in time such that the immunotherapy enhances the effect of one or more additional therapeutic agents, or vice versa. In some embodiments, the cells are administered prior to the one or more additional therapeutic agents. In some embodiments, the immunotherapy, e.g. engineered T cells, such as CAR-expressing T cells, are administered after the one or more additional therapeutic agents. In some embodiments, the combination therapy methods further include a lymphodepleting therapy, such as administration of a chemotherapeutic agent. In some embodiments, the combination therapy further comprises administering another therapeutic agent, such as an anti-cancer agent, a checkpoint inhibitor, or another immune modulating agent. Uses include uses of the combination therapies in such methods and treatments, and uses of such compositions in the preparation of a medicament in order to carry out such combination therapy methods. In some embodiments, the methods and uses thereby treat the disease or condition or disorder, such as a cancer or proliferative disease, in the subject.

In some embodiments, the immunotherapy, e.g. T cell therapy, and the inhibitor of a prosurvival BCL2 family protein are administered without any other combination treatment. In some embodiments, the immunotherapy, e.g. T cell therapy, and the inhibitor of a prosurvival BCL2 family protein are administered without any other combination treatment, such as ibrutinib and/or rituximab.

Prior to, during or following administration of the immunotherapy (e.g. T cell therapy, such as CAR-T cell therapy) and/or an inhibitor of a prosurvival BCL2 family protein, the biological activity of the immunotherapy, e.g. the biological activity of the engineered cell populations, in some embodiments is measured, e.g., by any of a number of known methods. Parameters to assess include the ability of the engineered cells to destroy target cells, persistence and other measures of T cell activity, such as measured using any suitable method known in the art, such as assays described further below in Section III below. In some embodiments, the biological activity of the cells, e.g., T cells administered for the T cell based therapy, is measured by assaying cytotoxic cell killing, expression and/or secretion of one or more cytokines, proliferation or expansion, such as upon restimulation with antigen. In some aspects the biological activity is measured by assessing the disease burden and/or clinical outcome, such as reduction in tumor burden or load. In some aspects the biological activity is measured by assessing the presence of neutropenia in a subject. In some embodiments, administration of one or both agents of the combination therapy and/or any repeated administration of the therapy, can be determined based on the results of the assays before, during, during the course of or after administration of one or both agents of the combination therapy.

In some embodiments, the combined effect of the inhibitor in combination with the cell therapy can be synergistic compared to treatments involving only the inhibitor or monotherapy with the cell therapy. In some embodiments, the combined effect of a subtherapeutically effective amount of the inhibitor in combination with the cell therapy can be synergistic compared to treatments involving only the inhibitor or monotherapy with the cell therapy. In some embodiments, the combined effect of a subtherapeutically effective amount of the inhibitor in combination with a subtherapeutically effective amount of the cell therapy can be synergistic compared to treatments involving only the inhibitor or monotherapy with the cell therapy. For example, in some embodiments, the provided methods, compositions and articles of manufacture herein result in an increase or an improvement in a desired therapeutic effect, such as an increased or an improvement in the reduction or inhibition of one or more symptoms associated with cancer.

In some embodiments, the inhibitor increases the expansion, proliferation, or cytotoxicity of the engineered T cells, such as CAR T cells. In some embodiments, the increase in expansion, proliferation, or cytotoxicity is observed in vivo upon administration to a subject. In some embodiments, the increase in the number of engineered T cells, e.g. CAR-T cells, is increased by greater than or greater than about 1.2-fold, 1.5-fold, 2.0-fold, 3.0-fold, 4.0-fold, 5.0-fold, 6.0-fold, 7.0-fold, 8.0-fold, 9.0-fold, 10.0 fold or more. In some embodiments, the increase in the cytotoxicity of the engineered T cells, e.g. CAR-T cells, against cancer cells is increased by greater than or greater than about 1.2-fold, 1.5-fold, 2.0-fold, 3.0-fold, 4.0-fold, 5.0-fold, 6.0-fold, 7.0-fold, 8.0-fold, 9.0-fold, 10.0 fold or more.

A. Administration of an Inhibitor of a Prosurvival BCL2 Family Protein

The provided combination therapy methods, combinations, kits and uses involve administration of an inhibitor of a prosurvival BCL2 family protein (e.g. venetoclax), which can be administered prior to, subsequently to, during, simultaneously or near simultaneously, sequentially and/or intermittently with administration of the immunotherapy or cell therapy, e.g., administration of T cells expressing a chimeric antigen receptor (CAR), and/or the administration of which can begin prior to administration of the T cell therapy and continue until the initiation of administration of the T cell therapy or after the initiation of administration of the T cell therapy. In some embodiments, a prosurvival BCL2 family protein is a BCL2 family protein that has the ability to promote survival and/or to mitigate pro-apoptotic signaling. In some embodiments, a prosurvival BCL2 family protein is a BCL2 family protein that is anti-apoptotic. In some embodiments, a prosurvival BCL2 family protein is a BCL2 family protein that has the ability to promote the survival of cancer cells. In some embodiments, a prosurvival BCL2 family protein is a BCL2 family protein that has the ability to dampen pro-apoptotic signaling of cancer cells.

In some embodiments, the inhibitor in the combination therapy is an inhibitor of a prosurvival BCL2 family protein (e.g. venetoclax), which, in some cases, are involved in the regulation and promulgation of anti-apoptotic (prosurvival) signaling in a cell, such as via a cell's intrinsic apoptotic pathway. In some cases, prosurvival BCL2 family proteins are involved in apoptotic signaling, including antiapoptotic (prosurvival) signaling via a cell's intrinsic apoptosis pathway, via mitochondria. In some cases, prosurvival BCL2 family proteins are involved in antiapoptotic (prosurvival) signaling via granzyme and/or perforin-mediated apoptosis in the mitochondria. In some embodiments, the inhibitor of a prosurvival BCL2 family protein (e.g. venetoclax) is an inhibitor of one or more prosurvival proteins of the BCL2 family, including BCL2, B-cell lymphoma extra-large (BCLXL), BCL2 related protein A1 (BFL1), BCL2-like protein 2 (BCLW), BCL2-like protein 10 (BCLB), and induced myeloid leukemia cell differentiation protein (MCL1).

In some embodiments, the inhibitor (e.g. venetoclax) interacts with a BCL2 homology (BH) domain. In some embodiments, the inhibitor is a mimetic of BH3. In some embodiments, the inhibitor is a BH3 mimetic that occupies a BH3-binding domain. In some embodiments, the inhibitor is a BH3 mimetic that occupies a BH3 binding domain and/or displaces pro-apoptotic BH3-only proteins from BCL2. In some embodiments, the inhibitor block or reduces the interaction between BCL2 and proteins having a BH3 domain. In some embodiments, the inhibitor occupies a BH3 binding domain to block or reduce heterodimerization of a prosurvival BCL2 family protein, such as BCL2 or BCLXL, with a pro-apoptotic BCL2 family protein, such as BAD, BAX, or BAK. In some embodiments, the inhibitor reduces or blocks the phosphorylation of BCL2.

In some embodiments, the inhibitor (e.g. venetoclax) reduces prosurvival (antiapoptotic) signaling. In some cases, the reduction of prosurvival signaling lowers the apoptotic threshold of a cell. In some cases, the apoptosis is achieved by the cell's intrinsic, mitochondrial-mediated apoptosis pathway. In some cases, the reduction of prosurvival signaling and/or lowering of the apoptotic threshold sensitizes a cell to cell death and/or increases cell death. In some cases, the reduction of prosurvival signaling and/or lowering of the apoptotic threshold sensitizes a cell to cell death by one or more other agents and/or increases cell death by one or more other agents. In some cases, the reduction of prosurvival signaling and/or lowering of the apoptotic threshold is achieved by inducing BAX and/or BAK-dependent apoptosis. In some cases, the reduction of prosurvival signaling and/or lowering of the apoptotic threshold sensitizes a cell to cell death by a cytotoxic therapy, such as an immunotherapy or cell therapy (e.g. CAR-expressing T cell therapy). In some cases, the reduction of prosurvival signaling and/or lowering of the apoptotic threshold sensitizes a cell to cell death pathways targeted CAR T cells, including perforin and granzyme-mediated pathways (Benmabarek et al. (2019) Intl. J. Mol. Sci. (20):1283).

In some embodiments, the inhibitor of a prosurvival BCL2 family protein (e.g. venetoclax) is a selective BCL2 inhibitor. In some embodiments, a selective BCL2 inhibitor is a compound or agent, such as an inhibitor of a prosurvival BCL2 family protein, that is capable of being provided at a dosing regimen (e.g. dose and/or duration) that reduces or blocks BCL2 activity and/or signaling to a greater extent than that of other prosurvival BCL2 family proteins (e.g. BCLXL, BCLW, BCLB, MCL1). In some cases, a selective BCL2 inhibitor reduces or blocks the activity of BCL2 signaling and/or activity when provided at a dosing regimen, but does not reduce or block the signaling and/or activity of other prosurvival BCL2 family proteins when provided at the same dosing regimen. In some cases, selective BCL2 inhibitors exert minimal or no effects on the activity and/or signaling of other prosurvival BCL2 family proteins, when provided at a dosing regimen.

In some embodiments, the inhibitor of a prosurvival BCL2 family protein is a nonselective BCL2 inhibitor. In some embodiments, a nonselective BCL2 inhibitor is a compound or agent, such as an inhibitor of a prosurvival BCL2 family protein, that reduces or blocks the activity of more than one prosurvival BCL2 family protein. In some cases, a nonselective BCL2 inhibitor is a compound or agent, such as an inhibitor of a prosurvival BCL2 family protein, that is capable of being provided at a dosing regimen (e.g. dose and/or duration) that reduces or blocks the activity and/or signaling of a prosurvival BCL2 family protein, e.g. BCL2 and additionally reduces or blocks the activity and/or signaling of one or more other prosurvival BCL2 family proteins (e.g. BCLXL, BCLW, BCLB, MCL1). In some cases, a nonselective BCL2 inhibitor reduces or blocks the activity and/or signaling of a prosurvival BCL2 family protein (e.g. BCL2) when provided at a dosing regimen, and also reduces or blocks the signaling and/or activity of one or more other prosurvival BCL2 family proteins (e.g. BCLXL, BCLW, BCLB, MCL1) when provided at the same dosing regimen.

In some embodiments, the inhibitor inhibits BCL2 with a half-maximal inhibitory concentration (IC₅₀) of less than or less than about 1000 nM, less than or less than about 900 nM, less than or less than about 800 nM, less than or less than about 700 nM, less than or less than about 600 nM, less than or less than about 500 nM, less than or less than about 400 nM, less than or less than about 300 nM, less than or less than about 200 nM, less than or less than about 100 nM, less than or less than about 90 nM, less than or less than about 80 nM, less than or less than about 70 nM, less than or less than about 60 nM, less than or less than about 50 nM, less than or less than about 40 nM, less than or less than about 30 nM, less than or less than about 20 nM, less than or less than about 10 nM, less than or less than about 9 nM, less than or less than about 8 nM, less than or less than about 7 nM, less than or less than about 6 nM, less than or less than about 5 nM, less than or less than about 4 nM, less than or less than about 3 nM, less than or less than about 2 nM, less than or less than about 1 nM, less than or less than about 0.9 nM, less than or less than about 0.8 nM, less than or less than about 0.7 nM, less than or less than about 0.6 nM, less than or less than about 0.5 nM, less than or less than about 0.4 nM, less than or less than about 0.3 nM, less than or less than about 0.2 nM, less than or less than about 0.1 nM, or less than or less than about 0.01 nM. In some embodiments, the inhibitor inhibits one or more other prosurvival BCL2 family proteins, such as BCLXL, BCLW, BCLB, and/or MCL1 with a half-maximal inhibitory concentration (IC₅₀) of less than or less than about 1000 nM, less than or less than about 900 nM, less than or less than about 800 nM, less than or less than about 700 nM, less than or less than about 600 nM, less than or less than about 500 nM, less than or less than about 400 nM, less than or less than about 300 nM, less than or less than about 200 nM, less than or less than about 100 nM, less than or less than about 90 nM, less than or less than about 80 nM, less than or less than about 70 nM, less than or less than about 60 nM, less than or less than about 50 nM, less than or less than about 40 nM, less than or less than about 30 nM, less than or less than about 20 nM, less than or less than about 10 nM, less than or less than about 9 nM, less than or less than about 8 nM, less than or less than about 7 nM, less than or less than about 6 nM, less than or less than about 5 nM, less than or less than about 4 nM, less than or less than about 3 nM, less than or less than about 2 nM, less than or less than about 1 nM, less than or less than about 0.9 nM, less than or less than about 0.8 nM, less than or less than about 0.7 nM, less than or less than about 0.6 nM, less than or less than about 0.5 nM, less than or less than about 0.4 nM, less than or less than about 0.3 nM, less than or less than about 0.2 nM, less than or less than about 0.1 nM, or less than or less than about 0.01 nM.

In some embodiments, the inhibition constant (Ki) of the inhibitor for BCL2 is lower than the inhibition constant (Ki) of the inhibitor for one or more other prosurvival BCL2 family proteins, such as BCLXL, BCLW, BCLB, and/or MCL1. In some embodiments, the inhibition constant (Ki) of the inhibitor for BCL2 is at least 10 times lower, at least 100 times lower, at least 1,000 times lower, at least 5,000 times lower, at least 10,000 times lower, or at least 20,000 times lower than the inhibition constant (Ki) of the inhibitor for one or more other prosurvival BCL2 family proteins, such as BCLXL, BCLW, BCLB, and/or MCL1. In some embodiments, the inhibition constant (Ki) of the inhibitor for BCL2 is at least 1,000 times lower than the inhibition constant (Ki) of the inhibitor for one or more other prosurvival BCL2 family proteins, such as BCLXL, BCLW, BCLB, and/or MCL1. In some embodiments, the inhibition constant (Ki) of the inhibitor for BCL2 is at least 5,000 times lower than the inhibition constant (Ki) of the inhibitor for one or more other prosurvival BCL2 family proteins, such as BCLXL, BCLW, BCLB, and/or MCL1. In some embodiments, the inhibition constant (Ki) of the inhibitor for BCL2 is at least 10,000 times lower than the inhibition constant (Ki) of the inhibitor for one or more other prosurvival BCL2 family proteins, such as BCLXL, BCLW, BCLB, and/or MCL1. In some embodiments, the inhibition constant (Ki) of the inhibitor for BCL2 is at least 20,000 times lower than the inhibition constant (Ki) of the inhibitor for one or more other prosurvival BCL2 family proteins, such as BCLXL, BCLW, BCLB, and/or MCL1. In some embodiments, the inhibition constant (Ki) of the inhibitor for BCL2 is at least 1,000 times lower than the inhibition constant (Ki) of the inhibitor for BCLXL. In some embodiments, the inhibition constant (Ki) of the inhibitor for BCL2 is at least 4,000 times lower than the inhibition constant (Ki) of the inhibitor for BCLXL. In some embodiments, the inhibition constant (Ki) of the inhibitor for BCL2 is at least 20,000 times lower than the inhibition constant (Ki) of the inhibitor for BCLW.

In some embodiments, the inhibition constant (Ki) of the inhibitor for BCL2 is less than about 10 μM. In In some embodiments, the inhibition constant (Ki) of the inhibitor for BCL2 is less than about 1 μM. In some embodiments, the inhibition constant (Ki) of the inhibitor for BCL2 is less than about 0.1 μM. In some embodiments, the inhibition constant (Ki) of the inhibitor for BCL2 is less than about 10 nM. In some embodiments, the inhibition constant (Ki) of the inhibitor for BCL2 is less than about 1.0 nM. In some embodiments, the inhibition constant (Ki) of the inhibitor for BCL2 is less than about 0.1 nM. In some embodiments, the inhibition constant (Ki) of the inhibitor for BCL2 is less than about 0.01 nM. In some embodiments, the inhibition constant (Ki) of the inhibitor for one or more other prosurvival BCL2 family proteins, such as BCLXL, BCLW, BCLB, and/or MCL1 is less than about 10 μM. In In some embodiments, the inhibition constant (Ki) of the inhibitor for one or more other prosurvival BCL2 family proteins, such as BCLXL, BCLW, BCLB, and/or MCL1 is less than about 1 μM. In some embodiments, the inhibition constant (Ki) of the inhibitor one or more other prosurvival BCL2 family proteins, such as BCLXL, BCLW, BCLB, and/or MCL1 is less than about 0.1 μM. In some embodiments, the inhibition constant (Ki) of the inhibitor for one or more other prosurvival BCL2 family proteins, such as BCLXL, BCLW, BCLB, and/or MCL1 is less than about 10 nM. In some embodiments, the inhibition constant (Ki) of the inhibitor for one or more other prosurvival BCL2 family proteins, such as BCLXL, BCLW, BCLB, and/or MCL1 is less than about 1.0 nM. In some embodiments, the inhibition constant (Ki) of the inhibitor for one or more other prosurvival BCL2 family proteins, such as BCLXL, BCLW, BCLB, and/or MCL1 is less than about 0.1 nM. In some embodiments, the inhibition constant (Ki) of the inhibitor for one or more other prosurvival BCL2 family proteins, such as BCLXL, BCLW, BCLB, and/or MCL1 is less than about 0.01 nM.

In some embodiments, the IC50, Kd and/or Ki is measured or determined using an in vitro assay. Assays to assess or quantitate or measure activity of protein tyrosine kinase inhibitors as described are known in the art. Such assays can be conducted in vitro and include assays to assess the ability of an agent to inhibit a specific biological or biochemical function. In some embodiments. In some embodiments, kinase activity studies can be performed. Protein tyrosine kinases catalyze the transfer of the terminal phosphate group from adenosine triphosphate (ATP) to the hydroxyl group of a tyrosine residue of the kinase itself or another protein substrate. In some embodiments, kinase activity can be measured by incubating the kinase with the substrate (e.g., inhibitor) in the presence of ATP. In some embodiments, measurement of the phosphorylated substrate by a specific kinase can be assessed by several reporter systems including colorimetric, radioactive, and fluorometric detection. (Johnson, S. A. & T. Hunter (2005) Nat. Methods 2:17.) In some embodiments, inhibitors can be assessed for their affinity for a particular kinase or kinases, such as by using competition ligand binding assays (Ma et al., Expert Opin Drug Discov. 2008 June; 3(6):607-621) From these assays, the half-maximal inhibitory concentration (IC₅₀) can be calculated. IC₅₀ is the concentration that reduces a biological or biochemical response or function by 50% of its maximum. In some cases, such as in kinase activity studies, IC₅₀ is the concentration of the compound that is required to inhibit the target kinase activity by 50%. In some cases, the dissociation constant (Kd) and/or the inhibition constant (Ki values) can be determined additionally or alternatively. IC₅₀ and Kd can be calculated by any number of means known in the art. The inhibition constant (Ki values) can be calculated from the IC50 and Kd values according to the Cheng-Prusoff equation: Ki=IC₅₀/(1+L/Kd), where L is the concentration of the inhibitor (Biochem Pharmacol 22: 3099-3108, 1973). Ki is the concentration of unlabeled inhibitor that would cause occupancy of 50% of the binding sites present in the absence of ligand or other competitors.

In some embodiments, the inhibitor is a small molecule.

In some embodiments, the inhibitor is an inhibitor of a prosurvival BCL2 family protein, including but not limited to those described in U.S. Pat. Nos. 9,174,982, 8,546,399, 7,030,115, 7,390,799, 7,709,467, 8,624,027, 7,906,505, 6,720,338, published PCT application WO 13/096060, published PCT application WO 02/097053, published US application US 2016/0220573, U.S. Pat. No. 7,354,928, published US application 2015/0056186, and published PCT application WO 05/049594, which are each incorporated by reference in their entireties.

In some embodiments, the inhibitor inhibits MCL1, such as maritoclax. In some embodiments, the inhibitor inhibits BCL2, BCLXL, and BCLW, such as navitoclax. In some embodiments, the inhibitor inhibits BCL2, such as venetoclax.

In some embodiments, the inhibitor inhibits or reduces the activity of BCL2, BCLXL, BCLW, BCLB, BFL1, and/or MCL1. In some cases, the inhibitor inhibits or reduces the activity of MCL1, such as maritoclax. In some cases, the inhibitor induces proteasomal degradation of MCL1. In some cases, the inhibitor induces accumulation of MCL1. In some cases the inhibitor is maritoclax. In some cases, the inhibitor has the structure

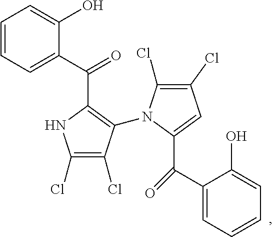 [SMILES: O=C(c1ccccc1O)c1[nH]c(Cl)c(Cl)c1-n1c(C(=O)c2ccccc2O)cc(Cl)c1Cl]

or a pharmaceutically acceptable salt, solvate, hydrate, stereoisomer, tautomer or racemic mixtures thereof, including and compositions thereof.

In some embodiments, the inhibitor inhibits or reduces the activity of BCL2, BCLXL, and BCLW, such as navitoclax. In some cases, the inhibitor is navitoclax. In some cases, the inhibitor has the structure

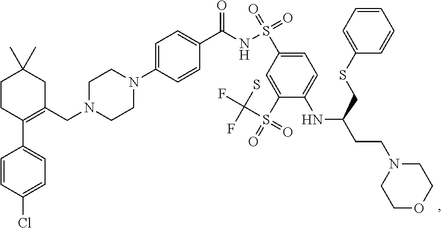 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)S)c4)cc3)CC2)C1]

or a pharmaceutically acceptable salt, solvate, hydrate, stereoisomer, tautomer or racemic mixtures thereof, including and compositions thereof, for the treatment of subjects with cancer.

In some embodiments, the inhibitor inhibits or reduces the activity of BCL2, such as venetoclax. In some cases, the inhibitor is venetoclax. In some cases, the inhibitor has the structure

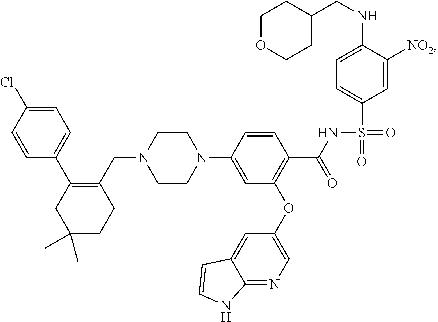 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

or a pharmaceutically acceptable salt, solvate, hydrate, stereoisomer, tautomer or racemic mixtures thereof, including and compositions thereof.

Exemplary prosurvival BCL2 family protein inhibitors include, but are not limited to venetoclax (ABT-199), navitoclax (ABT-263), ABT-737, AT-101/GDC-0199 (Gossypol), apogossypol, TW-37, G3139 (Genasense), GX15-070 (obatoclax), sabutoclax, HA14-1, antimycin A, BH3I-1, YC137, maritoclax (marinopyyrole A), clitocine, UMI-77, WEHI-539, and 544563.

1. Compostions and Formulations

In some embodiments of the combination therapy methods, combinations, kits and uses provided herein, the combination therapy can be administered in one or more compositions, e.g., a pharmaceutical composition containing an inhibitor of a prosurvival BCL2 family protein, e.g. a BCL2 inhibitor, and/or the cytotoxic therapy, e.g., T cell therapy.

In some embodiments, the composition, e.g., a pharmaceutical composition containing a prosurvival BCL2 family protein inhibitor, such as a BCL2 inhibitor, e.g., venetoclax, can include carriers such as a diluent, adjuvant, excipient, or vehicle with which a prosurvival BCL2 family protein inhibitor, such as a BCL2 inhibitor, e.g., venetoclax, and/or the cells are administered. Examples of suitable pharmaceutical carriers are described in “Remington's Pharmaceutical Sciences” by E. W. Martin. Such compositions will contain a therapeutically effective amount of a prosurvival BCL2 family protein inhibitor, such as a BCL2 inhibitor, e.g., venetoclax, generally in purified form, together with a suitable amount of carrier so as to provide the form for proper administration to the patient. Such pharmaceutical carriers can be sterile liquids, such as water and oils, including those of petroleum, animal, vegetable or synthetic origin, such as peanut oil, soybean oil, mineral oil, and sesame oil. Saline solutions and aqueous dextrose and glycerol solutions also can be employed as liquid carriers, particularly for injectable solutions. The pharmaceutical compositions can contain any one or more of a diluents(s), adjuvant(s), antiadherent(s), binder(s), coating(s), filler(s), flavor(s), color(s), lubricant(s), glidant(s), preservative(s), detergent(s), sorbent(s), emulsifying agent(s), pharmaceutical excipient(s), pH buffering agent(s), or sweetener(s) and a combination thereof. In some embodiments, the pharmaceutical composition can be liquid, solid, a lyophilized powder, in gel form, and/or combination thereof. In some aspects, the choice of carrier is determined in part by the particular inhibitor and/or by the method of administration.

Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), stabilizers and/or preservatives. The compositions containing a prosurvival BCL2 family protein inhibitor, such as a BCL2 inhibitor, e.g., venetoclax, can also be lyophilized.

In some embodiments, the pharmaceutical compositions can be formulated for administration by any route known to those of skill in the art including intramuscular, intravenous, intradermal, intralesional, intraperitoneal injection, subcutaneous, intratumoral, epidural, nasal, oral, vaginal, rectal, topical, local, otic, inhalational, buccal (e.g., sublingual), and transdermal administration or any route. In some embodiments, other modes of administration also are contemplated. In some embodiments, the administration is by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, administration is by parenteral, intrapulmonary, and intranasal, and, if desired for local treatment, intralesional administration. Parenteral infusions include intramuscular, intravenous, intraarterial, intraperitoneal, or subcutaneous administration.

In some embodiments, compositions also can be administered with other biologically active agents, either sequentially, intermittently or in the same composition. In some embodiments, administration also can include controlled release systems including controlled release formulations and device controlled release, such as by means of a pump. In some embodiments, the administration is oral.

In some embodiments, a prosurvival BCL2 family protein inhibitor, such as a BCL2 inhibitor, e.g., venetoclax, are typically formulated and administered in unit dosage forms or multiple dosage forms. Each unit dose contains a predetermined quantity of therapeutically active a prosurvival BCL2 family protein inhibitor, e.g., venetoclax, sufficient to produce the desired therapeutic effect, in association with the required pharmaceutical carrier, vehicle or diluent. In some embodiments, unit dosage forms, include, but are not limited to, tablets, capsules, pills, powders, granules, sterile parenteral solutions or suspensions, and oral solutions or suspensions, and oil water emulsions containing suitable quantities of a prosurvival BCL2 family protein inhibitor, e.g., venetoclax. Unit dose forms can be contained ampoules and syringes or individually packaged tablets or capsules. Unit dose forms can be administered in fractions or multiples thereof. In some embodiments, a multiple dose form is a plurality of identical unit dosage forms packaged in a single container to be administered in segregated unit dose form. Examples of multiple dose forms include vials, bottles of tablets or capsules or bottles of pints or gallons.

2. Dosing

In some embodiments, the provided combination therapy method involves administering to the subject a prosurvival BCL2 family protein inhibitor, such as a BCL2 inhibitor, e.g., venetoclax, and a cytotoxic therapy, such as an immunotherapy or cell therapy (e.g. CAR-expressing T cells).

In some embodiments, the provided combination therapy methods involve initiating administration of a prosurvival BCL2 family protein inhibitor, such as a BCL2 inhibitor, e.g., venetoclax, prior to, subsequently to, during, during the course of, simultaneously, near simultaneously, sequentially, concurrently and/or intermittently with the initiation of the cytotoxic therapy, such as a T cell therapy (e.g., CAR-expressing T cells). In some embodiments, the provided embodiments involve initiating the administration of a prosurvival BCL2 family protein inhibitor, such as a BCL2 inhibitor, e.g., venetoclax, prior to administration of the therapy and continue until the initiation of administration of the therapy or after the initiation of administration of the therapy. In some embodiments, “concurrently” indicates that the administration of the inhibitor and that of the cytotoxic therapy in a combination therapy overlap with each other, in that at least one dose of the inhibitor overlaps with one dose of the cytotoxic therapy, and/or that the initiation of administration of the inhibitor occurs at the same time (e.g. same day and/or simultaneously) as the initiation of administration of the cytotoxic therapy, and/or that the administration of the inhibitor and the initiation of the cytotoxic therapy are within about three to about four half-lives of the inhibitor (e.g. 2 to 5 days). In some embodiments, “concurrently” refers to an administration regimen such that the inhibitor is present in a subject at a steady state concentration (Css) at the same time that the cytotoxic therapy has been administered to a subject but has not yet achieved peak expansion.

In some embodiments, the provided combination therapy methods involve initiating administration of a prosurvival BCL2 family protein inhibitor, such as a BCL2 inhibitor, e.g., venetoclax at a time between about 7 days prior to or about 14 days after the initiation of the cytotoxic therapy, such as a T cell therapy (e.g., CAR-expressing T cells). In some embodiments, the provided combination therapy methods involve initiating administration of a prosurvival BCL2 family protein inhibitor, such as a BCL2 inhibitor, e.g., venetoclax at a time between about 3 days prior to or about 14 days after the initiation of the cytotoxic therapy, such as a T cell therapy (e.g., CAR-expressing T cells). In some embodiments, the provided combination therapy methods involve initiating administration of a prosurvival BCL2 family protein inhibitor, such as a BCL2 inhibitor, e.g., venetoclax at a time between about 1 day prior to or about 14 days after the initiation of the cytotoxic therapy, such as a T cell therapy (e.g., CAR-expressing T cells).

In some embodiments, the provided combination therapy methods involve initiating administration of a prosurvival BCL2 family protein inhibitor, such as a BCL2 inhibitor, e.g., venetoclax at a time between about 1 day prior to or about 8 days after the initiation of the cytotoxic therapy, such as a T cell therapy (e.g., CAR-expressing T cells).

In some embodiments, the provided combination therapy methods involve initiating administration of a prosurvival BCL2 family protein inhibitor, such as a BCL2 inhibitor, e.g., venetoclax concurrently (e.g. same day or simultaneously) with the initiation of the cytotoxic therapy, such as a T cell therapy (e.g., CAR-expressing T cells).

In some embodiments, the provided combination therapy methods involve initiating administration of a prosurvival BCL2 family protein inhibitor, such as a BCL2 inhibitor, e.g., venetoclax within no more than 14 days after initiation of administration of the cytotoxic therapy, such as a T cell therapy (e.g., CAR-expressing T cells).

In some embodiments, the provided combination therapy methods involve initiating administration of a prosurvival BCL2 family protein inhibitor, such as a BCL2 inhibitor, e.g., venetoclax within about 7 days after initiation of administration of the cytotoxic therapy, such as a T cell therapy (e.g., CAR-expressing T cells). In some embodiments, the prosurvival BCL2 family protein inhibitor, e.g., venetoclax is administered in multiple doses in regular intervals prior to, during, during the course of, and/or after the period of administration of the cytotoxic therapy (e.g. T cell therapy, such as CAR-T cell therapy).

In some embodiments, the method involves initiating administration of the prosurvival BCL2 family protein inhibitor, such as a BCL2 inhibitor, e.g., venetoclax, prior to administration of the cytotoxic therapy, optionally within 7 days before initiation of administration of the therapy, optionally within 3 days before initiation of administration of the therapy, optionally within 1 day before initiation of administration of the therapy.

In some embodiments, the method involves initiating administration of the prosurvival BCL2 family protein inhibitor, e.g., venetoclax, after administration of the cytotoxic therapy (e.g. CAR T cell therapy). In some embodiments, the method involves initiating administration of the prosurvival BCL2 family protein inhibitor, e.g., venetoclax, after administration of the cytotoxic therapy (e.g. CAR T cell therapy), optionally within 14 days after initiation of administration of the cytotoxic therapy, optionally within 11 days after initiation of administration of the cytotoxic therapy, optionally within 7 days after initiation of administration of the cytotoxic therapy, optionally within 3 days after initiation of administration of the cytotoxic therapy, optionally within 1 day after initiation of administration of the cytotoxic therapy.

In some embodiments, the method involves initiating administration of the prosurvival BCL2 family protein inhibitor, e.g., venetoclax, after administration of the cytotoxic therapy (e.g. CAR T cell therapy) within 14 days after initiation of administration of the cytotoxic therapy. In some embodiments, the method involves initiating administration of the prosurvival BCL2 family protein inhibitor, e.g., venetoclax, after administration of the cytotoxic therapy (e.g. CAR T cell therapy) within 11 days after initiation of administration of the cytotoxic therapy. In some embodiments, the method involves initiating administration of the prosurvival BCL2 family protein inhibitor, e.g., venetoclax, after administration of the cytotoxic therapy (e.g. CAR T cell therapy) within 7 days after initiation of administration of the cytotoxic therapy. In some embodiments, the method involves initiating administration of the prosurvival BCL2 family protein inhibitor, e.g., venetoclax, after administration of the cytotoxic therapy (e.g. CAR T cell therapy) within 3 days after initiation of administration of the cytotoxic therapy. In some embodiments, the method involves initiating administration of the prosurvival BCL2 family protein inhibitor, e.g., venetoclax, after administration of the cytotoxic therapy (e.g. CAR T cell therapy) within 2 days after initiation of administration of the cytotoxic therapy. In some embodiments, the method involves initiating administration of the prosurvival BCL2 family protein inhibitor, e.g., venetoclax, after administration of the cytotoxic therapy (e.g. CAR T cell therapy) within 1 day after initiation of administration of the cytotoxic therapy.

In some aspects, initiation of the administration of a prosurvival BCL2 family protein inhibitor, such as a BCL2 inhibitor, e.g., venetoclax, is between about 1 day and about 7 days after administration of the cytotoxic therapy (e.g. CAR T cell therapy). In some aspects, initiation of the administration of a prosurvival BCL2 family protein inhibitor, such as a BCL2 inhibitor, e.g., venetoclax, is about 7 days after administration of the cytotoxic therapy (e.g. CAR T cell therapy). In some aspects, initiation of the administration of a prosurvival BCL2 family protein inhibitor, such as a BCL2 inhibitor, e.g., venetoclax, is within 7 days after administration of the cytotoxic therapy (e.g. CAR T cell therapy). In some aspects, initiation of the administration of a prosurvival BCL2 family protein inhibitor, such as a BCL2 inhibitor, e.g., venetoclax, is at or about 1 day after administration of the cytotoxic therapy (e.g. CAR T cell therapy). In some aspects, initiation of the administration of a prosurvival BCL2 family protein inhibitor, such as a BCL2 inhibitor, e.g., venetoclax, is at or about 2 day after administration of the cytotoxic therapy (e.g. CAR T cell therapy). In some aspects, initiation of the administration of a prosurvival BCL2 family protein inhibitor, such as a BCL2 inhibitor, e.g., venetoclax, is at or about 3 days after administration of the cytotoxic therapy (e.g. CAR T cell therapy). In some aspects, initiation of the administration of a prosurvival BCL2 family protein inhibitor, such as a BCL2 inhibitor, e.g., venetoclax, is at or about 4 days after administration of the cytotoxic therapy (e.g. CAR T cell therapy). In some aspects, initiation of the administration of a prosurvival BCL2 family protein inhibitor, such as a BCL2 inhibitor, e.g., venetoclax, is at or about 5 days after administration of the cytotoxic therapy (e.g. CAR T cell therapy). In some aspects, initiation of the administration of a prosurvival BCL2 family protein inhibitor, such as a BCL2 inhibitor, e.g., venetoclax, is at or about 6 days after administration of the cytotoxic therapy (e.g. CAR T cell therapy). In some aspects, initiation of the administration of a prosurvival BCL2 family protein inhibitor, such as a BCL2 inhibitor, e.g., venetoclax, is at or about 7 days after administration of the cytotoxic therapy (e.g. CAR T cell therapy).

In some embodiments, methods can include initiation of administration of the inhibitor (e.g. venetoclax) after initiation of administration of the cytotoxic therapy, at or after the peak of activation-induced cell death (AICD) of cells of the cytotoxic therapy (e.g. CAR T cells). In some embodiments, methods can include initiation of administration of the inhibitor (e.g. venetoclax) after initiation of administration of the cytotoxic therapy, at the peak of activation-induced cell death (AICD) of cells of the cytotoxic therapy (e.g. CAR T cells). In some embodiments, methods can include initiation of administration of the inhibitor (e.g. venetoclax) after initiation of administration of the cytotoxic therapy, after the peak of activation-induced cell death (AICD) of cells of the cytotoxic therapy (e.g. CAR T cells).

In some embodiments, the dosage schedule comprises continuing to administer the prosurvival BCL2 family protein inhibitor, e.g., venetoclax, prior to and after initiation of the cytotoxic therapy. In some embodiments, the dosage schedule comprises initiating administration of the prosurvival BCL2 family protein inhibitor, e.g., venetoclax, within about 7 days prior to and within about 14 days after initiation of the cytotoxic therapy. In some embodiments, the dosage schedule comprises initiating administration of the prosurvival BCL2 family protein inhibitor, e.g., venetoclax, within about 3 days prior to and within about 14 days after initiation of the cytotoxic therapy. In some embodiments, the dosage schedule comprises initiating administration of the prosurvival BCL2 family protein inhibitor, e.g., venetoclax, within about 1 day prior to and within about 14 days after initiation of the cytotoxic therapy. In some embodiments, the dosage schedule comprises initiating administration of the prosurvival BCL2 family protein inhibitor, e.g., venetoclax, within about 14 days after initiation of the cytotoxic therapy. In some embodiments, the dosage schedule comprises initiating administration of the prosurvival BCL2 family protein inhibitor, e.g., venetoclax, within about 11 days after initiation of the cytotoxic therapy. In some embodiments, the dosage schedule comprises initiating administration of the prosurvival BCL2 family protein inhibitor, e.g., venetoclax, within about 7 days after initiation of the cytotoxic therapy. In some embodiments, the dosage schedule comprises initiating administration of the prosurvival BCL2 family protein inhibitor, e.g., venetoclax, within about 6 days after initiation of the cytotoxic therapy. In some embodiments, the dosage schedule comprises initiating administration of the prosurvival BCL2 family protein inhibitor, e.g., venetoclax, within about 5 days after initiation of the cytotoxic therapy. In some embodiments, the dosage schedule comprises initiating administration of the prosurvival BCL2 family protein inhibitor, e.g., venetoclax, within about 4 days after initiation of the cytotoxic therapy. In some embodiments, the dosage schedule comprises initiating administration of the prosurvival BCL2 family protein inhibitor, e.g., venetoclax, within about 3 days after initiation of the cytotoxic therapy. In some embodiments, the dosage schedule comprises initiating administration of the prosurvival BCL2 family protein inhibitor, e.g., venetoclax, within about 2 days after initiation of the cytotoxic therapy. In some embodiments, the dosage schedule comprises initiating administration of the prosurvival BCL2 family protein inhibitor, e.g., venetoclax, within about 1 day after initiation of the cytotoxic therapy.

In some embodiments, the dosage schedule comprises initiating administration of the prosurvival BCL2 family protein inhibitor, e.g., venetoclax, concurrently with initiation of the cytotoxic therapy. In some embodiments, the dosage schedule comprises administering the prosurvival BCL2 family protein inhibitor, e.g., venetoclax, simultaneously with the administration of the cytotoxic therapy.

In some embodiments, the administration of the prosurvival BCL2 family protein inhibitor, e.g., venetoclax, is continued and/or further administered over a period of time, e.g., until a determined time point or until a particular outcome is achieved.

In some cases, the combination therapy involves administration of the inhibitor (e.g. venetoclax) up to or about 3 months after the subject as received administration of the therapy, e.g. cytotoxic therapy. In some cases, the combination therapy involves administration of the inhibitor (e.g. venetoclax) for at least 3 months after the subject as received administration of the therapy, e.g. cytotoxic therapy. In some cases, the combination therapy involves administration of the inhibitor (e.g. venetoclax) up to or about 6 months after the subject as received administration of the therapy, e.g. cytotoxic therapy. In some cases, the combination therapy involves administration of the inhibitor f(e.g. venetoclax) or at least about 6 months after the subject as received administration of the therapy, e.g. cytotoxic therapy. In some cases, the combination therapy involves administration of the inhibitor (e.g. venetoclax) up to or about 12 months after the subject as received administration of the therapy, e.g. cytotoxic therapy. In some cases, the combination therapy involves administration of the inhibitor (e.g. venetoclax) for at least about 12 months after the subject as received administration of the therapy, e.g. cytotoxic therapy. In some cases, the combination therapy involves administration of the inhibitor (e.g. venetoclax) up to or about 24 months after the subject as received administration of the therapy, e.g. cytotoxic therapy. In some cases, the combination therapy involves administration of the inhibitor (e.g. venetoclax) for at least about 24 months after the subject as received administration of the therapy, e.g. cytotoxic therapy.

In some cases, the inhibitor is discontinued about 3 months after administration of the cytotoxic therapy, if the subject exhibits a desired response (e.g. complete response). In some cases, the inhibitor is discontinued about 3 months after administration of the cytotoxic therapy. In some cases, the inhibitor is discontinued about 6 months after administration of the cytotoxic therapy. In some cases, the inhibitor is discontinued about 12 months after administration of the cytotoxic therapy. In some cases, the inhibitor is discontinued about 24 months after administration of the cytotoxic therapy. In some cases, the outcome is a desired therapeutic response, as described below and in Section III.

In some embodiments, the administration of the prosurvival BCL2 family protein inhibitor, e.g., venetoclax, is continued and/or further administered until at least about or about 3 months after initiation of administration of the cytotoxic therapy (e.g. CAR T cell therapy). In some embodiments, the administration of the prosurvival BCL2 family protein inhibitor, e.g., venetoclax, is continued and/or further administered until at least about or about 6 months after initiation of administration of the cytotoxic therapy (e.g. CAR T cell therapy). In some embodiments, the administration of the prosurvival BCL2 family protein inhibitor, e.g., venetoclax, is continued and/or further administered until at least about or about 9 months after initiation of administration of the cytotoxic therapy (e.g. CAR T cell therapy). In some embodiments, the administration of the prosurvival BCL2 family protein inhibitor, e.g., venetoclax, is continued and/or further administered until at least about or about 12 months after initiation of administration of the cytotoxic therapy (e.g. CAR T cell therapy). In some embodiments, the administration of the prosurvival BCL2 family protein inhibitor, e.g., venetoclax, is continued and/or further administered until at least about or about 24 months after initiation of administration of the cytotoxic therapy (e.g. CAR T cell therapy). In some embodiments, the administration of the prosurvival BCL2 family protein inhibitor, e.g., venetoclax, is continued and/or further administered for more than 3 months after initiation of administration of the cytotoxic therapy (e.g. CAR T cell therapy) and until a desired therapeutic outcome (e.g. a complete response) is achieved. In some cases, the desired therapeutic (e.g. a complete response) outcome is achieved within 6 of initiation of administration of the CAR T cell therapy. In some cases, the desired therapeutic (e.g. a complete response) outcome is achieved within 12 of initiation of administration of the CAR T cell therapy. In some cases, the desired therapeutic (e.g. a complete response) outcome is achieved within 24 of initiation of administration of the CAR T cell therapy.

In some embodiments, the prosurvival BCL2 family protein inhibitor, e.g., venetoclax, is administered multiple times in multiple doses. In some embodiments, prosurvival BCL2 family protein inhibitor, e.g., venetoclax, is administered multiple times over a period of time, e.g., until a determined time point or until a particular outcome is achieved. In some cases, the outcome is a desired therapeutic response, as described below and in Section III.

Desired therapeutic results, such as for the treatment of cancer, include but are not limited to a reduction of tumor size, bulk, metastasis, percentage of blasts in the bone marrow or molecularly detectable B cell malignancy and/or an improvement in prognosis or survival or other symptom associated with tumor burden. Desired therapeutic results, such as for the treatment of cancer, may additionally include an ability of the inhibitor to induce apoptosis in cancer cells and/or an increase in apoptosis of cancer cells. Methods for identifying and determining whether a desired therapeutic result for the treatment of cancer is achieved include but are not limited to any as described in Section III.

In some embodiments, the prosurvival BCL2 family protein inhibitor, e.g., venetoclax, is administered six times daily, five times daily, four times daily, three times daily, twice daily, once daily, every other day, every three days, twice weekly, once weekly or once monthly prior to, concurrently with, and/or subsequently to initiation of administration of the cytotoxic therapy (e.g. T cell therapy, such as CAR-T cell therapy). In some embodiments, the prosurvival BCL2 family protein inhibitor, e.g., venetoclax, is administered once daily prior to, concurrently with, and/or or subsequently to initiation of administration of the cytotoxic therapy (e.g. T cell therapy, such as CAR-T cell therapy). In some embodiments, the prosurvival BCL2 family protein inhibitor, e.g., venetoclax, is administered once daily subsequent to initiation of administration of the cytotoxic therapy (e.g. T cell therapy, such as CAR-T cell therapy). In some embodiments, the prosurvival BCL2 family protein inhibitor, e.g., venetoclax is administered in multiple doses in regular intervals prior to, during, during the course of, and/or after the period of administration of the cytotoxic therapy (e.g. T cell therapy, such as CAR-T cell therapy). In some embodiments, the prosurvival BCL2 family protein inhibitor, e.g., venetoclax, is administered in one or more doses in regular intervals prior to the administration of the cytotoxic therapy (e.g. T cell therapy, such as CAR-T cell therapy). In some embodiments, the prosurvival BCL2 family protein inhibitor, e.g., venetoclax, is administered in one or more doses in regular intervals after the administration of the cytotoxic therapy (e.g. T cell therapy, such as CAR-T cell therapy). In some embodiments, one or more of the doses of the prosurvival BCL2 family protein inhibitor, e.g., venetoclax, can occur simultaneously with the administration of a dose of the cytotoxic therapy (e.g. T cell therapy, such as CAR-T cell therapy). In some embodiments, such methods can include administration of the inhibitor prior to, simultaneously with, during, during the course of (including once and/or periodically during the course of), and/or subsequently to, the administration (e.g., initiation of administration) of the cytotoxic therapy (e.g. CAR-expressing T cells). In some embodiments, the administrations can involve sequential or intermittent administrations of the inhibitor and/or the cytotoxic therapy, e.g. T cell therapy. In some embodiments, the administrations can involve sequential or intermittent once daily administrations of the inhibitor and/or the immunotherapy or cell therapy, e.g. T cell therapy.

In some embodiments, initiation of the administration of the cytotoxic therapy is at a time when the prosurvival BCL2 family protein inhibitor has reached a steady state concentration (Css). In some embodiments, the methods of administration of the prosurvival BCL2 family protein inhibitor result in the inhibitor reaching a steady state concentration (Css) within about 7 days prior to and about 14 days after initiation of the administration of the cytotoxic therapy. In some embodiments, the methods of administration of the prosurvival BCL2 family protein inhibitor result in the inhibitor reaching a steady state concentration (Css) within about 3 days prior to and about 14 days after initiation of the administration of the cytotoxic therapy. In some embodiments, the methods of administration of the prosurvival BCL2 family protein inhibitor result in the inhibitor reaching a steady state concentration (Css) within about 1 day prior to and about 14 days after initiation of the administration of the cytotoxic therapy. In some embodiments, the methods of administration of the prosurvival BCL2 family protein inhibitor result in the inhibitor reaching a steady state concentration (Css) within about 14 days after initiation of the administration of the cytotoxic therapy. In some embodiments, the methods of administration of the prosurvival BCL2 family protein inhibitor result in the inhibitor reaching a steady state concentration (Css) within about 7 days after initiation of the administration of the cytotoxic therapy. In some embodiments, the methods of administration of the prosurvival BCL2 family protein inhibitor result in the inhibitor reaching a steady state concentration (Css) within about 3 days after initiation of the administration of the cytotoxic therapy. In some embodiments, the methods of administration of the prosurvival BCL2 family protein inhibitor result in the inhibitor reaching a steady state concentration (Css) within about 1 day after initiation of the administration of the cytotoxic therapy. In some embodiments, the inhibitor has reached a steady state concentration (Css) at a time before a peak level of the cytotoxic therapy is detectable in the blood of the subject following administration of the cytotoxic therapy. In some embodiments, the inhibitor has reached a steady state concentration when it has been administered for a duration of about four half-lives of the inhibitor. In some embodiments, the half-life of the inhibitor is between about 10 hours and about 30 hours. In some embodiments, the half-life of the inhibitor is between about 14 hours and about 26 hours. In some embodiments, the half-life of the inhibitor is between about 14 hours and about 18 hours. In some embodiments, the half-life of the inhibitor is between about 16 hours and about 19 hours. In some embodiments, the inhibitor has reached a steady state concentration when it has been administered daily for about 2 days, about 3 days, about 4 days, or about 5 days. In some embodiments, the inhibitor has reached a steady state concentration when it has been administered daily for about 3 days. In some embodiments, the inhibitor has reached a steady state concentration when it has been administered daily for about 4 days. In some embodiments, a steady state concentration has been reached when the concentration of the inhibitor in the plasma of the subject or the total amount of the inhibitor in the subject's body is relatively stable with continued dosing.

In some embodiments, the dose, frequency, duration, timing and/or order of administration of the pro survival BCL2 family protein inhibitor, such as a BCL2 inhibitor, e.g., venetoclax, is determined, based on particular thresholds or criteria of results of the screening step and/or assessment of treatment outcomes described herein, such as in Section III.

In some embodiments, the methods involve administering the cytotoxic therapy to a subject that has been previously administered a therapeutically effective amount or one or more doses of the prosurvival BCL2 family protein inhibitor, e.g., venetoclax. In some embodiments, the methods involve administering the cytotoxic therapy to a subject that has been previously administered a subtherapeutically effective amount or one or more doses of the prosurvival BCL2 family protein inhibitor, e.g., venetoclax. In some embodiments, the prosurvival BCL2 family protein inhibitor, e.g., venetoclax, is administered to a subject before administering a dose of cells expressing a recombinant receptor to the subject. In some embodiments, one or more doses of the prosurvival BCL2 family protein inhibitor, e.g., venetoclax, is administered at the same time as the initiation of the administration of the dose of cells. In some embodiments, the prosurvival BCL2 family protein inhibitor, e.g., venetoclax, is administered after the initiation of the administration of the dose of cells. In some embodiments, the inhibitor is administered at a sufficient time prior to immunotherapy or cell therapy so that the therapeutic effect of the combination therapy is increased. In some embodiments, the method involves administering the prosurvival BCL2 family protein inhibitor, such as a BCL2 inhibitor, e.g., venetoclax, prior to administration of a T cell therapy. In some embodiments, the method involves administering the prosurvival BCL2 family protein inhibitor, e.g., venetoclax, after administration of a T cell therapy. In some embodiments, the method involves initiating the administration of the pro survival BCL2 family protein inhibitor, such as a BCL2 inhibitor, e.g., venetoclax, prior to initiation of administration of a T cell therapy. In some embodiments, the prosurvival BCL2 family protein inhibitor, e.g., venetoclax, is continued and/or further administered after a discontinuation or a pause during a lymphodepletion therapy, such as until or after initiation of the cell therapy. In some embodiments, the prosurvival BCL2 family protein inhibitor, e.g., venetoclax, is discontinued prior to a lymphodepletion therapy for a period of time equal to between about three and about four half-lives of the inhibitor. In some embodiments, the method involves continuing administration of the prosurvival BCL2 family protein inhibitor, such as a BCL2 inhibitor, e.g., venetoclax. In some embodiments, continuing and/or further administration of the prosurvival BCL2 family protein inhibitor, e.g., venetoclax, involves administration of multiple doses of the prosurvival BCL2 family protein inhibitor, e.g., venetoclax. In some embodiments, the prosurvival BCL2 family protein inhibitor, e.g., venetoclax, is not continued or further administered after initiation of the cell therapy. In some embodiments, the dosage schedule comprises administering the prosurvival BCL2 family protein inhibitor prior to and after initiation of the cell therapy. In some embodiments, the dosage schedule comprises administering the prosurvival BCL2 family protein inhibitor, e.g., venetoclax, simultaneously with the administration of the cell therapy. In some cases, the cell therapy is a T cell therapy, e.g. CAR-expressing T cells.

In some cases, the inhibitor is administered at a therapeutically effective amount. In some cases, a therapeutically effective amount achieves a desired therapeutic result, such as for treatment of a disease, condition, or disorder, and/or pharmacokinetic or pharmacodynamic effect of the treatment. Desired therapeutic results, such as for the treatment of cancer, include but are not limited to a reduction of tumor size, bulk, metastasis, percentage of blasts in the bone marrow or molecularly detectable B cell malignancy and/or an improvement in prognosis or survival or other symptom associated with tumor burden. Desired therapeutic results, such as for the treatment of cancer, may additionally include an ability of the inhibitor to induce apoptosis in cancer cells and/or an increase in apoptosis of cancer cells. In some cases, a therapeutically effective amount results in neutropenia, such as mild, moderate, or severe neutropenia. In some cases, mild neutropenia is defined as an absolute neutrophil count of 1,000 to 1,5000/μL. In some cases, moderate neutropenia is defined as an absolute neutrophil count of 500 to 1,000/μL. In some cases, severe neutropenia is defined as an absolute neutrophil count of fewer than 500/μL. Methods for identifying and determining whether a desired therapeutic result for the treatment of cancer is achieved include but are not limited to any as described in Section III. In some cases, the inhibitor is administered to a subject at a dose of between at or about 20 mg and at or about 800 mg, between at or about 20 mg and at or 400 mg, between at or about 20 mg and at or about 350 mg, between at or about 20 mg and at or about 300 mg, between at or about 20 mg and at or about 250 mg, between at or about 20 mg and at or about 200 mg, between at or about 20 mg and at or about 150 mg, between at or about 20 mg and at or about 100 mg, between at or about 20 mg and at or about 50 mg, between at or about 20 mg and at or about 40 mg, between at or about 40 mg and at or about 800 mg, between at or about 40 mg and at or 400 mg, between at or about 40 mg and at or about 350 mg, between at or about 40 mg and at or about 300 mg, between at or about 40 mg and at or about 250 mg, between at or about 40 mg and at or about 200 mg, between at or about 40 mg and at or about 150 mg, between at or about 40 mg and at or about 100 mg, between at or about 40 mg and at or about 50 mg, between at or about 50 mg and at or about 800 mg, between at or about 50 mg and at or about 400 mg, between at or about 50 mg and at or about 350 mg, between at or about 50 mg and at or about 300 mg, between at or about 50 mg and at or about 250 mg, between at or about 50 mg and at or about 200 mg, between at or about 50 mg and at or about 150 mg, between at or about 50 mg and at or about 100 mg, between at or about 100 mg and at or about 800 mg, between at or about 100 mg and at or about 400 mg, between at or about 100 mg and at or about 350 mg, between at or about 100 mg and at or about 300 mg, between at or about 100 mg and at or about 250 mg, between at or about 100 mg and at or about 200 mg, between at or about 100 mg and at or about 150 mg, between at or about 150 mg and at or about 800 mg, between at or about 150 mg and at or about 400 mg, between at or about 150 mg and at or about 350 mg, between at or about 150 mg and at or about 300 mg, between at or about 150 mg and at or about 250 mg, between at or about 150 mg and at or about 200 mg, between at or about 200 mg and at or about 800 mg, between at or about 200 mg and at or about 400 mg, between at or about 200 mg and at or about 350 mg, between at or about 200 mg and at or about 300 mg, between at or about 200 mg and at or about 250 mg, between at or about 250 mg and at or about 300 mg, between at or about 300 mg and at or about 350 mg, between at or about 300 mg and at or about 400 mg, between at or about 300 mg and at or about 800 mg, between at or about 350 mg and at or about 400 mg, and between at or about 350 mg and at or about 800 mg, each inclusive. In some cases, the inhibitor is administered to a subject at a dose of about 20 milligrams per day. In some cases, the inhibitor is administered to a subject at a dose of about 40 milligrams per day. In some cases, the inhibitor is administered to a subject at a dose of about 50 milligrams per day. In some cases, the inhibitor is administered to a subject at a dose of about 100 milligrams per day. In some cases, the inhibitor is administered to a subject at a dose of about 150 milligrams per day. In some cases, the inhibitor is administered to a subject at a dose of about 200 milligrams per day. In some cases, the inhibitor is administered to a subject at a dose of about 250 milligrams per day. In some cases, the inhibitor is administered to a subject at a dose of about 300 milligrams per day. In some cases, the inhibitor is administered to a subject at a dose of about 350 milligrams per day. In some cases, the inhibitor is administered to a subject at a dose of about 400 milligrams per day. In some cases, the inhibitor is administered to a subject at a dose of about 800 milligrams per day.

In some cases the inhibitor is administered at a steady dose (e.g. once daily dose) to a subject. In some cases, the steady dose is between about 20 mg per day and 1200 mg per day, between about 20 mg per day and 800 mg per day, between about 20 mg per day and 400 mg per day, between about 20 mg per day and 350 mg per day, between about 20 mg per day and 300 mg per day, between about 20 mg per day and 250 mg per day, between about 20 mg per day and 200 mg per day, between about 20 mg per day and 100 mg per day, between about 20 mg per day and 50 mg per day, between about 20 mg per day and 40 mg per day, between about 40 mg per day and 1200 mg per day, between about 40 mg per day and 800 mg per day, between about 40 mg per day and 400 mg per day, between about 40 mg per day and 350 mg per day, between about 40 mg per day and 300 mg per day, between about 40 mg per day and 250 mg per day, between about 40 mg per day and 200 mg per day, between about 40 mg per day and 100 mg per day, between about 40 mg per day and 50 mg per day, between about 50 mg and 1200 mg per day, between about 50 mg and 800 mg per day, between about 50 mg and 400 mg per day, between about 50 mg and 350 mg per day, between about 50 mg and 300 mg per day, between about 50 mg and 250 mg per day, between about 50 mg and 200 mg per day, between about 50 mg and 150 mg per day, between about 50 mg and 100 mg per day, between about 100 mg and 1200 mg per day, between about 100 mg and 800 mg per day, between about 100 mg and 400 mg per day, between about 100 mg and 350 mg per day, between about 100 mg and 300 mg per day, between about 100 mg and 250 mg per day, between about 100 mg and 200 mg per day, between about 100 mg and 150 mg per day, between about 150 mg and 1200 mg per day, between about 150 mg and 800 mg per day, between about 150 mg and 400 mg per day, between about 150 mg and 350 mg per day, between about 150 mg and 300 mg per day, between about 150 mg and 250 mg per day, between about 150 mg and 200 mg per day, between about 200 mg and 1200 mg per day, between about 200 mg and 800 mg per day, between about 200 mg and 400 mg per day, between about 200 mg and 350 mg per day, between about 200 mg and 300 mg per day, between about 200 mg and 250 mg per day, between about 250 mg and 1200 mg per day, between about 250 mg and 800 mg per day, between about 250 mg and 400 mg per day, between about 250 mg and 350 mg per day, between about 250 mg and 300 mg per day, between about 300 mg and 1200 mg per day, between about 300 mg and 800 mg per day, between about 300 mg and 400 mg per day, between about 300 mg and 350 mg per day, between about 350 mg and 1200 mg per day, between about 350 mg and 800 mg per day, between about 350 mg and 400 mg, between about 400 mg and 1200 mg per day, between about 400 mg and 800 mg per day, or between about 800 and 1200 mg per day, each inclusive. In some cases, the steady dose is 20 mg. In some cases, the steady dose is 40 mg. In some cases, the steady dose is 50 mg. In some cases, the steady dose is 100 mg. In some cases, the steady dose is 150 mg. In some cases, the steady dose is 200 mg. In some cases, the steady dose is 250 mg. In some cases, the steady dose is 300 mg. In some cases, the steady dose is 325 mg. In some cases, the steady dose is 350 mg. In some cases, the steady dose is 400 mg. In some cases, the steady dose is 500 mg. In some cases, the steady dose is 600 mg. In some cases, the steady dose is 700 mg. In some cases, the steady dose is 800 mg.

In some cases, the steady dose (e.g. once daily dose) of the inhibitor is administered to a subject for between one week and twenty-four months, between one week and twelve year, between one week and 6 months, between one week and 3 months, between one week and 2 months, between one week and six weeks, between one week and four weeks, between one week and three weeks, between one week and two weeks. In some cases, the steady dose (e.g. once daily dose) of the inhibitor is administered to a subject for one week. In some cases, the steady dose (e.g. once daily dose) of the inhibitor is administered to a subject for two weeks. In some cases, the steady dose (e.g. once daily dose) of the inhibitor is administered to a subject for three weeks. In some cases, the steady dose (e.g. once daily dose) of the inhibitor is administered to a subject for four weeks. In some cases, the steady dose (e.g. once daily dose) of the inhibitor is administered to a subject for six weeks. In some cases, the steady dose (e.g. once daily dose) of the inhibitor is administered to a subject for two months. In some cases, the steady dose (e.g. once daily dose) of the inhibitor is administered to a subject for three months. In some cases, the steady dose (e.g. once daily dose) of the inhibitor is administered to a subject for six months. In some cases, the steady dose (e.g. once daily dose) of the inhibitor is administered to a subject for twelve months. In some cases, the steady dose (e.g. once daily dose) of the inhibitor is administered to a subject for twenty-four months.

In some cases, the inhibitor (e.g. venetoclax) may be administered to a subject for between one week and two years, between one week and one year, between one week and 6 months, between one week and 3 months, between one week and five weeks, between one week and four weeks, between one week and three weeks, between one week and two weeks. In some embodiments, the inhibitor (e.g. venetoclax) is administered to a subject for at least one week. In some embodiments, the inhibitor (e.g. venetoclax) is administered to a subject for at least two weeks. In some embodiments, the inhibitor (e.g. venetoclax) is administered to a subject for at least three weeks. In some embodiments, the inhibitor (e.g. venetoclax) is administered to a subject for at least four weeks. In some embodiments, the inhibitor (e.g. venetoclax) is administered to a subject for at least five weeks. In some embodiments, the inhibitor (e.g. venetoclax) is administered to a subject for at least two months. In some embodiments, the inhibitor (e.g. venetoclax) is administered to a subject for at least three months. In some embodiments, the inhibitor (e.g. venetoclax) is administered to a subject for at least six months. In some embodiments, the inhibitor is administered to a subject for at least one year. In some embodiments, the inhibitor (e.g. venetoclax) is administered to a subject for at least two years.

In some cases, the inhibitor (e.g. venetoclax) may be administered to a subject for between one week and two years, between one week and one year, between one week and 6 months, between one week and 3 months, between one week and five weeks, between one week and four weeks, between one week and three weeks, or between one week and two weeks, following initiation of administration of the cytotoxic therapy. In some embodiments, following initiation of administration of the cytotoxic therapy, the inhibitor (e.g. venetoclax) is administered to a subject for at least one week, at least two, at least three weeks, at least four weeks, at least five weeks, at least 6 weeks, at least two months, at least three months, at least six months, at least one year, or at least two years. In some embodiments, following initiation of administration of the cytotoxic therapy, the inhibitor (e.g. venetoclax) is administered to a subject for about three weeks. In some embodiments, following initiation of administration of the cytotoxic therapy, the inhibitor (e.g. venetoclax) is administered to a subject for about three months. In some embodiments, following initiation of administration of the cytotoxic therapy, the inhibitor (e.g. venetoclax) is administered to a subject for about six months. In some embodiments, following initiation of administration of the cytotoxic therapy, the inhibitor (e.g. venetoclax) is administered to a subject for about twelve months. In some embodiments, following initiation of administration of the cytotoxic therapy, the inhibitor (e.g. venetoclax) is administered to a subject for about twenty-four months.

In some cases, the inhibitor is administered at a subtherapeutically effective amount. In some cases, in the context of administration, a subtherapeutically effective amount refers to an amount less effective, at dosages and/or for periods of time necessary, to achieve a desired therapeutic result, such as for treatment of a disease, condition, or disorder, and/or pharmacokinetic or pharmacodynamic effect of the treatment, as compared to a “therapeutically effective amount” of the same agent. In some cases, the subtherapeutically effective amount does not achieve or achieves less than a desired therapeutic result, such as for treatment of a disease, condition, or disorder, and/or pharmacokinetic or pharmacodynamic effect of the treatment. In some cases the desired therapeutic result is a reduction of tumor size, bulk, metastasis, percentage of blasts in the bone marrow or molecularly detectable B cell malignancy and/or an improvement in prognosis or survival or other symptom associated with tumor burden. In some cases the desired therapeutic result is an ability of the inhibitor to induce apoptosis in cancer cells and/or an increase in apoptosis of cancer cells. In some cases, a subtherapeutically effective amount results in mild or moderate neutropenia In some cases, a subtherapeutically effective amount does not result in severe neutropenia. In some cases, mild neutropenia is defined as an absolute neutrophil count of 1,000 to 1,5000/μL. In some cases, moderate neutropenia is defined as an absolute neutrophil count of 500 to 1,000/μL. In some cases, severe neutropenia is defined as an absolute neutrophil count of fewer than 500/μL.

Methods for identifying and determining whether a desired therapeutic result for the treatment of cancer is achieved include but are not limited to any as described in Section III. The subtherapeutically effective amount may vary according to factors such as the disease state, age, sex, and weight of the subject, and the populations of cells administered.

In some embodiments, the provided methods involve administering the cells and/or compositions at subeffective amounts, e.g., subtherapeutically effective amounts. In some embodiments, the provided methods involve administering an inhibitor of a prosurvival BCL2 family protein, such as a BCL2 inhibitor, e.g., venetoclax, engineered cells (e.g. cell therapy), or compositions at subeffective amounts, e.g., subtherapeutically effective amounts. In some cases, an inhibitor of a prosurvival BCL2 family protein is administered at a subtherapeutic amounts, i.e., the dosing regimen (e.g. dose and/or duration) is less than dosing regimen commonly prescribed by clinicians to achieve a therapeutic or prophylactic result.

In some cases, the inhibitor is administered at a steady (e.g. once daily) dose of between about 20 milligrams daily and about 100 milligrams daily. In some cases, the inhibitor is administered at a dose of between about 20 mg daily and about 40 mg daily. In some cases, the inhibitor is administered at a dose of between about 20 mg daily and about 50 mg daily. In some cases, the inhibitor is administered at a steady (e.g. once daily) dose of between about 40 milligrams daily and about 100 milligrams daily. In some cases, the inhibitor is administered at a dose of between about 40 mg daily and about 50 mg daily. In some cases, the inhibitor is administered at a steady (e.g. once daily) dose of between about 50 milligrams daily and about 100 milligrams daily. In some cases, the inhibitor is administered at a dose of not more than about 20 mg daily. In some cases, the inhibitor is administered at a dose of about 20 mg daily. In some cases, the inhibitor is administered at a dose of not more than about 40 mg daily. In some cases, the inhibitor is administered at a dose of about 40 mg daily. In some cases, the inhibitor is administered at a dose of not more than about 50 mg daily. In some cases, the inhibitor is administered at a dose of about 50 mg daily. In some cases, the inhibitor is administered at a dose of not more than about 100 mg daily. In some cases, the inhibitor is administered at a dose of about 100 mg daily. In some cases, the inhibitor is administered at a dose of not more than about 200 mg daily. In some cases, the inhibitor is administered at a dose of about 200 mg daily. In some cases, the inhibitor is administered at a dose of not more than about 400 mg daily. In some cases, the inhibitor is administered at a dose of about 400 mg daily.

In some embodiments, the steady dose (e.g. once daily dose) is administered to a subject for between two weeks and twenty-four months. In some embodiments, the steady dose (e.g. once daily dose) is administered to a subject for between four weeks and twenty-four months. In some embodiments, the steady dose (e.g. once daily dose) is administered to a subject for between six weeks and twenty-four months. In some embodiments, the steady dose (e.g. once daily dose) is administered to a subject for between two months and twenty-four months. In some embodiments, the steady dose (e.g. once daily dose) is administered to a subject for between three months and twenty-four months. In some embodiments, the steady dose (e.g. once daily dose) is administered to a subject for between six months and twenty-four months. In some embodiments, the steady dose (e.g. once daily dose) is administered to a subject for between twelve months and twenty-four months. In some embodiments, the steady dose (e.g. once daily dose) is administered to a subject for between eighteen months and twenty-four months. In some embodiments, the steady dose (e.g. once daily dose) is administered to a subject for at least two weeks. In some embodiments, the steady dose (e.g. once daily dose) is administered to a subject for at least three weeks. In some embodiments, the steady dose (e.g. once daily dose) is administered to a subject for at least four weeks. In some embodiments, the steady dose (e.g. once daily dose) is administered to a subject for at least six weeks. In some embodiments, the steady dose (e.g. once daily dose) is administered to a subject for at least two months. In some embodiments, the steady dose (e.g. once daily dose) is administered to a subject for at least three months. In some embodiments, the steady dose (e.g. once daily dose) is administered to a subject for at least six months. In some embodiments, the steady dose (e.g. once daily dose) is administered to a subject for at least twelve months. In some embodiments, the steady dose (e.g. once daily dose) is administered to a subject for at least twenty-four months.

Prior Administration of Prosurvivial BCL-2 Family Protein Inhibitor, e.g. Dose Ramp-Up

In any of the methods provided herein, the prosurvival BCL2 family protein inhibitor (e.g. venetoclax) is administered in a dose-ramp up schedule prior to administration of the dosing regimen of the inhibitor as provided herein. In some embodiments, the dose-ramp up schedule comprises administration of escalating doses of the inhibitor.

In some embodiments, the method involves initiating the administration of the prosurvival BCL2 family protein inhibitor, such as a BCL2 inhibitor, e.g., venetoclax, prior to initiation of administration of the cytotoxic therapy, such as a T cell therapy (e.g. CAR T cells). In some embodiments, the inhibitor, e.g. venetoclax, is administered prior to administration of the cytotoxic therapy, such as a T cell therapy (e.g. CAR T cells) in a dose ramp-up schedule, such as after collection of autologous cells from a subject to be treated and prior to administration of the lymphodepleting therapy (hereinafter, a “bridging therapy”). In some aspects, the inhibitor can be give prior to administration of a lymphodepleting therapy to the subject, as described in Section I.C. In some embodiments, the prosurvival BCL2 family protein inhibitor, e.g., venetoclax, is further administered, after a discontinuation or pause during a lymphodepleting therapy, such as until or after initiation of the cytotoxic therapy. Thus, in some cases, a subject undergoes collection of autologous cells (e.g. leukapheresis), bridging therapy with the inhibitor (e.g. venetoclax), lymphodepletion, initiation of administration of CAR T cells, and initiation of administration of the dosing regmen of the inhibitor (e.g., venetoclax), in that order.

In some embodiments, administration of the prosurvival BCL2 family protein inhibitor, e.g., venetoclax, is discontinued or paused prior to a lymphodepleting therapy, such as for a duration of between about three half lives and about four half lives of the inhibitor. In some embodiments, administration of the prosurvival BCL2 family protein inhibitor, e.g., venetoclax, is discontinued or paused prior to a lymphodepleting therapy, such as for a duration of between about three half lives and about four half lives of the inhibitor, and administration of the inhibitor is resumed at or after initiation of the cytotoxic therapy. In some embodiments, administration of the prosurvival BCL2 family protein inhibitor, e.g., venetoclax, is discontinued at least 1 day prior to a lymphodepleting therapy. In some embodiments, administration of the prosurvival BCL2 family protein inhibitor, e.g., venetoclax, is discontinued at least 2 days prior to a lymphodepleting therapy. In some embodiments, administration of the prosurvival BCL2 family protein inhibitor, e.g., venetoclax, is discontinued at least 3 days prior to a lymphodepleting therapy. In some embodiments, administration of the prosurvival BCL2 family protein inhibitor, e.g., venetoclax, is discontinued at least 4 days prior to a lymphodepleting therapy.

In some aspects, the methods involve administration of the prosurvival BCL2 family protein inhibitor, e.g., venetoclax, that is paused or concluded at or at least about 4 half lives of the inhibitor or a minimum of at or about 4 half lives of the inhibitor prior to obtaining a sample comprising T cells from the subject, e.g., for producing a T cell therapy for administration. In some aspects, the methods involve administration of the prosurvival BCL2 family protein inhibitor, e.g., venetoclax, that is paused or concluded at or at least about 3 days or a minimum of at or about 3 days prior to obtaining a sample comprising T cells from the subject, e.g., for producing a T cell therapy for administration. In some aspects, the methods involve administration of the prosurvival BCL2 family protein inhibitor, e.g., venetoclax, that is paused or concluded at or at least about 1 day or a minimum of at or about 1 day prior to obtaining a sample comprising T cells from the subject, e.g., for producing a T cell therapy for administration. In some aspects, the methods involve administration of the prosurvival BCL2 family protein inhibitor, e.g., venetoclax, that is paused or concluded at or at least about 4 days or a minimum of at or about 4 days prior to obtaining a sample comprising T cells from the subject, e.g., for producing a T cell therapy for administration. In some aspects, the T cell therapy is produced by a process that involves obtaining a sample comprising T cells from the subject and introducing a nucleic acid molecule encoding the CAR, such as any nucleic acid molecules described herein, into a composition comprising the T cells.

In some embodiments, the bridging therapy comprises escalating or “ramp-up” dosing. In some cases the inhibitor is administered to the subject in escalating doses. In some cases the inhibitor is administered to the subject in escalating doses before it is administered to the subject at a steady dose (e.g. a once daily dose). In some cases the inhibitor is administered to the subject at a low or “ramp-up” dose before it is administered to a subject at a steady dose. In some cases the inhibitor is administered to the subject in escalating doses before the cytotoxic therapy is administered to the subject. In some cases the inhibitor is administered to the subject at a steady dose before the cytotoxic therapy is administered to the subject. In some cases, prior to administration of the cytotoxic therapy to the subject, the inhibitor is administered to the subject at escalating doses and at a steady dose. In some cases, the inhibitor is administered to the subject concurrently with the cytotoxic therapy. In some cases the inhibitor is administered to the subject following administration of the cytotoxic therapy. In some cases the inhibitor is administered to the subject prior to and at the same time as the administration of the cytotoxic therapy. In some cases the inhibitor is administered to the subject prior to and following administration of the cytotoxic therapy. In some cases the inhibitor is administered concurrently with and following administration of the cytotoxic therapy. In some cases the inhibitor is administered prior to, concurrently with, and following administration of the cytotoxic therapy. In some cases the inhibitor is administered prior to, concurrently with, and/or following administration of the cytotoxic therapy.

In some embodiments, resuming administration of the prosurvival BCL2 family protein inhibitor, e.g., venetoclax, involves administration of multiple doses of the prosurvival BCL2 family protein inhibitor, e.g., venetoclax. In some embodiments, administration of the prosurvival BCL2 family protein inhibitor, e.g., venetoclax, is not resumed until within about 7 days prior to initiation of the cytotoxic therapy. In some embodiments, administration the prosurvival BCL2 family protein inhibitor, e.g., venetoclax, is not resumed until within about 3 days prior to initiation of the cytotoxic therapy. In some embodiments, administration of the prosurvival BCL2 family protein inhibitor, e.g., venetoclax, is not resumed until within about 1 days prior to initiation of the cytotoxic therapy. In some embodiments, administration the prosurvival BCL2 family protein inhibitor, e.g., venetoclax, is not resumed until or after initiation of the cytotoxic therapy. In some embodiments, administration of the prosurvival BCL2 family protein inhibitor, e.g., venetoclax, is not resumed until after initiation of the cytotoxic therapy. In some embodiments, administration of the prosurvival BCL2 family protein inhibitor, e.g., venetoclax, is not resumed until within about 14 days after initiation of the cytotoxic therapy. In some embodiments, administration of the prosurvival BCL2 family protein inhibitor, e.g., venetoclax, is not resumed until within about 11 days after initiation of the cytotoxic therapy. In some embodiments, administration of the prosurvival BCL2 family protein inhibitor, e.g., venetoclax, is not resumed until within about 7 days after initiation of the cytotoxic therapy. In some embodiments, administration of the prosurvival BCL2 family protein inhibitor, e.g., venetoclax, is not resumed until within about 6 days after initiation of the cytotoxic therapy. In some embodiments, administration of the prosurvival BCL2 family protein inhibitor, e.g., venetoclax, is not resumed until within about 5 days after initiation of the cytotoxic therapy. In some embodiments, administration of the prosurvival BCL2 family protein inhibitor, e.g., venetoclax, is not resumed until within about 4 days after initiation of the cytotoxic therapy. In some embodiments, administration of the prosurvival BCL2 family protein inhibitor, e.g., venetoclax, is not resumed until within about 3 days after initiation of the cytotoxic therapy. In some embodiments, administration of the prosurvival BCL2 family protein inhibitor, e.g., venetoclax, is not resumed until within about 2 days after initiation of the cytotoxic therapy. In some embodiments, administration of the prosurvival BCL2 family protein inhibitor, e.g., venetoclax, is not resumed until within about 1 day after initiation of the cytotoxic therapy.

In some cases, the inhibitor is administered to the subject in escalating or “ramp up” doses over the course of five weeks. Thus, in some cases, the bridging therapy comprises five weeks of escalating doses. In some cases, the inhibitor is administered to the subject in escalating doses over the course of five weeks to reach a maximum dose of 400 mg per day. In some cases, the inhibitor is administered to the subject at 20 milligrams per day for the first week, 40 or 50 milligrams per day for the second week, 100 milligrams per day for the third week, 200 milligrams per day for the fourth week, and 400 milligrams per day for the fifth week. In some cases, the inhibitor is administered to the subject in escalating doses until a maximum dose of 40 mg daily is reached. In some cases, the inhibitor is administered to the subject in escalating doses until a maximum dose of 50 mg daily is reached. In some cases, the inhibitor is administered to the subject in escalating doses until a maximum dose of 100 mg daily is reached. In some embodiments the inhibitor is administered to the subject in escalating doses for about 1 week, about 2 weeks, about 3 weeks, about 4 weeks or about 5 weeks.

In some cases, the inhibitor is administered to the subject in escalating doses over the course of three weeks. Thus, in some cases, the bridging therapy comprises three weeks of escalating doses. In some cases, the inhibitor is administered to the subject in escalating doses over the course of three weeks to reach a maximum dose of 400 mg per day. In some cases, the inhibitor is administered to the subject at 20 milligrams per day on the day one of the first week; increasing to 40 or 50 milligrams per day on day two or day three of the first week; increasing to 100 milligrams per day between day four and day seven of the first week; increasing to 200 milligrams per day on day one of the second week; and increasing to 400 milligrams per day on day one of the third week. In some cases, the inhibitor is administered to the subject in escalating doses until a maximum dose of 40 mg daily is reached. In some cases, the inhibitor is administered to the subject in escalating doses until a maximum dose of 50 mg daily is reached. In some cases, the inhibitor is administered to the subject in escalating doses until a maximum dose of 100 mg daily is reached. In some embodiments the inhibitor is administered to the subject in escalating doses for about 1 week, about 2 weeks, or about 3 weeks.

In some embodiments, the bridging therapy comprises administration of the inhibitor, e.g. venetoclax, to the subject at a first dose for a first week, at a second dose for a second week, and at a third dose for a third week. In some embodiments, the escalating doses comprise a first dose that is at about 20 mg per day, a second dose that is at about 50 mg per day, and a third dose that is at about 100 mg per day. In some embodiments, the escalating doses comprise a first dose that is at about 20 mg per day and is dosed for a first week, a second dose that is at about 50 mg per day and is dosed for a second week, and a third dose that is at about 100 mg per day and is dosed for a third week. Thus, in some cases, the bridging therapy comprises administration of the inhibitor, e.g. venetoclax, at 20 mg per day for a first week, 50 mg per day for a second week, and 100 mg per day for a third week. In some embodiments, each escalating dose is administered once daily for a week and/or the last escalating dose is administered once daily for a week or until the end of the bridging therapy.

In some cases, following administration of escalating doses of the inhibitor, the subject continues to receive the inhibitor at the maximum escalated dose until the bridging therapy is ceased. In some cases, following administration of the inhibitor, e.g. venetoclax, at 20 mg per day for a first week, 50 mg per day for a second week, and 100 mg per day for a third week, the subject continues to receive the inhibitor at the 100 mg per day dose until the bridging therapy is concluded. In some cases, following administration of escalating doses of the inhibitor, the subject receives the inhibitor at a higher dose than the maximum escalated dose until the bridging therapy is ceased. In some cases, following administration of the inhibitor, e.g. venetoclax, at 20 mg per day for a first week, 50 mg per day for a second week, and 100 mg per day for a third week, the subject receives the inhibitor at a higher dose than 100 mg per day (e.g. 200 mg/day or 400 mg/day) until the bridging therapy is ceased. In some embodiments, the bridging therapy is ceased at least 1 day prior to administration of the lymphodepleting therapy. In some embodiments, the bridging therapy is ceased at least 2 days prior to administration of the lymphodepleting therapy. In some embodiments, the bridging therapy is ceased at least 3 days prior to administration of the lymphodepleting therapy. In some embodiments, the bridging therapy is ceased at least 4 days prior to administration of the lymphodepleting therapy.

In some cases, a steady (e.g. once daily) dose of the inhibitor is administered to the subject after administration of the escalating dose. In some cases, a steady dose of between about 400 mg per day and 800 mg per day is administered to the subject after administration of the escalating dose. In some cases, a steady dose of about 400 mg per day is administered to the subject after administration of the escalating dose. In some cases, a steady dose of about 400 mg per day is administered to the subject for at least one week at a time that is after administration of the escalating dose. In some cases, a steady dose of about 400 mg per day is administered to the subject for at least two weeks at a time that is after administration of the escalating dose. In some cases, a steady dose of about 400 mg per day is administered to the subject for at least four weeks at a time that is after administration of the escalating dose. In some cases, a steady dose of about 400 mg per day is administered to the subject for at least two months at a time that is after administration of the escalating dose. In some cases, a steady dose of about 400 mg per day is administered to the subject for at least three months at a time that is after administration of the escalating dose. In some cases, a steady dose of about 400 mg per day is administered to the subject for at least six months at a time that is after administration of the escalating dose. In some cases, a steady dose of about 400 mg per day is administered to the subject for at least twelve months at a time that is after administration of the escalating dose. In some cases, a steady dose of about 400 mg per day is administered to the subject for at least eighteen months at a time that is after administration of the escalating dose. In some cases, a steady dose of about 400 mg per day is administered to the subject for at least twenty-four months at a time that is after administration of the escalating dose. In some cases a steady dose of 400 or 800 mg per day is administered to the subject after the administration of escalating doses over three weeks. In some cases a steady dose of 400 mg per day is administered to the subject after the administration of escalating doses over three weeks. In some cases a steady dose of 400 or 800 mg per day is administered to the subject after the administration of escalating doses over five weeks. In some cases a steady dose of 400 mg per day is administered to the subject after the administration of escalating doses over five weeks.

In some cases, the inhibitor is administered to the subject at a “ramp up” dose for between about one week and about five weeks. In some cases, the inhibitor is administered to the subject as a ramp up dose for between about one and about three weeks. In some cases, the inhibitor is administered to the subject as a ramp up dose for between about one and about two weeks. In some cases, the inhibitor is administered to the subject as a ramp up dose for about one week. In some cases, the inhibitor is administered to the subject as a ramp up dose for between 7 and 21 days. In some cases, the ramp us dose is between about 20 mg per day and 400 mg per day, between about 40 mg per day and 400 mg per day, between about 50 mg per day and 400 mg per day, between about 150 mg per day and 300 mg per day, between about 200 mg per day and 300 mg per day. In some cases, the ramp up dose is 150 mg per day. In some cases, the ramp up dose is 150 mg per day for 7 days. In some cases, the ramp up dose is 150 mg per day for 14 days. In some cases, the ramp up dose is 150 mg per day for 21 days. In some cases, the ramp up dose is 150 mg per day and administered continuously for between about 7 days and about 21 days. In some cases, the ramp up dose is no more than 20 mg daily. In some cases, the ramp up dose is no more than 40 mg daily. In some cases, the ramp up dose is no more than 50 mg daily. In some cases, the ramp up dose is no more than 100 mg daily.

In some cases, a steady (e.g. once daily) dose of the inhibitor is administered to the subject after administration of the ramp up dose. In some cases, a steady dose of between about 100 mg per day and 500 mg per day is administered to the subject after administration of the ramp up dose. In some cases, a steady dose of between about 150 mg per day and 425 mg per day is administered to the subject after administration of the ramp up dose. In some cases, a steady dose of at least about 150 mg per day is administered to the subject after administration of the ramp up dose. In some cases, a steady dose of at least about 200 mg per day is administered to the subject after administration of the ramp up dose. In some cases, a steady dose of about 325 mg per day is administered to the subject after administration of the ramp up dose. In some cases, a steady dose of about 425 mg per day is administered to the subject after administration of the ramp up dose. In some cases, a steady dose of about 325 mg per day is administered to the subject for at least one week at a time that is after administration of the ramp up dose. In some cases, a steady dose of about 325 mg per day is administered to the subject for at least two weeks at a time that is after administration of the ramp up dose. In some cases, a steady dose of about 325 mg per day is administered to the subject for at least three weeks at a time that is after administration of the ramp up dose. In some cases, a steady dose of about 325 mg per day is administered to the subject for at least two months at a time that is after administration of the ramp up dose. In some cases, a steady dose of about 325 mg per day is administered to the subject for at least three months at a time that is after administration of the ramp up dose. In some cases, a steady dose of about 325 mg per day is administered to the subject for at least six months at a time that is after administration of the ramp up dose. In some cases, a steady dose of about 325 mg per day is administered to the subject for at least twelve months at a time that is after administration of the ramp up dose. In some cases, a steady dose of about 325 mg per day is administered to the subject for at least eighteen months at a time that is after administration of the ramp up dose. In some cases, a steady dose of about 325 mg per day is administered to the subject for at least twenty-four months at a time that is after administration of the ramp up dose. In some cases, a steady dose of about 325 mg per day is administered to the subject following a ramp up dose of 150 mg per day. In some cases, a steady dose of about 325 mg per day is administered to the subject for one or more cycles of 14 days on and 7 days off, or 21 continuous days on, following a ramp up dose of 150 mg per day.

One skilled in the art will recognize that higher or lower dosages of the inhibitor could be used in any of the provided methods, for example depending on the particular agent and the route of administration. In some embodiments, the inhibitor may be administered alone or in the form of a pharmaceutical composition wherein the compound is in admixture or mixture with one or more pharmaceutically acceptable carriers, excipients, or diluents. In some embodiments, the inhibitor may be administered either systemically or locally to the organ or tissue to be treated. Exemplary routes of administration include, but are not limited to, topical, injection (such as subcutaneous, intramuscular, intradermal, intraperitoneal, intratumoral, and intravenous), oral, sublingual, rectal, transdermal, intranasal, vaginal and inhalation routes. In some embodiments, the route of administration is oral, parenteral, rectal, nasal, topical, or ocular routes, or by inhalation. In some embodiments, the inhibitor is administered orally. In some embodiments, the inhibitor is administered orally in solid dosage forms, such as capsules, tablets and powders, or in liquid dosage forms, such as elixirs, syrups and suspensions.

Once improvement of the patient's disease has occurred, the dose may be adjusted for preventative or maintenance treatment. For example, the dosage or the frequency of administration, or both, may be reduced as a function of the symptoms, to a level at which the desired therapeutic or prophylactic effect is maintained. If symptoms have been alleviated to an appropriate level, treatment may cease. Patients may, however, require intermittent treatment on a long-term basis upon any recurrence of symptoms. Patients may also require chronic treatment on a long-term basis.

B. Administration of an Immunotherapy or Cell Therapy

In some embodiments of the methods, compositions, combinations, kits and uses provided herein, the combination therapy includes administering to a subject a therapy, e.g. cytotoxic therapy, such as an immunotherapy or cell therapy. In some embodiments, the therapy is a T cell therapy (e.g. CAR-expressing T cells) or a T cell-engaging therapy. Such therapies can be administered prior to, subsequent to, simultaneously with administration of one or more inhibitor of a prosurvival BCL2 family protein as described.

1. T Cell-Engaging Therapy

In some embodiments, the immunotherapy is or comprises a T cell-engaging therapy that is or comprises a binding molecule capable of binding to a surface molecule expressed on a T cell. In some embodiments, the surface molecule is an activating component of a T cell, such as a component of the T cell receptor complex. In some embodiments, the surface molecule is CD3 or is CD2. In some embodiments, the T cell-engaging therapy is or comprises an antibody or antigen-binding fragment.

In some embodiments, the T cell-engaging therapy is a bispecific antibody containing at least one antigen-binding domain binding to an activating component of the T cell (e.g. a T cell surface molecule, e.g. CD3 or CD2) and at least one antigen-binding domain binding to a surface antigen on a target cell, such as a surface antigen on a tumor or cancer cell, for example any of the listed antigens as described herein, e.g. CD19. In some embodiments, the simultaneous or near simultaneous binding of such an antibody to both of its targets can result in a temporary interaction between the target cell and T cell, thereby resulting in activation, e.g. cytotoxic activity, of the T cell and subsequent lysis of the target cell.

In some embodiments, bi-specific T cell engagers (BiTE) are used in connection with the provided methods, uses, articles of manufacture. In some embodiments, bi-specific T cell engagers have specificity toward two particular antigens (or markers or ligands). In some embodiments, the antigens are expressed on the surface of a particular type of cell. In particular embodiments, the first antigen is associated with an immune cell or an engineered immune cell, and the second antigen is associated with a target cell of the particular disease or condition, such as a cancer.

Numerous methods of producing bi-specific T cell engagers are known, including fusion of two different hybridomas (Milstein and Cuello, Nature 1983; 305:537-540), and chemical tethering though heterobifunctional cross linkers (Staerz et al. Nature 1985; 314:628-631). Among exemplary bi-specific antibody T cell-engaging molecules are those which contain tandem scFv molecules fused by a flexible linker (see e.g. Nagorsen and Bauerle, Exp Cell Res 317, 1255-1260 (2011); tandem scFv molecules fused to each other via, e.g. a flexible linker, and that further contain an Fc domain composed of a first and a second subunit capable of stable association (WO2013026837); diabodies and derivatives thereof, including tandem diabodies (Holliger et al, Prot Eng 9, 299-305 (1996); Kipriyanov et al, J Mol Biol 293, 41-66 (1999)); dual affinity retargeting (DART) molecules that can include the diabody format with a C-terminal disulfide bridge; or triomabs that include whole hybrid mouse/rat IgG molecules (Seimetz et al, Cancer Treat Rev 36, 458-467 (2010).

In certain embodiments, the bi-specific T cell engager is a molecule encoded by a polypeptide construct. In certain embodiments, the polypeptide construct contains a first component comprising an antigen-binding domain binding to an activating portion of an immune cell or engineered immune cell, and a second component comprising an antigen-binding domain binding to a surface antigen (e.g. target or tumor associated antigen (TAA)) associated with a particular disease or condition (e.g. cancer). In some embodiments, the first and second components are coupled by a linker. In some embodiments, the first component is coupled to a leader sequence encoding a CD33 signal peptide.

In some embodiments, the polypeptide is a construct containing from N-terminus to C-terminus: a first component comprising an antigen-binding domain binding to an activating portion of the T cell, a peptide linker, and a second component comprising an antigen-binding domain binding to a surface antigen (e.g. target or tumor associated antigen (TAA)) associated with a disease or condition (e.g. cancer).

In some aspects, an activating component of the T cell I a T cell surface molecule, such as CD3 or CD2. In some embodiments, the surface antigen of the target cell is a tumor associated antigen (TAA). In some aspects, the TAA contains one or more epitopes. In some embodiments, the peptide linker is or comprises a cleavable peptide linker.

In some embodiments, the antigen binding domain of the first component of the bi-specific T cell engager engages a receptor on an endogenous immune cell in the periphery of the tumor. In some embodiments, the endogenous immune cell is a T cell. In some aspects, the engagement of the endogenous T cell receptor redirects the endogenous T cells to the tumor. In some aspects, the engagement of the endogenous T cell receptor recruits tumor infiltrating lymphocytes (TILs) to the tumor. In some aspects, the engagement of the endogenous T cell receptor activates the endogenous immune repertoire.

In some embodiments, the simultaneous or near simultaneous binding of the bi-specific T cell engager to both of its targets (e.g. the immune cell and the TAA) can result in a temporary interaction between the target cell and T cell, thereby resulting in activation (e.g. cytotoxic activity, cytokine release), of the T cell and subsequent lysis of the target cell.

In some embodiments, the first component of the bi-specific T cell engager is or comprises an antigen binding domain that binds to an activating component of a T cell. In some embodiments, the activating component of the T cell is a surface molecule. In some embodiments, the surface molecule is or comprises a T-cell antigen. Exemplary T-cell antigens include but are not limited to CD2, CD3, CD4, CD5, CD6, CD8, CD25, CD28, CD30, CD40, CD44, CD45, CD69 and CD90. In some aspects, the binding of the bispecific T cell engaging molecule with the T cell antigen stimulates and/or activates the T cell.

In some embodiments, the anti-T cell binding domain includes an antibody or an antigen-binding fragment thereof selected from the group consisting of a Fab fragment, a F(ab′)₂ fragment, an Fv fragment, an scFv, a scAb, a dAb, a single domain heavy chain antibody, and a single domain light chain antibody.

In some embodiments, the T cell binding domain on the bi-specific T cell engager is an anti-CD3. In some aspects, the anti-CD3 domain is an scFv. In some embodiments, the anti-CD3 domain of the bi-specific T cell engager binds to a subunit of the CD3 complex on a receptor on a T cell. In some aspects, the receptor is on an endogenous T cell. In some embodiments, the receptor is on an engineered immune cell further expressing a recombinant receptor. The effects of CD3 engagement of T cells is well known in the art, and include but are not limited to T cell activation and other downstream cell signaling. Any of such bi-specific T cell engagers can be used in the provided disclosure herein.

In some embodiments, the second component of the bi-specific T cell engager comprising an antigen-binding domain binding to a surface antigen associated with a disease or condition is a tumor or cancer antigen. In some embodiments, among the antigens targeted by the bi-specific T cell engager are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas.

In some embodiments, the antigen includes αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPCR5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha(IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30. In some embodiments, the antigen is CD19.

In some embodiments, both antigen binding domains, including the first antigen binding domain and the second antigen binding domain, comprise an antibody or an antigen-binding fragment.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, variable heavy chain (VH) regions capable of specifically binding the antigen, single chain antibody fragments, including single chain variable fragments (scFv), and single domain antibodies (e.g., sdAb, sdFv) or fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

In some embodiments, the antigen-binding proteins, antibodies and antigen binding fragments thereof specifically recognize an antigen of a full-length antibody. In some embodiments, the heavy and light chains of an antibody can be full-length or can be an antigen-binding portion (a Fab, F(ab′)2, Fv or a single chain Fv fragment (scFv)). In other embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4, more particularly, IgG1 (e.g., human IgG1). In another embodiment, the antibody light chain constant region is chosen from, e.g., kappa or lambda, particularly kappa.

Among the provided antibodies are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; variable heavy chain (V_{H}) regions, single-chain antibody molecules such as scFvs and single-domain V_{H} single antibodies; and multispecific antibodies formed from antibody fragments. In particular embodiments, the antibodies are single-chain antibody fragments comprising a variable heavy chain region and/or a variable light chain region, such as scFvs.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Single-domain antibodies (sdAb) are antibody fragments comprising all or a portion of the heavy chain variable domain or all or a portion of the light chain variable domain of an antibody. In certain embodiments, a single-domain antibody is a human single-domain antibody. In some embodiments, the bi-specific T cell engager comprises an antibody heavy chain domain that specifically binds the antigen, such as a cancer marker or cell surface antigen of a cell or disease to be targeted, such as a tumor cell or a cancer cell, such as any of the target antigens described herein or known. Exemplary single-domain antibodies include sdFv, nanobody, V_{H}H or V_{NAR}.

Antibody fragments can be made by various techniques, including but not limited to proteolytic digestion of an intact antibody as well as production by recombinant host cells. In some embodiments, the antibodies are recombinantly produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some embodiments, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

In certain embodiments, the antigen binding domains are single chain variable fragments (scFv). In some embodiments, the scFv is a tandem scFv containing a heavy and a light chain. In some embodiments, the heavy and light chains are connected by peptide linkers. In some embodiments, the linker is composed primarily of serines and glycines. In some aspects, the linkage of the heavy chain and the light chain forms a single polypeptide antigen binding domain.

In certain embodiments, the first antigen binding domain of the bi-specific T cell engager is an anti-CD3 scFv. In certain embodiments, the second antigen binding domain of the bi-specific T cell engager is an anti-CD19 scFv.

In some aspects, the bi-specific T cell engager polypeptide constructs contain a linker that joins the first component comprising the antigen-binding domain that binds to an activating portion of the T cell, to the second component comprising an antigen-binding domain binding to a surface antigen (e.g. target or tumor associated antigen (TAA)) associated with a particular disease or condition. In some aspects, the linker is a short, medium or long linker.

In some embodiments, the linker is a peptide linker which is cleavable. In some aspects, the cleavable linker includes a sequence that is a substrate for a protease. In some embodiments, the sequence comprises a bond that can be broken under in vivo conditions. In some cases, the linker sequence is selectively cleaved by a protease present in a physiological environment. In some aspects, the environment is separate from the tumor microenvironment. In some embodiments, the protease is found in the periphery of the tumor.

In some embodiments, the selectively cleavable linker is cleaved by a protease produced by cells that do not co-localize with the tumor. In some embodiments, the selectively cleavable linker is not cleaved by proteases that are in the proximity of the tumor microenvironment. In some embodiments, the cleavage of the linker by the protease renders the bi-specific T cell engaging molecule inactive. In some embodiments, the protease is found in the circulating blood of a subject. In some embodiments, the protease is a part of the intrinsic or extrinsic coagulation pathway. In some aspects, the protease is a serine protease. In some aspects, the protease comprises but is not limited to a thrombin, factor X, factor XI, factor XII, and plasmin.

Among such exemplary bispecific antibody T cell-engagers are bispecific T cell engager (BiTE) molecules, which contain tandem scFv molecules fused by a flexible linker (see e.g. Nagorsen and Bauerle, Exp Cell Res 317, 1255-1260 (2011); tandem scFv molecules fused to each other via, e.g. a flexible linker, and that further contain an Fc domain composed of a first and a second subunit capable of stable association (WO2013026837); diabodies and derivatives thereof, including tandem diabodies (Holliger et al, Prot Eng 9, 299-305 (1996); Kipriyanov et al, J Mol Biol 293, 41-66 (1999)); dual affinity retargeting (DART) molecules that can include the diabody format with a C-terminal disulfide bridge; or triomabs that include whole hybrid mouse/rat IgG molecules (Seimetz et al, Cancer Treat Rev 36, 458-467 (2010). In some embodiments, the T-cell engaging therapy is blinatumomab or AMG 330. Any of such T cell-engagers can be used in used in the provided methods.

The immune system stimulator and/or the T cell engaging therapy can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, the immunotherapy is administered by parenteral, intrapulmonary, and intranasal, and, if desired for local treatment, intralesional administration. Parenteral infusions include intramuscular, intravenous, intraarterial, intraperitoneal, intrathoracic, intracranial, or subcutaneous administration.

In certain embodiments, one or more doses of a T cell engaging therapy are administered. In particular embodiments, between or between about 0.001 μg and about 5,000 μg, inclusive, of the T cell engaging therapy is administered. In particular embodiments, between or between about 0.001 μg and 1,000 μg, 0.001 μg to 1 μg, 0.01 μg to 1 μg, 0.1 μg to 10 μg, 0.01 μg to 1 μg, 0.1 μg and 5 μg, 0.1 μg and 50 μg, 1 μg and 100 μg, 10 μg and 100 μg, 50 μg and 500 μg, 100 μg and 1,000 μg, 1,000 μg and 2,000 μg, or 2,000 μg and 5,000 μg of the T cell engaging therapy is administered. In some embodiments, the dose of the T cell engaging therapy is or includes between or between about 0.01 μg/kg and 100 mg/kg, 0.1 μg/kg and 10 μg/kg, 10 μg/kg and 50 μg/kg, 50 μg/kg and 100 μg/kg, 0.1 mg/kg and 1 mg/kg, 1 mg/kg and 10 mg/kg, 10 mg/kg and 100 mg/kg, 100 mg/kg and 500 mg/kg, 200 mg/kg and 300 mg/kg, 100 mg/kg and 250 mg/kg, 200 mg/kg and 400 mg/kg, 250 mg/kg and 500 mg/kg, 250 mg/kg and 750 mg/kg, 50 mg/kg and 750 mg/kg, 1 mg/kg and 10 mg/kg, or 100 mg/kg and 1,000 mg/kg, each inclusive. In some embodiments, the dose of the T cell engaging therapy is at least or at least about or is or is about 0.1 μg/kg, 0.5 μg/kg, 1 μg/kg, 5 μg/kg, 10 μg/kg, 20 μg/kg, 30 μg/kg, 40 μg/kg, 50 μg/kg, 60 μg/kg, 70 μg/kg, 80 μg/kg, 90 μg/kg, 0.1 mg/kg, 0.5 mg/kg, 1 mg/kg, 2.5 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 200 mg/kg, 300 mg/kg, 400 mg/kg, 500 mg/kg, 600 mg/kg, 700 mg/kg, 800 mg/kg, 900 mg/kg, or 1,000 mg/kg. In particular embodiments, the T cell engaging therapy is administered orally, intravenously, intraperitoneally, transdermally, intrathecally, intramuscularly, intranasally, transmucosally, subcutaneously, or rectally.

2. Cell Therapy

In some embodiments, the therapy, e.g. cytotoxic therapy, is a cell-based therapy that is or comprises administration of cells, such as immune cells, for example T cell or NK cells, that target a molecule expressed on the surface of a lesion, such as a tumor or a cancer. In some aspects, the cell therapy is a tumor infiltrating lymphocytic (TIL) therapy, a natural kill (NK) cell therapy, a transgenic TCR therapy, or a recombinant-receptor expressing cell therapy, which optionally is a T cell therapy, which optionally is a chimeric antigen receptor(CAR)-expressing cell therapy. In some embodiments, the T cell therapy includes administering T cells engineered to express a chimeric antigen receptor (CAR). In some aspects, the T cell therapy is an adoptive T cell therapy comprising T cells that specifically recognize and/or target an antigen associated with the cancer, such as an antigen associated with a B cell malignancy, e.g. a chronic lymphocytic leukemia (CLL) or a non-Hodgkin lymphoma (NHL) or a subtype thereof. In some aspects, the T cell therapy is an adoptive T cell therapy comprising T cells that specifically recognize and/or target an antigen associated with the cancer, such as an antigen associated with a B cell malignancy, e.g. a chronic lymphocytic leukemia (CLL). In some aspects, the T cell therapy is an adoptive T cell therapy comprising T cells that specifically recognize and/or target an antigen associated with the cancer, such as an antigen associated with a B cell malignancy, e.g. a small lymphocytic lymphoma (SLL). In some aspects, the T cell therapy comprises T cells engineered with a chimeric antigen receptor (CAR) comprising an antigen binding domain that binds, such as specifically binds, to the antigen. In some cases, the antigen targeted by the T cell therapy is CD19.

In some embodiments, the immune cells express a T cell receptor (TCR) or other antigen-binding receptor. In some embodiments, the immune cells express a recombinant receptor, such as a transgenic TCR or a chimeric antigen receptor (CAR). In some embodiments, the cells are autologous to the subject. In some embodiments, the cells are allogeneic to the subject. Exemplary of such cell therapies, e.g. T cell therapies, for use in the provided methods are described below.

In some embodiments, the provided cells express and/or are engineered to express receptors, such as recombinant receptors, including those containing ligand-binding domains or binding fragments thereof, and T cell receptors (TCRs) and components thereof, and/or functional non-TCR antigen receptors, such as chimeric antigen receptors (CARs). In some embodiments, the recombinant receptor contains an extracellular ligand-binding domain that specifically binds to an antigen. In some embodiments, the recombinant receptor is a CAR that contains an extracellular antigen-recognition domain that specifically binds to an antigen. In some embodiments, the ligand, such as an antigen, is a protein expressed on the surface of cells. In some embodiments, the CAR is a TCR-like CAR and the antigen is a processed peptide antigen, such as a peptide antigen of an intracellular protein, which, like a TCR, is recognized on the cell surface in the context of a major histocompatibility complex (MHC) molecule.

In some embodiments, the cells for use in or administered in connection with the provided methods contain or are engineered to contain an engineered receptor, e.g., an engineered antigen receptor, such as a chimeric antigen receptor (CAR), or a T cell receptor (TCR). Among the compositions are pharmaceutical compositions and formulations for administration, such as for adoptive cell therapy. Also provided are therapeutic methods for administering the cells and compositions to subjects, e.g., patients, in accord with the provided methods, and/or with the provided articles of manufacture or compositions.

Among the engineered cells, including engineered cells containing recombinant receptors, are described in Section II below. Exemplary recombinant receptors, including CARs and recombinant TCRs, as well as methods for engineering and introducing the receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In some aspects, the genetically engineered antigen receptors include a CAR as described in U.S. Pat. No. 7,446,190, and those described in International Patent Application Publication No.: WO/2014055668 A1.

Methods for administration of cells for adoptive cell therapy are known and may be used in connection with the provided methods, compositions and articles of manufacture and kits. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

The cells of the T cell therapy can be administered in a composition formulated for administration, or alternatively, in more than one composition (e.g., two compositions) formulated for separate administration. The dose(s) of the cells may include a particular number or relative number of cells or of the engineered cells, and/or a defined ratio or compositions of two or more sub-types within the composition, such as CD4 vs. CD8 T cells.

The cells can be administered by any suitable means. The cells are administered in a dosing regimen to achieve a therapeutic effect, such as a reduction in tumor burden. Dosing and administration may depend in part on the schedule of administration of the inhibitor of a prosurvival BCL2 family proteins, which can be administered prior to, subsequent to and/or simultaneously with initiation of administration of the cell therapy, such as T cell therapy, e.g. CAR T cell therapy. Various dosing schedules of the cell therapy include but are not limited to single or multiple administrations over various time-points, bolus administration, and pulse infusion.

A. Compositions and Formulations

In some embodiments, the dose of cells of the cell therapy, such as a T cell therapy comprising cells engineered with a recombinant antigen receptor, e.g. CAR or TCR, is provided as a composition or formulation, such as a pharmaceutical composition or formulation. Such compositions can be used in accord with the provided methods and/or with the provided articles of manufacture or compositions, such as in the treatment of a B cell malignancy.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some embodiments, the cell therapy, such as engineered T cells (e.g. CAR T cells), are formulated with a pharmaceutically acceptable carrier. In some aspects, the choice of carrier is determined in part by the particular cell or agent and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells or agents, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

The pharmaceutical composition in some embodiments contains cells in amounts effective to treat the disease or condition, such as a therapeutically effective or prophylactically effective amount. Therapeutic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The cells may be administered using standard administration techniques, formulations, and/or devices. Provided are formulations and devices, such as syringes and vials, for storage and administration of the compositions. With respect to cells, administration can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the agent or cell populations are administered parenterally. The term “parenteral,” as used herein, includes intravenous, intramuscular, subcutaneous, rectal, vaginal, and intraperitoneal administration. In some embodiments, the agent or cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like.

The formulations to be used for in vivo administration are generally sterile. Sterility may be readily accomplished, e.g., by filtration through sterile filtration membranes.

B. Dosing

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells. In some embodiments, administration of the cell dose or any additional therapies, e.g., the lymphodepleting therapy, intervention therapy and/or combination therapy, is carried out via outpatient delivery.

For the treatment of disease, the appropriate dosage may depend on the type of disease to be treated, the type of cells or recombinant receptors, the severity and course of the disease, previous therapy, the subject's clinical history and response to the cells, and the discretion of the attending physician. The compositions and cells are in some embodiments suitably administered to the subject at one time or over a series of treatments.

In some embodiments, a dose of cells is administered to subjects in accord with the provided methods, and/or with the provided articles of manufacture or compositions. In some embodiments, the size or timing of the doses is determined as a function of the particular disease or condition (e.g., cancer, e.g., B cell malignancy) in the subject. In some cases, the size or timing of the doses for a particular disease in view of the provided description may be empirically determined.

In some embodiments, the dose of cells comprises between at or about 2×10⁵ of the cells/kg and at or about 2×10⁶ of the cells/kg, such as between at or about 4×10⁵ of the cells/kg and at or about 1×10⁶ of the cells/kg or between at or about 6×10⁵ of the cells/kg and at or about 8×10⁵ of the cells/kg. In some embodiments, the dose of cells comprises no more than 2×10⁵ of the cells (e.g. antigen-expressing, such as CAR-expressing cells) per kilogram body weight of the subject (cells/kg), such as no more than at or about 3×10⁵ cells/kg, no more than at or about 4×10⁵ cells/kg, no more than at or about 5×10⁵ cells/kg, no more than at or about 6×10⁵ cells/kg, no more than at or about 7×10⁵ cells/kg, no more than at or about 8×10⁵ cells/kg, no more than at or about 9×10⁵ cells/kg, no more than at or about 1×10⁶ cells/kg, or no more than at or about 2×10⁶ cells/kg. In some embodiments, the dose of cells comprises at least or at least about or at or about 2×10⁵ of the cells (e.g. antigen-expressing, such as CAR-expressing cells) per kilogram body weight of the subject (cells/kg), such as at least or at least about or at or about 3×10⁵ cells/kg, at least or at least about or at or about 4×10⁵ cells/kg, at least or at least about or at or about 5×10⁵ cells/kg, at least or at least about or at or about 6×10⁵ cells/kg, at least or at least about or at or about 7×10⁵ cells/kg, at least or at least about or at or about 8×10⁵ cells/kg, at least or at least about or at or about 9×10⁵ cells/kg, at least or at least about or at or about 1×10⁶ cells/kg, or at least or at least about or at or about 2×10⁶ cells/kg.

In certain embodiments, the cells, or individual populations of sub-types of cells, are administered to the subject at a range of at or about one million to at or about 100 billion cells and/or that amount of cells per kilogram of body weight, such as, e.g., 1 million to at or about 50 billion cells (e.g., at or about 5 million cells, at or about 25 million cells, at or about 500 million cells, at or about 1 billion cells, at or about 5 billion cells, at or about 20 billion cells, at or about 30 billion cells, at or about 40 billion cells, or a range defined by any two of the foregoing values), at or about 1 million to at or about 50 billion cells (e.g., at or about 5 million cells, at or about 25 million cells, at or about 500 million cells, at or about 1 billion cells, at or about 5 billion cells, at or about 20 billion cells, at or about 30 billion cells, at or about 40 billion cells, or a range defined by any two of the foregoing values), such as at or about 10 million to at or about 100 billion cells (e.g., at or about 20 million cells, at or about 30 million cells, at or about 40 million cells, at or about 60 million cells, at or about 70 million cells, at or about 80 million cells, at or about 90 million cells, at or about 10 billion cells, at or about 25 billion cells, at or about 50 billion cells, at or about 75 billion cells, at or about 90 billion cells, or a range defined by any two of the foregoing values), and in some cases at or about 100 million cells to at or about 50 billion cells (e.g., at or about 120 million cells, at or about 250 million cells, at or about 350 million cells, at or about 450 million cells, at or about 650 million cells, at or about 800 million cells, at or about 900 million cells, at or about 3 billion cells, at or about 30 billion cells, at or about 45 billion cells) or any value in between these ranges and/or per kilogram of body weight. Dosages may vary depending on attributes particular to the disease or disorder and/or patient and/or other treatments.

In some embodiments, the dose of cells comprises from at or about 1×10⁵ to at or about 5×10⁸ total CAR-expressing T cells, from at or about 1×10⁵ to at or about 2.5×10⁸ total CAR-expressing T cells, from at or about 1×10⁵ to at or about 1×10⁸ total CAR-expressing T cells, from at or about 1×10⁵ to at or about 5×10⁷ total CAR-expressing T cells, from at or about 1×10⁵ to at or about 2.5×10⁷ total CAR-expressing T cells, from at or about 1×10⁵ to at or about 1×10⁷ total CAR-expressing T cells, from at or about 1×10⁵ to at or about 5×10⁶ total CAR-expressing T cells, from at or about 1×10⁵ to at or about 2.5×10⁶ total CAR-expressing T cells, from at or about 1×10⁵ to at or about 1×10⁶ total CAR-expressing T cells, from at or about 1×10⁶ to at or about 5×10⁸ total CAR-expressing T cells, from at or about 1×10⁶ to at or about 2.5×10⁸ total CAR-expressing T cells, from at or about 1×10⁶ to at or about 1×10⁸ total CAR-expressing T cells, from at or about 1×10⁶ to at or about 5×10⁷ total CAR-expressing T cells, from at or about 1×10⁶ to at or about 2.5×10⁷ total CAR-expressing T cells, from at or about 1×10⁶ to at or about 1×10⁷ total CAR-expressing T cells, from at or about 1×10⁶ to at or about 5×10⁶ total CAR-expressing T cells, from at or about 1×10⁶ to at or about 2.5×10⁶ total CAR-expressing T cells, from at or about 2.5×10⁶ to at or about 5×10⁸ total CAR-expressing T cells, from at or about 2.5×10⁶ to at or about 2.5×10⁸ total CAR-expressing T cells, from at or about 2.5×10⁶ to at or about 1×10⁸ total CAR-expressing T cells, from at or about 2.5×10⁶ to at or about 5×10⁷ total CAR-expressing T cells, from at or about 2.5×10⁶ to at or about 2.5×10⁷ total CAR-expressing T cells, from at or about 2.5×10⁶ to at or about 1×10⁷ total CAR-expressing T cells, from at or about 2.5×10⁶ to at or about 5×10⁶ total CAR-expressing T cells, from at or about 5×10⁶ to at or about 5×10⁸ total CAR-expressing T cells, from at or about 5×10⁶ to at or about 2.5×10⁸ total CAR-expressing T cells, from at or about 5×10⁶ to at or about 1×10⁸ total CAR-expressing T cells, from at or about 5×10⁶ to at or about 5×10⁷ total CAR-expressing T cells, from at or about 5×10⁶ to at or about 2.5×10⁷ total CAR-expressing T cells, from at or about 5×10⁶ to at or about 1×10⁷ total CAR-expressing T cells, from at or about 1×10⁷ to at or about 5×10⁸ total CAR-expressing T cells, from at or about 1×10⁷ to at or about 2.5×10⁸ total CAR-expressing T cells, from at or about 1×10⁷ to at or about 1×10⁸ total CAR-expressing T cells, from at or about 1×10⁷ to at or about 5×10⁷ total CAR-expressing T cells, from at or about 1×10⁷ to at or about 2.5×10⁷ total CAR-expressing T cells, from at or about 2.5×10⁷ to at or about 5×10⁸ total CAR-expressing T cells, from at or about 2.5×10⁷ to at or about 2.5×10⁸ total CAR-expressing T cells, from at or about 2.5×10⁷ to at or about 1×10⁸ total CAR-expressing T cells, from at or about 2.5×10⁷ to at or about 5×10⁷ total CAR-expressing T cells, from at or about 5×10⁷ to at or about 5×10⁸ total CAR-expressing T cells, from at or about 5×10⁷ to at or about 2.5×10⁸ total CAR-expressing T cells, from at or about 5×10⁷ to at or about 1×10⁸ total CAR-expressing T cells, from at or about 1×10⁸ to at or about 5×10⁸ total CAR-expressing T cells, from at or about 1×10⁸ to at or about 2.5×10⁸ total CAR-expressing T cells, from at or about or 2.5×10⁸ to at or about 5×10⁸ total CAR-expressing T cells.

In some embodiments, the dose of cells comprises at least or at least about 1×10⁵ CAR-expressing cells, at least or at least about 2.5×10⁵ CAR-expressing cells, at least or at least about 5×10⁵ CAR-expressing cells, at least or at least about 1×10⁶ CAR-expressing cells, at least or at least about 2.5×10⁶ CAR-expressing cells, at least or at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 2.5×10⁸ CAR-expressing cells, or at least or at least about 5×10⁸ CAR-expressing cells.

In some embodiments, the dose of cells is a flat dose of cells or fixed dose of cells such that the dose of cells is not tied to or based on the body surface area or weight of a subject.

In some embodiments, for example, where the subject is a human, the dose includes fewer than at or about 5×10⁸ total recombinant receptor (e.g., CAR)-expressing cells, T cells, or peripheral blood mononuclear cells (PBMCs), e.g., in the range of at or about 1×10⁶ to at or about 5×10⁸ such cells, such as at or about 2×10⁶, 5×10⁶, 1×10⁷, 5×10⁷, 1×10⁸ 2×10⁸, 3×10⁸, 4×10⁸ or 5×10⁸ total such cells, or the range between any two of the foregoing values. In some embodiments, where the subject is a human, the dose includes between at or about 1×10⁶ and at or 3×10⁸ total recombinant receptor (e.g., CAR)-expressing cells, e.g., in the range of at or about 1×10⁷ to at or about 2×10⁸ such cells, such as at or about 1×10⁷, 5×10⁷, 1×10⁸ or 1.5×10⁸ total such cells, or the range between any two of the foregoing values. In some embodiments, the patient is administered multiple doses, and each of the doses or the total dose can be within any of the foregoing values. In some embodiments, the dose of cells comprises the administration of from at or about 1×10⁵ to at or about 5×10⁸ total recombinant receptor (e.g. CAR)-expressing T cells or total T cells, from at or about 1×10⁵ to at or about 1×10⁸ total recombinant receptor (e.g. CAR)-expressing T cells or total T cells, from at or about 5×10⁵ to at or about 1×10⁷ total recombinant receptor (e.g. CAR)-expressing T cells or total T cells, or from at or about 1×10⁶ to at or about 1×10⁷ total recombinant receptor (e.g. CAR)-expressing T cells or total T cells, each inclusive. In some embodiments, the dose of cells comprises the administration of from at or about 2.5×10⁷ total recombinant receptor (e.g. CAR)-expressing T cells. In some embodiments, the dose of cells comprises the administration of from at or about 1×10⁸ total recombinant receptor (e.g. CAR)-expressing T cells.

In some embodiments, the T cells of the dose include CD4+ T cells, CD8+ T cells or CD4+ and CD8+ T cells.

In some embodiments, for example, where the subject is human, the CD8+ T cells of the dose, including in a dose including CD4+ and CD8+ T cells, includes between at or about 1×10⁶ and at or about 1×10⁸ total recombinant receptor (e.g., CAR)-expressing CD8+ cells, e.g., in the range of at or about 5×10⁶ to at or about 1×10⁸ such cells, such cells at or about 1×10⁷, 2.5×10⁷, 5×10⁷, 7.5×10⁷, 1×10⁸, 1.5×10⁸, or 5×10⁸ total such cells, or the range between any two of the foregoing values. In some embodiments, the patient is administered multiple doses, and each of the doses or the total dose can be within any of the foregoing values. In some embodiments, the dose of cells comprises the administration of from at or about 1×10⁷ to at or about 0.75×10⁸ total recombinant receptor-expressing CD8+ T cells, from at or about 1×10⁷ to at or about 2.5×10⁷ total recombinant receptor-expressing CD8+ T cells, from at or about 1×10⁷ to at or about 0.75×10⁸ total recombinant receptor-expressing CD8+ T cells, each inclusive. In some embodiments, the dose of cells comprises the administration of at or about 1×10⁷, 2.5×10⁷, 5×10⁷, 7.5×10⁷, 1×10⁸, 1.5×10⁸, or 5×10⁸ total recombinant receptor-expressing CD8+ T cells.

In some embodiments, for example, where the subject is human, the CD4+ T cells of the dose, including in a dose including CD4+ and CD8+ T cells, includes between at or about 1×10⁶ and at or about 1×10⁸ total recombinant receptor (e.g., CAR)-expressing CD4+ cells, e.g., in the range of at or about 5×10⁶ to 1×10⁸ such cells, such at or about 1×10⁷, 2.5×10⁷, 5×10⁷, 7.5×10⁷, 1×10⁸, 1.5×10⁸, or 5×10⁸ total such cells, or the range between any two of the foregoing values. In some embodiments, the patient is administered multiple doses, and each of the doses or the total dose can be within any of the foregoing values. In some embodiments, the dose of cells comprises the administration of from at or about 1×10⁷ to at or about 0.75×10⁸ total recombinant receptor-expressing CD4+ T cells, from at or about 1×10⁷ to at or about 2.5×10⁷ total recombinant receptor-expressing CD4+ T cells, from at or about 1×10⁷ to at or about 0.75×10⁸ total recombinant receptor-expressing CD4+ T cells, each inclusive. In some embodiments, the dose of cells comprises the administration of at or about 1×10⁷, 2.5×10⁷, 5×10⁷ 7.5×10⁷, 1×10⁸, 1.5×10⁸, or 5×10⁸ total recombinant receptor-expressing CD4+ T cells.

In some embodiments, the dose of cells, e.g., recombinant receptor-expressing T cells, is administered to the subject as a single dose or is administered only one time within a period of two weeks, one month, three months, six months, 1 year or more.

In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose or as a plurality of compositions, provided in multiple individual compositions or infusions, over a specified period of time, such as over no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells of the dose are administered in a single pharmaceutical composition. In some embodiments, the cells of the dose are administered in a plurality of compositions, collectively containing the cells of the dose.

In some embodiments, the term “split dose” refers to a dose that is split so that it is administered over more than one day. This type of dosing is encompassed by the present methods and is considered to be a single dose.

Thus, the dose of cells may be administered as a split dose, e.g., a split dose administered over time. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, cells of the dose may be administered by administration of a plurality of compositions or solutions, such as a first and a second, optionally more, each containing some cells of the dose. In some aspects, the plurality of compositions, each containing a different population and/or sub-types of cells, are administered separately or independently, optionally within a certain period of time. For example, the populations or sub-types of cells can include CD8+ and CD4+ T cells, respectively, and/or CD8+ and CD4+-enriched populations, respectively, e.g., CD4+ and/or CD8+ T cells each individually including cells genetically engineered to express the recombinant receptor. In some embodiments, the administration of the dose comprises administration of a first composition comprising a dose of CD8+ T cells or a dose of CD4+ T cells and administration of a second composition comprising the other of the dose of CD4+ T cells and the CD8+ T cells.

In some embodiments, the administration of the composition or dose, e.g., administration of the plurality of cell compositions, involves administration of the cell compositions separately. In some aspects, the separate administrations are carried out simultaneously, or sequentially, in any order. In some embodiments, the dose comprises a first composition and a second composition, and the first composition and second composition are administered from at or about 0 to at or about 12 hours apart, from at or about 0 to at or about 6 hours apart or from at or about 0 to at or about 2 hours apart. In some embodiments, the initiation of administration of the first composition and the initiation of administration of the second composition are carried out no more than at or about 2 hours, no more than at or about 1 hour, or no more than at or about 30 minutes apart, no more than at or about 15 minutes, no more than at or about 10 minutes or no more than at or about 5 minutes apart. In some embodiments, the initiation and/or completion of administration of the first composition and the completion and/or initiation of administration of the second composition are carried out no more than at or about 2 hours, no more than at or about 1 hour, or no more than at or about 30 minutes apart, no more than at or about 15 minutes, no more than at or about 10 minutes or no more than at or about 5 minutes apart.

In some embodiments, the first composition and the second composition is mixed prior to the administration into the subject. In some embodiments, the first composition and the second composition is mixed shortly (e.g., within at or about 6 hours, 5 hours, 4 hours, 3 hours, 2 hours, 1.5 hours, 1 hour, or 0.5 hour) before the administration, In some embodiments, the first composition and the second composition is mixed immediately before the administration.

In some composition, the first composition, e.g., first composition of the dose, comprises CD4+ T cells. In some composition, the first composition, e.g., first composition of the dose, comprises CD8+ T cells. In some embodiments, the first composition is administered prior to the second composition.

In some embodiments, the dose or composition of cells includes a defined or target ratio of CD4+ cells expressing a recombinant receptor to CD8+ cells expressing a recombinant receptor and/or of CD4+ cells to CD8+ cells, which ratio optionally is approximately 1:1 or is between approximately 1:3 and approximately 3:1, such as approximately 1:1. In some aspects, the administration of a composition or dose with the target or desired ratio of different cell populations (such as CD4+:CD8+ ratio or CAR+ CD4+:CAR+ CD8+ ratio, e.g., 1:1) involves the administration of a cell composition containing one of the populations and then administration of a separate cell composition comprising the other of the populations, where the administration is at or approximately at the target or desired ratio. In some aspects, administration of a dose or composition of cells at a defined ratio leads to improved expansion, persistence and/or antitumor activity of the T cell therapy.

In some embodiments, the subject receives multiple doses, e.g., two or more doses or multiple consecutive doses, of the cells. In some embodiments, two doses are administered to a subject. In some embodiments, the subject receives the consecutive dose, e.g., second dose, approximately 4, 5, 6, 7, 8, 9, 10, 11, 12, 13, 14, 15, 16, 17, 18, 19, 20 or 21 days after the first dose. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses.

In some aspects, the size of the first and/or consecutive dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some aspects, the time between the administration of the first dose and the administration of the consecutive dose is about 9 to about 35 days, about 14 to about 28 days, or 15 to 27 days. In some embodiments, the administration of the consecutive dose is at a time point more than about 14 days after and less than about 28 days after the administration of the first dose. In some aspects, the time between the first and consecutive dose is about 21 days. In some embodiments, an additional dose or doses, e.g. consecutive doses, are administered following administration of the consecutive dose. In some aspects, the additional consecutive dose or doses are administered at least about 14 and less than about 28 days following administration of a prior dose. In some embodiments, the additional dose is administered less than about 14 days following the prior dose, for example, 4, 5, 6, 7, 8, 9, 10, 11, 12, or 13 days after the prior dose. In some embodiments, no dose is administered less than about 14 days following the prior dose and/or no dose is administered more than about 28 days after the prior dose.

In some embodiments, the dose of cells, e.g., recombinant receptor-expressing cells, comprises two doses (e.g., a double dose), comprising a first dose of the T cells and a consecutive dose of the T cells, wherein one or both of the first dose and the second dose comprises administration of the split dose of T cells.

In some embodiments, the dose of cells is generally large enough to be effective in reducing disease burden.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In some embodiments, the cells are administered at or within a tolerated range of a desired output ratio of multiple cell populations or sub-types, such as CD4⁺ and CD8⁺ cells or sub-types. In some aspects, the desired ratio can be a specific ratio or can be a range of ratios. for example, in some embodiments, the desired ratio (e.g., ratio of CD4+ to CD8+ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9: 1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In particular embodiments, the numbers and/or concentrations of cells refer to the number of recombinant receptor (e.g., CAR)-expressing cells. In other embodiments, the numbers and/or concentrations of cells refer to the number or concentration of all cells, T cells, or peripheral blood mononuclear cells (PBMCs) administered.

In some aspects, the size of the dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some embodiments, the methods also include administering one or more additional doses of cells expressing a chimeric antigen receptor (CAR) and/or lymphodepleting therapy, and/or one or more steps of the methods are repeated. In some embodiments, the one or more additional dose is the same as the initial dose. In some embodiments, the one or more additional dose is different from the initial dose, e.g., higher, such as 2-fold, 3-fold, 4-fold, 5-fold, 6-fold, 7-fold, 8-fold, 9-fold or 10-fold or more higher than the initial dose, or lower, such as e.g., higher, such as 2-fold, 3-fold, 4-fold, 5-fold, 6-fold, 7-fold, 8-fold, 9-fold or 10-fold or more lower than the initial dose. In some embodiments, administration of one or more additional doses is determined based on response of the subject to the initial treatment or any prior treatment, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable known methods, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

C. Lymphodepleting Treatment

In some aspects, the provided methods can further include administering one or more lymphodepleting therapies, such as prior to or simultaneous with initiation of administration of the cytotoxic thearpy, such as a T cell therapy (e.g. CAR-expressing T cells). In some embodiments, the lymphodepleting therapy comprises administration of a phosphamide, such as cyclophosphamide. In some embodiments, the lymphodepleting therapy can include administration of fludarabine.

In some aspects, preconditioning subjects with immunodepleting (e.g., lymphodepleting) therapies can improve the effects of adoptive cell therapy (ACT). Preconditioning with lymphodepleting agents, including combinations of cyclosporine and fludarabine, have been effective in improving the efficacy of transferred tumor infiltrating lymphocyte (TIL) cells in cell therapy, including to improve response and/or persistence of the transferred cells. See, e.g., Dudley et al., Science, 298, 850-54 (2002); Rosenberg et al., Clin Cancer Res, 17(13):4550-4557 (2011). Likewise, in the context of CAR+ T cells, several studies have incorporated lymphodepleting agents, most commonly cyclophosphamide, fludarabine, bendamustine, or combinations thereof, sometimes accompanied by low-dose irradiation. See Han et al. Journal of Hematology & Oncology, 6:47 (2013); Kochenderfer et al., Blood, 119: 2709-2720 (2012); Kalos et al., Sci Transl Med, 3(95):95ra73 (2011); Clinical Trial Study Record Nos.: NCT02315612; NCT01822652.

Such preconditioning can be carried out with the goal of reducing the risk of one or more of various outcomes that could dampen efficacy of the therapy. These include the phenomenon known as “cytokine sink,” by which T cells, B cells, NK cells compete with TILs for homeostatic and activating cytokines, such as IL-2, IL-7, and/or IL-15; suppression of TILs by regulatory T cells, NK cells, or other cells of the immune system; impact of negative regulators in the tumor microenvironment. Muranski et al., Nat Clin Pract Oncol. December; 3(12): 668-681 (2006).

Thus in some embodiments, the provided method further involves administering a lymphodepleting therapy to the subject. In some embodiments, the method involves administering the lymphodepleting therapy to the subject prior to the initiation of the administration of the dose of cells. In some embodiments, the lymphodepleting therapy contains a chemotherapeutic agent such as fludarabine and/or cyclophosphamide. In some embodiments, the administration of the cells and/or the lymphodepleting therapy is carried out via outpatient delivery.

In some embodiments, the methods include administering a preconditioning agent, such as a lymphodepleting or chemotherapeutic agent, such as cyclophosphamide, fludarabine, or combinations thereof, to a subject prior to the initiation of the administration of the dose of cells. For example, the subject may be administered a preconditioning agent at least 2 days prior, such as at least 3, 4, 5, 6, or 7 days prior, to the first or subsequent dose. In some embodiments, the subject is administered a preconditioning agent no more than 7 days prior, such as no more than 6, 5, 4, 3, or 2 days prior, to the initiation of administration of the dose of cells. In some embodiments, the subject is administered a preconditioning agent between 2 and 7, inclusive, such as at 2, 3, 4, 5, 6, or 7, days prior to the initiation of the administration of the dose of cells.

In some embodiments, the subject is preconditioned with cyclophosphamide at a dose between or between about 20 mg/kg and 100 mg/kg, such as between or between about 40 mg/kg and 80 mg/kg. In some aspects, the subject is preconditioned with or with about 60 mg/kg of cyclophosphamide. In some embodiments, the cyclophosphamide can be administered in a single dose or can be administered in a plurality of doses, such as given daily, every other day or every three days. In some embodiments, the cyclophosphamide is administered once daily for one or two days. In some embodiments, where the lymphodepleting agent comprises cyclophosphamide, the subject is administered cyclophosphamide at a dose between or between about 100 mg/m² and 500 mg/m², such as between or between about 200 mg/m² and 400 mg/m², or 250 mg/m² and 350 mg/m², inclusive. In some instances, the subject is administered about 300 mg/m² of cyclophosphamide. In some embodiments, the cyclophosphamide can be administered in a single dose or can be administered in a plurality of doses, such as given daily, every other day or every three days. In some embodiments, cyclophosphamide is administered daily, such as for 1-5 days, for example, for 3 to 5 days. In some instances, the subject is administered about 300 mg/m² of cyclophosphamide, daily for 3 days, prior to initiation of the cell therapy.

In some embodiments, where the lymphodepleting agent comprises fludarabine, the subject is administered fludarabine at a dose between or between about 1 mg/m² and 100 mg/m², such as between or between about 10 mg/m² and 75 mg/m², 15 mg/m² and 50 mg/m², 20 mg/m² and 40 mg/m², 24 mg/m² and 35 mg/m², 20 mg/m² and 30 mg/m², or 24 mg/m² and 26 mg/m². In some instances, the subject is administered 25 mg/m² of fludarabine. In some instances, the subject is administered about 30 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 3 to 5 days. In some instances, the subject is administered about 30 mg/m² of fludarabine, daily for 3 days, prior to initiation of the cell therapy.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered 60 mg/kg (˜2 g/m²) of cyclophosphamide and 3 to 5 doses of 25 mg/m² fludarabine prior to the dose of cells. In some embodiments, the subject is administered about 300 mg/m² cyclophosphamide and about 30 mg/m² fludarabine each daily for 3 days. In some embodiments, the preconditioning administration schedule ends between 2 and 7, inclusive, such as at 2, 3, 4, 5, 6, or 7, days prior to the initiation of the administration of the dose of cells.

In one exemplary dosage regimen, prior to receiving the first dose, subjects receive a lymphodepleting preconditioning chemotherapy of cyclophosphamide and fludarabine (CY/FLU), which is administered at least two days before the first dose of CAR-expressing cells and generally no more than 7 days before administration of cells. In some cases a subject is treated with a prosurvival BCL2 family inhibitor prior to receiving a lymphodepleting preconditioning chemotherapy of cyclophosphamide and fludarabine (CY/FLU), wherein treatment of the inhibitor is paused or concluded at least about three days before the subject receives the lymphodepleting therapy. After preconditioning treatment, subjects are administered the dose of CAR-expressing T cells as described above.

In some embodiments, the administration of the preconditioning agent prior to infusion of the dose of cells improves an outcome of the treatment. For example, in some aspects, preconditioning improves the efficacy of treatment with the dose or increases the persistence of the recombinant receptor-expressing cells (e.g., CAR-expressing cells, such as CAR-expressing T cells) in the subject. In some embodiments, preconditioning treatment increases disease-free survival, such as the percent of subjects that are alive and exhibit no minimal residual or molecularly detectable disease after a given period of time following the dose of cells. In some embodiments, the time to median disease-free survival is increased.

Once the cells are administered to the subject (e.g., human), the biological activity of the engineered cell populations in some aspects is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD 107a, IFNγ, IL-2, and TNF. In some aspects the biological activity is measured by assessing clinical outcome, such as reduction in tumor burden or load. In some aspects, toxic outcomes, persistence and/or expansion of the cells, and/or presence or absence of a host immune response, are assessed.

In some embodiments, the administration of the preconditioning agent prior to infusion of the dose of cells improves an outcome of the treatment such as by improving the efficacy of treatment with the dose or increases the persistence of the recombinant receptor-expressing cells (e.g., CAR-expressing cells, such as CAR-expressing T cells) in the subject. Therefore, in some embodiments, the dose of preconditioning agent given in the method which is a combination therapy with the prosurvival BCL2 family protein inhibitor and cell therapy is higher than the dose given in the method without the inhibitor.

### Cell Therapy and Cell Engineering

In some embodiments, the cells contain or are engineered to contain an engineered receptor, e.g., an engineered antigen receptor, such as a chimeric antigen receptor (CAR), or a T cell receptor (TCR). Also provided are populations of such cells, compositions containing such cells and/or enriched for such cells, such as in which cells of a certain type such as T cells or CD8+ or CD4+ cells are enriched or selected. Among the compositions are pharmaceutical compositions and formulations for administration, such as for adoptive cell therapy. Also provided are therapeutic methods for administering the cells and compositions to subjects, e.g., patients.

Thus, in some embodiments, the cells include one or more nucleic acids introduced via genetic engineering, and thereby express recombinant or genetically engineered products of such nucleic acids. In some embodiments, gene transfer is accomplished by first stimulating the cells, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

A. Recombinant Receptors

In some embodiments, the cell therapy, e.g. T cell therapy, for use in accord with the provided combination therapy methods includes administering engineered cells expressing recombinant receptors designed to recognize and/or specifically bind to molecules associated with the disease or condition, such as a cancer, and result in a response, such as an immune response against such molecules upon binding to such molecules. The receptors may include chimeric receptors, e.g., chimeric antigen receptors (CARs), and other transgenic antigen receptors including transgenic T cell receptors (TCRs).

1. Chimeric Antigen Receptors

In some embodiments of the provided methods and uses, the engineered cells, such as T cells, express a chimeric receptors, such as a chimeric antigen receptors (CAR), that contains one or more domains that combine a ligand-binding domain (e.g. antibody or antibody fragment) that provides specificity for a desired antigen (e.g., tumor antigen) with intracellular signaling domains. In some embodiments, the intracellular signaling domain is an activating intracellular domain portion, such as a T cell activating domain, providing a primary activation signal. In some embodiments, the intracellular signaling domain contains or additionally contains a costimulatory signaling domain to facilitate effector functions. Upon specific binding to the molecule, e.g., antigen, the receptor generally delivers an immunostimulatory signal, such as an ITAM-transduced signal, into the cell, thereby promoting an immune response targeted to the disease or condition. In some embodiments, chimeric receptors when genetically engineered into immune cells can modulate T cell activity, and, in some cases, can modulate T cell differentiation or homeostasis, thereby resulting in genetically engineered cells with improved longevity, survival and/or persistence in vivo, such as for use in adoptive cell therapy methods.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061, U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In some aspects, the antigen receptors include a CAR as described in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282.

In some embodiments, the engineered cells, such as T cells, express a recombinant receptor such as a chimeric antigen receptor (CAR) with specificity for a particular antigen (or marker or ligand), such as an antigen expressed on the surface of a particular cell type. In some embodiments, the antigen targeted by the receptor is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

The chimeric receptors, such as CARs, generally include an extracellular antigen binding domain that is an antigen-binding portion or portions of an antibody molecule. In some embodiments, the antigen-binding domain is a portion of an antibody molecule, generally a variable heavy (V_{H}) chain region and/or variable light (V_{L}) chain region of the antibody, e.g., an scFv antibody fragment. In some embodiments, the antigen-binding domain is a single domain antibody (sdAb), such as sdFv, nanobody, V_{H}H and V_{NAR}. In some embodiments, an antigen-binding fragment comprises antibody variable regions joined by a flexible linker.

The chimeric receptors, such as CARs, generally include an extracellular antigen binding domain, such as a portion of an antibody molecule, generally a variable heavy (V_{H}) chain region and/or variable light (V_{L}) chain region of the antibody, e.g., an scFv antibody fragment. In some embodiments, the CAR contains an antibody or an antigen-binding fragment (e.g. scFv) that specifically recognizes an antigen, such as an intact antigen, expressed on the surface of a cell.

Among the antigen receptors are a CAR containing an extracellular antigen binding domain, such as antibody or antigen-binding fragment, that exhibits TCR-like specificity directed against peptide-MHC complexes, which also may be referred to as a TCR-like CAR. In some embodiments, the extracellular antigen binding domain specific for an MHC-peptide complex of a TCR-like CAR is linked to one or more intracellular signaling components, in some aspects via linkers and/or transmembrane domain(s). In some embodiments, such molecules can typically mimic or approximate a signal through a natural antigen receptor, such as a TCR, and, optionally, a signal through such a receptor in combination with a costimulatory receptor.

Reference to “Major histocompatibility complex” (MHC) refers to a protein, generally a glycoprotein, that contains a polymorphic peptide binding site or binding groove that can, in some cases, complex with peptide antigens of polypeptides, including peptide antigens processed by the cell machinery. In some cases, MHC molecules can be displayed or expressed on the cell surface, including as a complex with peptide, i.e. MHC-peptide complex, for presentation of an antigen in a conformation recognizable by an antigen receptor on T cells, such as a TCRs or TCR-like antibody. Generally, MHC class I molecules are heterodimers having a membrane spanning α chain, in some cases with three a domains, and a non-covalently associated β2 microglobulin. Generally, MHC class II molecules are composed of two transmembrane glycoproteins, α and β, both of which typically span the membrane. An MHC molecule can include an effective portion of an MHC that contains an antigen binding site or sites for binding a peptide and the sequences necessary for recognition by the appropriate antigen receptor. In some embodiments, MHC class I molecules deliver peptides originating in the cytosol to the cell surface, where a MHC-peptide complex is recognized by T cells, such as generally CD8⁺ T cells, but in some cases CD4+ T cells. In some embodiments, MHC class II molecules deliver peptides originating in the vesicular system to the cell surface, where they are typically recognized by CD4⁺ T cells. Generally, MHC molecules are encoded by a group of linked loci, which are collectively termed H-2 in the mouse and human leukocyte antigen (HLA) in humans. Hence, typically human MHC can also be referred to as human leukocyte antigen (HLA).

The term “MHC-peptide complex” or “peptide-MHC complex” or variations thereof, refers to a complex or association of a peptide antigen and an MHC molecule, such as, generally, by non-covalent interactions of the peptide in the binding groove or cleft of the MHC molecule. In some embodiments, the MHC-peptide complex is present or displayed on the surface of cells. In some embodiments, the MHC-peptide complex can be specifically recognized by an antigen receptor, such as a TCR, TCR-like CAR or antigen-binding portions thereof.

In some embodiments, a peptide, such as a peptide antigen or epitope, of a polypeptide can associate with an MHC molecule, such as for recognition by an antigen receptor. Generally, the peptide is derived from or based on a fragment of a longer biological molecule, such as a polypeptide or protein. In some embodiments, the peptide typically is about 8 to about 24 amino acids in length. In some embodiments, a peptide has a length of from or from about 9 to 22 amino acids for recognition in the MHC Class II complex. In some embodiments, a peptide has a length of from or from about 8 to 13 amino acids for recognition in the MHC Class I complex. In some embodiments, upon recognition of the peptide in the context of an MHC molecule, such as MHC-peptide complex, the antigen receptor, such as TCR or TCR-like CAR, produces or triggers an activation signal to the T cell that induces a T cell response, such as T cell proliferation, cytokine production, a cytotoxic T cell response or other response.

In some embodiments, a TCR-like antibody or antigen-binding portion, are known or can be produced by known methods (see e.g. US Published Application Nos. US 2002/0150914; US 2003/0223994; US 2004/0191260; US 2006/0034850; US 2007/00992530; US20090226474; US20090304679; and International PCT Publication No. WO 03/068201).

In some embodiments, an antibody or antigen-binding portion thereof that specifically binds to a MHC-peptide complex, can be produced by immunizing a host with an effective amount of an immunogen containing a specific MHC-peptide complex. In some cases, the peptide of the MHC-peptide complex is an epitope of antigen capable of binding to the MHC, such as a tumor antigen, for example a universal tumor antigen, myeloma antigen or other antigen as described below. In some embodiments, an effective amount of the immunogen is then administered to a host for eliciting an immune response, wherein the immunogen retains a three-dimensional form thereof for a period of time sufficient to elicit an immune response against the three-dimensional presentation of the peptide in the binding groove of the MHC molecule. Serum collected from the host is then assayed to determine if desired antibodies that recognize a three-dimensional presentation of the peptide in the binding groove of the MHC molecule is being produced. In some embodiments, the produced antibodies can be assessed to confirm that the antibody can differentiate the MHC-peptide complex from the MHC molecule alone, the peptide of interest alone, and a complex of MHC and irrelevant peptide. The desired antibodies can then be isolated.

In some embodiments, an antibody or antigen-binding portion thereof that specifically binds to an MHC-peptide complex can be produced by employing antibody library display methods, such as phage antibody libraries. In some embodiments, phage display libraries of mutant Fab, scFv or other antibody forms can be generated, for example, in which members of the library are mutated at one or more residues of a CDR or CDRs. See e.g. US published application No. US20020150914, US2014/0294841; and Cohen C J. et al. (2003) J Mol. Recogn. 16:324-332.

The term “antibody” herein is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, variable heavy chain (VH) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

In some embodiments, the antigen-binding proteins, antibodies and antigen binding fragments thereof specifically recognize an antigen of a full-length antibody. In some embodiments, the heavy and light chains of an antibody can be full-length or can be an antigen-binding portion (a Fab, F(ab′)2, Fv or a single chain Fv fragment (scFv)). In other embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4, more particularly, IgG1 (e.g., human IgG1). In another embodiment, the antibody light chain constant region is chosen from, e.g., kappa or lambda, particularly kappa.

Among the provided antibodies are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; variable heavy chain (V_{H}) regions, single-chain antibody molecules such as scFvs and single-domain V_{H} single antibodies; and multispecific antibodies formed from antibody fragments. In particular embodiments, the antibodies are single-chain antibody fragments comprising a variable heavy chain region and/or a variable light chain region, such as scFvs.

The terms “complementarity determining region,” and “CDR,” synonymous with “hypervariable region” or “HVR,” are known, in some cases, to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known, in some cases, to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme); Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

Table 10, below, lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 10
Boundaries of CDRs according to various numbering schemes.

| CDR | Kabat | Chothia | AbM | Contact |
|---|---|---|---|---|
| CDR-L1 | L24-L34 | L24-L34 | L24-L34 | L30-L36 |
| CDR-L2 | L50-L56 | L50-L56 | L50-L56 | L46-L55 |
| CDR-L3 | L89-L97 | L89-L97 | L89-L97 | L89-L96 |
| CDR-H1 | H31-H35B | H26-H32 . . . | H26-H35B | H30-H35B |
| (Kabat | | 34 | | |
| Numbering¹) | | | | |
| CDR-H1 | H31-H35 | H26-H32 | H26-H35 | H30-H35 |
| (Chothia | | | | |
| Numbering²) | | | | |
| CDR-H2 | H50-H65 | H52-H56 | H50-H58 | H47-H58 |
| CDR-H3 | H95-H102 | H95-H102 | H95-H102 | H93-H101 |

¹Kabat et al. (1991), “Sequences of Proteins of Immunological Interest,” 5th Ed. Public Health Service, National Institutes of Health, Bethesda, MD
²Al-Lazikani et al., (1997) JMB 273, 927-948

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes, or other known schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes, or other known schemes. In some embodiments, specific CDR sequences are specified. Exemplary CDR sequences of provided antibodies are described using various numbering schemes, although it is understood that a provided antibody can include CDRs as described according to any of the other aforementioned numbering schemes or other numbering schemes known to a skilled artisan.

Likewise, unless otherwise specified, a FR or individual specified FR(s) (e.g., FR-H1, FR-H2, FR-H3, FR-H4), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, AbM or Contact method, or other known schemes. In other cases, the particular amino acid sequence of a CDR or FR is given.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Single-domain antibodies are antibody fragments comprising all or a portion of the heavy chain variable domain or all or a portion of the light chain variable domain of an antibody. In certain embodiments, a single-domain antibody is a human single-domain antibody. In some embodiments, the CAR comprises an antibody heavy chain domain that specifically binds the antigen, such as a cancer marker or cell surface antigen of a cell or disease to be targeted, such as a tumor cell or a cancer cell, such as any of the target antigens described herein or known.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some embodiments, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

In some embodiments, the recombinant receptor, such as a chimeric receptor (e.g. CAR), includes an extracellular antigen binding domain, such as an antibody or antigen-binding fragment (e.g. scFv), that binds, such as specifically binds, to an antigen (or a ligand). Among the antigens targeted by the chimeric receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas.

In some embodiments, the antigen targeted by the receptor is or comprises selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen targeted by the receptor is or includes CD20, CD19, CD22, ROR1, CD45, CD21, CD5, CD33, Igkappa, Iglambda, CD79a, CD79b or CD30. In some embodiments, the disease or condition is a B cell malignancy, such as a large B cell lymphoma (e.g., DLBCL) and the antigen is CD19.

Antigens targeted by the receptors in some embodiments include antigens associated with a B cell malignancy, such as any of a number of known B cell marker. In some embodiments, the antigen targeted by the receptor is CD20, CD19, CD22, ROR1, CD45, CD21, CD5, CD33, Igkappa, Iglambda, CD79a, CD79b or CD30. In particular aspects, the antigen is CD19. In some embodiments, any of such antigens are antigens expressed on human B cells.

In some embodiments, the antibody or an antigen-binding fragment (e.g. scFv or V_{H} domain) specifically recognizes an antigen, such as CD19. In some embodiments, the antibody or antigen-binding fragment is derived from, or is a variant of, antibodies or antigen-binding fragment that specifically binds to CD19. In some embodiments, the antigen is CD19. In some embodiments, the scFv contains a V_{H} and a V_{L} derived from an antibody or an antibody fragment specific to CD19. In some embodiments, the antibody or antibody fragment that binds CD19 is a mouse derived antibody such as FMC63 and SJ25C1. In some embodiments, the antibody or antibody fragment is a human antibody, e.g., as described in U.S. Patent Publication No. US 2016/0152723.

In some embodiments the antigen-binding domain includes a V_{H} and/or V_{L} derived from FMC63, which, in some aspects, can be an scFv. FMC63 generally refers to a mouse monoclonal IgG1 antibody raised against Nalm-1 and -16 cells expressing CD19 of human origin (Ling, N. R., et al. (1987). Leucocyte typing III. 302). In some embodiments, the FMC63 antibody comprises CDR-H1 and CDR-H2 set forth in SEQ ID NO: 38 and 39, respectively, and CDR-H3 set forth in SEQ ID NO: 40 or 54 and CDR-L1 set forth in SEQ ID NO: 35 and CDR-L2 set forth in SEQ ID NO: 36 or 55 and CDR-L3 sequences set forth in SEQ ID NO: 37 or 56. In some embodiments, the FMC63 antibody comprises the heavy chain variable region (V_{H}) comprising the amino acid sequence of SEQ ID NO: 41 and the light chain variable region (V_{L}) comprising the amino acid sequence of SEQ ID NO: 42.

In some embodiments, the scFv comprises a variable light chain containing the CDR-L1 sequence of SEQ ID NO:35, a CDR-L2 sequence of SEQ ID NO:36, and a CDR-L3 sequence of SEQ ID NO:37 and/or a variable heavy chain containing a CDR-H1 sequence of SEQ ID NO:38, a CDR-H2 sequence of SEQ ID NO:39, and a CDR-H3 sequence of SEQ ID NO:40, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the scFv comprises a variable heavy chain region of FMC63 set forth in SEQ ID NO:41 and a variable light chain region of FMC63 set forth in SEQ ID NO:42, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the variable heavy and variable light chains are connected by a linker. In some embodiments, the linker is set forth in SEQ ID NO:59. In some embodiments, the scFv comprises, in order, a V_{H}, a linker, and a V_{L}. In some embodiments, the scFv comprises, in order, a V_{L}, a linker, and a V_{H}. In some embodiments, the scFv is encoded by a sequence of nucleotides set forth in SEQ ID NO:57 or a sequence that exhibits at least 85%, 86%, 87%, 88%, 89%, 90%, 91%, 92%, 93%, 94%, 95%, 96%, 97%, 98%, or 99% sequence identity to SEQ ID NO:57. In some embodiments, the scFv comprises the sequence of amino acids set forth in SEQ ID NO:43 or a sequence that exhibits at least 85%, 86%, 87%, 88%, 89%, 90%, 91%, 92%, 93%, 94%, 95%, 96%, 97%, 98%, or 99% sequence identity to SEQ ID NO:43.

In some embodiments the antigen-binding domain includes a V_{H} and/or V_{L} derived from SJ25C1, which, in some aspects, can be an scFv. SJ25C1 is a mouse monoclonal IgG1 antibody raised against Nalm-1 and -16 cells expressing CD19 of human origin (Ling, N. R., et al. (1987). Leucocyte typing III. 302). In some embodiments, the SJ25C1 antibody comprises CDR-H1, CDR-H2 and CDR-H3 set forth in SEQ ID NOS: 47-49, respectively, and CDR-L1, CDR-L2 and CDR-L3 sequences set forth in SEQ ID NOS: 44-46, respectively. In some embodiments, the SJ25C1 antibody comprises the heavy chain variable region (V_{H}) comprising the amino acid sequence of SEQ ID NO: 50 and the light chain variable region (V_{L}) comprising the amino acid sequence of SEQ ID NO: 51. In some embodiments, the scFv comprises a variable light chain containing a CDR-L1 sequence of SEQ ID NO:44, a CDR-L2 sequence of SEQ ID NO: 45, and a CDR-L3 sequence of SEQ ID NO:46 and/or a variable heavy chain containing a CDR-H1 sequence of SEQ ID NO:47, a CDR-H2 sequence of SEQ ID NO:48, and a CDR-H3 sequence of SEQ ID NO:49, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the scFv comprises a variable heavy chain region of SJ25C1 set forth in SEQ ID NO:50 and a variable light chain region of SJ25C1 set forth in SEQ ID NO:51, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the variable heavy and variable light chains are connected by a linker. In some embodiments, the linker is set forth in SEQ ID NO:52. In some embodiments, the scFv comprises, in order, a V_{H}, a linker, and a V_{L}. In some embodiments, the scFv comprises, in order, a V_{L}, a linker, and a V_{H}. In some embodiments, the scFv comprises the sequence of amino acids set forth in SEQ ID NO:53 or a sequence that exhibits at least 85%, 86%, 87%, 88%, 89%, 90%, 91%, 92%, 93%, 94%, 95%, 96%, 97%, 98%, or 99% sequence identity to SEQ ID NO:53.

In some embodiments, the antigen is CD20. In some embodiments, the scFv contains a V_{H} and a V_{L} derived from an antibody or an antibody fragment specific to CD20. In some embodiments, the antibody or antibody fragment that binds CD20 is an antibody that is or is derived from Rituximab, such as is Rituximab scFv.

In some embodiments, the antigen is CD22. In some embodiments, the scFv contains a V_{H} and a V_{L} derived from an antibody or an antibody fragment specific to CD22. In some embodiments, the antibody or antibody fragment that binds CD22 is an antibody that is or is derived from m971, such as is m971 scFv.

In some embodiments, the antigen or antigen binding domain is BCMA. In some embodiments, the scFv contains a V_{H} and a V_{L} derived from an antibody or an antibody fragment specific to BCMA. In some embodiments, the antibody or antibody fragment that binds BCMA is or contains a V_{H} and a V_{L} from an antibody or antibody fragment set forth in International Patent Applications, Publication Number WO 2016/090327 and WO 2016/090320.

In some embodiments, the antigen or antigen binding domain is GPRC5D. In some embodiments, the scFv contains a V_{H} and a V_{L} derived from an antibody or an antibody fragment specific to GPRC5D. In some embodiments, the antibody or antibody fragment that binds GPRC5D is or contains a V_{H} and a V_{L} from an antibody or antibody fragment set forth in International Patent Applications, Publication Number WO 2016/090329 and WO 2016/090312.

In some aspects, the recombinant receptor, e.g., a chimeric antigen receptor, includes an extracellular portion containing one or more ligand-(e.g., antigen-) binding domains, such as an antibody or fragment thereof, and one or more intracellular signaling region or domain (also interchangeably called a cytoplasmic signaling domain or region). In some embodiments, the antibody or fragment includes an scFv. In some aspects, the chimeric antigen receptor includes an extracellular portion containing an antibody or fragment and an intracellular signaling region. In some embodiments, the intracellular signaling region comprises an intracellular signaling domain. In some embodiments, the intracellular signaling domain is or comprises a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM). In some aspects, the recombinant receptor, e.g., CAR, further includes a spacer and/or a transmembrane domain or portion. In some aspects, the spacer and/or transmembrane domain can link the extracellular portion containing the ligand-(e.g., antigen-) binding domain and the intracellular signaling region(s) or domain(s)

In some embodiments, the recombinant receptor such as the CAR, further includes a spacer, which may be or include at least a portion of an immunoglobulin constant region or variant or modified version thereof, such as a hinge region, e.g., an IgG4 hinge region, and/or a C_{H}1/C_{L} and/or Fc region. In some embodiments, the recombinant receptor further comprises a spacer and/or a hinge region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. In some examples, the spacer is at or about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153, Hudecek et al. (2015) Cancer Immunol Res. 3(2): 125-135 or international patent application publication number WO2014031687, U.S. Pat. No. 8,822,647 or published app. No. US2014/0271635.

In some embodiments, the spacer contains only a hinge region of an IgG, such as only a hinge of IgG4 or IgG1, such as the hinge only spacer set forth in SEQ ID NO: 1, and encoded by the sequence set forth in SEQ ID NO: 2. In some embodiments, the spacer is an Ig hinge, e.g., and IgG4 hinge, linked to a C_{H}2 and/or C_{H}3 domains. In some embodiments, the spacer is an Ig hinge, e.g., an IgG4 hinge, linked to C_{H}2 and C_{H}3 domains, such as set forth in SEQ ID NO:4. In some embodiments, the spacer the spacer is an Ig hinge, e.g., an IgG4 hinge, linked to a C_{H}3 domain only, such as set forth in SEQ ID NO: 3. In some embodiments, the spacer is or comprises a glycine-serine rich sequence or other flexible linker such as known flexible linkers. In some embodiments, the constant region or portion is of IgD. In some embodiments, the spacer has the sequence set forth in SEQ ID NO: 5. In some embodiments, the spacer has a sequence of amino acids that exhibits at least or at least about 85%, 86%, 87%, 88%, 89%, 90%, 91%, 92%, 93%, 94%, 95%, 96%, 97%, 98%, 99% or more sequence identity to any of SEQ ID NOS: 1, 3, 4 and 5.

In some aspects, the spacer is a polypeptide spacer that (a) comprises or consists of all or a portion of an immunoglobulin hinge or a modified version thereof or comprises about 15 amino acids or less, and does not comprise a CD28 extracellular region or a CD8 extracellular region, (b) comprises or consists of all or a portion of an immunoglobulin hinge, optionally an IgG4 hinge, or a modified version thereof and/or comprises about 15 amino acids or less, and does not comprise a CD28 extracellular region or a CD8 extracellular region, or (c) is at or about 12 amino acids in length and/or comprises or consists of all or a portion of an immunoglobulin hinge, optionally an IgG4, or a modified version thereof; or (d) consists or comprises the sequence of amino acids set forth in SEQ ID NOS: 1, 3-5, 27-34 or 58, or a variant of any of the foregoing having at least 85%, 86%, 87%, 88%, 89%, 90%, 91%, 92%, 93%, 94%, 95%, 96%, 97%, 98%, 99% or more sequence identity thereto, or (e) comprises or consists of the formula X₁PPX₂P, where X₁ is glycine, cysteine or arginine and X₂ is cysteine or threonine.

In some embodiments, the antigen receptor comprises an intracellular domain linked directly or indirectly to the extracellular domain. In some embodiments, the chimeric antigen receptor includes a transmembrane domain linking the extracellular domain and the intracellular signaling domain. In some embodiments, the intracellular signaling domain comprises an ITAM. For example, in some aspects, the antigen recognition domain (e.g. extracellular domain) generally is linked to one or more intracellular signaling components, such as signaling components that mimic activation through an antigen receptor complex, such as a TCR complex, in the case of a CAR, and/or signal via another cell surface receptor. In some embodiments, the chimeric receptor comprises a transmembrane domain linked or fused between the extracellular domain (e.g. scFv) and intracellular signaling domain. Thus, in some embodiments, the antigen-binding component (e.g., antibody) is linked to one or more transmembrane and intracellular signaling domains.

In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 (4-1BB), or CD154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s). In some aspects, the transmembrane domain contains a transmembrane portion of CD28 or a variant thereof. The extracellular domain and transmembrane can be linked directly or indirectly. In some embodiments, the extracellular domain and transmembrane are linked by a spacer, such as any described herein.

In some embodiments, the transmembrane domain of the receptor, e.g., the CAR is a transmembrane domain of human CD28 or variant thereof, e.g., a 27-amino acid transmembrane domain of a human CD28 (Accession No.: P10747.1), or is a transmembrane domain that comprises the sequence of amino acids set forth in SEQ ID NO: 8 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO:8. In some embodiments, the transmembrane-domain containing portion of the recombinant receptor comprises the sequence of amino acids set forth in SEQ ID NO: 9 or a sequence of amino acids having at least or at least about 85%, 86%, 87%, 88%, 89%, 90%, 91%, 92%, 93%, 94%, 95%, 96%, 97%, 98%, 99% or more sequence identity thereto.

In some embodiments, the recombinant receptor, e.g., CAR, includes at least one intracellular signaling component or components, such as an intracellular signaling region or domain. T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components. Among the intracellular signaling region are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

In some embodiments, upon ligation of the CAR, the cytoplasmic domain or intracellular signaling region of the CAR activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling region of an antigen receptor component or costimulatory molecule is used in place of an intact immunostimulatory chain, for example, if it transduces the effector function signal. In some embodiments, the intracellular signaling regions, e.g., comprising intracellular domain or domains, include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability. In some embodiments, the intracellular signaling regions, e.g., comprising intracellular domain or domains, include the cytoplasmic sequences of a region or domain that is involved in providing costimulatory signal.

In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from CD3 zeta chain, FcR gamma, CD3 gamma, CD3 delta and CD3 epsilon. In some embodiments, cytoplasmic signaling molecule(s) in the CAR contain(s) a cytoplasmic signaling domain, portion thereof, or sequence derived from CD3 zeta.

In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8alpha, CD8beta, CD4, CD25, or CD16. For example, in some aspects, the CAR or other chimeric receptor includes a chimeric molecule between CD3-zeta (CD3-ζ) or Fc receptor γ and CD8alpha, CD8beta, CD4, CD25 or CD16.

In some embodiments, the intracellular (or cytoplasmic) signaling region comprises a human CD3 chain, optionally a CD3 zeta stimulatory signaling domain or functional variant thereof, such as an 112 AA cytoplasmic domain of isoform 3 of human CD3ζ (Accession No.: P20963.2) or a CD3 zeta signaling domain as described in U.S. Pat. Nos. 7,446,190 or 8,911,993. In some embodiments, the intracellular signaling region comprises the sequence of amino acids set forth in SEQ ID NO: 13, 14 or 15 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 13, 14 or 15.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

In some embodiments, the chimeric antigen receptor contains an intracellular domain of a T cell costimulatory molecule. In some embodiments, the CAR includes a signaling domain and/or transmembrane portion of a costimulatory receptor, such as CD28, 4-1BB, OX40 (CD134), CD27, DAP10, DAP12, ICOS and/or other costimulatory receptors. In some embodiments, the CAR includes a costimulatory region or domain of CD28 or 4-1BB, such as of human CD28 or human 4-1BB.

In some embodiments, the intracellular signaling region or domain comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular signaling domain can comprise the sequence of amino acids set forth in SEQ ID NO: 10 or 11 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 10 or 11. In some embodiments, the intracellular region comprises an intracellular costimulatory signaling domain of 4-1BB or functional variant or portion thereof, such as a 42-amino acid cytoplasmic domain of a human 4-1BB (Accession No. Q07011.1) or functional variant or portion thereof, such as the sequence of amino acids set forth in SEQ ID NO: 12 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 12.

In some aspects, the same CAR includes both the primary (or activating) cytoplasmic signaling regions and costimulatory signaling components.

In some embodiments, the activating domain is included within one CAR, whereas the costimulatory component is provided by another CAR recognizing another antigen. In some embodiments, the CARs include activating or stimulatory CARs, costimulatory CARs, both expressed on the same cell (see WO2014/055668). In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (December, 2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In some embodiments, the two receptors induce, respectively, an activating and an inhibitory signal to the cell, such that ligation of one of the receptor to its antigen activates the cell or induces a response, but ligation of the second inhibitory receptor to its antigen induces a signal that suppresses or dampens that response. Examples are combinations of activating CARs and inhibitory CARs (iCARs). Such a strategy may be used, for example, to reduce the likelihood of off-target effects in the context in which the activating CAR binds an antigen expressed in a disease or condition but which is also expressed on normal cells, and the inhibitory receptor binds to a separate antigen which is expressed on the normal cells but not cells of the disease or condition.

In some aspects, the chimeric receptor is or includes an inhibitory CAR (e.g. iCAR) and includes intracellular components that dampen or suppress an immune response, such as an ITAM- and/or co stimulatory-promoted response in the cell. Exemplary of such intracellular signaling components are those found on immune checkpoint molecules, including PD-1, CTLA4, LAG3, BTLA, OX2R, TIM-3, TIGIT, LAIR-1, PGE2 receptors, EP2/4 Adenosine receptors including A2AR. In some aspects, the engineered cell includes an inhibitory CAR including a signaling domain of or derived from such an inhibitory molecule, such that it serves to dampen the response of the cell, for example, that induced by an activating and/or costimulatory CAR.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR in some aspects is one that includes multiple costimulatory domains of different costimulatory receptors.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments, the antigen receptor further includes a marker and/or cells expressing the CAR or other antigen receptor further includes a surrogate marker, such as a cell surface marker, which may be used to confirm transduction or engineering of the cell to express the receptor. In some aspects, the marker includes all or part (e.g., truncated form) of CD34, a NGFR, or epidermal growth factor receptor, such as truncated version of such a cell surface receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in published patent application No. WO2014031687. For example, the marker can be a truncated EGFR (tEGFR) that is, optionally, linked to a linker sequence, such as a T2A cleavable linker sequence.

An exemplary polypeptide for a truncated EGFR (e.g. tEGFR) comprises the sequence of amino acids set forth in SEQ ID NO: 7 or 16 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7 or 16. An exemplary T2A linker sequence comprises the sequence of amino acids set forth in SEQ ID NO: 6 or 17 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 6 or 17.

In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some embodiments, the transmembrane domain of the recombinant receptor, e.g., the CAR, is or includes a transmembrane domain of human CD28 (e.g. Accession No. P01747.1) or variant thereof, such as a transmembrane domain that comprises the sequence of amino acids set forth in SEQ ID NO: 8 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 8; in some embodiments, the transmembrane-domain containing portion of the recombinant receptor comprises the sequence of amino acids set forth in SEQ ID NO: 9 or a sequence of amino acids having at least at or about 85%, 86%, 87%, 88%, 89%, 90%, 91%, 92%, 93%, 94%, 95%, 96%, 97%, 98%, 99% or more sequence identity thereto.

In some embodiments, the intracellular signaling component(s) of the recombinant receptor, e.g. the CAR, contains an intracellular costimulatory signaling domain of human CD28 or a functional variant or portion thereof, such as a domain with an LL to GG substitution at positions 186-187 of a native CD28 protein. For example, the intracellular signaling domain can comprise the sequence of amino acids set forth in SEQ ID NO: 10 or 11 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 10 or 11. In some embodiments, the intracellular domain comprises an intracellular costimulatory signaling domain of 4-1BB (e.g. (Accession No. Q07011.1) or functional variant or portion thereof, such as the sequence of amino acids set forth in SEQ ID NO: 12 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 12.

In some embodiments, the intracellular signaling domain of the recombinant receptor, e.g. the CAR, comprises a human CD3 zeta stimulatory signaling domain or functional variant thereof, such as an 112 AA cytoplasmic domain of isoform 3 of human CD3 (Accession No.: P20963.2) or a CD3 zeta signaling domain as described in U.S. Pat. Nos. 7,446,190 or 8,911,993. For example, in some embodiments, the intracellular signaling domain comprises the sequence of amino acids as set forth in SEQ ID NO: 13, 14 or 15 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 13, 14 or 15.

In some embodiments, the spacer contains only a hinge region of an IgG, such as only a hinge of IgG4 or IgG1, such as the hinge only spacer set forth in SEQ ID NO: 1, and encoded by the sequence set forth in SEQ ID NO: 2. In some embodiments, the spacer is an Ig hinge, e.g., and IgG4 hinge, linked to a C_{H}2 and/or C_{H}3 domains. In some embodiments, the spacer is an Ig hinge, e.g., an IgG4 hinge, linked to C_{H}2 and C_{H}3 domains, such as set forth in SEQ ID NO: 4. In some embodiments, the spacer the spacer is an Ig hinge, e.g., an IgG4 hinge, linked to a C_{H}3 domain only, such as set forth in SEQ ID NO: 3. In some embodiments, the spacer is or comprises a glycine-serine rich sequence or other flexible linker such as known flexible linkers. In some embodiments, the constant region or portion is of IgD. In some embodiments, the spacer has the sequence set forth in SEQ ID NO: 5. In some embodiments, the spacer has a sequence of amino acids that exhibits at least or at least about 85%, 86%, 87%, 88%, 89%, 90%, 91%, 92%, 93%, 94%, 95%, 96%, 97%, 98%, 99% or more sequence identity to any of SEQ ID NOS: 1, 3, 4 and 5.

For example, in some embodiments, the CAR includes an antibody such as an antibody fragment, including scFvs, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3 zeta signaling domain. In some embodiments, the CAR includes an antibody or fragment, such as scFv, a spacer such as any of the Ig-hinge containing spacers, a CD28-derived transmembrane domain, a 4-1BB-derived intracellular signaling domain, and a CD3 zeta-derived signaling domain.

In some embodiments, nucleic acid molecules encoding such CAR constructs further includes a sequence encoding a T2A ribosomal skip element and/or a tEGFR sequence, e.g., downstream of the sequence encoding the CAR. In some embodiments, the sequence encodes a T2A ribosomal skip element set forth in SEQ ID NO: 6 or 17, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 6 or 17. In some embodiments, T cells expressing an antigen receptor (e.g. CAR) can also be generated to express a truncated EGFR (EGFRt) as a non-immunogenic selection epitope (e.g. by introduction of a construct encoding the CAR and EGFRt separated by a T2A ribosome switch to express two proteins from the same construct), which then can be used as a marker to detect such cells (see e.g. U.S. Pat. No. 8,802,374). In some embodiments, the sequence encodes an tEGFR sequence set forth in SEQ ID NO: 7 or 16, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7 or 16. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known. Examples of 2A sequences that can be used in the methods and nucleic acids disclosed herein, without limitation, 2A sequences from the foot-and-mouth disease virus (F2A, e.g., SEQ ID NO: 21), equine rhinitis A virus (E2A, e.g., SEQ ID NO: 20), Thosea asigna virus (T2A, e.g., SEQ ID NO: 6 or 17), and porcine teschovirus-1 (P2A, e.g., SEQ ID NO: 18 or 19) as described in U.S. Patent Publication No. 20070116690.

In some of any of the embodiments, the CAR comprises, in order, an scFv specific for the antigen, a transmembrane domain, a cytoplasmic signaling domain derived from a costimulatory molecule, which optionally is or comprises a 4-1BB, and a cytoplasmic signaling domain derived from a primary signaling ITAM-containing molecule, which optionally is or comprises a CD3 zeta signaling domain and optionally further includes a spacer between the transmembrane domain and the scFv;

In some of any of the embodiments, the CAR includes, in order, an scFv specific for the antigen, a transmembrane domain, a cytoplasmic signaling domain derived from a costimulatory molecule, which optionally is or comprises a 4-1BB signaling domain, and a cytoplasmic signaling domain derived from a primary signaling ITAM-containing molecule, which optionally is a CD3 zeta signaling domain.

In some of any of the embodiments, the CAR comprises or consists of, in order, an scFv specific for the antigen, a spacer, a transmembrane domain, a cytoplasmic signaling domain derived from a costimulatory molecule, which optionally is a 4-1BB signaling domain, and a cytoplasmic signaling domain derived from a primary signaling ITAM-containing molecule, which optionally is or comprises a CD3 zeta signaling domain. In some aspects, the spacer is a polypeptide spacer that (a) comprises or consists of all or a portion of an immunoglobulin hinge or a modified version thereof or comprises about 15 amino acids or less, and does not comprise a CD28 extracellular region or a CD8 extracellular region, (b) comprises or consists of all or a portion of an immunoglobulin hinge, optionally an IgG4 hinge, or a modified version thereof and/or comprises about 15 amino acids or less, and does not comprise a CD28 extracellular region or a CD8 extracellular region, or (c) is at or about 12 amino acids in length and/or comprises or consists of all or a portion of an immunoglobulin hinge, optionally an IgG4, or a modified version thereof; or (d) has or consists of the sequence of SEQ ID NO: 1, a sequence encoded by SEQ ID NO: 2, SEQ ID NO: 30, SEQ ID NO: 31, SEQ ID NO: 32, SEQ ID NO: 33, SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity thereto, or (e) comprises or consists of the formula X₁PPX₂P, where X₁ is glycine, cysteine or arginine and X₂ is cysteine or threonine; and/or the costimulatory domain comprises SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto; and/or the primary signaling domain comprises SEQ ID NO: 13 or 14 or 15 or a variant thereof having at least 85%, 86%, 87%, 88%, 89%, 90%, 91%, 92%, 93%, 94%, 95%, 96%, 97%, 98%, 99% or more sequence identity thereto; and/or the scFv comprises a CDRL1 sequence of RASQDISKYLN (SEQ ID NO: 35), a CDRL2 sequence of SRLHSGV (SEQ ID NO: 36), and/or a CDRL3 sequence of GNTLPYTFG (SEQ ID NO: 37) and/or a CDRH1 sequence of DYGVS (SEQ ID NO: 38), a CDRH2 sequence of VIWGSETTYYNSALKS (SEQ ID NO: 39), and/or a CDRH3 sequence of YAMDYWG (SEQ ID NO: 40) or wherein the scFv comprises a variable heavy chain region of FMC63 and a variable light chain region of FMC63 and/or a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63 or binds to the same epitope as or competes for binding with any of the foregoing, and optionally wherein the scFv comprises, in order, a V_{H}, a linker, optionally comprising SEQ ID NO: 59, and a V_{L}, and/or the scFv comprises a flexible linker and/or comprises the amino acid sequence set forth as SEQ ID NO: 59.

In some embodiments, the spacer comprises or consists of SEQ ID NO: 1, the costimulatory domain comprises SEQ ID NO: 12 or variant thereof having at least 85%, 86%, 87%, 88%, 89%, 90%, 91%, 92%, 93%, 94%, 95%, 96%, 97%, 98%, 99% or more sequence identity thereto, the transmembrane domain is of CD28 or comprises SEQ ID NO: 9 or a variant thereof having at least 85%, 86%, 87%, 88%, 89%, 90%, 91%, 92%, 93%, 94%, 95%, 96%, 97%, 98%, 99% or more sequence identity thereto, the scFv contains the binding domain of or CDRs of or V_{H} and V_{L} of FMC63, the primary signaling domain contains SEQ ID NO: 13, 14, or 15, and/or a variant thereof having at least 85%, 86%, 87%, 88%, 89%, 90%, 91%, 92%, 93%, 94%, 95%, 96%, 97%, 98%, 99% or more sequence identity thereto.

In some embodiments, the spacer comprises or consists of SEQ ID NO: 30, the costimulatory domain comprises SEQ ID NO: 12 or variant thereof having at least 85%, 86%, 87%, 88%, 89%, 90%, 91%, 92%, 93%, 94%, 95%, 96%, 97%, 98%, 99% or more sequence identity thereto, the transmembrane domain is of CD28 or comprises SEQ ID NO: 9 or a variant thereof having at least 85%, 86%, 87%, 88%, 89%, 90%, 91%, 92%, 93%, 94%, 95%, 96%, 97%, 98%, 99% or more sequence identity thereto, the scFv contains the binding domain of or CDRs of or V_{H} and V_{L} of FMC63, the primary signaling domain contains SEQ ID NO: 13, 14, or 15, and/or a variant thereof having at least 85%, 86%, 87%, 88%, 89%, 90%, 91%, 92%, 93%, 94%, 95%, 96%, 97%, 98%, 99% or more sequence identity thereto.

In some embodiments, the spacer comprises or consists of SEQ ID NO: 31, the costimulatory domain comprises SEQ ID NO: 12 or variant thereof having at least 85%, 86%, 87%, 88%, 89%, 90%, 91%, 92%, 93%, 94%, 95%, 96%, 97%, 98%, 99% or more sequence identity thereto, the transmembrane domain is of CD28 or comprises SEQ ID NO: 9 or a variant thereof having at least 85%, 86%, 87%, 88%, 89%, 90%, 91%, 92%, 93%, 94%, 95%, 96%, 97%, 98%, 99% or more sequence identity thereto, the scFv contains the binding domain of or CDRs of or V_{H} and V_{L} of FMC63, the primary signaling domain contains SEQ ID NO: 13, 14, or 15, and/or a variant thereof having at least 85%, 86%, 87%, 88%, 89%, 90%, 91%, 92%, 93%, 94%, 95%, 96%, 97%, 98%, 99% or more sequence identity thereto.

In some embodiments, the spacer comprises or consists of SEQ ID NO: 33, the costimulatory domain comprises SEQ ID NO: 12 or variant thereof having at least 85%, 86%, 87%, 88%, 89%, 90%, 91%, 92%, 93%, 94%, 95%, 96%, 97%, 98%, 99% or more sequence identity thereto, the transmembrane domain is of CD28 or comprises SEQ ID NO: 9 or a variant thereof having at least 85%, 86%, 87%, 88%, 89%, 90%, 91%, 92%, 93%, 94%, 95%, 96%, 97%, 98%, 99% or more sequence identity thereto, the scFv contains the binding domain of or CDRs of or V_{H} and V_{L} of FMC63, the primary signaling domain contains SEQ ID NO: 13, 14, or 15, and/or a variant thereof having at least 85%, 86%, 87%, 88%, 89%, 90%, 91%, 92%, 93%, 94%, 95%, 96%, 97%, 98%, 99% or more sequence identity thereto.

In some embodiments, the spacer comprises or consists of SEQ ID NO: 34, the costimulatory domain comprises SEQ ID NO: 12 or variant thereof having at least 85%, 86%, 87%, 88%, 89%, 90%, 91%, 92%, 93%, 94%, 95%, 96%, 97%, 98%, 99% or more sequence identity thereto, the transmembrane domain is of CD28 or comprises SEQ ID NO: 9 or a variant thereof having at least 85%, 86%, 87%, 88%, 89%, 90%, 91%, 92%, 93%, 94%, 95%, 96%, 97%, 98%, 99% or more sequence identity thereto, the scFv contains the binding domain of or CDRs of or V_{H} and V_{L} of FMC63, the primary signaling domain contains SEQ ID NO: 13, 14, or 15, and/or a variant thereof having at least 85%, 86%, 87%, 88%, 89%, 90%, 91%, 92%, 93%, 94%, 95%, 96%, 97%, 98%, 99% or more sequence identity thereto.

The recombinant receptors, such as CARs, expressed by the cells administered to the subject generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the cells express a CAR that specifically binds to an antigen expressed by a cell or tissue of the disease or condition or associated with the disease or condition.

2. T Cell Receptors

In some embodiments, engineered cells, such as T cells, used in connection with the provided methods, uses, articles of manufacture or compositions are cells that express a T cell receptor (TCR) or antigen-binding portion thereof that recognizes an peptide epitope or T cell epitope of a target polypeptide, such as an antigen of a tumor, viral or autoimmune protein.

In some embodiments, a “T cell receptor” or “TCR” is a molecule that contains a variable α and β chains (also known as TCRα and TCRβ, respectively) or a variable γ and δ chains (also known as TCRα and TCRβ, respectively), or antigen-binding portions thereof, and which is capable of specifically binding to a peptide bound to an MHC molecule. In some embodiments, the TCR is in the αβ form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules.

Unless otherwise stated, the term “TCR” should be understood to encompass full TCRs as well as antigen-binding portions or antigen-binding fragments thereof. In some embodiments, the TCR is an intact or full-length TCR, including TCRs in the αβ form or γδ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable α chain and variable β chain of a TCR, sufficient to form a binding site for binding to a specific MHC-peptide complex. Generally, the variable chains of a TCR contain complementarity determining regions involved in recognition of the peptide, MHC and/or MHC-peptide complex.

In some embodiments, the variable domains of the TCR contain hypervariable loops, or complementarity determining regions (CDRs), which generally are the primary contributors to antigen recognition and binding capabilities and specificity. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the alpha chain can interact with the N-terminal part of certain antigenic peptides. In some contexts, CDR1 of the beta chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the β-chain can contain a further hypervariable region (CDR4 or HVR4), which generally is involved in superantigen binding and not antigen recognition (Kotb (1995) Clinical Microbiology Reviews, 8:411-426).

In some embodiments, a TCR also can contain a constant domain, a transmembrane domain and/or a short cytoplasmic tail (see, e.g., Janeway et al., Immunobiology: The Immune System in Health and Disease, 3rd Ed., Current Biology Publications, p. 4:33, 1997). In some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction.

In some embodiments, a TCR chain contains one or more constant domain. For example, the extracellular portion of a given TCR chain (e.g., α-chain or β-chain) can contain two immunoglobulin-like domains, such as a variable domain (e.g., Vα or Vβ; typically amino acids 1 to 116 based on Kabat numbering Kabat et al., “Sequences of Proteins of Immunological Interest, US Dept. Health and Human Services, Public Health Service National Institutes of Health, 1991, 5th ed.) and a constant domain (e.g., α-chain constant domain or Ca, typically positions 117 to 259 of the chain based on Kabat numbering or β chain constant domain or C_{β}, typically positions 117 to 295 of the chain based on Kabat) adjacent to the cell membrane. For example, in some cases, the extracellular portion of the TCR formed by the two chains contains two membrane-proximal constant domains, and two membrane-distal variable domains, which variable domains each contain CDRs. The constant domain of the TCR may contain short connecting sequences in which a cysteine residue forms a disulfide bond, thereby linking the two chains of the TCR. In some embodiments, a TCR may have an additional cysteine residue in each of the α and β chains, such that the TCR contains two disulfide bonds in the constant domains.

In some embodiments, the TCR chains contain a transmembrane domain. In some embodiments, the transmembrane domain is positively charged. In some cases, the TCR chain contains a cytoplasmic tail. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling apparatus or complex. The intracellular tails of CD3 signaling subunits (e.g. CD3γ, CD3δ, CD3ε and CD3ζ chains) contain one or more immunoreceptor tyrosine-based activation motif or ITAM that are involved in the signaling capacity of the TCR complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds.

In some embodiments, the TCR can be generated from a known TCR sequence(s), such as sequences of Vα,β chains, for which a substantially full-length coding sequence is readily available. Methods for obtaining full-length TCR sequences, including V chain sequences, from cell sources are well known. In some embodiments, nucleic acids encoding the TCR can be obtained from a variety of sources, such as by polymerase chain reaction (PCR) amplification of TCR-encoding nucleic acids within or isolated from a given cell or cells, or synthesis of publicly available TCR DNA sequences.

In some embodiments, the TCR is obtained from a biological source, such as from cells such as from a T cell (e.g. cytotoxic T cell), T-cell hybridomas or other publicly available source. In some embodiments, the T-cells can be obtained from in vivo isolated cells. In some embodiments, the TCR is a thymically selected TCR. In some embodiments, the TCR is a neoepitope-restricted TCR. In some embodiments, the T-cells can be a cultured T-cell hybridoma or clone. In some embodiments, the TCR or antigen-binding portion thereof or antigen-binding fragment thereof can be synthetically generated from knowledge of the sequence of the TCR.

In some embodiments, the TCR is generated from a TCR identified or selected from screening a library of candidate TCRs against a target polypeptide antigen, or target T cell epitope thereof. TCR libraries can be generated by amplification of the repertoire of Vα and Vβ from T cells isolated from a subject, including cells present in PBMCs, spleen or other lymphoid organ. In some cases, T cells can be amplified from tumor-infiltrating lymphocytes (TILs). In some embodiments, TCR libraries can be generated from CD4⁺ or CD8⁺ cells. In some embodiments, the TCRs can be amplified from a T cell source of a normal of healthy subject, i.e. normal TCR libraries. In some embodiments, the TCRs can be amplified from a T cell source of a diseased subject, i.e. diseased TCR libraries. In some embodiments, degenerate primers are used to amplify the gene repertoire of Vα and Vβ, such as by RT-PCR in samples, such as T cells, obtained from humans. In some embodiments, scTv libraries can be assembled from naïve Vα and Vβ libraries in which the amplified products are cloned or assembled to be separated by a linker. Depending on the source of the subject and cells, the libraries can be HLA allele-specific. Alternatively, in some embodiments, TCR libraries can be generated by mutagenesis or diversification of a parent or scaffold TCR molecule. In some aspects, the TCRs are subjected to directed evolution, such as by mutagenesis, e.g., of the α or β chain. In some aspects, particular residues within CDRs of the TCR are altered. In some embodiments, selected TCRs can be modified by affinity maturation. In some embodiments, antigen-specific T cells may be selected, such as by screening to assess CTL activity against the peptide. In some aspects, TCRs, e.g. present on the antigen-specific T cells, may be selected, such as by binding activity, e.g., particular affinity or avidity for the antigen.

In some embodiments, the TCR or antigen-binding portion thereof is one that has been modified or engineered. In some embodiments, directed evolution methods are used to generate TCRs with altered properties, such as with higher affinity for a specific MHC-peptide complex. In some embodiments, directed evolution is achieved by display methods including, but not limited to, yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), or T cell display (Chervin et al. (2008) J Immunol Methods, 339, 175-84). In some embodiments, display approaches involve engineering, or modifying, a known, parent or reference TCR. For example, in some cases, a wild-type TCR can be used as a template for producing mutagenized TCRs in which in one or more residues of the CDRs are mutated, and mutants with an desired altered property, such as higher affinity for a desired target antigen, are selected.

In some embodiments, peptides of a target polypeptide for use in producing or generating a TCR of interest are known or can be readily identified. In some embodiments, peptides suitable for use in generating TCRs or antigen-binding portions can be determined based on the presence of an HLA-restricted motif in a target polypeptide of interest, such as a target polypeptide described below. In some embodiments, peptides are identified using available computer prediction models. In some embodiments, for predicting MHC class I binding sites, such models include, but are not limited to, ProPred1 (Singh and Raghava (2001) Bioinformatics 17(12):1236-1237, and SYFPEITHI (see Schuler et al. (2007) Immunoinformatics Methods in Molecular Biology, 409(1): 75-93 2007). In some embodiments, the MHC-restricted epitope is HLA-A0201, which is expressed in approximately 39-46% of all Caucasians and therefore, represents a suitable choice of MHC antigen for use preparing a TCR or other MHC-peptide binding molecule.

HLA-A0201-binding motifs and the cleavage sites for proteasomes and immune-proteasomes using computer prediction models are known. For predicting MHC class I binding sites, such models include, but are not limited to, ProPred1 (described in more detail in Singh and Raghava, ProPred: prediction of HLA-DR binding sites. BIOINFORMATICS 17(12):1236-1237 2001), and SYFPEITHI (see Schuler et al. SYFPEITHI, Database for Searching and T-Cell Epitope Prediction. in Immunoinformatics Methods in Molecular Biology, vol 409(1): 75-93 2007)

In some embodiments, the TCR or antigen binding portion thereof may be a recombinantly produced natural protein or mutated form thereof in which one or more property, such as binding characteristic, has been altered. In some embodiments, a TCR may be derived from one of various animal species, such as human, mouse, rat, or other mammal. A TCR may be cell-bound or in soluble form. In some embodiments, for purposes of the provided methods, the TCR is in cell-bound form expressed on the surface of a cell.

In some embodiments, the TCR is a full-length TCR. In some embodiments, the TCR is an antigen-binding portion. In some embodiments, the TCR is a dimeric TCR (dTCR). In some embodiments, the TCR is a single-chain TCR (sc-TCR). In some embodiments, a dTCR or scTCR have the structures as described in WO 03/020763, WO 04/033685, WO2011/044186.

In some embodiments, the TCR contains a sequence corresponding to the transmembrane sequence. In some embodiments, the TCR does contain a sequence corresponding to cytoplasmic sequences. In some embodiments, the TCR is capable of forming a TCR complex with CD3. In some embodiments, any of the TCRs, including a dTCR or scTCR, can be linked to signaling domains that yield an active TCR on the surface of a T cell. In some embodiments, the TCR is expressed on the surface of cells.

In some embodiments a dTCR contains a first polypeptide wherein a sequence corresponding to a TCR α chain variable region sequence is fused to the N terminus of a sequence corresponding to a TCR α chain constant region extracellular sequence, and a second polypeptide wherein a sequence corresponding to a TCR β chain variable region sequence is fused to the N terminus a sequence corresponding to a TCR β chain constant region extracellular sequence, the first and second polypeptides being linked by a disulfide bond. In some embodiments, the bond can correspond to the native inter-chain disulfide bond present in native dimeric αβ TCRs. In some embodiments, the interchain disulfide bonds are not present in a native TCR. For example, in some embodiments, one or more cysteines can be incorporated into the constant region extracellular sequences of dTCR polypeptide pair. In some cases, both a native and a non-native disulfide bond may be desirable. In some embodiments, the TCR contains a transmembrane sequence to anchor to the membrane.

In some embodiments, a dTCR contains a TCR α chain containing a variable α domain, a constant a domain and a first dimerization motif attached to the C-terminus of the constant a domain, and a TCR β chain comprising a variable β domain, a constant β domain and a first dimerization motif attached to the C-terminus of the constant β domain, wherein the first and second dimerization motifs easily interact to form a covalent bond between an amino acid in the first dimerization motif and an amino acid in the second dimerization motif linking the TCR α chain and TCR β chain together.

In some embodiments, the TCR is a scTCR. Typically, a scTCR can be generated using methods known, See e.g., Soo Hoo, W. F. et al. PNAS (USA) 89, 4759 (1992); Wülfing, C. and Plückthun, A., J. Mol. Biol. 242, 655 (1994); Kurucz, I. et al. PNAS (USA) 90 3830 (1993); International published PCT Nos. WO 96/13593, WO 96/18105, WO99/60120, WO99/18129, WO 03/020763, WO2011/044186; and Schlueter, C. J. et al. J. Mol. Biol. 256, 859 (1996). In some embodiments, a scTCR contains an introduced non-native disulfide interchain bond to facilitate the association of the TCR chains (see e.g. International published PCT No. WO 03/020763). In some embodiments, a scTCR is a non-disulfide linked truncated TCR in which heterologous leucine zippers fused to the C-termini thereof facilitate chain association (see e.g. International published PCT No. WO99/60120). In some embodiments, a scTCR contain a TCRα variable domain covalently linked to a TCRβ variable domain via a peptide linker (see e.g., International published PCT No. WO99/18129).

In some embodiments, a scTCR contains a first segment constituted by an amino acid sequence corresponding to a TCR α chain variable region, a second segment constituted by an amino acid sequence corresponding to a TCR β chain variable region sequence fused to the N terminus of an amino acid sequence corresponding to a TCR β chain constant domain extracellular sequence, and a linker sequence linking the C terminus of the first segment to the N terminus of the second segment.

In some embodiments, a scTCR contains a first segment constituted by an α chain variable region sequence fused to the N terminus of an α chain extracellular constant domain sequence, and a second segment constituted by a β chain variable region sequence fused to the N terminus of a sequence β chain extracellular constant and transmembrane sequence, and, optionally, a linker sequence linking the C terminus of the first segment to the N terminus of the second segment.

In some embodiments, a scTCR contains a first segment constituted by a TCR β chain variable region sequence fused to the N terminus of a β chain extracellular constant domain sequence, and a second segment constituted by an α chain variable region sequence fused to the N terminus of a sequence α chain extracellular constant and transmembrane sequence, and, optionally, a linker sequence linking the C terminus of the first segment to the N terminus of the second segment.

In some embodiments, the linker of a scTCRs that links the first and second TCR segments can be any linker capable of forming a single polypeptide strand, while retaining TCR binding specificity. In some embodiments, the linker sequence may, for example, have the formula -P-AA-P- wherein P is proline and AA represents an amino acid sequence wherein the amino acids are glycine and serine. In some embodiments, the first and second segments are paired so that the variable region sequences thereof are orientated for such binding. Hence, in some cases, the linker has a sufficient length to span the distance between the C terminus of the first segment and the N terminus of the second segment, or vice versa, but is not too long to block or reduces bonding of the scTCR to the target ligand. In some embodiments, the linker can contain from or from about 10 to 45 amino acids, such as 10 to 30 amino acids or 26 to 41 amino acids residues, for example 29, 30, 31 or 32 amino acids. In some embodiments, the linker has the formula -PGGG-(SGGGG)₅-P- wherein P is proline, G is glycine and S is serine (SEQ ID NO:22). In some embodiments, the linker has the sequence GSADDAKKDAAKKDGKS (SEQ ID NO:23)

In some embodiments, the scTCR contains a covalent disulfide bond linking a residue of the immunoglobulin region of the constant domain of the α chain to a residue of the immunoglobulin region of the constant domain of the β chain. In some embodiments, the interchain disulfide bond in a native TCR is not present. For example, in some embodiments, one or more cysteines can be incorporated into the constant region extracellular sequences of the first and second segments of the scTCR polypeptide. In some cases, both a native and a non-native disulfide bond may be desirable.

In some embodiments of a dTCR or scTCR containing introduced interchain disulfide bonds, the native disulfide bonds are not present. In some embodiments, the one or more of the native cysteines forming a native interchain disulfide bonds are substituted to another residue, such as to a serine or alanine. In some embodiments, an introduced disulfide bond can be formed by mutating non-cysteine residues on the first and second segments to cysteine. Exemplary non-native disulfide bonds of a TCR are described in published International PCT No. WO2006/000830.

In some embodiments, the TCR or antigen-binding fragment thereof exhibits an affinity with an equilibrium binding constant for a target antigen of between or between about 10-5 and 10-12 M and all individual values and ranges therein. In some embodiments, the target antigen is an MHC-peptide complex or ligand.

In some embodiments, nucleic acid or nucleic acids encoding a TCR, such as α and β chains, can be amplified by PCR, cloning or other suitable means and cloned into a suitable expression vector or vectors. The expression vector can be any suitable recombinant expression vector, and can be used to transform or transfect any suitable host. Suitable vectors include those designed for propagation and expansion or for expression or both, such as plasmids and viruses.

In some embodiments, the vector can a vector of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), or the pEX series (Clontech, Palo Alto, Calif.). In some cases, bacteriophage vectors, such as λG10, λGT11, λZapII (Stratagene), λEMBL4, and λNM1149, also can be used. In some embodiments, plant expression vectors can be used and include pBI01, pBI101.2, pBI101.3, pBI121 and pBIN19 (Clontech). In some embodiments, animal expression vectors include pEUK-Cl, pMAM and pMAMneo (Clontech). In some embodiments, a viral vector is used, such as a retroviral vector.

In some embodiments, the recombinant expression vectors can be prepared using standard recombinant DNA techniques. In some embodiments, vectors can contain regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based. In some embodiments, the vector can contain a nonnative promoter operably linked to the nucleotide sequence encoding the TCR or antigen-binding portion (or other MHC-peptide binding molecule). In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other known promoters also are contemplated.

In some embodiments, to generate a vector encoding a TCR, the α and β chains are PCR amplified from total cDNA isolated from a T cell clone expressing the TCR of interest and cloned into an expression vector. In some embodiments, the α and β chains are cloned into the same vector. In some embodiments, the α and β chains are cloned into different vectors. In some embodiments, the generated α and β chains are incorporated into a retroviral, e.g. lentiviral, vector. Genetically Engineered Cells and Methods of Producing Cells

In some embodiments, the provided methods involve administering to a subject having a disease or condition cells expressing a recombinant antigen receptor. Various methods for the introduction of genetically engineered components, e.g., recombinant receptors, e.g., CARs or TCRs, are well known and may be used with the provided methods and compositions. Exemplary methods include those for transfer of nucleic acids encoding the receptors, including via viral, e.g., retroviral or lentiviral, transduction, transposons, and electroporation.

Among the cells expressing the receptors and administered by the provided methods are engineered cells. The genetic engineering generally involves introduction of a nucleic acid encoding the recombinant or engineered component into a composition containing the cells, such as by retroviral transduction, transfection, or transformation.

B. Methods of Engineering

In particular embodiments, the engineered cells are produced by a process that generates an output composition of enriched T cells from one or more input compositions and/or from a single biological sample. In certain embodiments, the output composition contains cells that express a recombinant receptor, e.g., a CAR, such as an anti-CD19 CAR. In particular embodiments, the cells of the output compositions are suitable for administration to a subject as a therapy, e.g., an autologous cell therapy. In some embodiments, the output composition is a composition of enriched CD4+ or CD8+ T cells.

In some embodiments, the process for generating or producing engineered cells is by a process that includes some or all of the steps of: collecting or obtaining a biological sample; isolating, selecting, or enriching input cells from the biological sample; cryopreserving and storing the input cells; thawing and/or incubating the input cells under stimulating conditions; engineering the stimulated cells to express or contain a recombinant polynucleotide, e.g., a polynucleotide encoding a recombinant receptor such as a CAR; cultivating the engineered cells, e.g. to a threshold amount, density, or expansion; formulating the cultivated cells in an output composition; and/or cryopreserving and storing the formulated output cells until the cells are released for infusion and/or are suitable to be administered to a subject. In certain embodiments, the process is performed with two or more input compositions of enriched T cells, such as a separate CD4+ composition and a separate CD8+ composition, that are separately processed and engineered from the same starting or initial biological sample and re-infused back into the subject at a defined ratio, e.g. 1:1 ratio of CD4+ to CD8+ T cells. In some embodiments, the enriched T cells are or include engineered T cells, e.g., T cells transduced to express a recombinant receptor.

In particular embodiments, an output composition of engineered cells expressing a recombinant receptor (e.g. anti-CD19 CAR) is produced from an initial and/or input composition of cells. In some embodiments, the input composition is a composition of enriched CD3+ T cells, enriched CD4+ T cells, and/or enriched CD8+ T cells (herein after also referred to as compositions of enriched T cells, compositions of enriched CD4+ T cells, and compositions of enriched CD8+ T cells, respectively). In some embodiments, a composition enriched in CD4+ T cells contains at least 60%, 65%, 70%, 75%, 80%, 85%, 90%, 95%, 98%, 99%, or 99.9% CD4+ T cells. In particular embodiments, the composition of enriched CD4+ T cells contains about 100% CD4+ T cells. In certain embodiments, the composition of enriched CD4+ T cells includes or contains less than 20%, less than 10%, less than 5%, less than 1%, less than 0.1%, or less than 0.01% CD8+ T cells, and/or contains no CD8+ T cells, and/or is free or substantially free of CD8+ T cells. In some embodiments, the populations of enriched CD4+ T cells consist essentially of CD4+ T cells. In some embodiments, a composition enriched in CD8+ T cells contains at least 75%, 80%, 85%, 90%, 95%, 98%, 99%, or 99.9% CD8+ T cells, or contains or contains about 100% CD8+ T cells. In certain embodiments, the composition of enriched CD8+ T cells includes or contains less than 20%, less than 10%, less than 5%, less than 1%, less than 0.1%, or less than 0.01% CD4+ T cells, and/or contains no CD4+ T cells, and/or is free or substantially free of CD4+ T cells. In some embodiments, the populations of enriched CD8+ T cells consist essentially of CD8+ T cells.

In some embodiments, a composition enriched in CD3+ T cells contains at least 60%, 65%, 70%, 75%, 80%, 85%, 90%, 95%, 98%, 99%, or 99.9% CD3+ T cells. In particular embodiments, the composition of enriched CD3+ T cells contains about 100% CD3+ T cells. In certain embodiments, the composition of enriched CD3+ T cells includes CD4+ and CD8+ T cells that are at a ratio of CD4+ T cells to CD8+ T cells of between approximately 1:3 and approximately 3:1, such as approximately 1:1.

In certain embodiments, the process for producing engineered cells further can include one or more of: activating and/or stimulating a cells, e.g., cells of an input composition; genetically engineering the activated and/or stimulated cells, e.g., to introduce a polynucleotide encoding a recombinant protein by transduction or transfection; and/or cultivating the engineered cells, e.g., under conditions that promote proliferation and/or expansion. In particular embodiments, the provided methods may be used in connection with harvesting, collecting, and/or formulating output compositions produced after the cells have been incubated, activated, stimulated, engineered, transduced, transfected, and/or cultivated.

In some embodiments, engineered cells, such as those that express an anti-CD19 CAR, used in accord with the provided methods are produced or generated by a process for selecting, isolating, activating, stimulating, expanding, cultivating, and/or formulating cells. In some embodiments, such methods include any as described.

In some embodiments, at least one separate composition of enriched CD4+ T cells and at least one separate composition of enriched CD8+ T cells are isolated, selected, enriched, or obtained from a single biological sample, e.g., a sample of PBMCs or other white blood cells from the same donor such as a patient or healthy individual. In some embodiments, a separate composition of enriched CD4+ T cells and a separate composition of enriched CD8+ T cells originated, e.g., are initially isolated, selected, and/or enriched, from the same biological sample, such as a single biological sample obtained, collected, and/or taken from a single subject. In some embodiments, a biological sample is first subjected to selection of CD4+ T cells, where both the negative and positive fractions are retained, and the negative fraction is further subjected to selection of CD8+ T cells. In other embodiments, a biological sample is first subjected to selection of CD8+ T cells, where both the negative and positive fractions are retained, and the negative fraction is further subjected to selection of CD4+ T cells. In some embodiments, methods of selection are carried out as described in International PCT publication No. WO2015/164675. In some aspects, a biological sample is first positively selected for CD8+ T cells to generate at least one composition of enriched CD8+ T cells, and the negative fraction is then positively selected for CD4+ T cells to generate at least one composition of enriched CD4+ T cells, such that the at least one composition of enriched CD8+ T cells and the at least one composition of enriched CD4+ T cells are separate compositions from the same biological sample, e.g., from the same donor patient or healthy individual. In some aspects, two or more separate compositions of enriched T cells, e.g., at least one being a composition of enriched CD4+ T cells and at least one being a separate composition of enriched CD8+ T cells from the same donor, are separately frozen, e.g., cryoprotected or cryopreserved in a cryopreservation media.

In some aspects, two or more separate compositions of enriched T cells, e.g., at least one being a composition of enriched CD4+ T cells and at least one being a separate composition of enriched CD8+ T cells from the same biological sample, are activated and/or stimulated by contacting with a stimulatory reagent (e.g., by incubation with CD3/CD28 conjugated magnetic beads for T cell activation). In some aspects, each of the activated/stimulated cell composition is engineered, transduced, and/or transfected, e.g., using a viral vector encoding a recombinant protein (e.g. CAR), to express the same recombinant protein in the CD4+ T cells and CD8+ T cells of each cell composition. In some aspects, the method comprises removing the stimulatory reagent, e.g., magnetic beads, from the cell composition. In some aspects, a cell composition containing engineered CD4+ T cells and a cell composition containing engineered CD8+ T cells are separately cultivated, e.g., for separate expansion of the CD4+ T cell and CD8+ T cell populations therein. In certain embodiments, a cell composition from the cultivation is harvested and/or collected and/or formulated, e.g., by washing the cell composition in a formulation buffer. In certain embodiments, a formulated cell composition comprising CD4+ T cells and a formulated cell composition comprising CD8+ T cells is frozen, e.g., cryoprotected or cryopreserved in a cryopreservation media. In some aspects, engineered CD4+ T cells and CD8+ T cells in each formulation originate from the same donor or biological sample and express the same recombination protein (e.g., CAR, such as anti-CD19 CAR). In some aspects, a separate engineered CD4+ formulation and a separate engineered CD8+ formulation are administered at a defined ratio, e.g. 1:1, to a subject in need thereof such as the same donor.

In some aspects, two or more separate compositions of enriched T cells, e.g., at least one being a composition of enriched CD4+ T cells and at least one being a separate composition of enriched CD8+ T cells from the same biological sample, selected from a sample from a subject and then are combined at a defined ratio, e.g. 1:1. In some embodiments, the combined composition enriched in CD4+ and CD8+ T cells are activated and/or stimulated by contacting with a stimulatory reagent (e.g., by incubation with CD3/CD28 conjugated magnetic beads for T cell activation). In some aspects, the activated/stimulated cell composition is engineered, transduced, and/or transfected, e.g., using a viral vector encoding a recombinant protein (e.g. CAR), to express the recombinant protein in the CD4+ T cells and CD8+ T cells of the cell composition. In some aspects, the method comprises removing the stimulatory reagent, e.g., magnetic beads, from the cell composition. In some aspects, the cell composition containing engineered CD4+ T cells and engineered CD8+ T cells are cultivated, e.g., for expansion of the CD4+ T cell and CD8+ T cell populations therein. In certain embodiments, a cell composition from the cultivation is harvested and/or collected and/or formulated, e.g., by washing the cell composition in a formulation buffer. In certain embodiments, a formulated cell composition comprising recombinant receptor (e.g. CAR) engineered CD4+ T cells and CD8+ T cells is frozen, e.g., cryoprotected or cryopreserved in a cryopreservation media. In some aspects, engineered CD4+ T cells and CD8+ T cells in the formulation originate from the same donor or biological sample and express the same recombinant protein (e.g., CAR, such as anti-CD19 CAR).

In some aspects, a composition of enriched CD3+ T cells is selected from a sample from a subject. In some embodiments, the composition enriched in CD3+ T cells is activated and/or stimulated by contacting with a stimulatory reagent (e.g., by incubation with CD3/CD28 conjugated magnetic beads for T cell activation). In some aspects, the activated/stimulated cell composition is engineered, transduced, and/or transfected, e.g., using a viral vector encoding a recombinant protein (e.g. CAR), to express the recombinant protein in the T cells of the cell composition. In some aspects, the method comprises removing the stimulatory reagent, e.g., magnetic beads, from the cell composition. In some aspects, the cell composition containing engineered CD3+ T cells are cultivated, e.g., for expansion of the T cells populations therein. In certain embodiments, a cell composition from the cultivation is harvested and/or collected and/or formulated, e.g., by washing the cell composition in a formulation buffer. In certain embodiments, a formulated cell composition comprising recombinant receptor (e.g. CAR) engineered CD3+ T cells is frozen, e.g., cryoprotected or cryopreserved in a cryopreservation media. In some aspects, engineered CD3+ T cells in the formulation express a CAR, such as anti-CD19 CAR.

1. Cells and Preparation of Cells for Genetic Engineering

In some embodiments, cells, such as T cells, used in connection with the provided methods, uses, articles of manufacture or compositions are cells have been genetically engineered to express a recombinant receptor, e.g., a CAR or a TCR described herein. In some embodiments, the engineered cells are used in the context of cell therapy, e.g., adoptive cell therapy. In some embodiments, the engineered cells are immune cells. In some embodiments, the engineered cells are T cells, such as CD4+ and CD8+ T cells, CD4+ T cells, or CD8+ T cells.

In some embodiments, the nucleic acids, such as nucleic acids encoding a recombinant receptor, are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, and re-introducing them into the same subject, before or after cryopreservation.

Among the sub-types and subpopulations of T cells and/or of CD4⁺ and/or of CD8⁺ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, preparation of the engineered cells includes one or more culture and/or preparation steps. The cells for introduction of the nucleic acid encoding the transgenic receptor such as the CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, at least a portion of the selection step includes incubation of cells with a selection reagent. The incubation with a selection reagent or reagents, e.g., as part of selection methods which may be performed using one or more selection reagents for selection of one or more different cell types based on the expression or presence in or on the cell of one or more specific molecules, such as surface markers, e.g., surface proteins, intracellular markers, or nucleic acid. In some embodiments, any known method using a selection reagent or reagents for separation based on such markers may be used. In some embodiments, the selection reagent or reagents result in a separation that is affinity- or immunoaffinity-based separation. For example, the selection in some aspects includes incubation with a reagent or reagents for separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

In some aspects of such processes, a volume of cells is mixed with an amount of a desired affinity-based selection reagent. The immunoaffinity-based selection can be carried out using any system or method that results in a favorable energetic interaction between the cells being separated and the molecule specifically binding to the marker on the cell, e.g., the antibody or other binding partner on the solid surface, e.g., particle. In some embodiments, methods are carried out using particles such as beads, e.g. magnetic beads, that are coated with a selection agent (e.g. antibody) specific to the marker of the cells. The particles (e.g. beads) can be incubated or mixed with cells in a container, such as a tube or bag, while shaking or mixing, with a constant cell density-to-particle (e.g., bead) ratio to aid in promoting energetically favored interactions. In other cases, the methods include selection of cells in which all or a portion of the selection is carried out in the internal cavity of a centrifugal chamber, for example, under centrifugal rotation. In some embodiments, incubation of cells with selection reagents, such as immunoaffinity-based selection reagents, is performed in a centrifugal chamber. In certain embodiments, the isolation or separation is carried out using a system, device, or apparatus described in International Patent Application, Publication Number WO2009/072003, or US 20110003380 A1. In one example, the system is a system as described in International Publication Number WO2016/073602.

In some embodiments, by conducting such selection steps or portions thereof (e.g., incubation with antibody-coated particles, e.g., magnetic beads) in the cavity of a centrifugal chamber, the user is able to control certain parameters, such as volume of various solutions, addition of solution during processing and timing thereof, which can provide advantages compared to other available methods. For example, the ability to decrease the liquid volume in the cavity during the incubation can increase the concentration of the particles (e.g. bead reagent) used in the selection, and thus the chemical potential of the solution, without affecting the total number of cells in the cavity. This in turn can enhance the pairwise interactions between the cells being processed and the particles used for selection. In some embodiments, carrying out the incubation step in the chamber, e.g., when associated with the systems, circuitry, and control as described herein, permits the user to effect agitation of the solution at desired time(s) during the incubation, which also can improve the interaction.

In some embodiments, at least a portion of the selection step is performed in a centrifugal chamber, which includes incubation of cells with a selection reagent. In some aspects of such processes, a volume of cells is mixed with an amount of a desired affinity-based selection reagent that is far less than is normally employed when performing similar selections in a tube or container for selection of the same number of cells and/or volume of cells according to manufacturer's instructions. In some embodiments, an amount of selection reagent or reagents that is/are no more than 5%, no more than 10%, no more than 15%, no more than 20%, no more than 25%, no more than 50%, no more than 60%, no more than 70% or no more than 80% of the amount of the same selection reagent(s) employed for selection of cells in a tube or container-based incubation for the same number of cells and/or the same volume of cells according to manufacturer's instructions is employed.

In some embodiments, for selection, e.g., immunoaffinity-based selection of the cells, the cells are incubated in the cavity of the chamber in a composition that also contains the selection buffer with a selection reagent, such as a molecule that specifically binds to a surface marker on a cell that it desired to enrich and/or deplete, but not on other cells in the composition, such as an antibody, which optionally is coupled to a scaffold such as a polymer or surface, e.g., bead, e.g., magnetic bead, such as magnetic beads coupled to monoclonal antibodies specific for CD3, CD4 and/or CD8. In some embodiments, as described, the selection reagent is added to cells in the cavity of the chamber in an amount that is substantially less than (e.g. is no more than 5%, 10%, 20%, 30%, 40%, 50%, 60%, 70% or 80% of the amount) as compared to the amount of the selection reagent that is typically used or would be necessary to achieve about the same or similar efficiency of selection of the same number of cells or the same volume of cells when selection is performed in a tube with shaking or rotation. In some embodiments, the incubation is performed with the addition of a selection buffer to the cells and selection reagent to achieve a target volume with incubation of the reagent of, for example, 10 mL to 200 mL, such as at least or at least about 10 mL, 20 mL, 30 mL, 40 mL, 50 mL, 60 mL, 70 mL, 80 mL, 90 mL, 100 mL, 150 mL or 200 mL. In some embodiments, the selection buffer and selection reagent are pre-mixed before addition to the cells. In some embodiments, the selection buffer and selection reagent are separately added to the cells. In some embodiments, the selection incubation is carried out with periodic gentle mixing condition, which can aid in promoting energetically favored interactions and thereby permit the use of less overall selection reagent while achieving a high selection efficiency.

In some embodiments, the total duration of the incubation with the selection reagent is from or from about 5 minutes to 6 hours, such as 30 minutes to 3 hours, for example, at least or at least about 30 minutes, 60 minutes, 120 minutes or 180 minutes.

In some embodiments, the incubation generally is carried out under mixing conditions, such as in the presence of spinning, generally at relatively low force or speed, such as speed lower than that used to pellet the cells, such as from or from about 600 rpm to 1700 rpm (e.g. at or about or at least 600 rpm, 1000 rpm, or 1500 rpm or 1700 rpm), such as at an RCF at the sample or wall of the chamber or other container of from or from about 80 g to 100 g (e.g. at or about or at least 80 g, 85 g, 90 g, 95 g, or 100 g). In some embodiments, the spin is carried out using repeated intervals of a spin at such low speed followed by a rest period, such as a spin and/or rest for 1, 2, 3, 4, 5, 6, 7, 8, 9, or 10 seconds, such as a spin at approximately 1 or 2 seconds followed by a rest for approximately 5, 6, 7, or 8 seconds.

In some embodiments, such process is carried out within the entirely closed system to which the chamber is integral. In some embodiments, this process (and in some aspects also one or more additional step, such as a previous wash step washing a sample containing the cells, such as an apheresis sample) is carried out in an automated fashion, such that the cells, reagent, and other components are drawn into and pushed out of the chamber at appropriate times and centrifugation effected, so as to complete the wash and binding step in a single closed system using an automated program.

In some embodiments, after the incubation and/or mixing of the cells and selection reagent and/or reagents, the incubated cells are subjected to a separation to select for cells based on the presence or absence of the particular reagent or reagents. In some embodiments, the separation is performed in the same closed system in which the incubation of cells with the selection reagent was performed. In some embodiments, after incubation with the selection reagents, incubated cells, including cells in which the selection reagent has bound are transferred into a system for immunoaffinity-based separation of the cells. In some embodiments, the system for immunoaffinity-based separation is or contains a magnetic separation column.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques.

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In particular embodiments, a biological sample, e.g., a sample of PBMCs or other white blood cells, are subjected to selection of CD4+ T cells, where both the negative and positive fractions are retained. In certain embodiments, CD8+ T cells are selected from the negative fraction. In some embodiments, a biological sample is subjected to selection of CD8+ T cells, where both the negative and positive fractions are retained. In certain embodiments, CD4+ T cells are selected from the negative fraction.

In some embodiments, T cells are separated from a PBMC sample by negative selection of markers expressed on non-T cells, such as B cells, monocytes, or other white blood cells, such as CD14. In some aspects, a CD4+ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al. (2012) Blood. 1:72-82; Wang et al. (2012) J Immunother. 35(9):689-701. In some embodiments, combining T_{CM}-enriched CD8⁺ T cells and CD4⁺ T cells further enhances efficacy.

In embodiments, memory T cells are present in both CD62L⁺ and CD62L⁻ subsets of CD8⁺ peripheral blood lymphocytes. PBMC can be enriched for or depleted of CD62L⁻CD8⁺ and/or CD62L⁺CD8⁺ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD127; in some aspects, it is based on negative selection for cells expressing or highly expressing CD45RA and/or granzyme B. In some aspects, isolation of a CD8+ population enriched for T_{CM} cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4⁺ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4⁺ T helper cells are sorted into naïve, central memory, and effector cells by identifying cell populations that have cell surface antigens. CD4⁺ lymphocytes can be obtained by standard methods. In some embodiments, naive CD4+ T lymphocytes are CD45RO⁻, CD45RA⁺, CD62L⁺, CD4⁺ T cells. In some embodiments, central memory CD4⁺ cells are CD62L⁺ and CD45RO⁺. In some embodiments, effector CD4⁺ cells are CD62L⁻ and CD45RO⁻.

In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinity magnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher© Humana Press Inc., Totowa, N.J.).

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotec, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakura et al. (2012) Blood. 1:72-82, and Wang et al. (2012) J Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° per minute and stored in the vapor phase of a liquid nitrogen storage tank.

In some embodiments, the isolation and/or selection results in one or more input compositions of enriched T cells, e.g., CD3+ T cells, CD4+ T cells, and/or CD8+ T cells. In some embodiments, two or more separate input composition are isolated, selected, enriched, or obtained from a single biological sample. In some embodiments, separate input compositions are isolated, selected, enriched, and/or obtained from separate biological samples collected, taken, and/or obtained from the same subject.

In certain embodiments, the one or more input compositions is or includes a composition of enriched T cells that includes at least 60%, at least 65%, at least 70%, at least 75%, at least 80%, at least 85%, at least 90%, at least 95%, at least 98%, at least 99%, at least 99.5%, at least 99.9%, or at or at about 100% CD3+ T cells. In particular embodiment, the input composition of enriched T cells consists essentially of CD3+ T cells.

In certain embodiments, the one or more input compositions is or includes a composition of enriched CD4+ T cells that includes at least 60%, at least 65%, at least 70%, at least 75%, at least 80%, at least 85%, at least 90%, at least 95%, at least 98%, at least 99%, at least 99.5%, at least 99.9%, or at or at about 100% CD4+ T cells. In certain embodiments, the input composition of CD4+ T cells includes less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free or substantially free of CD8+ T cells. In some embodiments, the composition of enriched T cells consists essentially of CD4+ T cells.

In certain embodiments, the one or more compositions is or includes a composition of CD8+ T cells that is or includes at least 60%, at least 65%, at least 70%, at least 75%, at least 80%, at least 85%, at least 90%, at least 95%, at least 98%, at least 99%, at least 99.5%, at least 99.9%, or at or at about 100% CD8+ T cells. In certain embodiments, the composition of CD8+ T cells contains less than 40%, less than 35%, less than 30%, less than 25%, less than 20%, less than 15%, less than 10%, less than 5%, less than 1%, less than 0.1%, or less than 0.01% CD4+ T cells, and/or contains no CD4+ T cells, and/or is free of or substantially free of CD4+ T cells. In some embodiments, the composition of enriched T cells consists essentially of CD8+ T cells.

2. Activation and Stimulation

In some embodiments, the cells are incubated and/or cultured prior to or in connection with genetic engineering. The incubation steps can include culture, cultivation, stimulation, activation, and/or propagation. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of stimulating or activating an intracellular signaling domain of a TCR complex. In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti-CD28 antibody to the culture medium (e.g., at a concentration of at least about 0.5 ng/ml). In some embodiments, the stimulating agents include IL-2, IL-15 and/or IL-7. In some aspects, the IL-2 concentration is at least about 10 units/mL.

For example, the stimulating conditions can include incubation using anti-CD3/anti-CD28 conjugated magnetic beads (e.g., DYNABEADS® M-450 CD3/CD28 T Cell Expander).

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, the T cells are expanded by adding to a culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4⁺ and/or CD8⁺ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

In some embodiments, at least a portion of the incubation in the presence of one or more stimulating conditions or a stimulatory agents is carried out in the internal cavity of a centrifugal chamber, for example, under centrifugal rotation, such as described in International Publication Number WO2016/073602. In some embodiments, at least a portion of the incubation performed in a centrifugal chamber includes mixing with a reagent or reagents to induce stimulation and/or activation. In some embodiments, cells, such as selected cells, are mixed with a stimulating condition or stimulatory agent in the centrifugal chamber. In some aspects of such processes, a volume of cells is mixed with an amount of one or more stimulating conditions or agents that is far less than is normally employed when performing similar stimulations in a cell culture plate or other system.

In some embodiments, the stimulating agent is added to cells in the cavity of the chamber in an amount that is substantially less than (e.g. is no more than 5%, 10%, 20%, 30%, 40%, 50%, 60%, 70% or 80% of the amount) as compared to the amount of the stimulating agent that is typically used or would be necessary to achieve about the same or similar efficiency of selection of the same number of cells or the same volume of cells when selection is performed without mixing in a centrifugal chamber, e.g. in a tube or bag with periodic shaking or rotation. In some embodiments, the incubation is performed with the addition of an incubation buffer to the cells and stimulating agent to achieve a target volume with incubation of the reagent of, for example, 10 mL to 200 mL, such as at least or at least about or about or 10 mL, 20 mL, 30 mL, 40 mL, 50 mL, 60 mL, 70 mL, 80 mL, 90 mL, 100 mL, 150 mL or 200 mL. In some embodiments, the incubation buffer and stimulating agent are pre-mixed before addition to the cells. In some embodiments, the incubation buffer and stimulating agent are separately added to the cells. In some embodiments, the stimulating incubation is carried out with periodic gentle mixing condition, which can aid in promoting energetically favored interactions and thereby permit the use of less overall stimulating agent while achieving stimulating and activation of cells.

In some embodiments, the incubation generally is carried out under mixing conditions, such as in the presence of spinning, generally at relatively low force or speed, such as speed lower than that used to pellet the cells, such as from or from about 600 rpm to 1700 rpm (e.g. at or about or at least 600 rpm, 1000 rpm, or 1500 rpm or 1700 rpm), such as at an RCF at the sample or wall of the chamber or other container of from or from about 80 g to 100 g (e.g. at or about or at least 80 g, 85 g, 90 g, 95 g, or 100 g). In some embodiments, the spin is carried out using repeated intervals of a spin at such low speed followed by a rest period, such as a spin and/or rest for 1, 2, 3, 4, 5, 6, 7, 8, 9, or 10 seconds, such as a spin at approximately 1 or 2 seconds followed by a rest for approximately 5, 6, 7, or 8 seconds.

In some embodiments, the total duration of the incubation, e.g. with the stimulating agent, is between or between about 1 hour and 96 hours, 1 hour and 72 hours, 1 hour and 48 hours, 4 hours and 36 hours, 8 hours and 30 hours or 12 hours and 24 hours, such as at least or at least about 6 hours, 12 hours, 18 hours, 24 hours, 36 hours or 72 hours. In some embodiments, the further incubation is for a time between or about between 1 hour and 48 hours, 4 hours and 36 hours, 8 hours and 30 hours or 12 hours and 24 hours, inclusive.

In particular embodiments, the stimulating conditions include incubating, culturing, and/or cultivating a composition of enriched T cells with and/or in the presence of one or more cytokines. In particular embodiments, the one or more cytokines are recombinant cytokines. In some embodiments, the one or more cytokines are human recombinant cytokines. In certain embodiments, the one or more cytokines bind to and/or are capable of binding to receptors that are expressed by and/or are endogenous to T cells. In particular embodiments, the one or more cytokines is or includes a member of the 4-alpha-helix bundle family of cytokines. In some embodiments, members of the 4-alpha-helix bundle family of cytokines include, but are not limited to, interleukin-2 (IL-2), interleukin-4 (IL-4), interleukin-7 (IL-7), interleukin-9 (IL-9), interleukin 12 (IL-12), interleukin 15 (IL-15), granulocyte colony-stimulating factor (G-CSF), and granulocyte-macrophage colony-stimulating factor (GM-CSF).

In some embodiments, the stimulation results in activation and/or proliferation of the cells, for example, prior to transduction.

3. Vectors and Methods For Genetic Engineering

In some embodiments, engineered cells, such as T cells, used in connection with the provided methods, uses, articles of manufacture or compositions are cells have been genetically engineered to express a recombinant receptor, e.g., a CAR or a TCR described herein. In some embodiments, the cells are engineered by introduction, delivery or transfer of nucleic acid sequences that encode the recombinant receptor and/or other molecules.

In some embodiments, methods for producing engineered cells includes the introduction of a polynucleotide encoding a recombinant receptor (e.g. anti-CD19 CAR) into a cell, e.g., such as a stimulated or activated cell. In particular embodiments, the recombinant proteins are recombinant receptors, such as any described. Introduction of the nucleic acid molecules encoding the recombinant protein, such as recombinant receptor, in the cell may be carried out using any of a number of known vectors. Such vectors include viral and non-viral systems, including lentiviral and gammaretroviral systems, as well as transposon-based systems such as PiggyBac or Sleeping Beauty-based gene transfer systems. Exemplary methods include those for transfer of nucleic acids encoding the receptors, including via viral, e.g., retroviral or lentiviral, transduction, transposons, and electroporation. In some embodiments, the engineering produces one or more engineered compositions of enriched T cells.

In certain embodiments, the one or more compositions of stimulated T cells are or include two separate stimulated compositions of enriched T cells. In particular embodiments, two separate compositions of enriched T cells, e.g., two separate compositions of enriched T cells that have been selected, isolated, and/or enriched from the same biological sample, are separately engineered. In certain embodiments, the two separate compositions include a composition of enriched CD4+ T cells. In particular embodiments, the two separate compositions include a composition of enriched CD8+ T cells. In some embodiments, two separate compositions of enriched CD4+ T cells and enriched CD8+ T cells are genetically engineered separately.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications. In certain embodiments, the gene transfer is accomplished by first incubating the cells under stimulating conditions, such as by any of the methods described.

In some embodiments, methods for genetic engineering are carried out by contacting one or more cells of a composition with a nucleic acid molecule encoding the recombinant protein, e.g. recombinant receptor. In some embodiments, the contacting can be effected with centrifugation, such as spinoculation (e.g. centrifugal inoculation). Such methods include any of those as described in International Publication Number WO2016/073602. Exemplary centrifugal chambers include those produced and sold by Biosafe SA, including those for use with the Sepax® and Sepax® 2 system, including an A-200/F and A-200 centrifugal chambers and various kits for use with such systems. Exemplary chambers, systems, and processing instrumentation and cabinets are described, for example, in U.S. Pat. Nos. 6,123,655, 6,733,433 and Published U.S. Patent Application, Publication No.: US 2008/0171951, and published international patent application, publication no. WO 00/38762, the contents of each of which are incorporated herein by reference in their entirety. Exemplary kits for use with such systems include, but are not limited to, single-use kits sold by BioSafe SA under product names CS-430.1, CS-490.1, CS-600.1 or CS-900.2.

In some embodiments, the contacting can be effected with centrifugation, such as spinoculation (e.g., centrifugal inoculation). In some embodiments, the composition containing cells, the vector, e.g., viral particles and reagent can be rotated, generally at relatively low force or speed, such as speed lower than that used to pellet the cells, such as from or from about 600 rpm to 1700 rpm (e.g., at or about or at least 600 rpm, 1000 rpm, or 1500 rpm or 1700 rpm). In some embodiments, the rotation is carried at a force, e.g., a relative centrifugal force, of from or from about 100 g to 3200 g (e.g., at or about or at least at or about 100 g, 200 g, 300 g, 400 g, 500 g, 1000 g, 1500 g, 2000 g, 2500 g, 3000 g or 3200 g), as measured for example at an internal or external wall of the chamber or cavity. The term “relative centrifugal force” or RCF is generally understood to be the effective force imparted on an object or substance (such as a cell, sample, or pellet and/or a point in the chamber or other container being rotated), relative to the earth's gravitational force, at a particular point in space as compared to the axis of rotation. The value may be determined using well-known formulas, taking into account the gravitational force, rotation speed and the radius of rotation (distance from the axis of rotation and the object, substance, or particle at which RCF is being measured).

In some embodiments, the system is included with and/or placed into association with other instrumentation, including instrumentation to operate, automate, control and/or monitor aspects of the transduction step and one or more various other processing steps performed in the system, e.g. one or more processing steps that can be carried out with or in connection with the centrifugal chamber system as described herein or in International Publication Number WO2016/073602. This instrumentation in some embodiments is contained within a cabinet. In some embodiments, the instrumentation includes a cabinet, which includes a housing containing control circuitry, a centrifuge, a cover, motors, pumps, sensors, displays, and a user interface. An exemplary device is described in U.S. Pat. Nos. 6,123,655, 6,733,433 and US 2008/0171951.

In some embodiments, the system comprises a series of containers, e.g., bags, tubing, stopcocks, clamps, connectors, and a centrifuge chamber. In some embodiments, the containers, such as bags, include one or more containers, such as bags, containing the cells to be transduced and the viral vector particles, in the same container or separate containers, such as the same bag or separate bags. In some embodiments, the system further includes one or more containers, such as bags, containing medium, such as diluent and/or wash solution, which is pulled into the chamber and/or other components to dilute, resuspend, and/or wash components and/or compositions during the methods. The containers can be connected at one or more positions in the system, such as at a position corresponding to an input line, diluent line, wash line, waste line and/or output line.

In some embodiments, the chamber is associated with a centrifuge, which is capable of effecting rotation of the chamber, such as around its axis of rotation. Rotation may occur before, during, and/or after the incubation in connection with transduction of the cells and/or in one or more of the other processing steps. Thus, in some embodiments, one or more of the various processing steps is carried out under rotation, e.g., at a particular force. The chamber is typically capable of vertical or generally vertical rotation, such that the chamber sits vertically during centrifugation and the side wall and axis are vertical or generally vertical, with the end wall(s) horizontal or generally horizontal.

In some embodiments, during at least a part of the genetic engineering, e.g. transduction, and/or subsequent to the genetic engineering the cells are transferred to a bioreactor bag assembly for culture of the genetically engineered cells, such as for cultivation or expansion of the cells.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV) or spleen focus forming virus (SFFV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, the viral vector particles contain a genome derived from a retroviral genome based vector, such as derived from a lentiviral genome based vector. In some aspects of the provided viral vectors, the heterologous nucleic acid encoding a recombinant receptor, such as an antigen receptor, such as a CAR, is contained and/or located between the 5′ LTR and 3′ LTR sequences of the vector genome.

In some embodiments, the viral vector genome is a lentivirus genome, such as an HIV-1 genome or an SIV genome. For example, lentiviral vectors have been generated by multiply attenuating virulence genes, for example, the genes env, vif, vpu and nef can be deleted, making the vector safer for therapeutic purposes. Lentiviral vectors are known. See Naldini et al., (1996 and 1998); Zufferey et al., (1997); Dull et al., 1998, U.S. Pat. Nos. 6,013,516; and 5,994,136). In some embodiments, these viral vectors are plasmid-based or virus-based, and are configured to carry the essential sequences for incorporating foreign nucleic acid, for selection, and for transfer of the nucleic acid into a host cell. Known lentiviruses can be readily obtained from depositories or collections such as the American Type Culture Collection (“ATCC”; 10801 University Blvd., Manassas, Va. 20110-2209), or isolated from known sources using commonly available techniques.

Non-limiting examples of lentiviral vectors include those derived from a lentivirus, such as Human Immunodeficiency Virus 1 (HIV-1), HIV-2, an Simian Immunodeficiency Virus (SIV), Human T-lymphotropic virus 1 (HTLV-1), HTLV-2 or equine infection anemia virus (El AV). For example, lentiviral vectors have been generated by multiply attenuating the HIV virulence genes, for example, the genes env, vif, vpr, vpu and nef are deleted, making the vector safer for therapeutic purposes. Lentiviral vectors are known in the art, see Naldini et al., (1996 and 1998); Zufferey et al., (1997); Dull et al., 1998, U.S. Pat. Nos. 6,013,516; and 5,994,136). In some embodiments, these viral vectors are plasmid-based or virus-based, and are configured to carry the essential sequences for incorporating foreign nucleic acid, for selection, and for transfer of the nucleic acid into a host cell. Known lentiviruses can be readily obtained from depositories or collections such as the American Type Culture Collection (“ATCC”; 10801 University Blvd., Manassas, Va. 20110-2209), or isolated from known sources using commonly available techniques.

In some embodiments, the viral genome vector can contain sequences of the 5′ and 3′ LTRs of a retrovirus, such as a lentivirus. In some aspects, the viral genome construct may contain sequences from the 5′ and 3′ LTRs of a lentivirus, and in particular can contain the R and U5 sequences from the 5′ LTR of a lentivirus and an inactivated or self-inactivating 3′ LTR from a lentivirus. The LTR sequences can be LTR sequences from any lentivirus from any species. For example, they may be LTR sequences from HIV, SIV, FIV or BIV. Typically, the LTR sequences are HIV LTR sequences.

In some embodiments, the nucleic acid of a viral vector, such as an HIV viral vector, lacks additional transcriptional units. The vector genome can contain an inactivated or self-inactivating 3′ LTR (Zufferey et al. J Virol 72: 9873, 1998; Miyoshi et al., J Virol 72:8150, 1998). For example, deletion in the U3 region of the 3′ LTR of the nucleic acid used to produce the viral vector RNA can be used to generate self-inactivating (SIN) vectors. This deletion can then be transferred to the 5′ LTR of the proviral DNA during reverse transcription. A self-inactivating vector generally has a deletion of the enhancer and promoter sequences from the 3′ long terminal repeat (LTR), which is copied over into the 5′ LTR during vector integration. In some embodiments enough sequence can be eliminated, including the removal of a TATA box, to abolish the transcriptional activity of the LTR. This can prevent production of full-length vector RNA in transduced cells. In some aspects, the U3 element of the 3′ LTR contains a deletion of its enhancer sequence, the TATA box, Sp1, and NF-kappa B sites. As a result of the self-inactivating 3′ LTR, the provirus that is generated following entry and reverse transcription contains an inactivated 5′ LTR. This can improve safety by reducing the risk of mobilization of the vector genome and the influence of the LTR on nearby cellular promoters. The self-inactivating 3′ LTR can be constructed by any method known in the art. In some embodiments, this does not affect vector titers or the in vitro or in vivo properties of the vector.

Optionally, the U3 sequence from the lentiviral 5′ LTR can be replaced with a promoter sequence in the viral construct, such as a heterologous promoter sequence. This can increase the titer of virus recovered from the packaging cell line. An enhancer sequence can also be included. Any enhancer/promoter combination that increases expression of the viral RNA genome in the packaging cell line may be used. In one example, the CMV enhancer/promoter sequence is used (U.S. Pat. Nos. 5,385,839 and 5,168,062).

In certain embodiments, the risk of insertional mutagenesis can be minimized by constructing the retroviral vector genome, such as lentiviral vector genome, to be integration defective. A variety of approaches can be pursued to produce a non-integrating vector genome. In some embodiments, a mutation(s) can be engineered into the integrase enzyme component of the pol gene, such that it encodes a protein with an inactive integrase. In some embodiments, the vector genome itself can be modified to prevent integration by, for example, mutating or deleting one or both attachment sites, or making the 3′ LTR-proximal polypurine tract (PPT) non-functional through deletion or modification. In some embodiments, non-genetic approaches are available; these include pharmacological agents that inhibit one or more functions of integrase. The approaches are not mutually exclusive; that is, more than one of them can be used at a time. For example, both the integrase and attachment sites can be non-functional, or the integrase and PPT site can be non-functional, or the attachment sites and PPT site can be non-functional, or all of them can be non-functional. Such methods and viral vector genomes are known and available (see Philpott and Thrasher, Human Gene Therapy 18:483, 2007; Engelman et al. J Virol 69:2729, 1995; Brown et al J Virol 73:9011 (1999); WO 2009/076524; McWilliams et al., J Virol 77:11150, 2003; Powell and Levin J Virol 70:5288, 1996).

In some embodiments, the vector contains sequences for propagation in a host cell, such as a prokaryotic host cell. In some embodiments, the nucleic acid of the viral vector contains one or more origins of replication for propagation in a prokaryotic cell, such as a bacterial cell. In some embodiments, vectors that include a prokaryotic origin of replication also may contain a gene whose expression confers a detectable or selectable marker such as drug resistance.

The viral vector genome is typically constructed in a plasmid form that can be transfected into a packaging or producer cell line. Any of a variety of known methods can be used to produce retroviral particles whose genome contains an RNA copy of the viral vector genome. In some embodiments, at least two components are involved in making a virus-based gene delivery system: first, packaging plasmids, encompassing the structural proteins as well as the enzymes necessary to generate a viral vector particle, and second, the viral vector itself, i.e., the genetic material to be transferred. Biosafety safeguards can be introduced in the design of one or both of these components.

In some embodiments, the packaging plasmid can contain all retroviral, such as HIV-1, proteins other than envelope proteins (Naldini et al., 1998). In other embodiments, viral vectors can lack additional viral genes, such as those that are associated with virulence, e.g., vpr, vif, vpu and nef, and/or Tat, a primary transactivator of HIV. In some embodiments, lentiviral vectors, such as HIV-based lentiviral vectors, comprise only three genes of the parental virus: gag, pol and rev, which reduces or eliminates the possibility of reconstitution of a wild-type virus through recombination.

In some embodiments, the viral vector genome is introduced into a packaging cell line that contains all the components necessary to package viral genomic RNA, transcribed from the viral vector genome, into viral particles. Alternatively, the viral vector genome may comprise one or more genes encoding viral components in addition to the one or more sequences, e.g., recombinant nucleic acids, of interest. In some aspects, in order to prevent replication of the genome in the target cell, however, endogenous viral genes required for replication are removed and provided separately in the packaging cell line.

In some embodiments, a packaging cell line is transfected with one or more plasmid vectors containing the components necessary to generate the particles. In some embodiments, a packaging cell line is transfected with a plasmid containing the viral vector genome, including the LTRs, the cis-acting packaging sequence and the sequence of interest, i.e. a nucleic acid encoding an antigen receptor, such as a CAR; and one or more helper plasmids encoding the virus enzymatic and/or structural components, such as Gag, pol and/or rev. In some embodiments, multiple vectors are utilized to separate the various genetic components that generate the retroviral vector particles. In some such embodiments, providing separate vectors to the packaging cell reduces the chance of recombination events that might otherwise generate replication competent viruses. In some embodiments, a single plasmid vector having all of the retroviral components can be used.

In some embodiments, the retroviral vector particle, such as lentiviral vector particle, is pseudotyped to increase the transduction efficiency of host cells. For example, a retroviral vector particle, such as a lentiviral vector particle, in some embodiments is pseudotyped with a VSV-G glycoprotein, which provides a broad cell host range extending the cell types that can be transduced. In some embodiments, a packaging cell line is transfected with a plasmid or polynucleotide encoding a non-native envelope glycoprotein, such as to include xenotropic, polytropic or amphotropic envelopes, such as Sindbis virus envelope, GALV or VSV-G.

In some embodiments, the packaging cell line provides the components, including viral regulatory and structural proteins, that are required in trans for the packaging of the viral genomic RNA into lentiviral vector particles. In some embodiments, the packaging cell line may be any cell line that is capable of expressing lentiviral proteins and producing functional lentiviral vector particles. In some aspects, suitable packaging cell lines include 293 (ATCC CCL X), 293T, HeLA (ATCC CCL 2), D17 (ATCC CCL 183), MDCK (ATCC CCL 34), BHK (ATCC CCL-10) and Cf2Th (ATCC CRL 1430) cells.

In some embodiments, the packaging cell line stably expresses the viral protein(s). For example, in some aspects, a packaging cell line containing the gag, pol, rev and/or other structural genes but without the LTR and packaging components can be constructed. In some embodiments, a packaging cell line can be transiently transfected with nucleic acid molecules encoding one or more viral proteins along with the viral vector genome containing a nucleic acid molecule encoding a heterologous protein, and/or a nucleic acid encoding an envelope glycoprotein.

In some embodiments, the viral vectors and the packaging and/or helper plasmids are introduced via transfection or infection into the packaging cell line. The packaging cell line produces viral vector particles that contain the viral vector genome. Methods for transfection or infection are well known. Non-limiting examples include calcium phosphate, DEAE-dextran and lipofection methods, electroporation and microinjection.

When a recombinant plasmid and the retroviral LTR and packaging sequences are introduced into a special cell line (e.g., by calcium phosphate precipitation for example), the packaging sequences may permit the RNA transcript of the recombinant plasmid to be packaged into viral particles, which then may be secreted into the culture media. The media containing the recombinant retroviruses in some embodiments is then collected, optionally concentrated, and used for gene transfer. For example, in some aspects, after cotransfection of the packaging plasmids and the transfer vector to the packaging cell line, the viral vector particles are recovered from the culture media and titered by standard methods used by those of skill in the art.

In some embodiments, a retroviral vector, such as a lentiviral vector, can be produced in a packaging cell line, such as an exemplary HEK 293T cell line, by introduction of plasmids to allow generation of lentiviral particles. In some embodiments, a packaging cell is transfected and/or contains a polynucleotide encoding gag and pol, and a polynucleotide encoding a recombinant receptor, such as an antigen receptor, for example, a CAR. In some embodiments, the packaging cell line is optionally and/or additionally transfected with and/or contains a polynucleotide encoding a rev protein. In some embodiments, the packaging cell line is optionally and/or additionally transfected with and/or contains a polynucleotide encoding a non-native envelope glycoprotein, such as VSV-G. In some such embodiments, approximately two days after transfection of cells, e.g., HEK 293T cells, the cell supernatant contains recombinant lentiviral vectors, which can be recovered and titered.

Recovered and/or produced retroviral vector particles can be used to transduce target cells using the methods as described. Once in the target cells, the viral RNA is reverse-transcribed, imported into the nucleus and stably integrated into the host genome. One or two days after the integration of the viral RNA, the expression of the recombinant protein, e.g., antigen receptor, such as CAR, can be detected.

In some embodiments, the provided methods involve methods of transducing cells by contacting, e.g., incubating, a cell composition comprising a plurality of cells with a viral particle. In some embodiments, the cells to be transfected or transduced are or comprise primary cells obtained from a subject, such as cells enriched and/or selected from a subject.

In some embodiments, the concentration of cells to be transduced of the composition is from or from about 1.0×10⁵ cells/mL to 1.0×10⁸ cells/mL, such as at least or at least about or about 1.0×10⁵ cells/mL, 5×10⁵ cells/mL, 1×10⁶ cells/mL, 5×10⁶ cells/mL, 1×10⁷ cells/mL, 5×10⁷ cells/mL or 1×10⁸ cells/mL.

In some embodiments, the viral particles are provided at a certain ratio of copies of the viral vector particles or infectious units (IU) thereof, per total number of cells to be transduced (IU/cell). For example, in some embodiments, the viral particles are present during the contacting at or about or at least at or about 0.5, 1, 2, 3, 4, 5, 10, 15, 20, 30, 40, 50, or 60 IU of the viral vector particles per one of the cells.

In some embodiments, the titer of viral vector particles is between or between about 1×10⁶ IU/mL and 1×10⁸ IU/mL, such as between or between about 5×10⁶ IU/mL and 5×10⁷ IU/mL, such as at least 6×10⁶ IU/mL, 7×10⁶ IU/mL, 8×10⁶ IU/mL, 9×10⁶ IU/mL, 1×10⁷ IU/mL, 2×10⁷ IU/mL, 3×10⁷ IU/mL, 4×10⁷ IU/mL, or 5×10⁷ IU/mL.

In some embodiments, transduction can be achieved at a multiplicity of infection (MOI) of less than 100, such as generally less than 60, 50, 40, 30, 20, 10, 5 or less.

In some embodiments, the method involves contacting or incubating, the cells with the viral particles. In some embodiments, the contacting is for 30 minutes to 72 hours, such as 30 minute to 48 hours, 30 minutes to 24 hours or 1 hour to 24 hours, such as at least or at least about 30 minutes, 1 hour, 2 hours, 6 hours, 12 hours, 24 hours, 36 hours or more.

In some embodiments, contacting is performed in solution. In some embodiments, the cells and viral particles are contacted in a volume of from or from about 0.5 mL to 500 mL, such as from or from about 0.5 mL to 200 mL, 0.5 mL to 100 mL, 0.5 mL to 50 mL, 0.5 mL to 10 mL, 0.5 mL to 5 mL, 5 mL to 500 mL, 5 mL to 200 mL, 5 mL to 100 mL, 5 mL to 50 mL, 5 mL to 10 mL, 10 mL to 500 mL, 10 mL to 200 mL, 10 mL to 100 mL, 10 mL to 50 mL, 50 mL to 500 mL, 50 mL to 200 mL, 50 mL to 100 mL, 100 mL to 500 mL, 100 mL to 200 mL or 200 mL to 500 mL.

In certain embodiments, the input cells are treated, incubated, or contacted with particles that comprise binding molecules that bind to or recognize the recombinant receptor that is encoded by the viral DNA.

In some embodiments, the incubation of the cells with the viral vector particles results in or produces an output composition comprising cells transduced with the viral vector particles.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No.: WO2014055668, and U.S. Pat. No. 7,446,190.

In some embodiments, the cells, e.g., T cells, may be transfected either during or after expansion e.g. with a T cell receptor (TCR) or a chimeric antigen receptor (CAR). This transfection for the introduction of the gene of the desired receptor can be carried out with any suitable retroviral vector, for example. The genetically modified cell population can then be liberated from the initial stimulus (the anti-CD3/anti-CD28 stimulus, for example) and subsequently be stimulated with a second type of stimulus e.g. via a de novo introduced receptor). This second type of stimulus may include an antigenic stimulus in form of a peptide/MHC molecule, the cognate (cross-linking) ligand of the genetically introduced receptor (e.g. natural ligand of a CAR) or any ligand (such as an antibody) that directly binds within the framework of the new receptor (e.g. by recognizing constant regions within the receptor). See, for example, Cheadle et al, “Chimeric antigen receptors for T-cell based therapy” Methods Mol Biol. 2012; 907:645-66 or Barrett et al., Chimeric Antigen Receptor Therapy for Cancer Annual Review of Medicine Vol. 65: 333-347 (2014).

In some cases, a vector may be used that does not require that the cells, e.g., T cells, are activated. In some such instances, the cells may be selected and/or transduced prior to activation. Thus, the cells may be engineered prior to, or subsequent to culturing of the cells, and in some cases at the same time as or during at least a portion of the culturing.

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

### 4. Cultivation, Expansion and Formulation of Engineered Cells

In some embodiments, the methods for generating the engineered cells, e.g., for cell therapy in accord with any of provided methods, uses, articles of manufacture or compositions, include one or more steps for cultivating cells, e.g., cultivating cells under conditions that promote proliferation and/or expansion. In some embodiments, cells are cultivated under conditions that promote proliferation and/or expansion subsequent to a step of genetically engineering, e.g., introducing a recombinant polypeptide to the cells by transduction or transfection. In particular embodiments, the cells are cultivated after the cells have been incubated under stimulating conditions and transduced or transfected with a recombinant polynucleotide, e.g., a polynucleotide encoding a recombinant receptor. Thus, in some embodiments, a composition of CAR-positive T cells that has been engineered by transduction or transfection with a recombinant polynucleotide encoding the CAR, is cultivated under conditions that promote proliferation and/or expansion.

In certain embodiments, the one or more compositions of engineered T cells are or include two separate compositions of enriched T cells, such as two separate compositions of enriched T cells that have been engineered with a polynucleotide encoding a recombinant receptor, e.g. a CAR. In particular embodiments, two separate compositions of enriched T cells, e.g., two separate compositions of enriched T cells selected, isolated, and/or enriched from the same biological sample, are separately cultivated under stimulating conditions, such as subsequent to a step of genetically engineering, e.g., introducing a recombinant polypeptide to the cells by transduction or transfection. In certain embodiments, the two separate compositions include a composition of enriched CD4+ T cells, such as a composition of enriched CD4+ T cells that have been engineered with a polynucleotide encoding a recombinant receptor, e.g. a CAR. In particular embodiments, the two separate compositions include a composition of enriched CD8+ T cells, such as a composition of enriched CD4+ T cells that have been engineered with a polynucleotide encoding a recombinant receptor, e.g. a CAR. In some embodiments, two separate compositions of enriched CD4+ T cells and enriched CD8+ T cells, such as a composition of enriched CD4+ T cells and a composition of enriched CD8+ T cells that have each been separately engineered with a polynucleotide encoding a recombinant receptor, e.g. a CAR, are separately cultivated, e.g., under conditions that promote proliferation and/or expansion.

In some embodiments, cultivation is carried out under conditions that promote proliferation and/or expansion. In some embodiments, such conditions may be designed to induce proliferation, expansion, activation, and/or survival of cells in the population. In particular embodiments, the stimulating conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to promote growth, division, and/or expansion of the cells.

In particular embodiments, the cells are cultivated in the presence of one or more cytokines. In particular embodiments, the one or more cytokines are recombinant cytokines. In some embodiments, the one or more cytokines are human recombinant cytokines. In certain embodiments, the one or more cytokines bind to and/or are capable of binding to receptors that are expressed by and/or are endogenous to T cells. In particular embodiments, the one or more cytokines, e.g. a recombinant cytokine, is or includes a member of the 4-alpha-helix bundle family of cytokines. In some embodiments, members of the 4-alpha-helix bundle family of cytokines include, but are not limited to, interleukin-2 (IL-2), interleukin-4 (IL-4), interleukin-7 (IL-7), interleukin-9 (IL-9), interleukin 12 (IL-12), interleukin 15 (IL-15), granulocyte colony-stimulating factor (G-CSF), and granulocyte-macrophage colony-stimulating factor (GM-CSF). In some embodiments, the one or more recombinant cytokine includes IL-2, IL-7 and/or IL-15. In some embodiments, the cells, e.g., engineered cells, are cultivated in the presence of a cytokine, e.g., a recombinant human cytokine, at a concentration of between 1 IU/mL and 2,000 IU/mL, between 10 IU/mL and 100 IU/mL, between 50 IU/mL and 200 IU/mL, between 100 IU/mL and 500 IU/mL, between 100 IU/mL and 1,000 IU/mL, between 500 IU/mL and 2,000 IU/mL, or between 100 IU/mL and 1,500 IU/mL.

In some embodiments, the cultivation is performed under conditions that generally include a temperature suitable for the growth of primary immune cells, such as human T lymphocytes, for example, at least about 25 degrees Celsius, generally at least about 30 degrees, and generally at or about 37 degrees Celsius. In some embodiments, the composition of enriched T cells is incubated at a temperature of 25 to 38° C., such as 30 to 37° C., for example at or about 37° C.±2° C. In some embodiments, the incubation is carried out for a time period until the culture, e.g. cultivation or expansion, results in a desired or threshold density, number or dose of cells. In some embodiments, the incubation is greater than or greater than about or is for about or 24 hours, 48 hours, 72 hours, 96 hours, 5 days, 6 days, 7 days, 8 days, 9 days or more.

In particular embodiments, the cultivation is performed in a closed system. In certain embodiments, the cultivation is performed in a closed system under sterile conditions. In particular embodiments, the cultivation is performed in the same closed system as one or more steps of the provided systems. In some embodiments the composition of enriched T cells is removed from a closed system and placed in and/or connected to a bioreactor for the cultivation. Examples of suitable bioreactors for the cultivation include, but are not limited to, GE Xuri W25, GE Xuri W5, Sartorius BioSTAT RM 20|50, Finesse SmartRocker Bioreactor Systems, and Pall XRS Bioreactor Systems. In some embodiments, the bioreactor is used to perfuse and/or mix the cells during at least a portion of the cultivation step.

In some embodiments, the mixing is or includes rocking and/or motioning. In some cases, the bioreactor can be subject to motioning or rocking, which, in some aspects, can increase oxygen transfer. Motioning the bioreactor may include, but is not limited to rotating along a horizontal axis, rotating along a vertical axis, a rocking motion along a tilted or inclined horizontal axis of the bioreactor or any combination thereof. In some embodiments, at least a portion of the incubation is carried out with rocking. The rocking speed and rocking angle may be adjusted to achieve a desired agitation. In some embodiments the rock angle is 20°, 19°, 18°, 17°, 16°, 15°, 14°, 13°, 12°, 11°, 10°, 9°, 8°, 7°, 6°, 5°, 4°, 3°, 2° or 1°. In certain embodiments, the rock angle is between 6-16°. In other embodiments, the rock angle is between 7-16°. In other embodiments, the rock angle is between 8-12°. In some embodiments, the rock rate is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 rpm. In some embodiments, the rock rate is between 4 and 12 rpm, such as between 4 and 6 rpm, inclusive.

In some embodiments, the bioreactor maintains the temperature at or near 37° C. and CO2 levels at or near 5% with a steady air flow at, at about, or at least 0.01 L/min, 0.05 L/min, 0.1 L/min, 0.2 L/min, 0.3 L/min, 0.4 L/min, 0.5 L/min, 1.0 L/min, 1.5 L/min, or 2.0 L/min or greater than 2.0 L/min. In certain embodiments, at least a portion of the cultivation is performed with perfusion, such as with a rate of 290 ml/day, 580 ml/day, and/or 1160 ml/day, e.g., depending on the timing in relation to the start of the cultivation and/or density of the cultivated cells. In some embodiments, at least a portion of the cell culture expansion is performed with a rocking motion, such as at an angle of between 5° and 10°, such as 6°, at a constant rocking speed, such as a speed of between 5 and 15 RPM, such as 6 RMP or 10 RPM.

In some embodiments, the methods for manufacturing, generating or producing a cell therapy and/or engineered cells, in accord with the provided methods, uses or articles of manufacture, may include formulation of cells, such as formulation of genetically engineered cells resulting from the processing steps prior to or after the incubating, engineering, and cultivating, and/or one or more other processing steps as described. In some embodiments, one or more of the processing steps, including formulation of cells, can be carried out in a closed system. In some cases, the cells are processed in one or more steps (e.g. carried out in the centrifugal chamber and/or closed system) for manufacturing, generating or producing a cell therapy and/or engineered cells may include formulation of cells, such as formulation of genetically engineered cells resulting from the transduction processing steps prior to or after the culturing, e.g. cultivation and expansion, and/or one or more other processing steps as described. In some embodiments, the genetically engineered cells are formulated as unit dose form compositions including the number of cells for administration in a given dose or fraction thereof.

In some embodiments, the dose of cells comprising cells engineered with a recombinant antigen receptor, e.g. CAR or TCR, is provided as a composition or formulation, such as a pharmaceutical composition or formulation. Such compositions can be used in accord with the provided methods, such as in the treatment of diseases, conditions, and disorders, or in detection, diagnostic, and prognostic methods, and uses and articles of manufacture. In some cases, the cells can be formulated in an amount for dosage administration, such as for a single unit dosage administration or multiple dosage administration.

In some embodiments, the cells can be formulated into a container, such as a bag or vial. In some embodiments, the vial may be an infusion vial. In some embodiments, the vial is formulated with a single unit dose of the engineered cells, such as including the number of cells for administration in a given dose or fraction thereof.

In some embodiments, the cells are formulated in a pharmaceutically acceptable buffer, which may, in some aspects, include a pharmaceutically acceptable carrier or excipient. In some embodiments, the processing includes exchange of a medium into a medium or formulation buffer that is pharmaceutically acceptable or desired for administration to a subject. In some embodiments, the processing steps can involve washing the transduced and/or expanded cells to replace the cells in a pharmaceutically acceptable buffer that can include one or more optional pharmaceutically acceptable carriers or excipients. Exemplary of such pharmaceutical forms, including pharmaceutically acceptable carriers or excipients, can be any described below in conjunction with forms acceptable for administering the cells and compositions to a subject. The pharmaceutical composition in some embodiments contains the cells in amounts effective to treat or prevent the disease or condition, such as a therapeutically effective or prophylactically effective amount.

In some embodiments, the formulation buffer contains a cryopreservative. In some embodiments, the cell are formulated with a cyropreservative solution that contains 1.0% to 30% DMSO solution, such as a 5% to 20% DMSO solution or a 5% to 10% DMSO solution. In some embodiments, the cryopreservation solution is or contains, for example, PBS containing 20% DMSO and 8% human serum albumin (HSA), or other suitable cell freezing media. In some embodiments, the cryopreservative solution is or contains, for example, at least or about 7.5% DMSO. In some embodiments, the processing steps can involve washing the transduced and/or expanded cells to replace the cells in a cryopreservative solution. In some embodiments, the cells are frozen, e.g., cryoprotected or cryopreserved, in media and/or solution with a final concentration of or of about 12.5%, 12.0%, 11.5%, 11.0%, 10.5%, 10.0%, 9.5%, 9.0%, 8.5%, 8.0%, 7.5%, 7.0%, 6.5%, 6.0%, 5.5%, or 5.0% DMSO, or between 1% and 15%, between 6% and 12%, between 5% and 10%, or between 6% and 8% DMSO. In particular embodiments, the cells are frozen, e.g., cryoprotected or cryopreserved, in media and/or solution with a final concentration of or of about 5.0%, 4.5%, 4.0%, 3.5%, 3.0%, 2.5%, 2.0%, 1.5%, 1.25%, 1.0%, 0.75%, 0.5%, or 0.25% HSA, or between 0.1% and 5%, between 0.25% and 4%, between 0.5% and 2%, or between 1% and 2% HSA.

In some embodiments, the formulation is carried out using one or more processing step including washing, diluting or concentrating the cells, such as the cultured or expanded cells. In some embodiments, the processing can include dilution or concentration of the cells to a desired concentration or number, such as unit dose form compositions including the number of cells for administration in a given dose or fraction thereof. In some embodiments, the processing steps can include a volume-reduction to thereby increase the concentration of cells as desired. In some embodiments, the processing steps can include a volume-addition to thereby decrease the concentration of cells as desired. In some embodiments, the processing includes adding a volume of a formulation buffer to transduced and/or expanded cells. In some embodiments, the volume of formulation buffer is from or from about 10 mL to 1000 mL, such as at least or at least about or about or 50 mL, 100 mL, 200 mL, 300 mL, 400 mL, 500 mL, 600 mL, 700 mL, 800 mL, 900 mL or 1000 mL.

In some embodiments, such processing steps for formulating a cell composition is carried out in a closed system. Exemplary of such processing steps can be performed using a centrifugal chamber in conjunction with one or more systems or kits associated with a cell processing system, such as a centrifugal chamber produced and sold by Biosafe SA, including those for use with the Sepax® or Sepax 2® cell processing systems. An exemplary system and process is described in International Publication Number WO2016/073602. In some embodiments, the method includes effecting expression from the internal cavity of the centrifugal chamber a formulated composition, which is the resulting composition of cells formulated in a formulation buffer, such as pharmaceutically acceptable buffer, in any of the above embodiments as described. In some embodiments, the expression of the formulated composition is to a container, such as the vials of the biomedical material vessels described herein, that is operably linked as part of a closed system with the centrifugal chamber. In some embodiments, the biomedical material vessels are configured for integration and or operable connection and/or is integrated or operably connected, to a closed system or device that carries out one or more processing steps. In some embodiments, the biomedical material vessel is connected to a system at an output line or output position. In some cases, the closed system is connected to the vial of the biomedical material vessel at the inlet tube. Exemplary close systems for use with the biomedical material vessels described herein include the Sepax® and Sepax® 2 system.

In some embodiments, the closed system, such as associated with a centrifugal chamber or cell processing system, includes a multi-port output kit containing a multi-way tubing manifold associated at each end of a tubing line with a port to which one or a plurality of containers can be connected for expression of the formulated composition. In some aspects, a desired number or plurality of vials, can be sterilely connected to one or more, generally two or more, such as at least 3, 4, 5, 6, 7, 8 or more of the ports of the multi-port output. For example, in some embodiments, one or more containers, e.g., biomedical material vessels, can be attached to the ports, or to fewer than all of the ports. Thus, in some embodiments, the system can effect expression of the output composition into a plurality of vials of the biomedical material vessels.

In some aspects, cells can be expressed to the one or more of the plurality of output containers, e.g., vials, in an amount for dosage administration, such as for a single unit dosage administration or multiple dosage administration. For example, in some embodiments, the vials, may each contain the number of cells for administration in a given dose or fraction thereof. Thus, each vial, in some aspects, may contain a single unit dose for administration or may contain a fraction of a desired dose such that more than one of the plurality of vials, such as two of the vials, or 3 of the vials, together constitute a dose for administration. In some embodiments, 4 vials together constitute a dose for administration.

Thus, the containers, e.g. bags or vials, generally contain the cells to be administered, e.g., one or more unit doses thereof. The unit dose may be an amount or number of the cells to be administered to the subject or twice the number (or more) of the cells to be administered. It may be the lowest dose or lowest possible dose of the cells that would be administered to the subject. In some aspects, the provided articles of manufacture includes one or more of the plurality of output containers.

In some embodiments, each of the containers, e.g. bags or vials, individually comprises a unit dose of the cells. Thus in some embodiments, each of the containers comprises the same or approximately or substantially the same number of cells. In some embodiments, each unit dose contains at or about or at least or at least about 1×10⁶, 2×10⁶, 5×10⁶, 1×10⁷, 5×10⁷, or 1×10⁸ engineered cells, total cells, T cells, or PBMCs. In some embodiments, each unit dose contains at or about or at least or at least about 1×10⁶, 2×10⁶, 5×10⁶, 1×10⁷, 5×10⁷, or 1×10⁸ CAR+ T cells that are CD3+, such as CD4+ or CD8+, or a viable subset thereof. In some embodiments, the volume of the formulated cell composition in each container, e.g. bag or vial, is between at or about 10 mL and at or about 100 mL, such as at or about or at least or at least about 20 mL, 30 mL, 40 mL, 50 mL, 60 mL, 70 mL, 80 mL, 90 mL or 100 mL. In some embodiments, the volume of the formulated cell composition in each container, e.g. bag or vial, is between at or about 1 mL and at or about 10 mL, such as between at or about 1 mL and at or about 5 mL. In some embodiments, the volume of the formulated cell composition in each container, e.g. bag or vial, is between at or about 4 mL and at or about 5 mL. In some embodiments, the volume of the formulated cell composition in each container, e.g. bag or vial, is or is about 4.4 mL. In some embodiments, the volume of the formulated cell composition in each container, e.g. bag or vial, is or is about 4.5 mL. In some embodiments, the volume of the formulated cell composition in each container, e.g. bag or vial, is or is about 4.6 mL. In some embodiments, the volume of the formulated cell composition in each container, e.g. bag or vial, is or is about 4.7 mL. In some embodiments, the volume of the formulated cell composition in each container, e.g. bag or vial, is or is about 4.8 mL. In some embodiments, the volume of the formulated cell composition in each container, e.g. bag or vial, is or is about 4.9 mL. In some embodiments, the volume of the formulated cell composition in each container, e.g. bag or vial, is or is about 5.0 mL.

In some embodiments, the formulated cell composition has a concentration of greater than at or about 0.5×10⁶ recombinant receptor-expressing (e.g. CAR⁺)/CD3+ cells or such viable cells per mL, greater than at or about 1.0×10⁶ recombinant receptor-expressing (e.g. CAR⁺)/CD3+ cells or such viable cells per mL, greater than at or about 1.5×10⁶ recombinant receptor-expressing (e.g. CAR⁺)/CD3+ cells or such viable cells per mL, greater than at or about 2.0×10⁶ recombinant receptor-expressing (e.g. CAR⁺)/CD3+ cells or such viable cells per mL. greater than at or about 2.5×10⁶ recombinant receptor-expressing (e.g. CAR⁺)/CD3+ cells or such viable cells per mL, greater than at or about 2.6×10⁶ recombinant receptor-expressing (e.g. CAR⁺)/CD3+ cells or such viable cells per mL, greater than at or about 2.7×10⁶ recombinant receptor-expressing (e.g. CAR⁺)/CD3+ cells or such viable cells per mL, greater than at or about 2.8×10⁶ recombinant receptor-expressing (e.g. CAR⁺)/CD3+ cells or such viable cells per mL, greater than at or about 2.9×10⁶ recombinant receptor-expressing (e.g. CAR⁺)/CD3+ cells or such viable cells per mL greater than at or about 3.0×10⁶ recombinant receptor-expressing (e.g. CAR⁺)/CD3+ cells or such viable cells per mL, greater than at or about 3.5×10⁶ recombinant receptor-expressing (e.g. CAR⁺)/CD3+ cells or such viable cells per mL, greater than at or about 4.0×10⁶ recombinant receptor-expressing (e.g. CAR⁺)/CD3+ cells or such viable cells per mL, greater than at or about 4.5×10⁶ recombinant receptor-expressing (e.g. CAR⁺)/CD3+ cells or such viable cells per mL or greater than at or about 5×10⁶ recombinant receptor-expressing (e.g. CAR⁺)/CD3+ cells or such viable cells per mL. In some embodiments, the CD3+ cells are CD4+ T cells. In some embodiments, the CD3+ cells are CD8+ T cells. In some embodiments, the CD3+ T cells are CD4+ and CD8+ T cells.

In some embodiments, the cells in the container, e.g. bag or vials, can be cryopreserved. In some embodiments, the container, e.g. vials, can be stored in liquid nitrogen until further use.

In some embodiments, such cells produced by the method, or a composition comprising such cells, are administered to a subject for treating a disease or condition, for example, in accord with the methods, uses and articles of manufacture described herein.

### Methods of Selecting Subjects for Treatment or Predicting or Monitoring Response to Treatment

Also provided herein are methods that include one or more assessing or screening steps to identify or select subjects for treatment with the combination therapy and/or for continuing the combination therapy, and/or to predict or assess a response to treatment (e.g. responsiveness or resistance to treatment) and/or for monitoring treatment outcomes. The provided methods are based on observations that the expression of one or more pro-survival gene (i.e. anti-apoptotic gene) can be associated with increased resistance to or lack of responsive to a cytotoxic therapy, such as CAR-T cells. In some embodiments, the provided methods improve the likelihood of response or efficacy of treatment of the subject, such as the likelihood or response or efficacy of the cell therapy in the subject.

In some embodiments, provided herein is a method that includes of selecting a subject for treatment with a cytotoxic therapy, such as any as described, e.g. CAR T cells. In some embodiments, the methods include (a) assessing the level or amount of one or more prosurvival gene in a biological sample from a subject having or suspected of having a cancer; (b) selecting the subject for treatment with a cytotoxic therapy if the level or amount of the one or more prosurvival gene is below a gene reference value; and (c) administering to the selected patient the cytotoxic therapy that binds an antigen associated with, expressed by, or present on cells of the cancer.

In some embodiments, provided herein is a method of selecting a subject for treatment with an inhibitor of a prosurvival BCL2 family protein, in which the subject is to receive administration of a cytotoxic therapy, such as any as described, e.g. CAR T cells. In some embodiments, the subject has a cancer. In some embodiments, the methods include (a) assessing the level or amount of one or more prosurvival gene in a biological sample from the subject, wherein the level or amount of the one or more prosurvival gene is the level or amount of a protein and/or a polynucleotide encoded by the one or more prosurvival gene, wherein the subject is to receive administration of a cytotoxic therapy that is an immunotherapy or a cell therapy that specifically binds to an antigen associated with, expressed by, or present on cells of the cancer, and wherein the biological sample is obtained from the subject prior to the administration of the cytotoxic therapy; and (b) selecting the subject having the cancer for treatment with an inhibitor of a prosurvival BCL2 family protein if the level or amount of the one or more prosurvival gene is above a gene reference value. In some cases, the method further comprises administering to the selected subject the inhibitor in combination with the cytotoxic therapy, such as in accord with any of the provided methods. In other cases, if the subject is not selected for treatment with the inhibitor in accord with the provided method, the subject is only administered the cytotoxic therapy without combination administration with the inhibitor.

In some embodiments, provided herein is a method of identifying a subject having a cancer that is predicted to be resistant to treatment with a cytotoxic therapy, where, (1) if so predicted, providing to the subject an alternative treatment than the planned or scheduled dosing of the cytotoxic therapy and (2) is not so predicted, providing to the subject the cytotoxic therapy, such as as the planned or scheduled dosing. In such embodiments, the method includes (a) assessing the level or amount of one or more prosurvival gene in a biological sample from the subject, wherein the level or amount of the one or more prosurvival gene is the level or amount of a protein and/or a polynucleotide encoded by the one or more prosurvival gene, wherein the subject is a candidate for administration of a dose of a cytotoxic therapy, wherein the cytotoxic therapy is an immunotherapy or a cell therapy that specifically binds to an antigen associated with, expressed by, or present on cells of the cancer, and wherein the biological sample is obtained from the subject prior to the administration of the cytoxic therapy; and (b) identifying the subject as having a cancer that is predicted to be resistant to treatment with the cytotoxic therapy if the level or amount of the one or more prosurvival gene is above a gene reference value.

In some embodiments, if the subject is identified as having a cancer that is predicted to be resistant to treatment with the cytotoxic therapy, the method further includes administering an alternative treatment to the identified subject, wherein the alternative treatment is selected from among the following: a combination treatment comprising the cytotoxic therapy and an additional agent that modulates or increases the activity of the cytotoxic therapy; an increased dose of the cytotoxic therapy; and/or a chemotherapeutic agent. In some embodiments, the alternative treatment is an increased dose of the cytotoxic therapy compared to a dose of the cytotoxic therapy given to a subject not identified as having a cancer that is predicted to be resistant to treatment with the cytotoxic therapy. In some aspects, the increased dose of the cytotoxic therapy comprises an increased number of cells of the cytotoxic therapy compared to a dose of the cytotoxic therapy given to a subject identified as having a cancer that is not predicted to be resistant to treatment with the cytotoxic therapy. In some embodiments, the alternative treatment treatment with a chemotherapeutic agent, such as cyclophosphamide, doxorubicin, prednisone, vincristine, fludarabine, bendamustine, and/or rituximab. In some embodiments, the alternative treatment is a combination treatment comprising the cytotoxic therapy and an additional agent that modulates or increases the activity of the T cell therapy, such as an the additional agent that is an immune checkpoint inhibitor, a modulator of a metabolic pathway, an adenosine receptor antagonist, a kinase inhibitor, an anti-TGFβ antibody or an anti-TGFβR antibody, a cytokine. In some embodiments, the alternative treatment includes combination treatment of the cytotoxic therapy and a prosurvival BCL2 family protein inhibitor, such as in accord with any of the provided methods. In some of any such embodiments, the cytotoxic therapy includes cells expressing a recombinant receptor that binds to an antigen associated with, expressed by, or present on the cells of the cancer.

In some embodiments, if the subject is identified as having a cancer that is not predicted to be resistant to treatment with the cytotoxic therapy, the subject is administered the planned dose or schedule of the cytotoxic therapy.

In some embodiments, provided herein is a method for determining responsiveness of a subject having a cancer to a cytotoxic therapy, in which the subject is one that has received administration of the cytotoxic therapy. In some embodiments, the method includes (a) assessing the level or amount of expression of one or more prosurvival gene in a biological sample from the subject, wherein the level or amount of the one or more prosurvival gene is the level or amount of a protein and/or a polynucleotide encoded by the one or more prosurvival gene, wherein the biological sample is obtained from the subject at a first time prior to the subject being administered the cytotoxic therapy, and wherein the subject is to receive treatment with the cytotoxic therapy; (b) assessing the level or amount of expression of the one or more prosurvival gene in a biological sample from the subject at a second time after administration of the cytotoxic therapy to the subject, wherein the level or amount of the one or more prosurvival gene is the level or amount of a protein and/or a polynucleotide encoded by the one or more prosurvival gene, wherein the biological sample is obtained at a second time after the administration of the cytotoxic therapy to the subject, and wherein the subject has been administered the cytotoxic therapy prior to the assessing in (b); and (c) determining that the subject is responsive to the therapy if the level or amount of the one or more prosurvival gene at the second time is lower than the level or amount of the one or more prosurvival gene at the first time. In such methods, the subject has received administration of the cytotoxic therapy before the administration in (b).

The following subsections provide particular features for carrying out any of the provided methods.

A. Samples

In certain embodiments, the expression of one or more gene products are measured, assessed, and/or determined in a sample. In provided embodiments, the sample is a biological sample that is taken, collected, and/or obtained from a subject. In particular embodiments, the sample is a tumor sample, e.g. tumor biopsy sample. In particular embodiments, the sample is a blood sample. In certain embodiments, the subject has a disease or condition and/or is suspected of having a disease or condition. In some embodiments, subject has received, will receive, or is a candidate to receive a therapy. In particular embodiments, the sample is taken, collected, and/or obtained from a subject who has been, who will be, or is a candidate to be administered a therapy. In particular embodiments, the sample is taken, collected, and/or obtained prior to treatment or administration with the therapy.

In particular embodiments, the subject has not yet received the therapy. In some embodiments, the subject is scheduled to or will receive the therapy at a subsequent time after the assessing. In other embodiments, the subject is a candidate for receiving the therapy and, depending on the results of the assessing in accord with the provided methods, may receive the therapy or may receive an alternative therapy or treatment. In any of such embodiments, the sample is a sample from the subject prior to receiving administration of the therapy. In some embodiments, the sample is a tumor sample, e.g. tumor biopsy sample. In some embodiments, the sample is a blood sample.

In certain embodiments, the methods involve monitoring response in a subject in which the subject has received administration of the therapy. In such embodiments, the methods include assessment of a first sample at a time prior to the administering of the therapy and a second sample at a time after administering the therapy. In some embodiments, the first sample is a sample from the subject prior to receiving administration of the therapy. In some embodiments, the second sample is a sample from the subject after receive administration of the therapy. In some embodiments, the sample is a tumor sample, e.g. biopsy sample. In some embodiments, the sample is a blood sample.

In some embodiments, the therapy is an administration of a cell therapy. In particular embodiments, the therapy is an administration of an immunotherapy. In particular embodiments, the therapy is an administration of a prosurvival BCL2 family protein inhibition. In particular embodiments, the therapy is a combination therapy comprising administration of a cell therapy and a prosurvival BCL2 family protein inhibitor. In particular embodiments, the therapy is a combination therapy comprising administration of an immunotherapy and a prosurvival BCL2 family protein inhibitor. In certain embodiments, the cell therapy treats and/or is capable of treating the disease or condition. In some embodiments, the therapy is a cell therapy that contains one or more engineered cells. In some embodiments, the engineered cells express a recombinant receptor. In particular embodiments, the recombinant receptor is a chimeric antigen receptor (CAR). In certain embodiments, the immunotherapy treats and/or is capable of treating the disease or condition. In some embodiments, the immunotherapy is a T cell-engaging therapy, e.g. a bi-specific T-cell engager (BiTE) therapy.

In particular embodiments, the sample is taken, collected, and/or obtained from a subject who has been, who will be, or is a candidate to be administered a therapy. In particular embodiments, the sample is taken, collected, and/or obtained prior to treatment or administration with the cytotoxic therapy, e.g., the cell therapy or immunotherapy. In accord with methods, kits and articles of manufacture described herein, the sample can be assessed for one or more gene products that is associated with and/or correlate to a clinical outcome. Exemplary gene products that are associated with and/or correlated with a likelihood and/or probability of a clinical outcome based on expression in a sample collected or obtained from a subject subsequent to receiving an immunotherapy or a cell therapy include one or more prosurvival genes (i.e. the gene has an anti-apoptotic effect), such as myc family genes (c-myc, l-myc, and n-myc,) p53, and EZH2. Thus, in some aspects, the provided methods relate to identifying subjects, prior to receiving a cytotoxic therapy (e.g. a cell therapy, such as CAR-T cells), who may be likely to achieve a particular clinical outcome, e.g. complete response (CR), partial response (PR), or progressive disease (PD). As described elsewhere herein, the methods can be used to determine if the subject is a candidate for administration of a cytotoxic therapy, if the subject is a candidate for administration of a combination therapy comprising an cytotoxic therapy and a prosurvival BCL2 family protein inhibitor, and/or if the subject is likely to exhibit a clinical outcome in response to a therapy, e.g. CR, PR, or PD in response to administration of a cytotoxic therapy.

In some aspects, the provided methods relate to identifying subjects, prior to receiving a therapy, such as a cell therapy (e.g. CAR-T cell therapy), who may exhibit a response to the cell therapy, such as a complete response (CR) or partial response (PR) or who may be likely to exhibit complete response (CR) or partial response (PR) to administration of the therapy. In some aspects, the provided methods relate to identifying subjects, prior to receiving a therapy, such as a cell therapy (e.g. CAR-T cell therapy), who may be or who are predicted to be resistant to the therapy, such as may be or are predicted to exhibit non-response/stable disease (NR/SD) to the therapy, incomplete response/stable disease (SD) to the therapy, or progressive disease (PD) following the therapy, and/or subjects who may not be likely to exhibit complete response (CR) or partial response (PR) to administration of the therapy. As described elsewhere herein, the methods can be used to determine if a subject is likely to exhibit complete response (CR), partial response (PR), non-response/stable disease (NR/SD), incompletely response/stable disease (SD), and/or progressive disease (PD) in response to administration of the therapy, e.g. a cell therapy or immunotherapy.

In some embodiments, the sample is taken, collected, and/or obtained prior to treatment or administration with the therapy, e.g., the immunotherapy or cell therapy. In accord with methods, kits and articles of manufacture described herein, the sample can be assessed for one or more gene products that are associated with and/or correlate to clinical outcomes (e.g. CR, PR, or PD) after receiving the therapy. Exemplary gene products that are associated with and/or correlated with a likelihood and/or probability of a clinical outcome based on expression in a sample collected or obtained from a subject subsequent to receiving an immunotherapy or a cell therapy include one or more prosurvival genes (i.e. that gene has an anti-apoptotic effect), such as myc family genes (c-myc, l-myc, and n-myc,) p53, and EZH2. In some embodiments, the sample is collected within or about within or about 0, 1, 2, 3, 4, 5, 6, 9, 12, 18 or 24 hours, or 2, 3, 4, 5, 6, 7, 8, 9, 10, 11, 12, 14, 21, 28 days, or more prior to initiation of administration of the therapy, e.g. the immunotherapy or cell therapy.

In some embodiments, the sample is taken, collected, and/or obtained subsequent to treatment or administration with the therapy, e.g., the immunotherapy or cell therapy. In accord with methods, kits and articles of manufacture described herein, the sample can be assessed for one or more gene products that are associated with and/or correlate to clinical outcomes (e.g. CR, PR, or PD) after receiving the therapy. Exemplary gene products that are associated with and/or correlated with a likelihood and/or probability of a clinical outcome based on expression in a sample collected or obtained from a subject subsequent to receiving an immunotherapy or a cell therapy include one or more prosurvival genes (i.e. that gene has an anti-apoptotic effect), such as myc family genes (c-myc, l-myc, and n-myc,) p53, and EZH2. In some embodiments, the sample is collected, taken, and/or obtained from a subject within or about within or about 0, 1, 2, 3, 4, 5, 6, 9, 12, 18 or 24 hours, or 2, 3, 4, 5, 6, 7, 8, 9, 10, 11, 12, 14, 21, 28 days or more following initiation of administration of the therapy. In some aspects, the sample is collected prior to the subject exhibiting a sign or symptom of a response following administration of the therapy, such as CR, PR, NR/SD, SD, and/or PD.

In some embodiments, the sample is taken, collected, and/or obtained from a subject that has or is suspected of having a condition or disease. In some embodiments, the subject has or is suspected of having a cancer or proliferative disease. In particular embodiments, the subject has a disease or condition, or is suspected of having a disease or condition, that is associated with an antigen and/or is associated with diseased cells that express the antigen. In some embodiments, the disease or condition, e.g., a cancer or proliferative disorder, is associated with αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, cyclin-dependent kinase 4 (CDK4), chondroitin sulfate proteoglycan 4 (CSPG4) epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, enhancer of zeste homolog 2 (EZH2), Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPRC5D), Her2/neu (receptor tyrosine kinase erbB2), Her3 (erb-B3), Her4 (erb-B4), erbB dimers, human high molecular weight-melanoma-associated antigen (HMW-MAA), hepatitis B surface antigen, Human leukocyte antigen A1 (HLA-A1), human leukocyte antigen A2 (HLA-A2), IL-22 receptor alpha(IL-22Rα), IL-13 receptor alpha 2 (IL-13Rα2), kinase insert domain receptor (kdr), kappa light chain, L1 cell adhesion molecule (L1CAM), CE7 epitope of L1-CAM, Leucine Rich Repeat Containing 8 Family Member A (LRRC8A), Lewis Y, melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, preferentially expressed antigen of melanoma (PRAME), progesterone receptor, a prostate specific antigen, prostate stem cell antigen (PSCA), prostate specific membrane antigen (PSMA), receptor tyrosine kinase like orphan receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms tumor 1 (WT-1), and/or a pathogen-specific or pathogen expressed antigen. In some embodiments, the antigen is a viral antigen (such as a viral antigen from HIV, HCV, HBV, etc.), bacterial antigens, and/or parasitic antigens. In certain embodiments, the subject has a disease or condition, or is suspected of having a disease or condition, that is associated with CD19 and/or is associated with diseased cells that express CD19. In certain embodiments, the subject has a disease or condition, or is suspected of having a disease or condition, that is associated with a prosurvival BCL2 family protein and/or is associated with diseased cells that express a prosurvival BCL2 family protein. In certain embodiments, the subject has a disease or condition, or is suspected of having a disease or condition, that is associated with overexpression of a prosurvival BCL2 family protein and/or is associated with diseased cells that overexpress a prosurvival BCL2 family protein.

In some embodiments, the sample is taken, collected, and/or obtained from a subject that has or is suspected of having a cancer or proliferative disease that is a B cell malignancy or hematological malignancy. In some embodiments, the cancer or proliferative disease is a myeloma, e.g., a multiple myeloma (MM), a lymphoma or a leukemia, lymphoblastic leukemia (ALL), non-Hodgkin's lymphoma (NHL), chronic lymphocytic leukemia (CLL), a diffuse large B-cell lymphoma (DLBCL), follicular lymphoma (FL) and/or acute myeloid leukemia (AML). In some embodiments, the cancer or proliferative disease is chronic lymphocytic leukemia (CLL). In some embodiments, the cancer or proliferative disease is small lymphocytic lymphoma (SLL). In some embodiments, the cancer or proliferative disorder is NHL. In some embodiments, the subject has, or is suspected of having NHL. In some embodiments, the NHL is DLBCL. In some embodiments, the NHL is germinal center B-cell like (GCB) subtype of DLBCL. In some embodiments, the NHL is not activated B-cell (ABC) subtype of DLBCL. In some embodiments, the NHL is adult DLBCL. In particular embodiments, the NHL is FL. In particular embodiments, the NHL is SLL. In particular embodiments, the NHL is pediatric FL.

In certain embodiments, the sample is a biological sample. In certain embodiments, the sample is a tissue sample. In particular embodiments, the sample is or includes a tissue affected, or suspected of being affected, by a disease or condition. In some embodiments, the sample is or includes a tissue affected, or suspected of being affected by a cancer or a proliferative disease. In some embodiments, the sample is a biopsy.

In certain embodiments, the sample is collected from a tissue having or suspected of having a tumor. In particular embodiments, the sample is or includes a tumor and/or a tumor microenvironment. In particular embodiments, the tumor is precancerous or cancerous, or is suspected of being cancerous or precancerous. In certain embodiments, the tumor is a primary tumor, i.e., the tumor is found at the anatomical site where the lesion initially developed or appeared. In some embodiments, the tumor is a secondary tumor, e.g., a cancerous tumor that originated from a cell within a primary tumor located within a different site in the body. In some embodiments, the sample contains one or more cells that are cancer cells and/or tumor cells.

In particular embodiments, the sample is collected from a lesion and/or a tumor that is associated with or caused by, or is suspected of being associated with or caused by, a non-hematologic cancer, e.g., a solid tumor. In some embodiments, the tumor is associated with or caused by, or is suspected of being associated with or caused by, a bladder, a lung, a brain, a melanoma (e.g. small-cell lung, melanoma), a breast, a cervical, an ovarian, a colorectal, a pancreatic, an endometrial, an esophageal, a kidney, a liver, a prostate, a skin, a thyroid, a lymph node, or a uterine cancer. In some embodiments, the lesion is associated with or caused by a pancreatic cancer, bladder cancer, colorectal cancer, breast cancer, prostate cancer, renal cancer, hepatocellular cancer, lung cancer, ovarian cancer, cervical cancer, pancreatic cancer, rectal cancer, thyroid cancer, uterine cancer, gastric cancer, esophageal cancer, head and neck cancer, melanoma, neuroendocrine cancers, CNS cancers, brain tumors, bone cancer, or soft tissue sarcoma. In certain embodiments, the sample contains lymph node tissue, e.g. a lymph node biopsy. In certain embodiments, the sample contains one or more cancer cells. In some embodiments, the sample contains one or more cells that are suspected of being cancerous.

In some embodiments, the sample is collected from a lesion or tumor that is associated with or caused by a B cell malignancy or hematological malignancy. In some embodiments, the lesion or tumor is associated with a myeloma, e.g., a multiple myeloma (MM), a lymphoma or a leukemia, lymphoblastic leukemia (ALL), non-Hodgkin's lymphoma (NHL), chronic lymphocytic leukemia (CLL), and/or acute myeloid leukemia (AML). In some embodiments, the lesion or tumor is associated with a CLL. In some embodiments, the lesion or tumor is associated with or caused by NHL, e.g., DLBCL or FL. In some embodiments, the lesion or tumor is associated with or caused by a small lymphocytic lymphoma (SLL). In some embodiments, the lesion or tumor is DLBCL. In some embodiments, the lesion or tumor is FL. In some embodiments, the lesion or tumor is SLL.

In some embodiments, the sample is a tissue sample, e.g., a tissue biopsy. In particular embodiments, the sample is obtained, collected, or taken from connective tissue, muscle tissue, nervous tissue, or epithelial tissue. In certain embodiments, the lesion is present on the heart, vasculature, salivary glands, esophagus, stomach, liver, gallbladder, pancreas, intestines, colon, rectum, hypothalamus, pituitary gland, pineal gland, thyroid, parathyroid, adrenal gland, kidney, ureter, bladder, breast, urethra, lymphatic system, skin, muscle, brain, spinal cord, nerves, ovaries, uterus, testes, prostate, pharynx, larynx, trachea, bronchi, lungs, diaphragm, bone, cartilage, ligaments, or tendons. In particular embodiments, the sample is obtained, collected, or taken from bone marrow. In some embodiments, the sample is a bone marrow aspirate.

In some embodiments, the sample is a body fluid from the subject. In some embodiments, the sample is a blood, serum, plasma or urine sample. In some embodiments, the sample is a plasma sample.

In particular embodiments, the sample does not contain the therapy, e.g., the cell therapy or immunotherapy. In particular embodiments, the sample does not contain any cells, e.g., engineered cells, of a cell therapy. In particular embodiments, the therapy includes a T cell therapy and the sample does not contain any engineered T cells and/or any T cells of the therapy. In particular embodiments, the sample does not contain any engineered cells that express a recombinant receptor, e.g., a CAR. In some embodiments, the sample does not contain cells expressing a CAR.

In any of the provided embodiments, the sample is a bone marrow aspirate from a subject with NHL, or a subject that is likely or suspected of having NHL, and the gene product is a polynucleotide, such as RNA, e.g. mRNA. In any of the provided embodiments, the sample is a bone marrow aspirate from a subject with NHL or that is likely or suspected of having NHL, and the gene product is a protein. In any of the provided embodiments, the sample is a bone marrow aspirate from a subject with DLBCL, or a subject that is likely or suspected of having DLBCL, and the gene product is a polynucleotide, such as RNA, e.g. mRNA. In any of the provided embodiments, the sample is a bone marrow aspirate from a subject with DLBCL or that is likely or suspected of having DLBCL, and the gene product is a protein. In any of the provided embodiments, the sample is a bone marrow aspirate from a subject with FL, or a subject that is likely or suspected of having FL, and the gene product is a polynucleotide, such as RNA, e.g. mRNA. In any of the provided embodiments, the sample is a bone marrow aspirate from a subject with FL or that is likely or suspected of having FL, and the gene product is a protein. In any of the provided embodiments, the sample is a bone marrow aspirate from a subject with SLL, or a subject that is likely or suspected of having SLL, and the gene product is a polynucleotide, such as RNA, e.g. mRNA. In any of the provided embodiments, the sample is a bone marrow aspirate from a subject with SLL or that is likely or suspected of having SLL, and the gene product is a protein.

In any of the provided embodiments, the sample is a bone marrow aspirate from a subject with a leukemia, or a subject that is likely or suspected of having a leukemia, and the gene product is a polynucleotide, such as RNA, e.g. mRNA. In any of the provided embodiments, the sample is a bone marrow aspirate from a subject with a leukemia or that is likely or suspected of having a leukemia, and the gene product is a protein. In any of the provided embodiments, the sample is a bone marrow aspirate from a subject with CLL, or a subject that is likely or suspected of having CLL, and the gene product is a polynucleotide, such as RNA, e.g. mRNA. In any of the provided embodiments, the sample is a bone marrow aspirate from a subject with CLL or that is likely or suspected of having CLL, and the gene product is a protein.

In any of the provided embodiments, the sample is a lymph node biopsy from a subject with NHL, or a subject that is likely or suspected of having NHL, and the gene product is a polynucleotide, such as RNA, e.g. mRNA. In any of the provided embodiments, the sample is a lymph node biopsy from a subject with NHL or that is likely or suspected of having NHL, and the gene product is a protein. In any of the provided embodiments, the sample is a lymph node biopsy from a subject with DLBCL, or a subject that is likely or suspected of having DLBCL, and the gene product is a polynucleotide, such as RNA, e.g. mRNA. In any of the provided embodiments, the sample is a lymph node biopsy from a subject with DLBCL or that is likely or suspected of having DLBCL, and the gene product is a protein. In any of the provided embodiments, the sample is a lymph node biopsy from a subject with FL, or a subject that is likely or suspected of having FL, and the gene product is a polynucleotide, such as RNA, e.g. mRNA. In any of the provided embodiments, the sample is a lymph node biopsy from a subject with FL or that is likely or suspected of having FL, and the gene product is a protein. In any of the provided embodiments, the sample is a lymph node biopsy from a subject with SLL, or a subject that is likely or suspected of having SLL, and the gene product is a polynucleotide, such as RNA, e.g. mRNA. In any of the provided embodiments, the sample is a lymph node biopsy from a subject with SLL or that is likely or suspected of having SLL, and the gene product is a protein.

In any of the provided embodiments, the sample is a lymph node biopsy from a subject with a leukemia, or a subject that is likely or suspected of having a leukemia, and the gene product is a polynucleotide, such as RNA, e.g. mRNA. In any of the provided embodiments, the sample is a lymph node biopsy from a subject with a leukemia or that is likely or suspected of having a leukmia, and the gene product is a protein. In any of the provided embodiments, the sample is a lymph node biopsy from a subject with CLL, or a subject that is likely or suspected of having CLL, and the gene product is a polynucleotide, such as RNA, e.g. mRNA. In any of the provided embodiments, the sample is a lymph node biopsy from a subject with CLL or that is likely or suspected of having CLL, and the gene product is a protein.

In any of the provided embodiments, the sample is a body fluid sample from a subject with NHL, or a subject that is likely or suspected of having NHL, and the gene product is a protein. In any of the provided embodiments, the sample is a body fluid sample from a subject with NHL, or a subject that is likely or suspected of having NHL, and the gene product is a polynucleotide. In any of the provided embodiments, the sample is a body fluid sample from a subject with DLBCL, or a subject that is likely or suspected of having DLBCL, and the gene product is a protein. In any of the provided embodiments, the sample is a body fluid sample from a subject with DLBCL, or a subject that is likely or suspected of having DLBCL, and the gene product is a polynucleotide. In any of the provided embodiments, the sample is a body fluid sample from a subject with FL, or a subject that is likely or suspected of having FL, and the gene product is a protein. In any of the provided embodiments, the sample is a body fluid sample from a subject with FL, or a subject that is likely or suspected of having FL, and the gene product is a polynucleotide. In any of the provided embodiments, the sample is a body fluid sample from a subject with SLL, or a subject that is likely or suspected of having SLL, and the gene product is a protein. In any of the provided embodiments, the sample is a body fluid sample from a subject with SLL, or a subject that is likely or suspected of having SLL, and the gene product is a polynucleotide. In some embodiments, the body fluid sample is a plasma sample. In some embodiments, the body fluid sample is a blood sample.

In any of the provided embodiments, the sample is a body fluid sample from a subject with a leukemia, or a subject that is likely or suspected of having a leukemia, and the gene product is a protein. In any of the provided embodiments, the sample is a body fluid sample from a subject with a leukemia, or a subject that is likely or suspected of having a leukemia, and the gene product is a polynucleotide. In any of the provided embodiments, the sample is a body fluid sample from a subject with CLL, or a subject that is likely or suspected of having CLL, and the gene product is a protein. In any of the provided embodiments, the sample is a body fluid sample from a subject with CLL, or a subject that is likely or suspected of having CLLL, and the gene product is a polynucleotide. In some embodiments, the body fluid sample is a plasma sample. In some embodiments, the body fluid sample is a blood sample.

B. Measuring or Assessing Gene Expression or Gene Products

In certain embodiments, the methods provided herein include one or more steps of assessing, measuring, determining, and/or quantifying the expression of one or more genes in a sample. In some embodiments, the expression of a gene, e.g., a gene with an expression that positively or negatively correlates with a clinical outcome, is or includes assessing, measuring, determining, and/or quantifying a level, amount, or concentration of a gene product in the sample. In some embodiments, gene expression is or includes a process by which information of the gene is used in the synthesis of a gene product. Thus, in some embodiments, a gene product is any biomolecule that is assembled, generated, and/or synthesized with information encoded by a gene, and may include polynucleotides and/or polypeptides. In particular embodiments, assessing, measuring, and/or determining gene expression is or includes determining or measuring the level, amount, or concentration of the gene product. In certain embodiments, the level, amount, or concentration of the gene product may be transformed (e.g., normalized) or directly analyzed (e.g., raw). In some embodiments, the gene product is a protein that is encoded by the gene. In certain embodiments, the gene product is a polynucleotide, e.g., an mRNA or a protein, that is encoded by the gene.

In some embodiments, the gene product is a polynucleotide that is expressed by and/or encoded by the gene. In certain embodiments, the polynucleotide is an RNA. In some embodiments, the gene product is a messenger RNA (mRNA), a transfer RNA (tRNA), a ribosomal RNA, a small nuclear RNA, a small nucleolar RNA, an antisense RNA, long non-coding RNA, a microRNA, a Piwi-interacting RNA, a small interfering RNA, and/or a short hairpin RNA. In particular embodiments, the gene product is an mRNA.

In particular embodiments, the amount or level of a polynucleotide in a sample may be assessed, measured, determined, and/or quantified by any suitable means known in the art. For example, in some embodiments, the amount or level of a polynucleotide gene product can be assessed, measured, determined, and/or quantified by polymerase chain reaction (PCR), including reverse transcriptase (rt) PCR, droplet digital PCR, real-time and quantitative PCR (qPCR) methods (including, e.g., TAQMAN®, molecular beacon, LIGHTUP™, SCORPION™ SIMPLEPROBES®; see, e.g., U.S. Pat. Nos. 5,538,848; 5,925,517; 6,174,670; 6,329,144; 6,326,145 and 6,635,427); northern blotting; Southern blotting, e.g., of reverse transcription products and derivatives; array based methods, including blotted arrays, microarrays, or in situ-synthesized arrays; and sequencing, e.g., sequencing by synthesis, pyrosequencing, dideoxy sequencing, or sequencing by ligation, or any other methods known in the art, such as discussed in Shendure et al., Nat. Rev. Genet. 5:335-44 (2004) or Nowrousian, Euk. Cell 9(9): 1300-1310 (2010), including such specific platforms as HELICOS®, ROCHE® 454, ILLUMINA®/SOLEXA®, ABI SOLiD®, and POLONATOR® sequencing. In particular embodiments, the levels of nucleic acid gene products are measured by quantitative PCR (qPCR) methods, such qRT-PCR. In some embodiments, the qRT-PCR uses three nucleic acid sets for each gene, where the three nucleic acids comprise a primer pair together with a probe that binds between the regions of a target nucleic acid where the primers bind—known commercially as a TAQMAN® assay.

In particular embodiments, assessing, measuring, determining, and/or quantifying amount or level of an RNA gene product includes a step of generating, polymerizing, and/or deriving a cDNA polynucleotide and/or a cDNA oligonucleotide from the RNA gene product. In certain embodiments, the RNA gene product is assessed, measured, determined, and/or quantified by directly assessing, measuring, determining, and/or quantifying a cDNA polynucleotide and/or a cDNA oligonucleotide that is derived from the RNA gene product.

In some embodiments, one or more oligonucleotide primers is contacted to an RNA gene product and/or a cDNA polynucleotide or oligonucleotide derived from the RNA gene product, to assess, measure, determine, and/or quantify the level, amount, or concentration of the RNA gene product. In some embodiments, provided herein are oligonucleotide primers that are suitable for assessing, measuring, detecting, and/or quantifying the level, amount, or concentration of an RNA gene product (or a cDNA derived therefrom). In certain embodiments, the oligonucleotide primers hybridize, and/or are capable of hybridizing to an RNA gene product and/or a cDNA derived therefrom. In certain embodiments, the oligonucleotide hybridizes and/or is capable of hybridizing to an RNA gene product, or cDNA derived therefrom, that is expressed and/or encoded by one or more prosurvival gene (i.e. the gene confers an anti-apoptotic effect), such as myc family genes (c-myc, l-myc, and n-myc), p53, and EZH2. In some embodiments, sets of oligonucleotide primers may be prepared for any of RNA gene products that are encoded by one or more prosurvival gene (i.e. the gene confers an anti-apoptotic effect), such as myc family genes (c-myc, l-myc, and n-myc), p53, and EZH2, or described anywhere in the application. In some embodiments, the oligonucleotide primers can readily be designed using ordinary skill in the art of molecular biology to arrive at primers that are specific for a given RNA gene product. In some embodiments, the oligonucleotide primer has a length of about 10-100 nucleotides, e.g., about 10, 11, 12, 13, 14, 15, 16, 17, 18, 19, 20, 21, 22, 23, 24, 25, 26, 27, 28, 29, 30, 35, 40, 45, 50, 55, 60, 70, 80, 90, 100 nucleotides, or more) and a sequence of the primers can readily be adjusted to achieve a desired melting temperature (“Tm”; e.g., about 45-72° C., e.g., about 45, 50, 55, 60, 65, 70, 72° C. or more) and specificity. One of skill in the art will readily account for factors such as secondary structures, primer dimers, salt concentrations, nucleic acid concentrations, et cetera. Oligonucleotide primers provided herein may consist of (or consist essentially of) naturally occurring deoxribonucleotides or, optionally, may include modifications such as non-natural nucleotides, artificial backbones (such as PNAs), and detectable labels, such as florescent labels. In particular embodiments, a florescent label is attached, e.g., covalently attached, to the oligonucleotide primer.

In particular embodiments, the expression of two or more of the genes are measured or assessed simultaneously. In certain embodiments, a multiplex PCR, e.g., a multiplex rt-PCR assessing or a multiplex quantitative PCR (qPCR) for, measuring, determining, and/or quantifying the level, amount, or concentration of two or more gene products. In some embodiments, microarrays (e.g., AFFYMETRIX®, AGILENT® and ILLUMINA®-style arrays) are used for assessing, measuring, determining, and/or quantifying the level, amount, or concentration of two or more gene products. In some embodiments, microarrays are used for assessing, measuring, determining, and/or quantifying the level, amount, or concentration of a cDNA polynucleotide that is derived from an RNA gene product.

In some embodiments, the expression of one or more gene products, e.g., polynucleotide gene products, is determined by sequencing the gene product and/or by sequencing a cDNA polynucleotide that is derived from the from the gene product. In some embodiments, the sequencing is performed by a non-Sanger sequencing method and/or a next generation sequencing (NGS) technique. Examples of Next Generation Sequencing techniques include, but are not limited to Massively Parallel Signature Sequencing (MPSS), Polony sequencing, pyrosequencing, Reversible dye-terminator sequencing, SOLiD sequencing, Ion semiconductor sequencing, DNA nanoball sequencing, Helioscope single molecule sequencing, Single molecule real time (SMRT) sequencing, Single molecule real time (RNAP) sequencing, and Nanopore DNA sequencing.

In some embodiments, the NGS technique is RNA sequencing (RNA-Seq). In particular embodiments, the expression of the one or more polynucleotide gene products is measured, determined, and/or quantified by RNA-Seq. RNA-Seq, also called whole transcriptome shotgun sequencing determines the presence and quantity of RNA in a sample. RNA sequencing methods have been adapted for the most common DNA sequencing platforms [HiSeq systems (Illumina), 454 Genome Sequencer FLX System (Roche), Applied Biosystems SOLiD (Life Technologies), IonTorrent (Life Technologies)]. These platforms require initial reverse transcription of RNA into cDNA. Conversely, the single molecule sequencer HeliScope (Helicos BioSciences) is able to use RNA as a template for sequencing. A proof of principle for direct RNA sequencing on the PacBio RS platform has also been demonstrated (Pacific Bioscience). In some embodiments, the one or more RNA gene products are assessed, measured, determined, and/or quantified by RNA-seq.

In some embodiments, the RNA-seq is a tag-based RNA-seq. In tag-based methods, each transcript is represented by a unique tag. Initially, tag-based approaches were developed as a sequence-based method to measure transcript abundance and identify differentially expressed genes, assuming that the number of tags (counts) directly corresponds to the abundance of the mRNA molecules. The reduced complexity of the sample, obtained by sequencing a defined region, was essential to make the Sanger-based methods affordable. When NGS technology became available, the high number of reads that could be generated facilitated differential gene expression analysis. A transcript length bias in the quantification of gene expression levels, such as observed for shotgun methods, is not encountered in tag-based methods. All tag-based methods are by definition strand specific. In particular embodiments, the one or more RNA gene products are assessed, measured, determined, and/or quantified by tag-based RNA-seq.

In some embodiments, the RNA-seq is a shotgun RNA-seq. Numerous protocols have been described for shotgun RNA-seq, but they have many steps in common: fragmentation (which can occur at RNA level or cDNA level, conversion of the RNA into cDNA (performed by oligo dT or random primers), second-strand synthesis, ligation of adapter sequences at the 3′ and 5′ ends (at RNA or DNA level) and final amplification. In some embodiments, RNA-seq can focus only on polyadenylated RNA molecules (mainly mRNAs but also some lncRNAs, snoRNAs, pseudogenes and histones) if poly(A)⁺ RNAs are selected prior to fragmentation, or may also include non-polyadenylated RNAs if no selection is performed. In the latter case, ribosomal RNA (more than 80% of the total RNA pool) needs to be depleted prior to fragmentation. It is, therefore, clear that differences in capturing of the mRNA part of the transcriptome lead to a partial overlap in the type of detected transcripts. Moreover, different protocols may affect the abundance and the distribution of the sequenced reads. This makes it difficult to compare results from experiments with different library preparation protocols.

In some embodiments, RNA from each sample, such as each lymph node biopsy sample, is obtained, fragmented and used to generate complementary DNA (cDNA) samples, such as cDNA libraries for sequencing Reads may be processed and aligned to the human genome and the expected number of mappings per gene/isoform are estimated and used to determine read counts. In some embodiments, read counts are normalized by the length of the genes/isoforms and number of reads in a library to yield FPKM normalized, e.g., by length of the genes/isoforms and number of reads in the library, to yield fragments per kilobase of exon per million mapped reads (FPKM) according to the gene length and total mapped reads. In some aspects, between-sample normalization is achieved by normalization, such as 75th quantile normalization, where each sample is scaled by the median of 75th quantiles from all samples, e.g., to yield quantile-normalized FPKM (FPKQ) values. The FPKQ values may be log-transformed (log 2).

In some embodiments, techniques and methods involving nucleotide aptamers are used to measure, assess, quantify, and/or determine the level, amount, or concentration of a polynucleotide gene product. Suitable nucleotide aptamers are known, and include those described in Cox and Ellington, Bioorganic & Medicinal Chemistry. (2001) 9 (10): 2525-2531; Cox et al., Combinatorial Chemistry & High Throughput Screening. (2002) 5 (4): 289-29; Cox et al., Nucleic Acids Research. (2002) 30(20): e108.

In some embodiments, RNA-seq is performed to sequence total RNA, e.g., the total RNA of a sample. In particular embodiments, the RNA-seq is performed to sequence one or more of mRNA, tRNA, ribosomal RNA, small nuclear RNA, small nucleolar RNA, antisense RNA, long non-coding RNA, microRNA, Piwi-interacting RNA, small interfering RNA, and/or a short hairpin RNA. In certain embodiments, the RNA-seq is performed to sequence only mRNA, tRNA, ribosomal RNA, small nuclear RNA, small nucleolar RNA, antisense RNA, long non-coding RNA, microRNA, Piwi-interacting RNA, small interfering RNA, and/or a short hairpin RNA. In particular embodiments, the RNA-seq is performed to sequence mRNA gene products.

In some embodiments, the gene product is or includes a protein, i.e., a polypeptide, that is encoded by and/or expressed by the gene. In particular embodiments, the gene product encodes a protein that is localized and/or exposed on the surface of a cell. In some embodiments, the protein is a soluble protein. In certain embodiments, the protein is secreted by a cell.

In particular embodiments, the gene expression is the amount, level, and/or concentration of a protein that is encoded by the gene. In certain embodiments, one or more protein gene products are measured by any suitable means known in the art. Suitable methods for assessing, measuring, determining, and/or quantifying the level, amount, or concentration or more or more protein gene products include, but are not limited to detection with immunoassays, nucleic acid-based or protein-based aptamer techniques, HPLC (high precision liquid chromatography), peptide sequencing (such as Edman degradation sequencing or mass spectrometry (such as MS/MS), optionally coupled to HPLC), and microarray adaptations of any of the foregoing (including nucleic acid, antibody or protein-protein (i.e., non-antibody) arrays). In some embodiments, the immunoassay is or includes methods or assays that detect proteins based on an immunological reaction, e.g., by detecting the binding of an antibody or antigen binding antibody fragment to a gene product. Immunoassays include, but are not limited to, quantitative immunocytochemisty or immunohistochemisty, ELISA (including direct, indirect, sandwich, competitive, multiple and portable ELISAs (see, e.g., U.S. Pat. No. 7,510,687), western blotting (including one, two or higher dimensional blotting or other chromatographic means, optionally including peptide sequencing), enzyme immunoassay (EIA), RIA (radioimmunoassay), and SPR (surface plasmon resonance).

In some embodiments, the gene expression product is a protein. In particular embodiments, the gene expression product is a fraction, portion, variant, version, and/or isoform of a protein, e.g., a protein encoded by one or more prosurvival gene (i.e. the gene confers an anti-apoptotic effect), such as myc family genes (c-myc, l-myc, and n-myc), p53, and EZH2. In particular embodiments, the fraction, portion, variant, version, and/or isoform of the protein is soluble. In some embodiments, the fraction, portion, variant, version, and/or isoform of the protein lacks a transmembrane domain. In certain embodiments, the fraction, portion, variant, version, and/or isoform of a protein is not expressed on or within the surface of a cell. In some embodiments, the fraction, portion, variant, version, and/or isoform of the protein has been cleaved from the surface of a cell.

The practice of the methods, kits, and compositions provided herein may also employ conventional biology methods, software and systems. For example, means for measuring the expression level of transcripts or partial transcripts of one or more of prosurvival gene (i.e. the gene confers an anti-apoptotic effect), such as myc family genes (c-myc, l-myc, and n-myc), p53, and EZH2; means for correlating the expression level with a classification of probability and/or likelihood (such as, of a clinical outcome, e.g. CR, PR or PD), following administration of and/or associated with the therapy; and means for outputting the probability and/or likelihood may employ conventional biology methods, software and systems as described herein or as otherwise known. Computer software products for use with the provided methods, compositions, and kits, typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2.sup.nd ed., 2001). See U.S. Pat. No. 6,420,108.

In some embodiments, the methods provided herein include a step of assessing one or more genes in a sample by assessing, measuring, determining, and/or quantifying the amount of the corresponding one or more gene products in the sample. In certain embodiments, the expression of one or more genes in a sample that negatively correlates and/or is negatively associated with a clinical response, e.g., CR, PR, or PD, is measured by determining the amount or level of one of more corresponding gene products in the sample. In certain embodiments, the gene expression in a sample is the level, amount, or concentration of a gene product that is encoded by the gene.

In particular embodiments, the expression of one or more genes that negatively correlate and/or are negatively associated with a clinical outcome, e.g., CR, PR, or PD, are measured in a sample. In some embodiments, the expression of one or more genes that negatively correlate and/or are negatively associated with a clinical outcome is assessed, measured, determined, and/or quantified by determining the amount or level of a product encoded, produced, and/or expressed by the gene

In some embodiments, the one or more gene products are encoded, produced, and/or expressed by one or more prosurvival gene (i.e. the gene confers an anti-apoptotic effect), such as myc family genes (c-myc, l-myc, and n-myc), p53, and EZH2. In certain embodiments, the gene product is one of two or more isoforms that are encoded by a gene. In particular embodiments, the one or more gene products are products of one or more prosurvival gene (i.e. the gene confers an anti-apoptotic effect), such as myc family genes (c-myc, l-myc, and n-myc), p53, and EZH2, or a portion thereof.

In particular embodiments, the expression of one or more genes that negatively correlate and/or are negatively associated with a clinical outcome, e.g. PD, are assessed, measured, determined, and/or quantified by determining the amount or level of an RNA product encoded, produced, and/or expressed by the one or more genes. In certain embodiments, the gene product is an mRNA. In certain embodiments, the one or more gene products are mRNA produced or encoded by one or more prosurvival gene (i.e. the gene confers an anti-apoptotic effect), such as myc family genes (c-myc, l-myc, and n-myc), p53, and EZH2.

In particular embodiments, the expression of one or more genes that negatively correlate and/or are negatively associated with a clinical outcome, e.g. PD, are assessed, measured, determined, and/or quantified by determining the amount or level of a protein encoded by or expressed by the one or more genes. In some embodiments, the one or more gene products are proteins, or portions or variants thereof, that are encoded, produced, and/or expressed by one or more prosurvival gene (i.e. the gene confers an anti-apoptotic effect), such as myc family genes (c-myc, l-myc, and n-myc), p53, and EZH2.

In particular embodiments, the expression of one or more genes that positively correlate and/or are positively associated with a clinical outcome, e.g., CR or PR, are measured in a sample. In some embodiments, the expression of one or more gene that positively correlate and/or are positively associated with a toxicity are assessed, measured, determined, and/or quantified by determining the amount or level of a product encoded, produced, and/or expressed by the gene. In some embodiments, the one or more gene products are encoded, produced, and/or expressed one or more prosurvival gene (i.e. the gene confers an anti-apoptotic effect), such as myc family genes (c-myc, l-myc, and n-myc), p53, and EZH2. In certain embodiments, the gene product is one of two or more isoforms that are encoded by a gene. In particular embodiments, the one or more gene products are products of one or more prosurvival gene (i.e. the gene confers an anti-apoptotic effect), such as myc family genes (c-myc, l-myc, and n-myc), p53, and EZH2.

In particular embodiments, the expression of one or more genes that positively correlate and/or are positively associated with a clinical outcome, e.g. CR or PR, are assessed, measured, determined, and/or quantified by determining the amount or level of an RNA product encoded, produced, and/or expressed by the one or more genes. In certain embodiments, the one or more gene products are one or more of an mRNA or a portion or partial transcript thereof of one or more prosurvival gene (i.e. the gene confers an anti-apoptotic effect), such as myc family genes (c-myc, l-myc, and n-myc), p53, and EZH2.

In particular embodiments, the expression of one or more genes that positively correlate and/or are positively associated with a clinical outcome, e.g. CR or PR, are assessed, measured, determined, and/or quantified by determining the amount or level of a protein encoded by or expressed by the gene. In some embodiments, the gene product is a protein encoded, produced, and/or expressed by one or more prosurvival gene (i.e. the gene confers an anti-apoptotic effect), such as myc family genes (c-myc, l-myc, and n-myc), p53, and EZH2.

In some embodiments, measuring, assessing, determining, and/or quantifying one or more of the gene products in a sample is not predictive, and/or is not associated or correlated with a clinical outcome (e.g. CR, PR, or PD) at the time at which the sample is collected from the subject. In some embodiments, the gene expression profile of any of the one or more prosurvival gene (i.e. the gene confers an anti-apoptotic effect), such as myc family genes (c-myc, l-myc, and n-myc), p53, and EZH2, are not predictive, correlated with, and/or associated with a clinical outcome (e.g. CR, PR, or PD), when the sample is collected during or after the subject has received treatment with a therapy, e.g., a cell therapy containing CAR-T cells.

1. Normalization to Control Values

In some embodiments, the assessment, determination, measurement, and/or quantification of a gene product, e.g., an RNA or protein gene product, of a sample is normalized to a control value. In certain embodiments, normalization to one or more control values may be performed to analyze, assess, or determine if an amount or level of the gene product indicates if the expression of the gene is elevated or decreased, and/or high or low. In particular embodiments, normalization to control values may be used to compare the gene expression of a gene a sample to the gene expression of a different sample.

In particular embodiments, the control value is a measurement, or a value of a measurement, of a different gene product. In some embodiments, the different gene product is a gene product of a housekeeping gene. In certain embodiments, the housekeeping gene is a constitutively active gene, e.g., a gene that is required for maintenance of basic cellular function. Examples of suitable housekeeping genes are known in the art, and include, but are not limited to, genes encoding ACTB (Beta-actin), B2M (Beta-2-microglobulin), GAPDH (Glyceraldehyde 3-phosphate dehydrogenase), RPLP0 (60S acidic ribosomal protein P0), GUSB (beta-glucuronidase), HMBS (Hydroxymethyl-bilane synthase), HPRT1 (Hypoxanthine phosphoribosyl-transferase 1), RPL13A (Ribosomal protein L13a), SDHA, succinate dehydrogenase complex subunit A), TBP (TATA box binding protein), TFRC (transferring receptor 1), and UBC (Ubiquitin C). In some embodiments, the control value is measured in the same sample as the gene product.

In certain embodiments, the gene product is compared and/or normalized to a control value that is a measurement, or a value of a measurement, of the gene product from the same gene. In some embodiments, the control value is a measurement, or a value of a measurement, that is obtained from one or more control samples. In certain embodiments, the gene product and the control value are measured in different samples. In some embodiments, the one or more control samples have an identical, a same, or a similar tissue composition and/or cellular composition as the sample. In some embodiments, the sample and control sample are different samples from the same, similar, and/or identical tissue from the same subject. In particular embodiments, the sample and the control sample different samples from the same tissue in different subjects. In particular embodiments, the sample and control sample are different samples from the same, similar, and/or identical tissue from different subjects. In some embodiments, the one or more samples are lymph node biopsy samples and the one or more control samples are lymph node biopsy samples. In particular embodiments, the one or more samples are blood samples, e.g., peripheral blood samples, and the one or more control samples are blood samples.

In certain embodiments, the control sample is obtained from a subject that does not have a condition and/or a cancer. In some embodiments, the control sample does not have and/or is not suspected of having one or more tumor cells. In particular embodiments, the sample and control sample are different samples from the same, similar, and/or identical tissue from different subjects. In certain embodiments, the control sample is obtained from a subject that is treated with a prosurvival BCL2 family protein inhibitor. In certain embodiments, the control sample is obtained from a subject that is not treated with a prosurvival BCL2 family protein inhibitor.

In certain embodiments, the control sample is obtained from a subject that exhibits progressive disease (PD) following treatment with a therapy, e.g. a cell therapy or an immunotherapy. In some embodiments, the control samples are obtained from a subject exhibiting PD at least 1 month, 2 months, 3 months, 4 months, 5 months, 6 months, 7 months, 8 months, 9 months, or more following administration of the therapy. In certain embodiments, the control sample is obtained from a subject that exhibits partial response (PR) or complete response (CR) following treatment with a therapy, e.g. an immunotherapy or a cell therapy. In some embodiments, the control samples are obtained from a subject exhibiting PR or CR at least 1 month, 2 months, 3 months, 4 months, 5 months, 6 months, 7 months, 8 months, 9 months, or more following administration of the therapy.

In certain embodiments, the assessment, determination, measurement, and/or quantification of a gene product, e.g., an RNA or protein gene product, of a sample is normalized to and/or compared to two or more control values. In some embodiments, the two or more control values include a control value that is a measurement, or a value of a measurement, that of the same gene product and a control value that is a measurement, or a value of a measurement, that of a different gene product.

In some embodiments, the control value has been previous determined. In certain embodiments, the one or more control values are measured or obtained in parallel with the assessment, measurement, determination, and/or quantification of the one or gene products in the sample.

In particular embodiments, the control value is an average (e.g. an arithmetic mean) or a median amount or level of expression of the one or more gene products obtained from a plurality of control samples. In some embodiments, the plurality of control samples is obtained from individual control subjects. In particular embodiments, the plurality of individual control subjects are subjects that do not have and/or are not suspected of having a condition or a disease. In some embodiments, the plurality of individual control subjects are subjects that do not have and/or are not suspected of having a cancer. In some embodiments, the plurality of individual control subjects are subjects that do not have and/or are not suspected of having a NHL. In particular embodiments, the subtype of NHL is the follicular lymphoma (FL) subtype of NHL. In particular embodiments, the subtype of NHL is the diffuse large B-cell lymphoma (DLBCL) subtype of NHL. In particular embodiments, the subtype of NHL is the small lymphocytic lymphoma (SLL) subtype of NHL. In some embodiments, the plurality of individual control subjects are subjects that do not have and/or are not suspected of having a leukemia. In particular embodiments, the subtype of the leukemia is chronic lymphocytic leukemia (CLL).

In certain embodiments, the plurality of individual control subjects is a plurality of subjects that have and/or are suspected of having a cancer. In some embodiments, the plurality of individual control subjects is or includes subjects that have and/or are suspected of having non-Hodgkin lymphoma (NHL). In some embodiments, the plurality of individual control subjects is or includes subjects that have and/or are suspected of having a specific subtype of NHL. In some embodiments, the plurality of individual control subjects is or includes subjects that have and/or are suspected of having NHL, but not a specific subtype of NHL. In particular embodiments, the subtype of NHL is the DLBCL subtype of ALL. In some embodiments, the plurality of individual control subjects is or includes DLBCL subject. In particular embodiments, the subtype of NHL is the FL subtype of ALL. In some embodiments, the plurality of individual control subjects is or includes FL subject. In particular embodiments, the subtype of NHL is SLL. In some embodiments, the plurality of individual control subjects is or includes SLL subjects. In some embodiments, the plurality of individual control subjects is or includes subjects that have and/or are suspected of having a leukemia. In some embodiments, the plurality of individual control subjects is or includes subjects that have and/or are suspected of having a specific type of leukemia. In some embodiments, the plurality of individual control subjects is or includes subjects that have and/or are suspected of having chronic lymphocytic leukemia (CLL). In particular embodiments, the type of leukemia is CLL.

In certain embodiments, the plurality of individual control subjects is a plurality of subjects that have and/or are suspected of having a cancer. In some embodiments, the plurality of individual control subjects is or includes subjects that have and/or are suspected of having diffuse large B-cell lymphoma (DLBCL). In some embodiments, the plurality of individual control subjects is or includes subjects that have and/or are suspected of having a specific subtype of DLBCL. In some embodiments, the plurality of individual control subjects is or includes subjects that have and/or are suspected of having DLBCL, but not a specific subtype of DLBCL. In particular embodiments, the subtype of DLBCL is the germinal center B-cell (GCB) subtype of DLBCL. In some embodiments, the plurality of individual control subjects is or includes GCB DLBCL subjects. In particular embodiments, the subtype of DLBCL is the activated B-cell (ABC) subtype of DLBCL. In some embodiments, the plurality of individual control subjects is or includes ABC DLBCL subjects.

In some embodiments, the control value is obtained from a plurality of control samples. In certain embodiments, the plurality of control samples contains at least 2, at least 3, at least 5, at least 10, at least 15, at least 20, at least 25, at least 30, at least 40, at least 50, at least 100, at least 200, at least 300, at least 400, or at least 500 control samples.

In certain embodiments, an assessment, measurement, determination, or quantification of gene expression, e.g., the amount or level of one or more gene products, can be analyzed by any means in the art. In some embodiments, prior to an analysis, raw gene expression data, e.g., the value of the measured and/or quantified level, amount, or concentration of the gene product can be normalized or transformed, e.g., log-normalized, expressed as an expression ratio, percentile-ranked, and/or quantile-scaled. In some embodiments, gene expression data may further be modified by any nonparametric data scaling approach. In some embodiments, the transformation of the measurement or assessment of the expression of the one or more gene products occurs prior to any normalization to a control. In certain embodiments, the transformation of the measurement or assessment of the expression of the one or more gene products occurs after a normalization to a control value. In some embodiments, the transformation is a logarithmic transformation, a power transformation, or a logit transformation. In some embodiments, the logarithmic transformation is a common log (log₁₀(x)), a natural log (ln(x)), or a binary log (log₂(x)).

Expression patterns can be evaluated and classified by a variety of means, such as general linear model (GLM), ANOVA, regression (including logistic regression), support vector machines (SVM), linear discriminant analysis (LDA), principal component analysis (PCA), k-nearest neighbor (kNN), neural network (NN), nearest mean/centroid (NM), and Bayesian covariate predictor (BCP). A model, such as SVM, can be developed using any of the subsets and combinations of genes described herein based on the teachings of the invention. In more particular embodiments, an expression pattern is evaluated as the mean of log-normalized expression levels of the genes.

In some embodiments, a combination of one or more genes that positively correlate and one or more genes that negatively correlate are measured to determine a likelihood and/or probability of a clinical outcome (e.g. CR, PR, or PD). In certain embodiments, an expression profile and/or a gene expression profile is or is indicated by assessing, measuring, determining, and/or quantifying the expression of at least two genes. For example, in some embodiments, assessing or determining the gene expression profile of a sample may include assessing, measuring, determining, and/or quantifying of at least two genes that are associated with and/or correlated to a clinical outcome, e.g., CR, PR or PD. In certain embodiments, a gene expression profile is obtained by measuring, determining, and/or quantifying the expression of two or more genes, e.g., by measuring, determining, and/or quantifying the gene products of two or more genes, that are positively correlated with likelihood and/or probability of exhibiting a clinical outcome, e.g. CR, PR, or PD.

C. Gene Reference Value

In some embodiments, the comparison of a measurement of one or more gene products to a reference value of the one or more gene products allows for the assessment, measurement, and/or determination of the probability and/or likelihood of a clinical outcome (e.g. CR, PR, or PD) following administration of and/or associated with a therapy. In some embodiments, the expression of a gene product in a sample is compared to a reference value, e.g., a gene reference value. In some embodiments, the gene reference value is a value of a level, amount, or concentration of the gene product, and/or a transformation thereof. In some embodiments, the gene reference value is or is derived from an amount or level of an RNA gene product or a protein gene product. In particular embodiments, the gene reference value is an amount or level of the gene product, or a transformation thereof, that is a boundary between or a threshold value that separates the amounts or levels of the gene product, or transformations thereof, that indicate a likelihood of a clinical outcome (e.g. CR, PR, or PD), and/or an increased, elevated, or high probability of a clinical outcome (e.g. CR, PR, or PD), following administration of a therapy and values or measurements of gene expression that indicate an absent or low likelihood and/or a decreased, reduced, or low probability of a clinical outcome (e.g. CR, PR, or PD), following administration of a therapy. In some embodiments, the gene reference value is a boundary, divide, and/or threshold value between the amounts or levels of the gene product where a majority of one or more clinical responses take place or have previously taken place and amounts or levels of the gene product where a minority of one or more clinical responses take place or previously taken place.

In certain embodiments, the gene reference value is an amount or level of the gene product, or a transformation thereof, that is a boundary between or a threshold value that separates the amounts or levels of the gene product, or transformations thereof, associated with a particular type of clinical response from amounts or levels associated with one or more other types of clinical response. In particular embodiments, the gene reference value is an amount or level of the gene product, or a transformation thereof, that is a boundary between or a threshold value that separates the amounts or levels of the gene product, or transformations thereof, associated with CR and/or PR from the amounts or levels that are associated with other clinical responses, e.g. CRU, NR/SD, SD, and/or PD. In particular embodiments, the gene reference value is an amount or level of the gene product, or a transformation thereof, that is a boundary between or a threshold value that separates the amounts or levels of the gene product, or transformations thereof, associated with PD from the amounts or levels that are associated with other clinical responses, e.g. CRU, PR, NR/SD, SD, and/or CR.

In some embodiments, the expression of a gene product is compared to a reference value and/or a gene reference value and an elevated, increased and/or high probability and/or likelihood of a particular clinical response (e.g. CR, PR, or PD) is indicated. In particular embodiments, the expression of a gene product is compared to a gene reference value and a reduced, decreased and/or low probability and/or likelihood of a particular clinical response is indicated. In certain embodiments, the expression of a gene product that has been normalized to a control is compared to a reference value and/or a gene reference value and an elevated, increased and/or high probability and/or likelihood of a particular clinical response is indicated. In particular embodiments, the expression of a gene product that has been normalized to a control is compared to a gene reference value and a reduced, decreased and/or low likelihood and/or probability of a particular clinical response is indicated. In certain embodiments, a value of the expression of a gene product that have been normalized or transformed is compared to a reference value and/or a gene reference value and an elevated, increased and/or high probability and/or likelihood of a particular clinical response is indicated. In particular embodiments, the value of the expression of a gene product that have been normalized or transformed is compared to a gene reference value and a reduced, decreased and/or low probability and/or likelihood of a particular clinical response is indicated. In some embodiments, the particular clinical response is selected from among the following: CR, CRU, PR, NR/SD, SD, and/or PD.

In some embodiments, the expression of a gene product that is negatively correlated to and/or negatively associated with a clinical response (e.g. CR, PR, or PD) is compared to a gene reference value. In certain embodiments, when the expression of one or more prosurvival genes (i.e. that gene has an anti-apoptotic effect), such as myc family genes (c-myc, l-myc, and n-myc,) p53, and EZH2 is greater than, over, and/or above the gene reference value, then a decreased, reduced, and/or low probability and/or likelihood of PR or CR is indicated. In particular embodiments, when the expression of one or more prosurvival genes (i.e. that gene has an anti-apoptotic effect), such as myc family genes (c-myc, l-myc, and n-myc,) p53, and EZH2 is less than, under, and/or below the gene reference value, then an elevated, increased, and/or high probability and/or likelihood of PR or CR is indicated.

In particular embodiments, the expression of a gene product that is positively correlated to and/or positively associated with a particular clinical response (e.g. CR or PD) is compared to a gene reference value. In some embodiments, when the expression of a one or more prosurvival genes (i.e. that gene has an anti-apoptotic effect), such as myc family genes (c-myc, l-myc, and n-myc,) p53, and EZH2 is greater than, over, and/or above the gene reference value, than an increased, elevated, and/or high probability and/or likelihood of PD is indicated. In certain embodiments, when the expression of one or more prosurvival genes (i.e. that gene has an anti-apoptotic effect), such as myc family genes (c-myc, l-myc, and n-myc,) p53, and EZH2 is less than, under, and/or below the gene reference value, then a decreased, reduced, and/or low probability and/or likelihood of a PD is indicated.

In some embodiments, the gene reference value is a predetermined value. In particular embodiments, the gene reference value has been calculated and/or derived from data from a study. In some embodiments, the study is a clinical study. In particular embodiments, the clinical study is a completed clinical study. In certain embodiments, the data from the study included gene expression, e.g., expression of a gene product, in samples taken or obtained from subjects in the study. In particular embodiments, the data from the study includes the number and types of clinical responses experienced by subjects during the study. In certain embodiments, the subjects in the clinical study had or have a clinical response, such as CR, CRU, PR, NR/SD, SD, and/or PD. In some embodiments, the clinical response is CR. In some embodiments, the clinical response is not CR. In certain embodiments, the data from the study includes the number and types of diseases or conditions, such as cancer (e.g. NHL). In particular embodiments, the data from the study includes the number and types of treatment experienced by subjects during the study. In certain embodiments, the subjects are or were treated with a prosurvival BCL2 family protein inhibitor. In certain embodiments, the subjects are not or were not treated with a prosurvival BCL2 family protein inhibitor.

In some embodiments, the gene reference value is a predetermined value. In particular embodiments, the gene reference value has been calculated and/or derived from data from a study. In some embodiments, the study is a clinical study. In particular embodiments, the clinical study is a completed clinical study. In certain embodiments, the data from the study included gene expression, e.g., expression of a gene product, in samples taken or obtained from subjects in the study. In particular embodiments, the data from the study includes the number and types of therapy experienced by subjects during the study. In certain embodiments, the subjects in the clinical study are or were treated with a prosurvival BCL2 family protein inhibitor. In some embodiments, subjects in the clinical study are not or were not treated with a prosurvival BCL2 family inhibitor. In certain embodiments, the data from the study includes the number and types of diseases or conditions, such as cancer (e.g. NHL). In particular embodiments, the data from the study includes the number and types of clinical responses experienced by subjects during the study, such as CR, CRU, PR, NR/SD, SD, and/or PD.

In some embodiments, the reference value is or reflects a minimum detectable level, value, or amount of gene expression, e.g., a value that serves as a boundary of positive or negative expression. In certain embodiments, a measurement of the expression of a gene product is compared to a reference value that is or reflects a minimum detectable level, value, or amount of gene expression, e.g., expression of a gene product, and the gene is determined to be positively expressed if the measurement is a value above the reference value, and/or the gene is determined to be negatively expressed if the measurement is a value that is below the reference value.

In particular embodiments, the expression of a gene product in a sample taken or obtained from a subject is compared to a gene reference value that was calculated and/or derived from a study that included subjects with the same disease or condition as the subject. In certain embodiments, the same disease or condition is a cancer. In particular embodiments the same disease or condition is NHL. In particular embodiments, the expression of a gene product in a sample taken or obtained from a subject is compared to a gene reference value that was calculated and/or derived from a study that included subjects with the same disease or condition as the subject, but with a different subtype of the same disease or condition as the subject. In certain embodiments, the same disease or condition is a cancer. In particular embodiments the same disease or condition is NHL. In particular embodiments, the expression of a gene product in a sample taken or obtained from a subject with the DLBCL subtype of NHL is compared to a gene reference value that was calculated and/or derived from a study that included subjects with the FL subtype of NHL. In particular embodiments, the expression of a gene product in a sample taken or obtained from a subject with the FL subtype of NHL is compared to a gene reference value that was calculated and/or derived from a study that included subjects with the DLBCL subtype of NHL.

In particular embodiments, the expression of a gene product in a sample taken or obtained from a subject is compared to a gene reference value that was calculated and/or derived from a study that included subjects with the same clinical response as the subject. In certain embodiments, the same clinical response is CR and/or PR. certain embodiments, the same clinical response is PD. In particular embodiments, the expression of a gene product in a sample taken or obtained from a subject with CR and/or PR is compared to a gene reference value that was calculated and/or derived from a study that included subjects with PD. In particular embodiments, the expression of a gene product in a sample taken or obtained from a subject with PD is compared to a gene reference value that was calculated and/or derived from a study that included subjects with CR and/or PR.

In particular embodiments, the expression of a gene product in a sample taken or obtained from a subject is compared to a gene reference value that was calculated and/or derived from a study that included subjects with the same treatment as the subject. In certain embodiments, the same treatment is use of or treatment with a prosurvival BCL2 family protein inhibitor. In certain embodiments, the same treatment is no use or no treatment with a prosurvival BCL2 family protein inhibitor. In particular embodiments, the expression of a gene product in a sample taken or obtained from a subject treated with a prosurvival BCL2 family protein inhibitor is compared to a gene reference value that was calculated and/or derived from a study that included subjects not treated with a prosurvival BCL2 family protein inhibitor. In particular embodiments, the expression of a gene product in a sample taken or obtained from a subject not treated with a prosurvival BCL2 family protein inhibitor is compared to a gene reference value that was calculated and/or derived from a study that included subjects treated with a prosurvival BCL2 family protein inhibitor

In some embodiments, the gene reference value is determined by the application of an algorithm to the level, concentration, or amount of expression in a control sample or a plurality of control samples. In some embodiments, the control sample or plurality of control samples is obtained from a subject or group of subjects of a completed study, e.g., a completed clinical trial, where the subjects were monitored for clinical response. In particular embodiments, the sample or the plurality of samples were collected prior to the subjects receiving the therapy. In some embodiments, the subject or group of subjects went on to exhibit clinical responses after the therapy was administered. In some embodiments, the subject or group of subjects went on to exhibit a clinical response within 1 month, within 2 months, within 3 months, within 4 months, within 5 months, within 6 months, within 7 months, within 8 months, within 9 months, or more, of initiation of administration of the therapy. In certain embodiments, the subject or group of subjects developed and/or experienced a clinical response of CR, CRU, PR, NR/SD, SD, or PD. In some embodiments, the subject or group of subjects developed and/or experienced a clinical response of CR. In certain embodiments, the subject or group of subjects developed and/or experienced a response of CRU. In certain embodiments, the subject or group of subjects developed and/or experienced a response of PR. In certain embodiments, the subject or group of subjects developed and/or experienced a response of NR/SD. In certain embodiments, the subject or group of subjects developed and/or experienced a response of SD. In certain embodiments, the subject or group of subjects developed and/or experienced a response of PD. In some embodiments, the gene reference value is determined by the application of an algorithm to two or more control samples or pluralities that are obtained from two or more different subjects or different groups of subjects.

In some embodiments, the gene reference value is determined by the application of an algorithm to the level, concentration, or amount of expression in a control sample or a plurality of control samples. In some embodiments, the control sample or plurality of control samples is obtained from a subject or group of subjects of a completed study, e.g., a completed clinical trial, where the subjects were monitored for type of treatment, e.g., use of or treatment with a prosurvival BCL2 family inhibitor. In particular embodiments, the sample or the plurality of samples were collected prior to the subjects receiving the treatment. In particular embodiments, the sample or the plurality of samples were collected after the subjects received the treatment. In some embodiments, the gene reference value is determined by the application of an algorithm to two or more control samples or pluralities that are obtained from two or more different subjects or different groups of subjects.

In certain embodiments, illustrative algorithms include but are not limited to methods that reduce the number of variables such as principal component analysis algorithms, partial least squares methods, and independent component analysis algorithms. Illustrative algorithms further include but are not limited to methods that handle large numbers of variables directly such as statistical methods and methods based on machine learning techniques. Statistical methods include penalized logistic regression, prediction analysis of microarrays (PAM), methods based on shrunken centroids, support vector machine analysis, and regularized linear discriminant analysis. Machine learning techniques include bagging procedures, boosting procedures, random forest algorithms, and combinations thereof. In some embodiments of the present invention a support vector machine (SVM) algorithm, a random forest algorithm, or a combination thereof is used for classification of microarray data or RNA-seq data. In some embodiments, identified markers that distinguish samples or subtypes are selected based on statistical significance. In some cases, the statistical significance selection is performed after applying a Benjamini Hochberg correction for false discovery rate (FDR). In certain embodiments, the algorithmic techniques may be applied to the expression profiles of one or more gene products in a sample, such as one or more prosurvival genes (i.e. that gene has an anti-apoptotic effect), such as myc family genes (c-myc, l-myc, and n-myc,) p53, and EZH2.

In some embodiments, the algorithm may be supplemented with a meta-analysis approach such as that described by Fishel and Kaufman et al. 2007 Bioinformatics 23(13): 1599-606. Also, the classifier algorithm may be supplemented with a meta-analysis approach such as a repeatability analysis. In some cases, the repeatability analysis selects markers that appear in at least one predictive expression product marker set.

In some embodiments, the gene reference value is an amount or level of the gene product, or a transformation thereof, that is a boundary between or a threshold value that separates the amounts or levels of the gene product, or transformations thereof, where all or a majority of a clinical response takes place or have previously taken place, from amounts or levels of the gene product, or transformations thereof, where a minority of the clinical responses take place or previously taken place. In particular embodiments, the gene reference value partitions or separates values or measurements of the gene expression associated with more than half, and/or greater than 50%, 60%, 70%, 80%, 90%, 95%, or at or about 100% of the instances of a clinical response, e.g., CR, PR, or PD, that occurred in a study. In some embodiments, the clinical response is CR, CRU, PR, NR/SD, SD, or PD. In some embodiments, the clinical response is CR. In some embodiments, the clinical response is PR. In some embodiments, the a clinical response is PD. In some embodiments, the clinical response is not CR or PR. In some embodiments, the gene reference value partitions or separates values or measurements of the gene expression that are associated with at least a 25%, at least a 30%, at least a 40%, at least a 45%, at least a 50%, at least a 55%, at least a 60%, at least a 65%, at least a 70%, at least a 75%, at least a 80%, at least a 85%, at least a 90%, at least a 95%, or at or about a 100% frequency of a clinical response, such as CR, CRU, PR, NR/SD, SD, or PD.

In some embodiments, the gene reference value is an amount or level of the gene product, or a transformation thereof, that is a boundary between or a threshold value that separates the amounts or levels of the gene product, or transformations thereof, where all or a majority of a type of treatment takes place or have previously taken place, from amounts or levels of the gene product, or transformations thereof, where a minority of a type of treatment takes place or previously taken place. In particular embodiments, the gene reference value partitions or separates values or measurements of the gene expression associated with more than half, and/or greater than 50%, 60%, 70%, 80%, 90%, 95%, or at or about 100% of the instances of use of or treatment with a prosurvival BCL2 family protein inhibitor that occurred in a study. In some embodiments, the gene reference value partitions or separates values or measurements of the gene expression that are associated with at least a 25%, at least a 30%, at least a 40%, at least a 45%, at least a 50%, at least a 55%, at least a 60%, at least a 65%, at least a 70%, at least a 75%, at least a 80%, at least a 85%, at least a 90%, at least a 95%, or at or about a 100% frequency of use of or treatment with a prosurvival BCL2 family protein inhibitor.

In particular embodiments, the gene reference value is within 25%, within 20%, within 15%, within 10% or within 5% of the gene expression in a control sample. In some embodiments, the gene reference value is within 25%, within 20%, within 15%, within 10% or within 5% an average or median level, concentration or amount of the gene expression in a plurality of control samples. In particular embodiments, the gene reference value is within 2, 1.5, 1.25, 1, 0.75, 0.5, 0.25, or 0.1 standard deviations of an average or median level, concentration or amount of the gene expression in a plurality of control samples, wherein each of the subjects of the group went on to exhibit a clinical response, e.g. CR, PR, or PD, after receiving the cytoxic therapy for treating the same disease or condition.

In some embodiments, the reference value is obtained from and/or derived from control samples that were obtained from subjects prior to the administration of the therapy, wherein the subjects of the group went on to exhibit PD. In some embodiments, the gene reference value is a value of a gene product of a gene that positively correlates and/or is positively associated with PD following administration of and/or associated with a therapy, e.g., an immunotherapy or cell therapy, such as one or more prosurvival genes (i.e. that gene has an anti-apoptotic effect), such as myc family genes (c-myc, l-myc, and n-myc,) p53, and EZH2.

In certain embodiments, the gene reference value is within 2-fold, within 1.5 fold, 1.0 fold, within 100%, within 50%, within 40%, within 30%, within 25%, within 20%, within 15%, within 10%, or within 5% above the average level, concentration or amount, and/or is within 2.0, 1.5, 1.25, 1.0, 0.75, 0.5, or 0.25 standard deviations above the average level, concentration, or amount of the gene product in a plurality of the control samples. In certain embodiments, the gene reference value is above the highest level, concentration or amount of the gene product observed in a sample from among the plurality of control samples. In particular embodiments, the gene reference value is within 100%, 75%, 50%, 40%, 30%, 25%, 20%, 10%, or 5% above the highest level, concentration or amount of the gene product observed in a sample from among the plurality of control samples. In some embodiments, the reference value is above the level, concentration or amount observed in at least 50%, at least 60%, at least 70%, at least 75%, at least 80%, at least 85%, at least 90%, or at least 95%, or at least 98% of samples from among a plurality of control samples.

In some embodiments, the gene reference value is a value of a gene product of a gene that negatively correlates and/or is negatively associated with PD following administration of and/or associated with a therapy, and the plurality of control samples are obtained from a group of subjects prior to receiving therapy, wherein each of the subjects of the group did not go on to exhibit PD. In some embodiments, the subjects went on to exhibit CR. In some embodiments, the value is below the lowest level, concentration, or amount, of the at least one gene product observed in a sample from among a plurality of control samples. In certain embodiments, the reference value is below the lowest level, concentration, or amount of the gene product observed in a sample from among the plurality of control samples that did not go on to exhibit PD. In some embodiments, the reference value is within 50%, within 40%, within 30%, within 25%, within 20%, within 15%, within 10%, or within 5% below the lowest level, concentration, or amount of the gene product observed in a sample from among the plurality of control samples that did not go on to exhibit PD. In some embodiments, the reference value is below the level, concentration, or amount observed in at least 75%, at least 80%, at least 85%, at least 90%, or at least 95%, or at least 98% of samples from among a plurality of control samples obtained from a group of subjects prior to receiving a cell therapy that did not go on to exhibit PD.

In some embodiments, the gene reference value is obtained from and/or derived from control samples that were obtained from subjects prior to the administration of the therapy, wherein the subjects of the group went on to exhibit CR. In some embodiments, the gene reference value is a value of a gene product of a gene that negatively correlates and/or is negatively associated with CR following administration of and/or associated with a therapy, e.g., an immunotherapy or cell therapy, such as a one or more prosurvival genes (i.e. that gene has an anti-apoptotic effect), such as myc family genes (c-myc, l-myc, and n-myc,) p53, and EZH2. In certain embodiments, the gene reference value is within 2-fold, within 1.5 fold, 1.0 fold, within 100%, within 50%, within 40%, within 30%, within 25%, within 20%, within 15%, within 10%, or within 5% above the average level, concentration or amount, and/or is within 2.0, 1.5, 1.25, 1.0, 0.75, 0.5, or 0.25 standard deviations above the average level, concentration, or amount of the gene product in a plurality of the control samples. In certain embodiments, the gene reference value is above the highest level, concentration or amount of the gene product observed in a sample from among the plurality of control samples. In particular embodiments, the gene reference value is within 100%, 75%, 50%, 40%, 30%, 25%, 20%, 10%, or 5% above the highest level, concentration or amount of the gene product observed in a sample from among the plurality of control samples. In some embodiments, the reference value is above the level, concentration or amount observed in at least 50%, at least 60%, at least 70%, at least 75%, at least 80%, at least 85%, at least 90%, or at least 95%, or at least 98% of samples from among a plurality of control samples.

In some embodiments, the gene reference value is a value of a gene product of a gene that negatively correlates and/or is negatively associated with CR following administration of and/or associated with a therapy, and the plurality of control samples are obtained from a group of subjects prior to receiving therapy, wherein each of the subjects of the group did not go on to exhibit CR. In some embodiments, the subjects went on to exhibit PD. In some embodiments, the value is below the lowest level, concentration, or amount, of the at least one gene product observed in a sample from among a plurality of control samples. In certain embodiments, the reference value is below the lowest level, concentration, or amount of the gene product observed in a sample from among the plurality of control samples that did not go on to exhibit CR. In some embodiments, the reference value is within 50%, within 40%, within 30%, within 25%, within 20%, within 15%, within 10%, or within 5% below the lowest level, concentration, or amount of the gene product observed in a sample from among the plurality of control samples that did not go on to exhibit CR. In some embodiments, the reference value is below the level, concentration, or amount observed in at least 75%, at least 80%, at least 85%, at least 90%, or at least 95%, or at least 98% of samples from among a plurality of control samples obtained from a group of subjects prior to receiving a cell therapy that did not go on to exhibit CR.

In some embodiments, the gene reference value is a value of a gene product of a gene that negatively correlates and/or is negatively associated with a clinical response following administration of and/or associated with a therapy, and the plurality of control samples are obtained from a group of subjects prior to receiving therapy, wherein each of the subjects of the group did not develop or go on to develop PD. In some embodiments, the subjects exhibited or went on to exhibit CR, after receiving the therapy. In some embodiments, the value is below the lowest level, concentration, or amount, of the at least one gene product observed in a sample from among a plurality of control samples. In certain embodiments, the reference value is below the lowest level, concentration, or amount of the gene product observed in a sample from among the plurality of control samples that did not experience severe neurotoxicity. In some embodiments, the reference value is within 50%, within 40%, within 30%, within 25%, within 20%, within 15%, within 10%, or within 5% below the lowest level, concentration, or amount of the gene product observed in a sample from among the plurality of control samples that did not exhibit PD. In some embodiments, the reference value is below the level, concentration, or amount observed in at least 75%, at least 80%, at least 85%, at least 90%, or at least 95%, or at least 98% of samples from among a plurality of control samples obtained from a group of subjects prior to receiving a cell therapy that did not exhibit or go on to exhibit PD.

In certain embodiments, the reference value is obtained from and/or derived from control samples that were obtained from subjects prior to the administration of the therapy, wherein the subjects of the group went on to exhibit PD. In some embodiments, the gene reference value is a value of a gene product of a gene that positively correlates and/or is positively associated with PD following administration of and/or associated with a therapy, e.g., an immunotherapy or cell therapy, such as one or more prosurvival genes (i.e. that gene has an anti-apoptotic effect), such as myc family genes (c-myc, l-myc, and n-myc,) p53, and EZH2. In certain embodiments, the gene reference value is within 2-fold, within 1.5 fold, 1.0 fold, within 100%, within 50%, within 40%, within 30%, within 25%, within 20%, within 15%, within 10%, or within 5% below the average level, concentration or amount, and/or is within 2.0, 1.5, 1.25, 1.0, 0.75, 0.5, or 0.25 standard deviations below the average level, concentration, or amount of the gene product in a plurality of the control samples. In certain embodiments, the gene reference value is below the lowest level, concentration or amount of the gene product observed in a sample from among the plurality of control samples. In particular embodiments, the gene reference value is within 100%, 75%, 50%, 40%, 30%, 25%, 20%, 10%, or 5% below the lowest level, concentration or amount of the gene product observed in a sample from among the plurality of control samples. In some embodiments, the reference value is below the level, concentration or amount observed in at least 50%, at least 60%, at least 70%, at least 75%, at least 80%, at least 85%, at least 90%, or at least 95%, or at least 98% of samples from among a plurality of control samples.

In some embodiments, the gene reference value is a value of a gene product of a gene that negatively correlates and/or is negatively associated with a clinical response following administration of and/or associated with a therapy, and the plurality of control samples are obtained from a group of subjects prior to receiving a cell therapy containing cells genetically engineered with a recombinant receptor, wherein each of the subjects of the group went on to exhibit CR. In some embodiments, the value is below the lowest level, concentration, or amount, of the at least one gene product observed in a sample from among a plurality of control samples. In certain embodiments, the value is within 100%, within 75%, within 50%, within 40%, within 30%, within 25%, within 20%, within 10%, or within 5% below the lowest level, concentration, or amount, of the at least one gene product observed in a sample from among a plurality of control samples. In some embodiments, the reference value is above the level, concentration, or amount observed in at least 75%, at least 80%, at least 85%, at least 90%, or at least 95%, or at least 98% of samples from among a plurality of control samples obtained from a group of subjects prior to receiving a therapy that did not go on to exhibit PD.

In some embodiments, the expression of at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least twenty, at least twenty-one, at least twenty-two, at least twenty-three, at least twenty-four, at least twenty-five, at least twenty-six, at least twenty-seven, at least twenty-eight, at least twenty-nine, at least thirty, at least thirty-five, at least forty, at least fifty, at least sixty, at least seventy, at least eighty, at least ninety, at least one hundred, or at least one hundred and twenty gene products in a sample obtained from a subject are compared to corresponding gene reference values, e.g., gene reference values to the same gene, to determine the probability, risk, or likelihood, that the subject will exhibit a clinical outcome (e.g. CR, PR, or PD). In some embodiments, the subject is at an elevated, increased, and/or high risk of a clinical outcome (e.g. CR, PR or PD) if comparison of the expression of at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least twenty, at least twenty-one, at least twenty-two, at least twenty-three, at least twenty-four, at least twenty-five, at least twenty-six, at least twenty-seven, at least twenty-eight, at least twenty-nine, at least thirty, at least thirty-five, at least forty, at least fifty, at least sixty, at least seventy, at least eighty, at least ninety, at least one hundred, or at least one hundred and twenty gene products with the corresponding gene reference value indicate that the expression is associated with an elevated, increased, and/or high risk of a clinical outcome (e.g. CR, PR, or PD).

In some embodiments, the expression of at least one gene product that positively correlates and/or is positively associated with a clinical outcome (e.g. CR, PR, or PD), and the expression of at least one gene product that negatively correlates to and/or is negatively associated with a clinical outcome (e.g. CR, PR, or PD), are compared to the corresponding reference values to determine the probability, risk, or likelihood, that the subject will experience a clinical outcome (e.g. CR, PR, or PD).

In particular embodiments, a subject is and/or is considered to have a high, elevated, and/or increased risk of a clinical outcome (e.g. CR, PR, or PD), if the expression of one or more gene products that are negatively correlated to and/or negatively associated with the outcome, response, and/or subtype are below the reference value. In certain embodiments, a subject is and/or is considered to have a high, elevated, and/or increased risk of developing a clinical outcome (e.g. CR, PR or PD), if the expression of expression of at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-five, at least fifty, at least one hundred, or at least one hundred and twenty gene products that are negatively correlated to and/or negatively associated with the clinical outcome are below the reference value.

In some embodiments, a subject is and/or is considered to have a high, elevated, and/or increased risk of developing a clinical outcome (e.g. CR, PR, or PD), if the expression of one or more gene products that are positively correlated to and/or positively associated with the clinical outcome, are above the reference value. In certain embodiments, a subject is and/or is considered to have a high, elevated, and/or increased risk of developing a clinical outcome (e.g. CR, PR or PD), if the expression of expression of at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-five, at least fifty, at least one hundred, or at least one hundred and twenty gene products that are positively correlated to and/or positively associated with the clinical outcome are above the reference value.

In particular embodiments, a subject is and/or is considered to have a low, decreased, and/or reduced risk of a clinical outcome (e.g. CR, PR, or PD), if the expression of one or more gene products that are negatively correlated to and/or negatively associated with the clinical outcome are above the reference value. In certain embodiments, a subject is and/or is considered to have a low, decreased, and/or reduced risk of developing a clinical outcome (e.g. CR, PR, or PD) if the expression of expression of at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-five, at least fifty, at least one hundred, or at least one hundred and twenty gene products that are negatively correlated to and/or negatively associated with the clinical outcome are above the reference value.

In some embodiments, the gene product is a protein, e.g., a protein measured from a plasma sample that is obtained from a subject prior to or subsequent to administration of a cell therapy, and the gene reference value is a concentration of the protein in serum. In certain embodiments, protein is a gene product that is negatively correlated to and/or negatively associated with PR and/or CR. In certain embodiments, protein is a gene product that is positively correlated to and/or positively associated with PR and/or CR. In certain embodiments, the plasma sample is obtained from a subject prior to administration of the cell therapy, such as 1 day prior. In certain embodiments, the plasma sample is obtained from a subject subsequent to administration of the cell therapy, such as 2 days, 4 days, or 7 days after administration of the cell therapy.

In certain embodiments, the plasma sample is obtained from a subject prior to administration of the cell therapy. In certain embodiments, the protein is a protein or portion of one or more prosurvival gene (i.e. the gene confers an anti-apoptotic effect), such as a myc family gene (c-myc, l-myc, and n-myc), p53, and EZH2. In some embodiments, the gene reference value is a concentration of the gene product, e.g., the protein or portion thereof, in serum.

D. Selection of Subjects

In some embodiments, a subject with a high, elevated, and/or increased likelihood of exhibiting or going on to exhibit partial response (PR) or complete response (CR) following administration of a cytotoxic therapy (e.g. a cell therapy, such as a CAR-T cell therapy) is not administered a prosurvival BCL2 family protein inhibitor to treat, prevent, delay, or attenuate progressive disease (PD). In some embodiments, a subject with a high, elevated, and/or increased risk of exhibiting or going on to exhibit progressive disease (PD) following administration of a cytotoxic therapy (e.g. a cell therapy, such as a CAR-T cell therapy) is administered a prosurvival BCL2 family protein inhibitor to treat, prevent, delay, or attenuate (PD). Thus, in some embodiments, provided herein is a combination therapy, comprising administration of a cytotoxic therapy and a prosurvival BCL2 family protein inhibitor to treat, prevent, delay, or attenuate the development of or risk for developing progressive disease (PD). Also provided are compositions and formulations, e.g., pharmaceutical formulations, comprising one or more of the agents.

In some embodiments, the methods provided herein allow for selection of a subject for a combination therapy, e.g., administration of a cytotoxic therapy and a prosurvival BCL2 family protein inhibitor for treating, preventing, delaying, reducing or attenuating the development or risk of development of progressive disease (PD) following administration of the cytotoxic therapy, by identifying subject at with a likelihood and/or an increased risk, probability, or likelihood of developing or experiencing progressive disease (PD) following administration of the cytotoxic therapy. In some embodiments, the inhibitor is administered (i) prior to, (ii) within one, two, or three days of, (iii) concurrently with and/or (iv) subsequent to, the initiation of administration of the immunotherapy or cell therapy to the subject.

In some embodiments, the subject is not administered or provided with a prosurvival BCL2 family protein inhibitor capable of treating, preventing, delaying, reducing or attenuating the development or risk of development of progressive disease (PD) prior to the administration of the cytotoxic therapy. In some embodiments, the subject is not administered or provided with the prosurvival BCL2 family protein inhibitor for a period of time prior to the initiation of the administration of the cytotoxic therapy. In some embodiments, the period of time is or is about 1, 2, 3, 4, 5, 6, 7, 8, 9, 10, 11, 12, 13, 14, or 15 days, and/or is or is about 1, 2, 3, 4, 5, 6, or longer than 6 weeks prior to the administration of the cytotoxic therapy. In some embodiments, the subject is not administered or provided with the prosurvival BCL2 family inhibitor prior to administration of the cytotoxic therapy, prior to the subject exhibiting, or unless the subject exhibits, a sign or symptom of progressive disease (PD).

In some embodiments, the subject is administered or provided with a prosurvival BCL2 family protein inhibitor capable of treating, preventing, delaying, reducing or attenuating the development or risk of development of progressive disease (PD), prior to the administration of the immunotherapy or cell therapy. In some embodiments, the subject has been determined to have a high, increased, or elevated risk for PD. In some embodiments, the subject is administered or provided with a prosurvival BCL2 family protein inhibitor within a period of time prior to the initiation of the administration of the cytotoxic therapy, such as within or within about 1, 2, 3, 4, 5, 6, 7, 8, 9, 10, 11, 12, 13, 14, or 15 days, and/or within or within about 1, 2, 3, 4, 5, 6, or longer than 6 weeks prior to the administration of the cytotoxic therapy. In some embodiments, the subject is administered or provided with the inhibitor at the first sign or symptom of PD.

In some embodiments, a subject is identified as having a likelihood and/or an increased probability or likelihood of developing or experiencing progressive disease (PD) following administration of the cytotoxic therapy based on amounts or levels of gene transcription or protein expression in the subject. In some embodiments, the amounts or levels of gene transcription or protein expression of one or more prosurvival gene (i.e. the gene confers an anti-apoptotic effect) identify a subject as having a likelihood and/or an increased probability or likelihood of developing or experiencing progressive disease (PD) following administration of the therapy. In some embodiments, the amounts or levels of gene transcription or protein expression of one or more prosurvival gene identify a subject as having a likelihood and/or an increased probability or likelihood of developing or experiencing progressive disease (PD) following administration of the therapy when the amounts or levels are increased or higher compared to a control value. In some embodiments, the amounts or levels of gene transcription or protein expression of one or more prosurvival gene identify a subject as not having a likelihood and/or an increased probability or likelihood of developing or experiencing progressive disease (PD) following administration of the immunotherapy or cell therapy when the amounts or levels are decreased or lower compared to a control value. In some embodiments, the one or more prosurvival genes include myc family genes (c-myc, l-myc, n-myc), p53, and EZH2. In some embodiments, the one or more prosurvival genes include genes associated with myc family genes (c-myc, l-myc, n-myc), p53, and EZH2.

In some embodiments, the assessment, determination, measurement, and/or quantification of a gene product, e.g., an RNA or protein gene product, of a sample is normalized to a control value. In certain embodiments, normalization to one or more control values may be performed to analyze, assess, or determine if an amount or level of the gene product indicates if the expression of the gene is elevated or decreased, and/or high or low. In particular embodiments, normalization to control values may be used to compare the gene expression of a gene a sample to the gene expression of a different sample.

In particular embodiments, the control value is a measurement, or a value of a measurement, of a different gene product. In some embodiments, the different gene product is a gene product of a housekeeping gene. In certain embodiments, the housekeeping gene is a constitutively active gene, e.g., a gene that is required for maintenance of basic cellular function. Examples of suitable housekeeping genes are known in the art, and include, but are not limited to, genes encoding ACTB (Beta-actin), B2M (Beta-2-microglobulin), GAPDH (Glyceraldehyde 3-phosphate dehydrogenase), RPLP0 (60S acidic ribosomal protein P0), GUSB (beta-glucuronidase), HMBS (Hydroxymethyl-bilane synthase), HPRT1 (Hypoxanthine phosphoribosyl-transferase 1), RPL13A (Ribosomal protein L13a), SDHA, succinate dehydrogenase complex subunit A), TBP (TATA box binding protein), TFRC (transferring receptor 1), and UBC (Ubiquitin C). In some embodiments, the control value is measured in the same sample as the gene product.

In certain embodiments, the gene product is compared and/or normalized to a control value that is a measurement, or a value of a measurement, of the gene product from the same gene. In some embodiments, the control value is a measurement, or a value of a measurement, that is obtained from one or more control samples. In certain embodiments, the gene product and the control value are measured in different samples. In some embodiments, the one or more control samples have an identical, a same, or a similar tissue composition and/or cellular composition as the sample. In some embodiments, the sample and control sample are different samples from the same, similar, and/or identical tissue from the same subject. In particular embodiments, the sample and the control sample different samples from the same tissue in different subjects. In particular embodiments, the sample and control sample are different samples from the same, similar, and/or identical tissue from different subjects. In some embodiments, the one or more samples are lymph node biopsy samples and the one or more control samples are lymph node biopsy samples. In particular embodiments, the one or more samples are blood samples, e.g., peripheral blood samples, and the one or more control samples are blood samples.

In certain embodiments, the control sample is obtained from a subject that does not have a condition and/or a cancer. In particular embodiments, the control sample is obtained from a subject that does not have a NHL, such as FL or DLBCL. In some embodiments, the control sample does not have and/or is not suspected of having one or more tumor cells. In particular embodiments, the sample and control sample are different samples from the same, similar, and/or identical tissue from different subjects. In certain embodiments, the control sample is obtained from a subject that is treated with an EZH2 inhibitor. In certain embodiments, the control sample is obtained from a subject that is not treated with an EZH2 inhibitor. In certain embodiments, the control sample is obtained from a subject that does not exhibit complete response (CR) following treatment with a therapy, e.g. a cell therapy or an immunotherapy. In certain embodiments, the control sample is obtained from a subject that does not exhibit progressive disease (PD) following treatment with a therapy, e.g. an immunotherapy or a cell therapy. In certain embodiments, the control sample is obtained from a subject that does not exhibit T cell infiltration into a tumor microenvironment (TME) following treatment with a therapy, e.g. an immunotherapy or a cell therapy. In certain embodiments, the control sample is obtained from a subject that does exhibit T cell infiltration into a tumor microenvironment (TME) following treatment with a therapy, e.g. an immunotherapy or a cell therapy.

In certain embodiments, the assessment, determination, measurement, and/or quantification of a gene product, e.g., an RNA or protein gene product, of a sample is normalized to and/or compared to two or more control values. In some embodiments, the two or more control values include a control value that is a measurement, or a value of a measurement, that of the same gene product and a control value that is a measurement, or a value of a measurement, that of a different gene product.

In some embodiments, the control value has been previous determined. In certain embodiments, the one or more control values are measured or obtained in parallel with the assessment, measurement, determination, and/or quantification of the one or gene products in the sample.

In particular embodiments, the control value is an average (e.g. an arithmetic mean) or a median amount or level of expression of the one or more gene products obtained from a plurality of control samples. In some embodiments, the plurality of control samples is obtained from individual control subjects. In particular embodiments, the plurality of individual control subjects are subjects that do not have and/or are not suspected of having a condition or a disease. In some embodiments, the plurality of individual control subjects are subjects that do not have and/or are not suspected of having a cancer. In some embodiments, the plurality of individual control subjects are subjects that do not have and/or are not suspected of having a NHL. In some embodiments, the plurality of individual control subjects are subjects that do not have and/or are not suspected of having a specific subtype of NHL, such as FL or DLBCL. In some embodiments, the plurality of individual control subjects are subjects that do not have and/or are not suspected of having a specific subtype of NHL, such as SLL. In particular embodiments, the subtype of NHL is the follicular lymphoma (FL) subtype of NHL. In particular embodiments, the subtype of NHL is the diffuse large B-cell lymphoma (DLBCL) subtype of NHL. In particular embodiments, the subtype of NHL is the small lymphocytic lymphoma (SLL) subtype of NHL. In some embodiments, the plurality of individual control subjects are subjects that do not have and/or are not suspected of having a NHL. In some embodiments, the plurality of individual control subjects are subjects that do not have and/or are not suspected of having a specific type of leukemia, such as CLL. In some embodiments, the plurality of individual control subjects are subjects that do not have and/or are not suspected of having CLL.

In certain embodiments, the plurality of individual control subjects is a plurality of subjects that have and/or are suspected of having a cancer. In some embodiments, the plurality of individual control subjects is or includes subjects that have and/or are suspected of having non-Hodgkin lymphoma (NHL). In some embodiments, the plurality of individual control subjects is or includes subjects that have and/or are suspected of having a specific subtype of NHL. In some embodiments, the plurality of individual control subjects is or includes subjects that have and/or are suspected of having NHL, but not a specific subtype of NHL. In particular embodiments, the subtype of NHL is the DLBCL subtype of ALL. In some embodiments, the plurality of individual control subjects is or includes DLBCL subject. In particular embodiments, the subtype of NHL is the FL subtype of ALL. In particular embodiments, the subtype of NHL is the FL subtype of SLL. In some embodiments, the plurality of individual control subjects is or includes FL subject. In some embodiments, the plurality of individual control subjects is or includes subjects that have and/or are suspected of having a leukemia. In some embodiments, the leukemia is chronic lymphocytic leukemia (CLL).

In certain embodiments, the plurality of individual control subjects is a plurality of subjects that have and/or are suspected of having a cancer. In some embodiments, the plurality of individual control subjects is or includes subjects that have and/or are suspected of having diffuse large B-cell lymphoma (DLBCL). In some embodiments, the plurality of individual control subjects is or includes subjects that have and/or are suspected of having a specific subtype of DLBCL. In some embodiments, the plurality of individual control subjects is or includes subjects that have and/or are suspected of having DLBCL, but not a specific subtype of DLBCL. In particular embodiments, the subtype of DLBCL is the germinal center B-cell (GCB) subtype of DLBCL. In some embodiments, the plurality of individual control subjects is or includes GCB DLBCL subjects. In particular embodiments, the subtype of DLBCL is the activated B-cell (ABC) subtype of DLBCL. In some embodiments, the plurality of individual control subjects is or includes ABC DLBCL subjects.

In some embodiments, the plurality of individual control subjects are subjects that are not treated or have not been treated with an EZH2 inhibitor. In some embodiments, the plurality of individual control subjects are subjects that are treated or have been treated with an EZH2 inhibitor. In some embodiments, the plurality of individual control subjects are subjects that do not exhibit or have not exhibited CR following treatment with a therapy, e.g. an immunotherapy or a cell therapy. In some embodiments, the plurality of individual control subjects are subjects that do not exhibit or have not exhibited PD following treatment with a therapy, e.g. an immunotherapy or a cell therapy. In some embodiments, the plurality of individual control subjects are subjects that do not exhibit or have not exhibited a T cell response, e.g. T cell infiltration into a tumor microenvironment, following treatment with a therapy, e.g. an immunotherapy or a cell therapy. In some embodiments, the plurality of individual control subjects are subjects that exhibit or have exhibited a T cell response, e.g. T cell infiltration into a tumor microenvironment, following treatment with a therapy, e.g. an immunotherapy or a cell therapy.

In some embodiments, the control value is obtained from a plurality of control samples. In certain embodiments, the plurality of control samples contains at least 2, at least 3, at least 5, at least 10, at least 15, at least 20, at least 25, at least 30, at least 40, at least 50, at least 100, at least 200, at least 300, at least 400, or at least 500 control samples.

In certain embodiments, an assessment, measurement, determination, or quantification of gene expression, e.g., the amount or level of one or more gene products, can be analyzed by any means in the art. In some embodiments, prior to an analysis, raw gene expression data, e.g., the value of the measured and/or quantified level, amount, or concentration of the gene product can be normalized or transformed, e.g., log-normalized, expressed as an expression ratio, percentile-ranked, and/or quantile-scaled. In some embodiments, gene expression data may further be modified by any nonparametric data scaling approach. In some embodiments, the transformation of the measurement or assessment of the expression of the one or more gene products occurs prior to any normalization to a control. In certain embodiments, the transformation of the measurement or assessment of the expression of the one or more gene products occurs after a normalization to a control value. In some embodiments, the transformation is a logarithmic transformation, a power transformation, or a logit transformation. In some embodiments, the logarithmic transformation is a common log (log₁₀(x)), a natural log (ln(x)), or a binary log (log₂(x)).

Expression patterns can be evaluated and classified by a variety of means, such as general linear model (GLM), ANOVA, regression (including logistic regression), support vector machines (SVM), linear discriminant analysis (LDA), principal component analysis (PCA), k-nearest neighbor (kNN), neural network (NN), nearest mean/centroid (NM), and Bayesian covariate predictor (BCP). A model, such as SVM, can be developed using any of the subsets and combinations of genes described herein based on the teachings of the invention. In more particular embodiments, an expression pattern is evaluated as the mean of log-normalized expression levels of the genes.

### Exemplary Treatment Outcomes and Methods for Assessing the Same

In some embodiments of the methods, combinations, uses, kits and articles of manufacture provided herein, the provided combination therapy results in one or more treatment outcomes, such as a feature associated with any one or more of the parameters associated with the therapy or treatment, as described below. In some embodiments, the method includes assessment of the cytotoxicity of the T cells toward cancer cells, e.g., T cells administered for the T cell based therapy. In some embodiments, the method includes assessment of the exposure, persistence and proliferation of the T cells, e.g., T cells administered for the T cell based therapy. In some embodiments, the exposure, or prolonged expansion and/or persistence of the cells, and/or changes in cell phenotypes or functional activity of the cells, e.g., cells administered for immunotherapy, e.g. T cell therapy, in the methods provided herein, can be measured by assessing the characteristics of the T cells in vitro or ex vivo. In some embodiments, such assays can be used to determine or confirm the function of the T cells, e.g. T cell therapy, before, during, or after administering the combination therapy provided herein.

In some embodiments, the combination therapy can further include one or more screening steps to identify subjects for treatment with the combination therapy and/or continuing the combination therapy, and/or a step for assessment of treatment outcomes and/or monitoring treatment outcomes. In some embodiments, the step for assessment of treatment outcomes can include steps to evaluate and/or to monitor treatment and/or to identify subjects for administration of further or remaining steps of the therapy and/or for repeat therapy. In some embodiments, the screening step and/or assessment of treatment outcomes can be used to determine the dose, frequency, duration, timing and/or order of the combination therapy provided herein.

In some embodiments, any of the screening steps and/or assessment of treatment of outcomes described herein can be used prior to, during, during the course of, or subsequent to administration of one or more steps of the provided combination therapy, e.g., administration of the T cell therapy (e.g. CAR-expressing T cells), and/or a prosurvival BCL2 family protein inhibitor, such as a BCL2 inhibitor, e.g., venetoclax. In some embodiments, assessment is made prior to, during, during the course of, or after performing any of the methods provided herein. In some embodiments, the assessment is made prior to performing the methods provided herein. In some embodiments, assessment is made after performing one or more steps of the methods provided herein. In some embodiments, the assessment is performed prior to administration of administration of one or more steps of the provided combination therapy, for example, to screen and identify patients suitable and/or susceptible to receive the combination therapy. In some embodiments, the assessment is performed during, during the course of, or subsequent to administration of one or more steps of the provided combination therapy, for example, to assess the intermediate or final treatment outcome, e.g., to determine the efficacy of the treatment and/or to determine whether to continue or repeat the treatments and/or to determine whether to administer the remaining steps of the combination therapy.

In some embodiments, treatment of outcomes includes improved immune function, e.g., immune function of the T cells administered for cell based therapy and/or of the endogenous T cells in the body. In some embodiments, exemplary treatment outcomes include, but are not limited to, enhanced T cell proliferation, enhanced T cell functional activity, changes in immune cell phenotypic marker expression, such as such features being associated with the engineered T cells, e.g. CAR-T cells, administered to the subject. In some embodiments, exemplary treatment outcomes include decreased disease burden, e.g., tumor burden, improved clinical outcomes and/or enhanced efficacy of therapy.

In some embodiments, the screening step and/or assessment of treatment of outcomes includes assessing the survival and/or function of the T cells administered for cell based therapy. In some embodiments, the screening step and/or assessment of treatment of outcomes includes assessing the levels of cytokines or growth factors. In some embodiments, the screening step and/or assessment of treatment of outcomes includes assessing disease burden and/or improvements, e.g., assessing tumor burden and/or clinical outcomes. In some embodiments, either of the screening step and/or assessment of treatment of outcomes can include any of the assessment methods and/or assays described herein and/or known in the art, and can be performed one or more times, e.g., prior to, during, during the course of, or subsequently to administration of one or more steps of the combination therapy. Exemplary sets of parameters associated with a treatment outcome, which can be assessed in some embodiments of the methods provided herein, include peripheral blood immune cell population profile and/or tumor burden.

In some embodiments, the methods affect efficacy of the cell therapy in the subject. In some embodiments, the cytotoxicity of recombinant receptor-expressing, e.g., CAR-expressing, cells in the subject following administration of the dose of cells in the method with an inhibitor of a prosurvival BCL2 family protein, such as a BCL2 inhibitor, e.g., venetoclax, is greater as compared to that achieved via a method without the administration of the inhibitor, e.g., venetoclax. In some embodiments, the cytotoxicity of recombinant receptor-expressing, e.g., CAR-expressing, cells in the subject following administration of the dose of cells in the method with a subtherapeutically effective amount of an inhibitor of a prosurvival BCL2 family protein, such as a BCL2 inhibitor, e.g., venetoclax, is greater as compared to that achieved via a method without the administration of the inhibitor, e.g., venetoclax. In some embodiments, cytotoxicity in the subject of the administered T cell therapy, e.g., CAR-expressing T cells is assessed as compared to a method in which the T cell therapy is administered to the subject in the absence of an inhibitor of a prosurvival BCL2 family protein, e.g., venetoclax. In some embodiments, the methods result in the administered T cells exhibiting increased or prolonged cytotoxicity in the subject as compared to a method in which the T cell therapy is administered to the subject in the absence of the inhibitor, e.g., venetoclax.

In some embodiments, the administration of an inhibitor of a prosurvival BCL2 family protein, e.g., venetoclax, decreases disease burden, e.g., tumor burden, in the subject as compared to a method in which the dose of cells expressing the recombinant receptor is administered to the subject in the absence of a pro survival BCL2 family protein inhibitor, e.g., venetoclax. In some embodiments, the administration of a subtherapeutically effective amount of an inhibitor of a prosurvival BCL2 family protein, e.g., venetoclax, decreases disease burden, e.g., tumor burden, in the subject as compared to a method in which the dose of cells expressing the recombinant receptor is administered to the subject in the absence of a prosurvival BCL2 family protein inhibitor, e.g., venetoclax. In some embodiments, the administration of a prosurvival BCL2 family protein inhibitor, e.g., venetoclax, decreases blast marrow in the subject as compared to a method in which the dose of cells expressing the recombinant receptor is administered to the subject in the absence of a prosurvival BCL2 family protein inhibitor, e.g., venetoclax. In some embodiments, the administration of a prosurvival BCL2 family protein inhibitor, e.g., venetoclax, results in improved clinical outcomes, e.g., objective response rate (ORR), progression-free survival (PFS) and overall survival (OS), compared to a method in which the dose of cells expressing the recombinant receptor is administered to the subject in the absence of a prosurvival BCL2 family protein inhibitor, e.g., venetoclax.

In some embodiments, the subject can be screened prior to the administration of one or more steps of the combination therapy. For example, the subject can be screened for characteristics of the disease and/or disease burden, e.g., tumor burden, prior to administration of the combination therapy, to determine suitability, responsiveness and/or susceptibility to administering the combination therapy. For example, the subject can be screened for characteristics of the disease, e.g., overexpression or aberrant expression of a prosurvival or proapoptotic BCL2 family protein, prior to administration of the combination therapy, to determine suitability, responsiveness and/or susceptibility to administering the combination therapy. In some embodiments, the screening step and/or assessment of treatment outcomes can be used to determine the dose, frequency, duration, timing and/or order of the combination therapy provided herein.

In some embodiments, the subject can be screened after administration of one of the steps of the combination therapy, to determine and identify subjects to receive the remaining steps of the combination therapy and/or to monitor efficacy of the therapy. In some embodiments, the number, level or amount of administered T cells and/or proliferation and/or activity of the administered T cells is assessed prior to administration and/or after administration of an inhibitor of a prosurvival BCL2 family protein, such as a BCL2 inhibitor, e.g., venetoclax.

In some embodiments, a change and/or an alteration, e.g., an increase, an elevation, a decrease or a reduction, in levels, values or measurements of a parameter or outcome compared to the levels, values or measurements of the same parameter or outcome in a different time point of assessment, a different condition, a reference point and/or a different subject is determined or assessed. For example, in some embodiments, a fold change, e.g., an increase or decrease, in particular parameters, e.g., expression of a BCL2 family protein, compared to the same parameter in a different condition, e.g., before administration of a prosurvival BCL2 family protein inhibitor, such as a BCL2 inhibitor, e.g., venetoclax, can be determined. In some embodiments, the levels, values or measurements of two or more parameters are determined, and relative levels are compared. In some embodiments, the determined levels, values or measurements of parameters are compared to the levels, values or measurements from a control sample or an untreated sample. In some embodiments, the determined levels, values or measurements of parameters are compared to the levels from a sample from the same subject but at a different time point. The values obtained in the quantification of individual parameter can be combined for the purpose of disease assessment, e.g., by forming an arithmetical or logical operation on the levels, values or measurements of parameters by using multi-parametric analysis. In some embodiments, a ratio of two or more specific parameters can be calculated.

Assessment and determination of parameters associated with T cell health, function, activity, and/or outcomes, such as response, efficacy and/or toxicity outcomes, can be assessed at various time points. In some aspects, the assessment can be performed multiple times, e.g., prior to administration of the cell therapy, prior to, during or after manufacturing of the cells, and/or at the initiation of administration of the prosurvival BCL2 family protein inhibitor, e.g., venetoclax, during the continued, resumed and/or further administration of the prosurvival BCL2 family protein inhibitor, e.g., venetoclax, at the initiation of administration of the cell therapy and/or prior to, during or after the initiation of administration of the cell therapy.

In some embodiments, functional attributes of the administered cells and/or cell compositions include monitoring pharmacokinetic (PK) parameters, expansion and persistence of the cells, cell functional assays (e.g., any described herein, such as cytotoxicity assay, cytokine secretion assay and in vivo assays), high-dimensional T cell signaling assessment, and assessment of exhaustion phenotypes and/or signatures of the T cells. In some aspects, other attributes that can be assessed or monitored include monitoring and assessment of minimal residual disease (MRD). In some aspects, other attributes that can be assessed or monitored include pharmacodynamics parameters of the prosurvival BCL2 family protein inhibitor, e.g., venetoclax.

A. Response, Efficacy, and Survival

In some embodiments, parameters associated with therapy or a treatment outcome, which include parameters that can be assessed for the screening steps and/or assessment of treatment of outcomes and/or monitoring treatment outcomes, includes tumor or disease burden. The administration of the therapy that is an immunotherapy or cell therapy, such as a T cell therapy (e.g. CAR-expressing T cells) and/or a prosurvival BCL2 family protein inhibitor, such as a BCL2 inhibitor, e.g., venetoclax, can reduce or prevent the expansion or burden of the disease or condition in the subject. For example, where the disease or condition is a tumor, the methods generally reduce tumor size, bulk, metastasis, percentage of blasts in the bone marrow or molecularly detectable B cell malignancy and/or improve prognosis or survival or other symptom associated with tumor burden.

In some aspects, the administration in accord with the provided methods, and/or with the provided articles of manufacture or compositions, generally reduces or prevents the expansion or burden of the disease or condition in the subject. For example, where the disease or condition is a tumor, the methods generally reduce tumor size, bulk, metastasis, percentage of blasts in the bone marrow or molecularly detectable B cell malignancy and/or improve prognosis or survival or other symptom associated with tumor burden.

In some embodiments, the provided methods result in a decreased tumor burden in treated subjects compared to alternative methods in which the therapy, such as a T cell therapy (e.g. CAR-expressing T cells) is given without administration of a prosurvival BCL2 family protein inhibitor, such as a BCL2 inhibitor, e.g., venetoclax. In some embodiments, the provided methods result in a decreased tumor burden in subjects treated with a subtherapeutically effective amount of a prosurvival BCL2 family protein inhibitor compared to alternative methods in which the immunotherapy, such as a T cell therapy (e.g. CAR-expressing T cells) is given without administration of a prosurvival BCL2 family protein inhibitor, such as a BCL2 inhibitor, e.g., venetoclax. It is not necessary that the tumor burden actually be reduced in all subjects receiving the combination therapy, but that tumor burden is reduced on average in subjects treated, such as based on clinical data, in which a majority of subjects treated with such a combination therapy exhibit a reduced tumor burden, such as at least 50%, 60%, 70%, 80%, 90%, 95% or more of subjects treated with the combination therapy, exhibit a reduced tumor burden.

In some embodiments, the provided methods result in increased cytotoxic activity of the cytotoxic therapy as compared to alternative methods in which the therapy, such as a T cell therapy (e.g. CAR-expressing T cells) is given without administration of a prosurvival BCL2 family protein inhibitor, such as a BCL2 inhibitor, e.g., venetoclax. In some embodiments, the provided methods result in increased cytotoxicity in subjects treated with a subtherapeutically effective amount of a prosurvival BCL2 family protein inhibitor compared to alternative methods in which the immunotherapy, such as a T cell therapy (e.g. CAR-expressing T cells) is given without administration of a prosurvival BCL2 family protein inhibitor, such as a BCL2 inhibitor, e.g., venetoclax. In some cases, the provided methods result in an increased cytotoxicity of the cytotoxic therapy, optionally via perforin- and/or granzyme-mediate apoptosis, of one or more cancer cells, compared to alternative methods in which the immunotherapy, such as a T cell therapy (e.g. CAR-expressing T cells) is given without administration of a prosurvival BCL2 family protein inhibitor, such as a BCL2 inhibitor, e.g., venetoclax. It is not necessary that the cytotoxicity actually be increased in all subjects receiving the combination therapy, but that cytotoxicity is increased on average in subjects treated, such as based on clinical data, in which a majority of subjects treated with such a combination therapy exhibit a reduced tumor burden, such as at least 50%, 60%, 70%, 80%, 90%, 95% or more of subjects treated with the combination therapy, exhibit a reduced tumor burden.

Disease burden can encompass a total number of cells of the disease in the subject or in an organ, tissue, or bodily fluid of the subject, such as the organ or tissue of the tumor or another location, e.g., which would indicate metastasis. For example, tumor cells may be detected and/or quantified in the blood, lymph or bone marrow in the context of certain hematological malignancies. Disease burden can include, in some embodiments, the mass of a tumor, the number or extent of metastases and/or the percentage of blast cells present in the bone marrow.

In some embodiments, the subject has a myeloma, a lymphoma or a leukemia. The extent of disease burden can be determined by assessment of residual leukemia in blood or bone marrow. In some embodiments, the subject has a non-Hodgkin lymphoma (NHL), an acute lymphoblastic leukemia (ALL), a chronic lymphocytic leukemia (CLL), a small lymphocytic lymphoma (SLL) a diffuse large B-cell lymphoma (DLBCL) or a myeloma, e.g., a multiple myeloma (MM). In some embodiments, the subject has a leukemia or lymphoma. In some embodiments, the subject has a leukemia. In some cases, the leukemia is CLL. In some embodiments, the subject has a lymphoma. In some cases, the subject has a NHL, including DBCBL. In some cases, the lymphoma is SLL.

In some aspects, response rates in subjects, such as subjects with NHL, are based on the Lugano criteria. (Cheson et al., (2014) JCO., 32(27):3059-3067; Johnson et al., (2015) Radiology 2:323-338; Cheson, B. D. (2015) Chin. Clin. Oncol. 4(1):5). In some aspects, response assessment utilizes any of clinical, hematologic, and/or molecular methods. In some aspects, response assessed using the Lugano criteria involves the use of positron emission tomography (PET)-computed tomography (CT) and/or CT as appropriate. PET-CT evaluations may further comprise the use of fluorodeoxyglucose (FDG) for FDG-avid lymphomas. In some aspects, where PET-CT will be used to assess response in FDG-avid histologies, a 5-point scale may be used. In some respects, the 5-point scale comprises the following criteria: 1, no uptake above background; 2, uptake≤mediastinum; 3, uptake≥mediastinum but≤liver; 4, uptake moderately>liver; 5, uptake markedly higher than liver and/or new lesions; X, new areas of uptake unlikely to be related to lymphoma.

In some aspects, a complete response as described using the Lugano criteria involves a complete metabolic response and a complete radiologic response at various measureable sites. In some aspects, these sites include lymph nodes and extralymphatic sites, wherein a CR is described as a score of 1, 2, or 3 with or without a residual mass on the 5-point scale, when PET-CT is used. In some aspects, in Waldeyer's ring or extranodal sites with high physiologic uptake or with activation within spleen or marrow (e.g., with chemotherapy or myeloid colony-stimulating factors), uptake may be greater than normal mediastinum and/or liver. In this circumstance, complete metabolic response may be inferred if uptake at sites of initial involvement is no greater than surrounding normal tissue even if the tissue has high physiologic uptake. In some aspects, response is assessed in the lymph nodes using CT, wherein a CR is described as no extralymphatic sites of disease and target nodes/nodal masses must regress to <1.5 cm in longest transverse diameter of a lesion (LDi). Further sites of assessment include the bone marrow wherein PET-CT-based assessment should indicate a lack of evidence of FDG-avid disease in marrow and a CT-based assessment should indicate a normal morphology, which if indeterminate should be IHC negative. Further sites may include assessment of organ enlargement, which should regress to normal. In some aspects, nonmeasured lesions and new lesions are assessed, which in the case of CR should be absent (Cheson et al., (2014) JCO., 32(27):3059-3067; Johnson et al., (2015) Radiology 2:323-338; Cheson, B. D. (2015) Chin. Clin. Oncol. 4(1):5).

In some aspects, a partial response (PR) as described using the Lugano criteria involves a partial metabolic and/or radiological response at various measureable sites. In some aspects, these sites include lymph nodes and extralymphatic sites, wherein a PR is described as a score of 4 or 5 with reduced uptake compared with baseline and residual mass(es) of any size, when PET-CT is used. At interim, such findings can indicate responding disease. At the end of treatment, such findings can indicate residual disease. In some aspects, response is assessed in the lymph nodes using CT, wherein a PR is described as ≥50% decrease in SPD of up to 6 target measureable nodes and extranodal sites. If a lesion is too small to measure on CT, 5 mm×5 mm is assigned as the default value; if the lesion is no longer visible, the value is 0 mm×0 mm; for a node >5 mm×5 mm, but smaller than normal, actual measurements are used for calculation. Further sites of assessment include the bone marrow wherein PET-CT-based assessment should indicate residual uptake higher than uptake in normal marrow but reduced compared with baseline (diffuse uptake compatible with reactive changes from chemotherapy allowed). In some aspects, if there are persistent focal changes in the marrow in the context of a nodal response, consideration should be given to further evaluation with MRI or biopsy, or an interval scan. In some aspects, further sites may include assessment of organ enlargement, where the spleen must have regressed by >50% in length beyond normal. In some aspects, nonmeasured lesions and new lesions are assessed, which in the case of PR should be absent/normal, regressed, but no increase. No response/stable disease (SD) or progressive disease (PD) can also be measured using PET-CT and/or CT based assessments. (Cheson et al., (2014) JCO., 32(27):3059-3067; Johnson et al., (2015) Radiology 2:323-338; Cheson, B. D. (2015) Chin. Clin. Oncol., 4(1):5).

In some respects, progression-free survival (PFS) is described as the length of time during and after the treatment of a disease, such as a B cell malignancy, that a subject lives with the disease but it does not get worse. In some aspects, objective response (OR) is described as a measurable response. In some aspects, objective response rate (ORR) is described as the proportion of patients who achieved CR or PR. In some aspects, overall survival (OS) is described as the length of time from either the date of diagnosis or the start of treatment for a disease, such as a B cell malignancy, that subjects diagnosed with the disease are still alive. In some aspects, event-free survival (EFS) is described as the length of time after treatment for a B cell malignancy ends that the subject remains free of certain complications or events that the treatment was intended to prevent or delay. These events may include the return of the B cell malignancy or the onset of certain symptoms, such as bone pain from B cell malignancy that has spread to the bone, or death.

In some embodiments, the measure of duration of response (DOR) includes the time from documentation of tumor response to disease progression. In some embodiments, the parameter for assessing response can include durable response, e.g., response that persists after a period of time from initiation of therapy. In some embodiments, durable response is indicated by the response rate at approximately 1, 2, 3, 4, 5, 6, 7, 8, 9, 10, 11, 12, 18 or 24 months after initiation of therapy. In some embodiments, the response is durable for greater than 3 months or greater than 6 months.

In some aspects, the RECIST criteria is used to determine objective tumor response. (Eisenhauer et al., European Journal of Cancer 45 (2009) 228-247.) In some aspects, the RECIST criteria is used to determine objective tumor response for target lesions. In some respects, a complete response as determined using RECIST criteria is described as the disappearance of all target lesions and any pathological lymph nodes (whether target or non-target) must have reduction in short axis to <10 mm. In other aspects, a partial response as determined using RECIST criteria is described as at least a 30% decrease in the sum of diameters of target lesions, taking as reference the baseline sum diameters. In other aspects, progressive disease (PD) is described as at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm (in some aspects the appearance of one or more new lesions is also considered progression). In other aspects, stable disease (SD) is described as neither sufficient shrinkage to qualify for PR nor sufficient increase to qualify for PD, taking as reference the smallest sum diameters while on study.

In the case of MM, exemplary parameters to assess the extent of disease burden include such parameters as number of clonal plasma cells (e.g., >10% on bone marrow biopsy or in any quantity in a biopsy from other tissues; plasmacytoma), presence of monoclonal protein (paraprotein) in either serum or urine, evidence of end-organ damage felt related to the plasma cell disorder (e.g., hypercalcemia (corrected calcium >2.75 mmol/1); renal insufficiency attributable to myeloma; anemia (hemoglobin <10 g/dl); and/or bone lesions (lytic lesions or osteoporosis with compression fractures)).

In the case of DLBCL, exemplary parameters to assess the extent of disease burden include such parameters as cellular morphology (e.g., centroblastic, immunoblastic, and anaplastic cells), gene expression, miRNA expression and protein expression (e.g., expression of BCL2, BCL6, MUM1, LMO2, MYC, and p21).

In some aspects, response rates in subjects, such as subjects with CLL, are based on the International Workshop on Chronic Lymphocytic Leukemia (IWCLL) response criteria (Hallek, et al., Blood 2008, Jun. 15; 111(12): 5446-5456). In some aspects, these criteria are described as follows: complete remission (CR), which in some aspects requires the absence of peripheral blood clonal lymphocytes by immunophenotyping, absence of lymphadenopathy, absence of hepatomegaly or splenomegaly, absence of constitutional symptoms and satisfactory blood counts; complete remission with incomplete marrow recovery (CRi), which in some aspects is described as CR above, but without normal blood counts; partial remission (PR), which in some aspects is described as ≥50% fall in lymphocyte count, ≥50% reduction in lymphadenopathy or ≥50% reduction in liver or spleen, together with improvement in peripheral blood counts; progressive disease (PD), which in some aspects is described as ≥50% rise in lymphocyte count to >5×10⁹/L, ≥50% increase in lymphadenopathy, ≥50% increase in liver or spleen size, Richter's transformation, or new cytopenias due to CLL; and stable disease, which in some aspects is described as not meeting criteria for CR, CRi, PR or PD.

In some embodiments, the subjects exhibits a CR or OR if, within 1 month of the administration of the dose of cells, lymph nodes in the subject are less than at or about 20 mm in size, less than at or about 10 mm in size or less than at or about 10 mm in size.

In some embodiments, an index clone of the CLL is not detected in the bone marrow of the subject (or in the bone marrow of greater than 50%, 60%, 70%, 80%, 90% or more of the subjects treated according to the methods. In some embodiments, an index clone of the CLL is assessed by IgH deep sequencing. In some embodiments, the index clone is not detected at a time that is at or about or at least at or about 1, 2, 3, 4, 5, 6, 12, 18 or 24 months following the administration of the cells.

In some embodiments, a subject exhibits morphologic disease if there are greater than or equal to 5% blasts in the bone marrow, for example, as detected by light microscopy, such as greater than or equal to 10% blasts in the bone marrow, greater than or equal to 20% blasts in the bone marrow, greater than or equal to 30% blasts in the bone marrow, greater than or equal to 40% blasts in the bone marrow or greater than or equal to 50% blasts in the bone marrow. In some embodiments, a subject exhibits complete or clinical remission if there are less than 5% blasts in the bone marrow.

In some embodiments, a subject may exhibit complete remission, but a small proportion of morphologically undetectable (by light microscopy techniques) residual leukemic cells are present. A subject is said to exhibit minimum residual disease (MRD) if the subject exhibits less than 5% blasts in the bone marrow and exhibits molecularly detectable B cell malignancy. In some embodiments, molecularly detectable B cell malignancy can be assessed using any of a variety of molecular techniques that permit sensitive detection of a small number of cells. In some aspects, such techniques include PCR assays, which can determine unique Ig/T-cell receptor gene rearrangements or fusion transcripts produced by chromosome translocations. In some embodiments, flow cytometry can be used to identify B cell malignancy cell based on leukemia-specific immunophenotypes. In some embodiments, molecular detection of B cell malignancy can detect as few as 1 leukemia cell in 100,000 normal cells. In some embodiments, a subject exhibits MRD that is molecularly detectable if at least or greater than 1 leukemia cell in 100,000 cells is detected, such as by PCR or flow cytometry. In some embodiments, the disease burden of a subject is molecularly undetectable or MRD⁻, such that, in some cases, no leukemia cells are able to be detected in the subject using PCR or flow cytometry techniques.

In the case of leukemia, the extent of disease burden can be determined by assessment of residual leukemia in blood or bone marrow. In some embodiments, a subject exhibits morphologic disease if there are greater than or equal to 5% blasts in the bone marrow, for example, as detected by light microscopy. In some embodiments, a subject exhibits complete or clinical remission if there are less than 5% blasts in the bone marrow.

In some embodiments, for leukemia, a subject may exhibit complete remission, but a small proportion of morphologically undetectable (by light microscopy techniques) residual leukemic cells are present. A subject is said to exhibit minimum residual disease (MRD) if the subject exhibits less than 5% blasts in the bone marrow and exhibits molecularly detectable B cell malignancy. In some embodiments, molecularly detectable B cell malignancy can be assessed using any of a variety of molecular techniques that permit sensitive detection of a small number of cells. In some aspects, such techniques include PCR assays, which can determine unique Ig/T-cell receptor gene rearrangements or fusion transcripts produced by chromosome translocations. In some embodiments, flow cytometry can be used to identify B cell malignancy cell based on leukemia-specific immunophenotypes. In some embodiments, molecular detection of B cell malignancy can detect as few as 1 leukemia cell in 100,000 normal cells. In some embodiments, a subject exhibits MRD that is molecularly detectable if at least or greater than 1 leukemia cell in 100,000 cells is detected, such as by PCR or flow cytometry. In some embodiments, the disease burden of a subject is molecularly undetectable or MRD⁻, such that, in some cases, no leukemia cells are able to be detected in the subject using PCR or flow cytometry techniques.

In some embodiments, the methods and/or administration of a cell therapy, such as a T cell therapy (e.g. CAR-expressing T cells) and/or a prosurvival BCL2 family protein inhibitor, such as a BCL2 inhibitor, e.g., venetoclax, decrease(s) disease burden as compared with disease burden at a time immediately prior to the administration of the immunotherapy, e.g., T cell therapy and/or a prosurvival BCL2 family protein inhibitor, e.g., venetoclax.

In some aspects, administration of the immunotherapy, e.g. T cell therapy and/or a prosurvival BCL2 family protein inhibitor, e.g., venetoclax, may prevent an increase in disease burden, and this may be evidenced by no change in disease burden.

In some embodiments, the method reduces the burden of the disease or condition, e.g., number of tumor cells, size of tumor, duration of patient survival or event-free survival, to a greater degree and/or for a greater period of time as compared to the reduction that would be observed with a comparable method using an alternative therapy, such as one in which the subject receives immunotherapy, e.g. T cell therapy alone, in the absence of administration of a prosurvival BCL2 family protein inhibitor, e.g., venetoclax. In some embodiments, disease burden is reduced to a greater extent or for a greater duration following the combination therapy of administration of the immunotherapy, e.g., T cell therapy, and a prosurvival BCL2 family protein inhibitor, e.g., venetoclax, compared to the reduction that would be effected by administering each of the agent alone, e.g., administering a prosurvival BCL2 family protein inhibitor, e.g., venetoclax, to a subject having not received the immunotherapy, e.g. T cell therapy; or administering the immunotherapy, e.g. T cell therapy, to a subject having not received a prosurvival BCL2 family protein inhibitor, e.g., venetoclax.

In some embodiments, the burden of a disease or condition in the subject is detected, assessed, or measured. Disease burden may be detected in some aspects by detecting the total number of disease or disease-associated cells, e.g., tumor cells, in the subject, or in an organ, tissue, or bodily fluid of the subject, such as blood or serum. In some embodiments, disease burden, e.g. tumor burden, is assessed by measuring the number or extent of metastases. In some aspects, survival of the subject, survival within a certain time period, extent of survival, presence or duration of event-free or symptom-free survival, or relapse-free survival, is assessed. In some embodiments, any symptom of the disease or condition is assessed. In some embodiments, the measure of disease or condition burden is specified. In some embodiments, exemplary parameters for determination include particular clinical outcomes indicative of amelioration or improvement in the disease or condition, e.g., tumor. Such parameters include: duration of disease control, including complete response (CR), partial response (PR) or stable disease (SD) (see, e.g., Response Evaluation Criteria In Solid Tumors (RECIST) guidelines), objective response rate (ORR), progression-free survival (PFS) and overall survival (OS). Specific thresholds for the parameters can be set to determine the efficacy of the method of combination therapy provided herein.

In some aspects, disease burden is measured or detected prior to administration of the immunotherapy, e.g. T cell therapy, following the administration of the immunotherapy, e.g. T cell therapy but prior to administration of a prosurvival BCL2 family protein inhibitor, e.g., venetoclax, and/or following the administration of both the immunotherapy, e.g. T cell therapy and a prosurvival BCL2 family protein inhibitor, e.g., venetoclax. In the context of multiple administration of one or more steps of the combination therapy, disease burden in some embodiments may be measured prior to, or following administration of any of the steps, doses and/or cycles of administration, or at a time between administration of any of the steps, doses and/or cycles of administration. In some embodiments, the administration of a prosurvival BCL2 family protein inhibitor, e.g., venetoclax, is carried out at least two cycles (e.g., 28-day cycle), and disease burden is measured or detected prior to, during, and/or after each cycle.

In some embodiments, the burden is decreased by or by at least at or about 10, 20, 30, 40, 50, 60, 70, 80, 90, or 100 percent by the provided methods compared to immediately prior to the administration of a prosurvival BCL2 family protein inhibitor, e.g., venetoclax, and the immunotherapy, e.g. T cell therapy. In some embodiments, disease burden, tumor size, tumor volume, tumor mass, and/or tumor load or bulk is reduced following administration of the immunotherapy, e.g. T cell therapy and a prosurvival BCL2 family protein inhibitor, e.g., venetoclax, by at least at or about 10, 20, 30, 40, 50, 60, 70, 80, 90% or more compared to that immediately prior to the administration of the immunotherapy, e.g. T cell therapy and/or a prosurvival BCL2 family protein inhibitor, e.g., venetoclax.

In some embodiments, reduction of disease burden by the method comprises an induction in morphologic complete remission, for example, as assessed at 1 month, 2 months, 3 months, 4 months, 5 months, 6 months, or more than 6 months, after administration of, e.g., initiation of, the combination therapy.

In some aspects, an assay for minimal residual disease, for example, as measured by multiparametric flow cytometry, is negative, or the level of minimal residual disease is less than about 0.3%, less than about 0.2%, less than about 0.1%, or less than about 0.05%.

In some embodiments, the event-free survival rate or overall survival rate of the subject is improved by the methods, as compared with other methods. For example, in some embodiments, event-free survival rate or probability for subjects treated by the methods at 6 months following the method of combination therapy provided herein, is greater than about 40%, greater than about 50%, greater than about 60%, greater than about 70%, greater than about 80%, greater than about 90%, or greater than about 95%. In some aspects, overall survival rate is greater than about 40%, greater than about 50%, greater than about 60%, greater than about 70%, greater than about 80%, greater than about 90%, or greater than about 95%. In some embodiments, the subject treated with the methods exhibits event-free survival, relapse-free survival, or survival to at least 6 months, or at least 1, 2, 3, 4, 5, 6, 7, 8, 9, or 10 years. In some embodiments, the time to progression is improved, such as a time to progression of greater than at or about 6 months, or at least 1, 2, 3, 4, 5, 6, 7, 8, 9, or 10 years.

In some embodiments, following treatment by the method, the probability of relapse is reduced as compared to other methods. For example, in some embodiments, the probability of relapse at 6 months following the method of combination therapy, is less than about 80%, less than about 70%, less than about 60%, less than about 50%, less than about 40%, less than about 30%, less than about 20%, or less than about 10%.

In some cases, the pharmacokinetics of administered cells, e.g., adoptively transferred cells are determined to assess the availability, e.g., bioavailability of the administered cells. Methods for determining the pharmacokinetics of adoptively transferred cells may include drawing peripheral blood from subjects that have been administered engineered cells, and determining the number or ratio of the engineered cells in the peripheral blood. Approaches for selecting and/or isolating cells may include use of chimeric antigen receptor (CAR)-specific antibodies (e.g., Brentjens et al., Sci. Transl. Med. 2013 March; 5(177): 177ra38) Protein L (Zheng et al., J. Transl. Med. 2012 February; 10:29), epitope tags, such as Strep-Tag sequences, introduced directly into specific sites in the CAR, whereby binding reagents for Strep-Tag are used to directly assess the CAR (Liu et al. (2016) Nature Biotechnology, 34:430; international patent application Pub. No. WO2015095895) and monoclonal antibodies that specifically bind to a CAR polypeptide (see international patent application Pub. No. WO2014190273). Extrinsic marker genes may in some cases be utilized in connection with engineered cell therapies to permit detection or selection of cells and, in some cases, also to promote cell suicide. A truncated epidermal growth factor receptor (EGFRt) in some cases can be co-expressed with a transgene of interest (e.g., a CAR) in transduced cells (see e.g. U.S. Pat. No. 8,802,374). EGFRt may contain an epitope recognized by the antibody cetuximab (Erbitux®) or other therapeutic anti-EGFR antibody or binding molecule, which can be used to identify or select cells that have been engineered with the EGFRt construct and another recombinant receptor, such as a chimeric antigen receptor (CAR), and/or to eliminate or separate cells expressing the receptor. See U.S. Pat. No. 8,802,374 and Liu et al., Nature Biotech. 2016 April; 34(4): 430-434).

In some embodiments, the number of CAR+ T cells in a biological sample obtained from the patient, e.g., blood, can be determined at a period of time after administration of the cell therapy, e.g., to determine the pharmacokinetics of the cells. In some embodiments, number of CAR+ T cells, optionally CAR+ CD8+ T cells and/or CAR+ CD4+ T cells, detectable in the blood of the subject, or in a majority of subjects so treated by the method, is greater than 1 cells per μL, greater than 5 cells per μL or greater than per 10 cells per μL.

### Articles of Manufacture and Kits

Also provided provided are articles of manufacture containing an inhibitor of a prosurvival BCL2 family protein, such as a BCL2 inhibitor, e.g., venetoclax, and components for the immunotherapy or cell therapy, e.g., antibody or antigen binding fragment thereof or T cell therapy, e.g. engineered cells, and/or compositions thereof. The articles of manufacture may include a container and a label or package insert on or associated with the container. Suitable containers include, for example, bottles, vials, syringes, IV solution bags, etc. The containers may be formed from a variety of materials such as glass or plastic. The container in some embodiments holds a composition which is by itself or combined with another composition effective for treating, preventing and/or diagnosing the condition. In some embodiments, the container has a sterile access port. Exemplary containers include an intravenous solution bags, vials, including those with stoppers pierceable by a needle for injection, or bottles or vials for orally administered agents. The label or package insert may indicate that the composition is used for treating a disease or condition.

The article of manufacture may include (a) a first container with a composition contained therein, wherein the composition includes the engineered cells used for the immunotherapy, e.g. T cell therapy; and (b) a second container with a composition contained therein, wherein the composition includes an inhibitor of a prosurvival BCL2 family protein, such as a BCL2 inhibitor, e.g., venetoclax.

In some embodiments, the first container comprises a first composition and a second composition, wherein the first composition comprises a first population of the engineered cells used for the immunotherapy, e.g., CD4+ T cell therapy, and the second composition comprises a second population of the engineered cells, wherein the second population may be engineered separately from the first population, e.g., CD8+ T cell therapy. In some embodiments, the first and second cell compositions contain a defined ratio of the engineered cells, e.g., CD4+ and CD8+ cells (e.g., 1:1 ratio of CD4+:CD8+ CAR+ T cells).

The article of manufacture may further include a package insert indicating that the compositions can be used to treat a particular condition. Alternatively, or additionally, the article of manufacture may further include another or the same container comprising a pharmaceutically-acceptable buffer. It may further include other materials such as other buffers, diluents, filters, needles, and/or syringes.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polypeptide” and “protein” are used interchangeably to refer to a polymer of amino acid residues, and are not limited to a minimum length. Polypeptides, including the provided receptors and other polypeptides, e.g., linkers or peptides, may include amino acid residues including natural and/or non-natural amino acid residues. The terms also include post-expression modifications of the polypeptide, for example, glycosylation, sialylation, acetylation, and phosphorylation. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

As used herein, a “subject” is a mammal, such as a human or other animal, and typically is human. In some embodiments, the subject, e.g., patient, to whom an inhibitor of a prosurvival BCL2 family protein, such as a BCL2 inhibitor, e.g., venetoclax, engineered cells, or compositions are administered, is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. In some embodiments, sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., engineered cells or an inhibitor of a prosurvival BCL2 family protein, or a pharmaceutical formulation or composition thereof, in the context of administration, refers to an amount effective, at dosages/amounts and for periods of time necessary, to achieve a desired result, such as a therapeutic or prophylactic result.

A “therapeutically effective amount” of an agent, e.g., engineered cells or an inhibitor of a prosurvival BCL2 family protein, or a pharmaceutical formulation or composition thereof, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the cells and/or compositions at effective amounts, e.g., therapeutically effective amounts. In some embodiments, the provided methods involve administering an inhibitor of a prosurvival BCL2 family protein, such as a BCL2 inhibitor, e.g., venetoclax, engineered cells (e.g. cell therapy), or compositions at effective amounts, e.g., therapeutically effective amounts. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount. In the context of lower tumor burden, the prophylactically effective amount in some aspects will be higher than the therapeutically effective amount.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “about” as used herein refers to the usual error range for the respective value readily known to the skilled person in this technical field. Reference to “about” a value or parameter herein includes (and describes) embodiments that are directed to that value or parameter per se.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

As used herein, a composition refers to any mixture of two or more products, substances, or compounds, including cells. It may be a solution, a suspension, liquid, powder, a paste, aqueous, non-aqueous or any combination thereof.

As used herein, “enriching” when referring to one or more particular cell type or cell population, refers to increasing the number or percentage of the cell type or population, e.g., compared to the total number of cells in or volume of the composition, or relative to other cell types, such as by positive selection based on markers expressed by the population or cell, or by negative selection based on a marker not present on the cell population or cell to be depleted. The term does not require complete removal of other cells, cell type, or populations from the composition and does not require that the cells so enriched be present at or even near 100% in the enriched composition.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control or fluorescence minus one (FMO) gating control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control or fluorescence minus one (FMO) gating control under otherwise identical conditions, and/or at a level substantially lower than that for cell known to be positive for the marker, and/or at a level substantially similar as compared to that for a cell known to be negative for the marker.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

As used herein, a composition refers to any mixture of two or more products, substances, or an inhibitor of a prosurvival BCL2 family protein, such as a BCL2 inhibitor, e.g., venetoclax, including cells. It may be a solution, a suspension, liquid, powder, a paste, aqueous, non-aqueous or any combination thereof.

As used herein, a “subject” is a mammal, such as a human or other animal, and typically is human.

### EXEMPLARY EMBODIMENTS

Among the provided embodiments are:

1. A method of treating cancer, the method comprising:
(1) administering a cytotoxic therapy to a subject having a cancer, wherein the cytotoxic therapy is an immunotherapy or a cell therapy and specifically binds to an antigen associated with, expressed by, or present on cells of the cancer; and
(2) administering to the subject an inhibitor of a prosurvival BCL2 family protein in a dosing regimen sufficient to achieve a steady state concentration (Css) of the inhibitor at a time between at or about 1 day and at or about 14 days after initiation of administration of the cytotoxic therapy and/or at a time before peak levels of the cytotoxic therapy is detectable in the blood of the subject following administration of the cytotoxic therapy.

2. A method of treating cancer, the method comprising administering to a subject having a cancer an inhibitor of a prosurvival BCL2 family protein in a dosing regimen sufficient to achieve a steady state concentration (Css) of the inhibitor at a time between at or about 1 day and at or about 14 days after initiation of administration of a cytotoxic therapy and/or at a time before peak levels of the cytotoxic therapy is detectable in the blood of the subject following administration of the cytotoxic therapy, wherein the cytotoxic therapy is an immunotherapy or a cell therapy and specifically binds to an antigen associated with, expressed by, or present on cells of the cancer.

3. A method of treating cancer, the method comprising:
(1) administering a cytotoxic therapy to a subject having a cancer, wherein the cytotoxic therapy is an immunotherapy or a cell therapy and specifically binds to an antigen associated with, expressed by, or present on cells of the cancer; and
(2) administering to the subject an inhibitor of a prosurvival BCL2 family protein in a dosing regimen comprising initiation of administration of the inhibitor at a time between at or about 7 days prior to and at or about 14 days after initiation of administration of the cytotoxic therapy.

4. A method of treating cancer, the method comprising administering to a subject having a cancer an inhibitor of a prosurvival BCL2 family protein, wherein the inhibitor is administered in a dosing regimen comprising initiation of administration of the inhibitor at a time between at or about 7 days prior to and at or about 14 days after initiation of administration of a cytotoxic therapy, wherein the cytotoxic therapy is an immunotherapy or a cell therapy and specifically binds to an antigen associated with, expressed by, or present on cells of the cancer.

5. A method of treating a cancer in a subject, the method comprising administering a cytotoxic therapy to a subject having a cancer, wherein the cytotoxic therapy is an immunotherapy or a cell therapy and specifically binds to an antigen associated with, expressed by, or present on cells of the cancer, wherein the subject is administered or is to be administered an inhibitor of a prosurvival BCL2 family protein for a period of time in a dosing regimen comprising initiation of administration of the inhibitor at a time between at or about 7 days prior to and at or about 14 days after initiation of administration of the cytotoxic therapy.

6. The method of embodiment 1 or embodiment 2, wherein the dosing regimen of the inhibitor comprises initiation of administration of the inhibitor at a time between at or about 7 days prior to and at or about 14 days after initiation of administration of the cytotoxic therapy.

7. The method of any of embodiments 1-6, wherein the dosing regimen of the inhibitor comprises initiation of administration of the inhibitor at a time between at or about 3 days prior to and at or about 14 days after initiation of administration of the cytotoxic therapy.

8. The method of any of embodiments 1-7, wherein the dosing regimen of the inhibitor comprises initiation of administration of the inhibitor at a time between at or about 1 day prior to and at or about 8 days after initiation of administration of the cytotoxic therapy.

9. The method of any of embodiments 1-8, wherein at least one dose of the inhibitor in the dosing regimen is administered concurrently with the cytotoxic therapy and/or on the same day as the cytotoxic therapy.

10. The method of any of embodiments 3-9, wherein the dosing regimen of the inhibitor is sufficient to achieve a steady state concentration (Css) of the inhibitor at a time between at or about 1 day and at or about 14 days after initiation of administration of the cytotoxic therapy and/or at a time before peak levels of the cytotoxic therapy is detectable in the blood of the subject following administration of the cytotoxic therapy.

11. The method of any of embodiments 1-10, wherein the subject is not administered or has not received administration of rituximab and/or ibrutinib within 7 days prior to the initiation of administration of the cytotoxic therapy.

12. The method of any of embodiments 1-11, wherein the cytotoxic therapy is capable of or results in cell-mediated cytotoxicity of one of more of cells of the cancer.

13. The method of any of embodiments 1-11, wherein the cytotoxic therapy is capable of or mediates perforin- and/or granzyme-mediated apoptosis of one or more cells of the cancer.

14. The method of any of embodiments 1-13, wherein the cytotoxic therapy is an immunotherapy.

15. The method of any of embodiments 1-14, wherein the immunotherapy is a T cell engaging therapy that increases the activity of T cells.

16. The method of any of embodiments 1-15, wherein the cytotoxic therapy is a bispecific T cell engager (BiTE) therapy.

17. The method of any of embodiments 1-14, wherein the cytotoxic therapy is a cell therapy.

18. The method of any of embodiments 1-17, wherein the cell therapy comprises cells that are autologous to the subject.

19. The method of any of embodiments 1-18, wherein the cell therapy is selected from among the group consisting of a tumor infiltrating lymphocytic (TIL) therapy, an endogenous T cell therapy, a natural kill (NK) cell therapy, a transgenic TCR therapy, and a recombinant receptor-expressing cell therapy, which optionally is a chimeric antigen receptor (CAR)-expressing cell therapy.

20. The method of any of embodiments 1-19, wherein the cell therapy comprises a dose of cells expressing a recombinant receptor that specifically binds to the antigen.

21. The method of any of embodiments 1-20, wherein administration of the cell therapy comprises administration of from or from about 1×105 to 5×108 total cells of the cell therapy, recombinant receptor-expressing cells, total T cells, or total peripheral blood mononuclear cells (PBMCs); from or from about 1×105 to 2×108 total cells of the cell therapy, recombinant receptor-expressing cells, total T cells, or total peripheral blood mononuclear cells (PBMCs); from or from about 1×106 to 1×108 total cells of the cell therapy, recombinant receptor-expressing cells, total T cells, or total peripheral blood mononuclear cells (PBMCs); or 1×106 to 5×107 total cells of the cell therapy, recombinant receptor-expressing cells, total T cells, or total peripheral blood mononuclear cells (PBMCs).

22. The method of any of embodiments 1-21, wherein cell therapy comprises or is enriched in T cells.

23. The method of any of embodiments 1-22, wherein the cell therapy comprises or is enriched in CD3+, CD4+, CD8+ or CD4+ and CD8+ T cells.

24. The method of any of embodiments 1-23, wherein the cell therapy comprises or is enriched in CD4+ and CD8+ T cells.

25. The method of embodiment 24, wherein the CD4+ and CD8+ T cells of the cell therapy comprises a defined ratio of CD4+ CAR-expressing T cells to CD8+ CAR-expressing T cells and/or of CD4+ CAR-expressing T cells to CD8+ CAR-expressing T cells, that is or is approximately 1:1 or is between approximately 1:3 and approximately 3:1.

26. The method of any of embodiments 1-25, wherein the cell therapy comprises administering CD4+ and CD8+ T cells, wherein T cells of each dose comprises a receptor, optionally a CAR, that specifically binds to the antigen, wherein the administration comprises administering a plurality of separate compositions, the plurality of separate compositions comprising a first composition comprising or enriched in the CD8+ T cells and a second composition comprising or enriched in the CD4+ T cells.

27. The method of embodiment 26, wherein:

the CD4+ T cells comprising the receptor in the one of the first and second compositions and the CD8+ T cells comprising the receptor in the other of the first and second compositions are present at a defined ratio that is or is approximately 1:1 or is between approximately 1:3 and approximately 3:1; and/or

the CD4+ T cells comprising the receptor and the CD8+ T cells comprising the receptor administered in the first and second compositions are present at a defined ratio, which ratio is or is approximately 1:1 or is between approximately 1:3 and approximately 3:1.

28. The method of any of embodiments 1-27, wherein the cell therapy comprises or is enriched in natural killer (NK) cells.

29. The method of any of embodiments 1-28, wherein the cell therapy comprises or is enriched in iPS-derived cells.

30. The method of any of embodiments 20-29, wherein the recombinant receptor is a T cell receptor (TCR) or a functional non-T cell receptor.

31. The method of any of embodiments 20-30, wherein the recombinant receptor is a chimeric antigen receptor (CAR).

32. The method of any of embodiments 1-31, wherein the cell therapy comprises administration of from or from about 1×105 to 5×108 total CAR-expressing T cells, 1×106 to 2.5×108 total CAR-expressing T cells, 5×106 to 1×108 total CAR-expressing T cells, 1×107 to 2.5×108 total CAR-expressing T cells, 5×107 to 1×108 total CAR-expressing T cells, each inclusive.

33. The method of any of embodiments 1-32, wherein the cell therapy comprises administration of at least or at least about 1×105 CAR-expressing cells, at least or at least about 2.5×105 CAR-expressing cells, at least or at least about 5×105 CAR-expressing cells, at least or at least about 1×106 CAR-expressing cells, at least or at least about 2.5×106 CAR-expressing cells, at least or at least about 5×106 CAR-expressing cells, at least or at least about 1×107 CAR-expressing cells, at least or at least about 2.5×107 CAR-expressing cells, at least or at least about 5×107 CAR-expressing cells, at least or at least about 1×108 CAR-expressing cells, at least or at least about 2.5×108 CAR-expressing cells, or at least or at least about 5×108 CAR-expressing cells.

34. The method of any of embodiments 1-33, wherein the cell therapy comprises administration of at or about 5×107 total CAR-expressing T cells.

35. The method of any of embodiments 1-34, wherein the cell therapy comprises administration of at or about 1×108 CAR-expressing cells.

36. The method of any of embodiments 1-35, wherein the cell therapy comprises cells expressing a chimeric antigen receptor (CAR) comprising an extracellular antigen-recognition domain that specifically binds to the antigen and an intracellular signaling domain comprising an ITAM.

37. The method of embodiment 36, wherein the antigen is a tumor antigen or is expressed on cells of the cancer.

38. The method of any of embodiments 1-37, wherein the antigen is selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha(IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1).

39. The method of any of embodiments 1-38, wherein the antigen is CD19.

40. The method of any of embodiments 36-39, wherein the intracellular signaling domain comprises an intracellular domain of a CD3-zeta (CD3) chain.

41. The method of any of embodiments 36-40, wherein the intracellular signaling region further comprises a costimulatory signaling region.

42. The method of embodiment 41, wherein the costimulatory signaling region comprises a signaling domain of CD28 or 4-1BB, optionally human CD28 or human 4-1BB.

43. The method of embodiment 42, wherein the costimulatory domain is or comprises a signaling domain of CD28.

44. The method of any of embodiments 1-43, wherein the cell therapy comprises autologous cells from the subject.

45. The method of embodiment 44, wherein the method comprises collecting a biological sample from the subject comprising the autologous cells prior to initiation of administration of the inhibitor.

46. The method of embodiment 45, wherein the biological sample from the subject is or comprises a whole blood sample, a buffy coat sample, a peripheral blood mononuclear cells (PBMC) sample, an unfractionated T cell sample, a lymphocyte sample, a white blood cell sample, an apheresis product, or a leukapheresis product.

47. The method of any of embodiments 1-46, wherein the method comprises, prior to administration of the cytotoxic therapy, administering a lymphodepleting agent or therapy to the subject.

48. The method of any of embodiments 1-47, wherein the subject has been previously treated with an inhibitor of a prosurvival Bcl-2 family protein, optionally wherein the subject has been previously treated with venetoclax.

49. The method of embodiment 48, wherein the previous treatment with the inhibitor is administered at a time between the collecting of the autologous cells and prior to a lymphodepleting therapy.

50. The method of embodiment 49, wherein the previous treatment with the inhibitor is ceased for at least at or about 3 days or for at least at or about 4 days prior to administration of a lymphodepleting therapy and/or for at least an amount of time until the concentration of the inhibitor in the subject's bloodstream is reduced by about three half-lives or about four half-lives and/or for at least an amount of time until the inhibitor is eliminated from the bloodstream of the subject.

51. The method of any of embodiments 47-50, wherein the lymphodepleting therapy is completed between 2 and 7 days before the initiation of administration of the cytotoxic therapy.

52. The method of any of embodiments 47-51, wherein the lymphodepleting therapy comprises the administration of fludarabine and/or cyclophosphamide.

53. The method of any of embodiments 47-52, wherein the lymphodepleting therapy comprises administration of cyclophosphamide at about 200-400 mg/m2, optionally at or about 300 mg/m2, inclusive, and/or fludarabine at about 20-40 mg/m2, optionally 30 mg/m2, daily for 2-4 days, optionally for 3 days, or wherein the lymphodepleting therapy comprises administration of cyclophosphamide at about 500 mg/m2.

54. The method of any one of embodiments 47-53, wherein:

the lymphodepleting therapy comprises administration of cyclophosphamide at or about 300 mg/m2 and fludarabine at about 30 mg/m2 daily for 3 days; and/or

the lymphodepleting therapy comprises administration of cyclophosphamide at or about 500 mg/m2 and fludarabine at about 30 mg/m2 daily for 3 days.

55. The method of any of embodiments 1-54, wherein the initiation of administration of the inhibitor is within at or about 3 days prior to initiation of administration of the cytotoxic therapy.

56. The method of any of embodiments 1-55, wherein the initiation of administration of the inhibitor is within at or about 2 days prior to initiation of administration of the cytotoxic therapy.

57. The method of any of embodiments 1-56, wherein the initiation of administration of the inhibitor is within at or about 1 day prior to initiation of administration of the cytotoxic therapy.

58. The method of any of embodiments 1-57, wherein the initiation of administration of the inhibitor is concurrent with or on the same day as initiation of administration of the inhibitor.

59. The method of any of embodiments 1-54, wherein the initiation of administration of the inhibitor is no more than 2 days after initiation of administration of the cytotoxic therapy, optionally wherein the initiation of administration of the inhibitor is within 1 day after the initiation of administration of the cytotoxic therapy.

60. The method of any of embodiments 1-59, wherein:

the dosing regimen of the inhibitor comprises a subtherapeutic amount of the inhibitor;

the dosing regimen of the inhibitor is not sufficient to reduce tumor burden in the subject or reduces tumor burden by less than 10% when administered as a monotherapy in the absence of the combined administration with the cytotoxic therapy; and/or

the dosing regimen of the inhibitor does not result in a complete or partial response in a group of similarly treated subjects, or results in such a response in no more than 10% of such subjects, when administered as a monotherapy in the absence of the combined administration with the cytotoxic therapy.

61. The method of any of embodiments 1-60, wherein the dosing regimen of the inhibitor comprises once daily dosing.

62. The method of any of embodiments 1-61, wherein the once daily dose is an amount of the inhibitor of between at or about 20 mg and at or about 800 mg, between at or about 20 mg and at or 400 mg, between at or about 20 mg and at or about 350 mg, between at or about 20 mg and at or about 300 mg, between at or about 20 mg and at or about 250 mg, between at or about 20 mg and at or about 200 mg, between at or about 20 mg and at or about 150 mg, between at or about 20 mg and at or about 100 mg, between at or about 20 mg and at or about 50 mg, between at or about 20 mg and at or about 40 mg, between at or about 40 mg and at or about 800 mg, between at or about 40 mg and at or 400 mg, between at or about 40 mg and at or about 350 mg, between at or about 40 mg and at or about 300 mg, between at or about 40 mg and at or about 250 mg, between at or about 40 mg and at or about 200 mg, between at or about 40 mg and at or about 150 mg, between at or about 40 mg and at or about 100 mg, between at or about 40 mg and at or about 50 mg, between at or about 50 mg and at or about 800 mg, between at or about 50 mg and at or about 400 mg, between at or about 50 mg and at or about 350 mg, between at or about 50 mg and at or about 300 mg, between at or about 50 mg and at or about 250 mg, between at or about 50 mg and at or about 200 mg, between at or about 50 mg and at or about 150 mg, between at or about 50 mg and at or about 100 mg, between at or about 100 mg and at or about 800 mg, between at or about 100 mg and at or about 400 mg, between at or about 100 mg and at or about 350 mg, between at or about 100 mg and at or about 300 mg, between at or about 100 mg and at or about 250 mg, between at or about 100 mg and at or about 200 mg, between at or about 100 mg and at or about 150 mg, between at or about 150 mg and at or about 800 mg, between at or about 150 mg and at or about 400 mg, between at or about 150 mg and at or about 350 mg, between at or about 150 mg and at or about 300 mg, between at or about 150 mg and at or about 250 mg, between at or about 150 mg and at or about 200 mg, between at or about 200 mg and at or about 800 mg, between at or about 200 mg and at or about 400 mg, between at or about 200 mg and at or about 350 mg, between at or about 200 mg and at or about 300 mg, between at or about 200 mg and at or about 250 mg, between at or about 250 mg and at or about 300 mg, between at or about 300 mg and at or about 350 mg, between at or about 300 mg and at or about 400 mg, between at or about 300 mg and at or about 800 mg, between at or about 350 mg and at or about 400 mg, and between at or about 350 mg and at or about 800 mg, each inclusive.

63. The method of any of embodiments 1-62, wherein the once daily dose is an amount of the inhibitor of between at or about 20 mg and at or about 100 mg, inclusive.

64. The method of any of embodiments 1-63, wherein the once daily dose is an amount of the inhibitor of between at or about 40 mg and at or about 100 mg, inclusive.

65. The method of any of embodiments 1-64, wherein the once daily dose is an amount of the inhibitor of between at or about 50 mg and at or about 100 mg, inclusive.

66. The method of any of embodiments 1-65, wherein the once daily dose is an amount of the inhibitor of at or about 20 mg.

67. The method of any of embodiments 1-65, wherein the once daily dose is an amount of the inhibitor of at or about 40 mg.

68. The method of any of embodiments 1-65, wherein the once daily dose is an amount of the inhibitor of at or about 50 mg.

69. The method of any of embodiments 1-65, wherein the once daily dose is an amount of the inhibitor of at or about 100 mg.

70. The method of any of embodiments 1-57, wherein the inhibitor is administered in a dose-ramp up schedule prior to administration of the dosing regimen of the inhibitor, optionally wherein the dose-ramp up schedule comprises administration of increasing amounts of the inhibitor up to the amount of the once daily dose.

71. The method of any of embodiments 1-70, wherein the inhibitor inhibits one or more prosurvival BCL2 family protein selected from among the group consisting of BCL2, BCLXL, BCLW, BCLB, MCL1, and combinations thereof.

72. The method of any of embodiments 1-71, wherein the one or more prosurvival BCL2 family protein is BCL2, BCLXL, and/or BCLW.

73. The method of any of embodiments 1-72, wherein the inhibitor inhibits the one or more prosurvival BCL2 family protein with a half-maximal inhibitory concentration (IC50) of less than or less than about 1000 nM, 900 nM, 800 nM, 600 nM, 500 nM, 400 nM, 300 nM, 200 nM, 100 nM, 50 nM or less than or less than about 10 nM.

74. The method of any of embodiments 1-73, wherein the inhibitor is selected from among the group consisting of venetoclax, navitoclax, ABT737, maritoclax, obatoclax, and clitocine.

75. The method of any of embodiments 1-74, wherein the inhibitor is navitoclax.

76. The method of any of embodiments 1-74, wherein the inhibitor is venetoclax.

77. The method of any of embodiments 1-74, wherein the cancer is a hematological malignancy.

78. The method of any of embodiments 1-77, wherein the cancer is a B cell malignancy.

79. The method of any of embodiments 1-78, wherein the cancer is a myeloma, leukemia or lymphoma.

80. The method of any of embodiments 1-79, wherein the cancer is an acute lymphoblastic leukemia (ALL), adult ALL, chronic lymphoblastic leukemia (CLL), a small lymphocytic lymphoma (SLL), non-Hodgkin lymphoma (NHL), a large B cell lymphoma.

81. The method of any of embodiments 1-80, wherein the cancer is a chronic lymphocytic leukemia (CLL).

82. The method of any of embodiments 1-80, wherein the cancer is a non-Hodgkin lymphoma (NHL).

83. The method of embodiment 82, wherein the NHL is a diffuse large B-cell lymphoma (DLBCL).

84. The method of any of embodiments 1-80, wherein the cancer is primary mediastinal B-cell lymphoma (PMBCL) or a follicular lymphoma (FL), optionally follicular lymphoma grade 3B (FL3B).

85. The method of any of embodiments 1-84, wherein the subject has relapsed following remission after treatment with, or become refractory to, failed and/or was intolerant to treatment with the one or more prior therapies for treating the cancer.

86. The method of any of embodiments 1-85, wherein the cancer is resistant to treatment with the cytotoxic therapy alone.

87. The method of any of embodiments 1-86, wherein the cancer exhibits overexpression or aberrant expression of a prosurvival BCL2 family protein.

88. The method of any of embodiments 1-87, wherein the dosing regimen of the inhibitor comprises administration of the inhibitor, optionally once daily, for up to 6 months after the initiation of the administration of the cytotoxic therapy.

89. The method of any of embodiments 1-88, wherein the dosing regimen of inhibitor comprises administration of the inhibitor, optionally once daily, for up to 3 months after the initiation of the administration of the cytotoxic therapy.

90. The method of any of embodiments 1-89, wherein administration of the inhibitor in the dosing regimen is discontinued if the subject exhibits clinical remission.

91. The method of any of embodiments 1-90, wherein:
the method increases the cytotoxic activity of the cytotoxic therapy compared to a method that does not involve the administration of the inhibitor; and/or
the method increases cytolytic killing, optionally via perforin- and/or granzyme-mediated apoptosis, of one or more of the cancer cells compared to a method that does not involve the administration of the inhibitor.

92. The method of any of embodiments 1-91, wherein:

at least 35%, at least 40% or at least 50% of subjects treated according to the method achieve a complete response (CR) that is durable, or is durable in at least 60, 70, 80, 90, or 95% of subjects achieving the CR, for at or greater than 6 months or at or greater than 9 months; and/or

wherein at least 60, 70, 80, 90, or 95% of subjects achieving a CR by six months remain in response, remain in CR, and/or survive or survive without progression, for greater at or greater than 3 months and/or at or greater than 6 months and/or at greater than nine months; and/or

at least 50%, at least 60% or at least 70% of the subjects treated according to the method achieve objective response (OR) optionally wherein the OR is durable, or is durable in at least 60, 70, 80, 90, or 95% of subjects achieving the OR, for at or greater than 6 months or at or greater than 9 months; and/or

wherein at least 60, 70, 80, 90, or 95% of subjects achieving an OR by six months remain in response or surviving for greater at or greater than 3 months and/or at or greater than 6 months.

93. The method of any one of embodiments 1-92, wherein the subject is a human.

94. A method of treatment with a cytotoxic therapy, the method comprising:

(a) assessing the level or amount of one or more prosurvival gene in a biological sample from a subject having or suspected of having a cancer;

(b) selecting the subject for treatment with a cytotoxic therapy if the level or amount of the one or more prosurvival gene is below a gene reference value; and

(c) administering to the selected patient the cytotoxic therapy that binds an antigen associated with, expressed by, or present on cells of the cancer.

95. A method of selecting a subject having a cancer for administering an inhibitor of a prosurvival BCL2 family protein, the method comprising:

(a) assessing the level or amount of one or more prosurvival gene in a biological sample from the subject,

wherein the level or amount of the one or more prosurvival gene is the level or amount of a protein and/or a polynucleotide encoded by the one or more prosurvival gene, wherein the subject is to receive administration of a cytotoxic therapy that is an immunotherapy or a cell therapy that specifically binds to an antigen associated with, expressed by, or present on cells of the cancer, and wherein the biological sample is obtained from the subject prior to the administration of the cytotoxic therapy; and

(b) selecting the subject having the cancer for treatment with an inhibitor of a prosurvival BCL2 family protein if the level or amount of the one or more prosurvival gene is above a gene reference value.

96. The method of embodiment 95, further comprising administering to the selected subject the inhibitor in combination with the cytotoxic therapy.

97. The method of embodiment 95, wherein if the subject is not selected for treatment with the inhibitor, administering only the cytotoxic therapy to the subject.

98. A method of identifying a subject having a cancer that is predicted to be resistant to treatment with a cytotoxic therapy, the method comprising:

(a) assessing the level or amount of one or more prosurvival gene in a biological sample from the subject,

wherein the level or amount of the one or more prosurvival gene is the level or amount of a protein and/or a polynucleotide encoded by the one or more prosurvival gene, wherein the subject is a candidate for administration of a dose of a cytotoxic therapy, wherein the cytotoxic therapy is an immunotherapy or a cell therapy that specifically binds to an antigen associated with, expressed by, or present on cells of the cancer, and wherein the biological sample is obtained from the subject prior to the administration of the cytoxic therapy; and

(b) identifying the subject as having a cancer that is predicted to be resistant to treatment with the cytotoxic therapy if the level or amount of the one or more prosurvival gene is above a gene reference value.

99. The method of embodiment 98, wherein if the subject is identified as having a cancer that is predicted to be resistant to treatment with the cytotoxic therapy, further comprising administering an alternative treatment to the identified subject, wherein the alternative treatment is selected from among the following: a combination treatment comprising the cytotoxic therapy and an additional agent that modulates or increases the activity of the cytotoxic therapy; an increased dose of the cytotoxic therapy; and/or a chemotherapeutic agent.

100. The method of embodiment 99, wherein the alternative treatment is a combination treatment comprising the cytotoxic therapy and an additional agent that modulates or increases the activity of the T cell therapy, optionally wherein the additional agent is an immune checkpoint inhibitor, a modulator of a metabolic pathway, an adenosine receptor antagonist, a kinase inhibitor, an anti-TGFb antibody or an anti-TGFbR antibody, a cytokine, and/or a prosurvival BCL2 family protein inhibitor.

101. The method of embodiment 99 or embodiment 100, wherein the alternative treatment is a combination treatment comprising the cytotoxic therapy and prosurvival BCL2 family protein inhibitor.

102. The method of embodiment 99, wherein the alternative treatment is an increased dose of the cytotoxic therapy compared to a dose of the cytotoxic therapy given to a subject identified as having a cancer that is not predicted to be resistant to treatment with the cytotoxic therapy, optionally wherein the cytotoxic therapy comprises cells expressing a recombinant receptor that binds to an antigen associated with, expressed by, or present on the cells of the cancer.

103. The method of embodiment 102, wherein the increased dose of the cytotoxic therapy comprises an increased number of cells of the cytotoxic therapy compared to a dose of the cytotoxic therapy given to a subject identified as having a cancer that is not predicted to be resistant to treatment with the cytotoxic therapy.

104. The method of embodiment 99, wherein the alternative treatment is a chemotherapeutic agent, optionally wherein the chemotherapeutic agent is cyclophosphamide, doxorubicin, prednisone, vincristine, fludarabine, bendamustine, and/or rituximab.

105. The method of embodiment 98, wherein if the subject is identified as having a cancer that is not predicted to be resistant to treatment with the cytotoxic therapy, administering only the dose of the cytotoxic therapy to the subject.

106. The method of any of embodiments 98-100, further comprising administering to the identified subject a prosurvival BCL2 family protein inhibitor.

107. The method of any of embodiments 94-106, wherein the gene reference value is within 25%, within 20%, within 15%, within 10%, or within 5% of an average level or amount of the one or more gene in (a) a population of subjects not having the cancer or (b) a population of subjects having the cancer and administered the therapy, who went on to exhibit a partial response (PR) or complete response (CR) following administration of the therapy.

108. The method of embodiment 107, wherein the population of subjects having the cancer went on to exhibit the PR or CR at least 1 month, 2 months, 3 months, 4 months, 5 months, 6 months, 7 months, 8 months, 9 months, or more following administration of the therapy.

109. The method of any of embodiments 94-108, wherein the level or amount of the one or more prosurvival genes is assessed in the biological sample before a lymphodepleting therapy is administered to the subject, optionally within 7 days before, 6 days before, 5 days before, 4 days before, 3 days before, 2 days before, 1 day before, 16 hours before, 12 hours before, 6 hours before, 2 hours before, or 1 hour before the lymphodepleting therapy is administered to the subject.

110. A method of determining responsiveness of a subject having a cancer to a cytotoxic therapy, the method comprising:

(a) assessing the level or amount of expression of one or more prosurvival gene in a biological sample from the subject,

wherein the level or amount of the one or more prosurvival gene is the level or amount of a protein and/or a polynucleotide encoded by the one or more prosurvival gene, wherein the biological sample is obtained from the subject at a first time prior to the subject being administered the cytotoxic therapy, and wherein the subject is to receive treatment with the cytotoxictherapy;

(b) assessing the level or amount of expression of the one or more prosurvival gene in a biological sample from the subject at a second time after administration of the cytotoxic therapy to the subject,

wherein the level or amount of the one or more prosurvival gene is the level or amount of a protein and/or a polynucleotide encoded by the one or more prosurvival gene, wherein the biological sample is obtained at a second time after the administration of the cytotoxic therapy to the subject, and wherein the subject has been administered the cytotoxic therapy prior to the assessing in (b); and

(c) determining that the subject is responsive to the therapy if the level or amount of the one or more prosurvival gene at the second time is lower than the level or amount of the one or more prosurvival gene at the first time.

111. The method of embodiment 110, further comprising prior to the assessing in (b), administering to the subject the cytotoxic therapy.

112. The method of embodiment 110 or embodiment 111, wherein the biological sample is obtained from the subject at a time before a lymphodepleting therapy is administered to the subject, optionally within 7 days before, 6 days before, 5 days before, 4 days before, 3 days before, 2 days before, 1 day before, 16 hours before, 12 hours before, 6 hours before, 2 hours before, or 1 hour before the lymphodepleting therapy is administered to the subject.

113. The method of any of embodiments 94-112, wherein the one or more pro-survival gene is selected from among the following: a myc family gene, p53, and enhancer of zeste homolog 2 (EZH2).

114. The method of embodiment 113, wherein the one or more pro-survival gene is a myc family gene.

115. The method of embodiment 114, wherein a myc family gene comprises one or more of c-myc, l-myc, and n-myc.

116. The method of any of embodiments 113-115, wherein the one or more pro-survival gene is p53.

117. The method of any of embodiments 113-116, wherein the one or more pro-survival gene is EZH2.

118. The method of any of embodiments 94-117, wherein the cytotoxic therapy is a cell therapy.

119. The method of any of embodiments 94-118, wherein the cytotoxic therapy comprises cells that are autologous to the subject.

120. The method of any of embodiments 94-119, wherein the cytotoxic therapy is selected from among the group consisting of a tumor infiltrating lymphocytic (TIL) therapy, an endogenous T cell therapy, a natural kill (NK) cell therapy, a transgenic TCR therapy, and a recombinant receptor-expressing cell therapy, which optionally is a chimeric antigen receptor (CAR)-expressing cell therapy.

121. The method of any of embodiments 94-120, wherein the cytotoxic therapy comprises a dose of cells expressing a recombinant receptor that specifically binds to the antigen.

122. The method of any of embodiments 94-121, wherein if the subject is administered the cytotoxic therapy, administration of the cytotoxic therapy comprises administration of from or from about 1×105 to 5×108 total cells of the cell therapy, recombinant receptor-expressing cells, total T cells, or total peripheral blood mononuclear cells (PBMCs); from or from about 1×105 to 2×108 total cells of the cell therapy, recombinant receptor-expressing cells, total T cells, or total peripheral blood mononuclear cells (PBMCs); from or from about 1×106 to 1×108 total cells of the cell therapy, recombinant receptor-expressing cells, total T cells, or total peripheral blood mononuclear cells (PBMCs); or 1×106 to 5×107 total cells of the cell therapy, recombinant receptor-expressing cells, total T cells, or total peripheral blood mononuclear cells (PBMCs).

123. The method of any of embodiments 94-122, wherein the cytotoxic therapy comprises or is enriched in T cells.

124. The method of any of embodiments 94-123, wherein the cytotoxic therapy comprises or is enriched in CD3+, CD4+, CD8+ or CD4+ and CD8+ T cells.

125. The method of any of embodiments 94-124, wherein the cytotoxic therapy comprises or is enriched in CD4+ and CD8+ T cells.

126. The method of embodiment 125, wherein the CD4+ and CD8+ T cells of the cytotoxic therapy comprises a defined ratio of CD4+ CAR-expressing T cells to CD8+ CAR-expressing T cells and/or of CD4+ CAR-expressing T cells to CD8+ CAR-expressing T cells, that is or is approximately 1:1 or is between approximately 1:3 and approximately 3:1.

127. The method of any of embodiments 94-126, wherein the cytotoxic therapy comprises administering CD4+ and CD8+ T cells, wherein T cells of each dose comprises a receptor, optionally a CAR, that specifically binds to the antigen, wherein the administration comprises administering a plurality of separate compositions, the plurality of separate compositions comprising a first composition comprising or enriched in the CD8+ T cells and a second composition comprising or enriched in the CD4+ T cells.

128. The method of embodiment 127, wherein:

the CD4+ T cells comprising the receptor in the one of the first and second compositions and the CD8+ T cells comprising the receptor in the other of the first and second compositions are present at a defined ratio that is or is approximately 1:1 or is between approximately 1:3 and approximately 3:1; and/or

the CD4+ T cells comprising the receptor and the CD8+ T cells comprising the receptor administered in the first and second compositions are present at a defined ratio, which ratio is or is approximately 1:1 or is between approximately 1:3 and approximately 3:1.

129. The method of any of embodiments 94-128, wherein the cytotoxic therapy comprises or is enriched in natural killer (NK) cells.

130. The method of any of embodiments 94-129, wherein the cytotoxic therapy comprises or is enriched in iPS-derived cells.

131. The method of any of embodiments 120-130, wherein the recombinant receptor is a T cell receptor (TCR) or a functional non-T cell receptor.

132. The method of any of embodiments 120-130, wherein the recombinant receptor is a chimeric antigen receptor (CAR).

133. The method of any of embodiments 94-132, wherein the cytotoxic therapy comprises administration of from or from about 1×105 to 5×108 total CAR-expressing T cells, 1×106 to 2.5×108 total CAR-expressing T cells, 5×106 to 1×108 total CAR-expressing T cells, 1×107 to 2.5×108 total CAR-expressing T cells, 5×107 to 1×108 total CAR-expressing T cells, each inclusive.

134. The method of any of embodiments 94-133, wherein the cytotoxic therapy comprises administration of at least or at least about 1×105 CAR-expressing cells, at least or at least about 2.5×105 CAR-expressing cells, at least or at least about 5×105 CAR-expressing cells, at least or at least about 1×106 CAR-expressing cells, at least or at least about 2.5×106 CAR-expressing cells, at least or at least about 5×106 CAR-expressing cells, at least or at least about 1×107 CAR-expressing cells, at least or at least about 2.5×107 CAR-expressing cells, at least or at least about 5×107 CAR-expressing cells, at least or at least about 1×108 CAR-expressing cells, at least or at least about 2.5×108 CAR-expressing cells, or at least or at least about 5×108 CAR-expressing cells.

135. The method of any of embodiments 94-134, wherein the cytotoxic therapy comprises administration of at or about 5×107 total CAR-expressing T cells.

136. The method of any of embodiments 94-135, wherein the cytotoxic therapy comprises administration of at or about 1×108 CAR-expressing cells.

137. The method of any of embodiments 94-136, wherein the cytotoxic therapy comprises cells expressing a chimeric antigen receptor (CAR) comprising an extracellular antigen-recognition domain that specifically binds to the antigen and an intracellular signaling domain comprising an ITAM.

138. The method of any of embodiments 94-137, wherein the antigen is selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha(IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1).

139. The method of any of embodiments 94-138, wherein the antigen is CD19.

140. The method of any of embodiments 137-139, wherein the intracellular signaling domain comprises an intracellular domain of a CD3-zeta (CD3) chain.

141. The method of any of embodiments 137-140, wherein the intracellular signaling region further comprises a costimulatory signaling region.

142. The method of embodiment 141, wherein the costimulatory signaling region comprises a signaling domain of CD28 or 4-1BB, optionally human CD28 or human 4-1BB.

143. The method of embodiment 142, wherein the costimulatory domain is or comprises a signaling domain of CD28.

144. The method of any of embodiments 94-143, wherein the cytotoxic therapy comprises autologous cells from the subject.

145. The method of embodiment 144, wherein if the subject is administered a prosurvival BCL2 family protein, the method comprises collecting a biological sample from the subject comprising the autologous cells prior to initiation of administration of the inhibitor.

146. The method of embodiment 145, wherein the biological sample from the subject is or comprises a whole blood sample, a buffy coat sample, a peripheral blood mononuclear cells (PBMC) sample, an unfractionated T cell sample, a lymphocyte sample, a white blood cell sample, an apheresis product, or a leukapheresis product.

147. The method of any of embodiments 94-146, wherein if the subject is administered a prosurvival BCL2 family protein, the method further comprises, prior to administration of the cytotoxic therapy, administering a lymphodepleting agent or therapy to the subject.

148. The method of any of embodiments 94-147, wherein the subject has been previously treated with an inhibitor of a prosurvival BCL2 family protein, optionally wherein the subject has been previously treated with venetoclax.

149. The method of embodiment 148, wherein the previous treatment with the inhibitor is administered at a time between the collecting of the autologous cells and prior to a lymphodepleting therapy.

150. The method of embodiment 149, wherein the previous treatment with the inhibitor is ceased for at least at or about 3 days or for at least at or about 4 days prior to administration of a lymphodepleting therapy and/or for at least an amount of time until the concentration of the inhibitor in the subject's bloodstream is reduced by about three half-lives or about four half-lives and/or for at least an amount of time until the inhibitor is eliminated from the bloodstream of the subject.

151. The method of any of embodiments 147-150, wherein the lymphodepleting therapy is completed between 2 and 7 days before the initiation of administration of the cytotoxic therapy.

152. The method of any of embodiments 147-151, wherein the lymphodepleting therapy comprises the administration of fludarabine and/or cyclophosphamide.

153. The method of any of embodiments 147-152, wherein the lymphodepleting therapy comprises administration of cyclophosphamide at about 200-400 mg/m2, optionally at or about 300 mg/m2, inclusive, and/or fludarabine at about 20-40 mg/m2, optionally 30 mg/m2, daily for 2-4 days, optionally for 3 days, or wherein the lymphodepleting therapy comprises administration of cyclophosphamide at about 500 mg/m2.

154. The method of any one of embodiments 147-153, wherein:

the lymphodepleting therapy comprises administration of cyclophosphamide at or about 300 mg/m2 and fludarabine at about 30 mg/m2 daily for 3 days; and/or

the lymphodepleting therapy comprises administration of cyclophosphamide at or about 500 mg/m2 and fludarabine at about 30 mg/m2 daily for 3 days.

155. The method of any of embodiments 94-154, wherein if the subject is administered a prosurvival BCL2 family protein inhibitor, the initiation of administration of the inhibitor is within at or about 3 days prior to initiation of administration of the cytotoxic therapy.

156. The method of any of embodiments 95-155, wherein if the subject is administered a prosurvival BCL2 family protein inhibitor, the initiation of administration of the inhibitor is within at or about 2 days prior to initiation of administration of the cytotoxic therapy.

157. The method of any of embodiments 95-156, wherein if the subject is administered a prosurvival BCL2 family protein inhibitor, the initiation of administration of the inhibitor is within at or about 1 day prior to initiation of administration of the cytotoxic therapy.

158. The method of any of embodiments 95-157, wherein if the subject is administered a prosurvival BCL2 family protein inhibitor, the initiation of administration of the inhibitor is concurrent with or on the same day as initiation of administration of the inhibitor.

159. The method of any of embodiments 95-154, wherein if the subject is administered a prosurvival BCL2 family protein inhibitor, the initiation of administration of the inhibitor is no more than 2 days after initiation of administration of the cytotoxic therapy, optionally wherein the initiation of administration of the inhibitor is within 1 day after the initiation of administration of the cytotoxic therapy.

160. The method of any of embodiments 95-159, wherein if the subject is administered a prosurvival BCL2 family protein inhibitor:

the dosing regimen of the inhibitor comprises a subtherapeutic amount of the inhibitor;

the dosing regimen of the inhibitor is not sufficient to reduce tumor burden in the subject or reduces tumor burden by less than 10% when administered as a monotherapy in the absence of the combined administration with the cytotoxic therapy; and/or

the dosing regimen of the inhibitor does not result in a complete or partial response in a group of similarly treated subjects, or results in such a response in no more than 10% of such subjects, when administered as a monotherapy in the absence of the combined administration with the cytotoxic therapy.

161. The method of any of embodiments 95-160, wherein if the subject is administered a prosurvival BCL2 family protein inhibitor, the dosing regimen of the inhibitor comprises once daily dosing.

162. The method of any of embodiments 95-161, wherein if the subject is administered a prosurvival BCL2 family protein inhibitor, the once daily dose is an amount of the inhibitor of between at or about 20 mg and at or about 800 mg, between at or about 20 mg and at or 400 mg, between at or about 20 mg and at or about 350 mg, between at or about 20 mg and at or about 300 mg, between at or about 20 mg and at or about 250 mg, between at or about 20 mg and at or about 200 mg, between at or about 20 mg and at or about 150 mg, between at or about 20 mg and at or about 100 mg, between at or about 20 mg and at or about 50 mg, between at or about 20 mg and at or about 40 mg, between at or about 40 mg and at or about 800 mg, between at or about 40 mg and at or 400 mg, between at or about 40 mg and at or about 350 mg, between at or about 40 mg and at or about 300 mg, between at or about 40 mg and at or about 250 mg, between at or about 40 mg and at or about 200 mg, between at or about 40 mg and at or about 150 mg, between at or about 40 mg and at or about 100 mg, between at or about 40 mg and at or about 50 mg, between at or about 50 mg and at or about 800 mg, between at or about 50 mg and at or about 400 mg, between at or about 50 mg and at or about 350 mg, between at or about 50 mg and at or about 300 mg, between at or about 50 mg and at or about 250 mg, between at or about 50 mg and at or about 200 mg, between at or about 50 mg and at or about 150 mg, between at or about 50 mg and at or about 100 mg, between at or about 100 mg and at or about 800 mg, between at or about 100 mg and at or about 400 mg, between at or about 100 mg and at or about 350 mg, between at or about 100 mg and at or about 300 mg, between at or about 100 mg and at or about 250 mg, between at or about 100 mg and at or about 200 mg, between at or about 100 mg and at or about 150 mg, between at or about 150 mg and at or about 800 mg, between at or about 150 mg and at or about 400 mg, between at or about 150 mg and at or about 350 mg, between at or about 150 mg and at or about 300 mg, between at or about 150 mg and at or about 250 mg, between at or about 150 mg and at or about 200 mg, between at or about 200 mg and at or about 800 mg, between at or about 200 mg and at or about 400 mg, between at or about 200 mg and at or about 350 mg, between at or about 200 mg and at or about 300 mg, between at or about 200 mg and at or about 250 mg, between at or about 250 mg and at or about 300 mg, between at or about 300 mg and at or about 350 mg, between at or about 300 mg and at or about 400 mg, between at or about 300 mg and at or about 800 mg, between at or about 350 mg and at or about 400 mg, and between at or about 350 mg and at or about 800 mg, each inclusive.

163. The method of any of embodiments 95-162, wherein if the subject is administered a prosurvival BCL2 family protein inhibitor, the once daily dose is an amount of the inhibitor of between at or about 20 mg and at or about 100 mg, inclusive.

164. The method of any of embodiments 95-163, wherein if the subject is administered a prosurvival BCL2 family protein inhibitor, the once daily dose is an amount of the inhibitor of between at or about 40 mg and at or about 100 mg, inclusive.

165. The method of any of embodiments 95-164, wherein if the subject is administered a prosurvival BCL2 family protein inhibitor, the once daily dose is an amount of the inhibitor of between at or about 50 mg and at or about 100 mg, inclusive.

166. The method of any of embodiments 94-165, wherein if the subject is administered a prosurvival BCL2 family protein inhibitor, the once daily dose is an amount of the inhibitor of at or about 20 mg.

167. The method of any of embodiments 94-165, wherein if the subject is administered a prosurvival BCL2 family protein inhibitor, the once daily dose is an amount of the inhibitor of at or about 40 mg.

168. The method of any of embodiments 94-165, wherein if the subject is administered a prosurvival BCL2 family protein inhibitor, the once daily dose is an amount of the inhibitor of at or about 50 mg.

169. The method of any of embodiments 95-165, wherein if the subject is administered a prosurvival BCL2 family protein inhibitor, the once daily dose is an amount of the inhibitor of at or about 100 mg.

170. The method of any of embodiments 95-157, wherein if the subject is administered a prosurvival BCL2 family protein inhibitor, the inhibitor is administered in a dose-ramp up schedule prior to administration of the dosing regimen of the inhibitor, optionally wherein the dose-ramp up schedule comprises administration of increasing amounts of the inhibitor up to the amount of the once daily dose.

171. The method of any of embodiments 95-170, wherein if the subject is administered a prosurvival BCL2 family protein inhibitor, the inhibitor inhibits one or more prosurvival BCL2 family protein selected from among the group consisting of BCL2, BCLXL, BCLW, BCLB, MCL1, and combinations thereof.

172. The method of embodiment 171, wherein the one or more prosurvival BCL2 family protein is BCL2, BCLXL, and/or BCLW.

173. The method of any of embodiments 95-172, wherein the inhibitor inhibits the one or more prosurvival BCL2 family protein with a half-maximal inhibitory concentration (IC50) of less than or less than about 1000 nM, 900 nM, 800 nM, 600 nM, 500 nM, 400 nM, 300 nM, 200 nM, 100 nM, 50 nM or less than or less than about 10 nM.

174. The method of any of embodiments 95-173, wherein the inhibitor is selected from among the group consisting of venetoclax, navitoclax, ABT737, maritoclax, obatoclax, and clitocine.

175. The method of any of embodiments 94-174, wherein the inhibitor is navitoclax.

176. The method of any of embodiments 95-174, wherein the inhibitor is venetoclax.

177. The method of any of embodiments 95-174, wherein the cancer is a hematological malignancy.

178. The method of any of embodiments 94-177, wherein the cancer is a B cell malignancy.

179. The method of any of embodiments 94-178, wherein the cancer is a myeloma, leukemia or lymphoma.

180. The method of any of embodiments 94-179, wherein the cancer is an acute lymphoblastic leukemia (ALL), adult ALL, chronic lymphoblastic leukemia (CLL), a small lymphocytic lymphoma (SLL), non-Hodgkin lymphoma (NHL), a large B cell lymphoma.

181. The method of any of embodiments 94-810, wherein the cancer is a chronic lymphocytic leukemia (CLL).

182. The method of any of embodiments 94-180, wherein the cancer is a non-Hodgkin lymphoma (NHL).

183. The method of embodiment 182, wherein the NHL is a diffuse large B-cell lymphoma (DLBCL). 184. The method of any of embodiments 94-180, wherein the cancer is primary mediastinal B-cell lymphoma (PMBCL) or a follicular lymphoma (FL), optionally follicular lymphoma grade 3B (FL3B).

185. The method of any of embodiments 94-184, wherein the subject has relapsed following remission after treatment with, or become refractory to, failed and/or was intolerant to treatment with the one or more prior therapies for treating the cancer.

186. The method of any of embodiments 94-185, wherein the cancer is resistant to treatment with the cytotoxic therapy alone.

187. The method of any of embodiments 96-186, wherein the cancer exhibits overexpression or aberrant expression of a prosurvival BCL2 family protein.

188. The method of any of embodiments 95-187, wherein if the subject is administered a prosurvival BCL2 family protein inhibitor, the dosing regimen of the inhibitor comprises administration of the inhibitor, optionally once daily, for up to 6 months after the initiation of the administration of the cytotoxic therapy.

189. The method of any of embodiments 95-188, wherein if the subject is administered a prosurvival BCL2 family protein inhibitor, the dosing regimen of inhibitor comprises administration of the inhibitor, optionally once daily, for up to 3 months after the initiation of the administration of the cytotoxic therapy

190. The method of any of embodiments 95-189, wherein if the subject is administered a prosurvival BCL2 family protein inhibitor, administration of the inhibitor in the dosing regimen is discontinued if the subject exhibits clinical remission.

191. The method of any of embodiments 95-190, wherein if the subject is administered a prosurvival BCL2 family protein inhibitor;
the method increases the cytotoxic activity of the cytotoxic therapy compared to a method that does not involve the administration of the inhibitor; and/or
the method increases cytolytic killing, optionally via perforin- and/or granzyme-mediated apoptosis, of one or more of the cancer cells compared to a method that does not involve the administration of the inhibitor.

192. The method of any of embodiments 94-191, wherein:

at least 35%, at least 40% or at least 50% of subjects treated according to the method achieve a complete response (CR) that is durable, or is durable in at least 60, 70, 80, 90, or 95% of subjects achieving the CR, for at or greater than 6 months or at or greater than 9 months; and/or

wherein at least 60, 70, 80, 90, or 95% of subjects achieving a CR by six months remain in response, remain in CR, and/or survive or survive without progression, for greater at or greater than 3 months and/or at or greater than 6 months and/or at greater than nine months; and/or

at least 50%, at least 60% or at least 70% of the subjects treated according to the method achieve objective response (OR) optionally wherein the OR is durable, or is durable in at least 60, 70, 80, 90, or 95% of subjects achieving the OR, for at or greater than 6 months or at or greater than 9 months; and/or

wherein at least 60, 70, 80, 90, or 95% of subjects achieving an OR by six months remain in response or surviving for greater at or greater than 3 months and/or at or greater than 6 months.

193. The method of any of embodiments 94-192, wherein the biological sample is a tumor biopsy, optionally a lymph node biopsy.

194. The method of any one of embodiments 94-193, wherein the subject is a human.

### EXAMPLES

The following examples are included for illustrative purposes only and are not intended to limit the scope of the invention.

### Example 1: Assessment of CAR T-Mediated Killing of Tumor Cells

T cell compositions containing anti-CD19 CAR-expressing T cells were generated from leukapheresis samples from two human adult donors by a process including immunoaffinity-based selection of T cells (including CD4+ and CD8+ cells) from the samples, resulting in two compositions, enriched for CD8+ and CD4+ cells, respectively. Cells of the enriched CD4+ and CD8+ compositions were separately activated with anti-CD3/anti-CD28 beads and subjected to lentiviral transduction with a vector encoding an anti-CD19 CAR. The anti-CD19 CAR contained an anti-CD19 scFv derived from a murine antibody (variable region derived from FMC63), an immunoglobulin-derived spacer, a transmembrane domain derived from CD28, a costimulatory region derived from 4-1BB, and a CD3-zeta intracellular signaling domain. The expression construct in the viral vector further contained sequences encoding a truncated receptor, which served as a surrogate marker for CAR expression, which was separated from the CAR sequence by a T2A ribosome skip sequence. Transduced populations then were separately incubated in the presence of stimulating reagents for cell expansion. Expanded CD8+ and CD4+ cells were formulated and cryopreserved separately and stored.

The cyropreserved CD4+ and CD8+ anti-CD19 CAR-expressing cells from each donor were thawed, and combined at approximately a 1:1 CAR+ CD4+:CD8+ ratio prior to use.

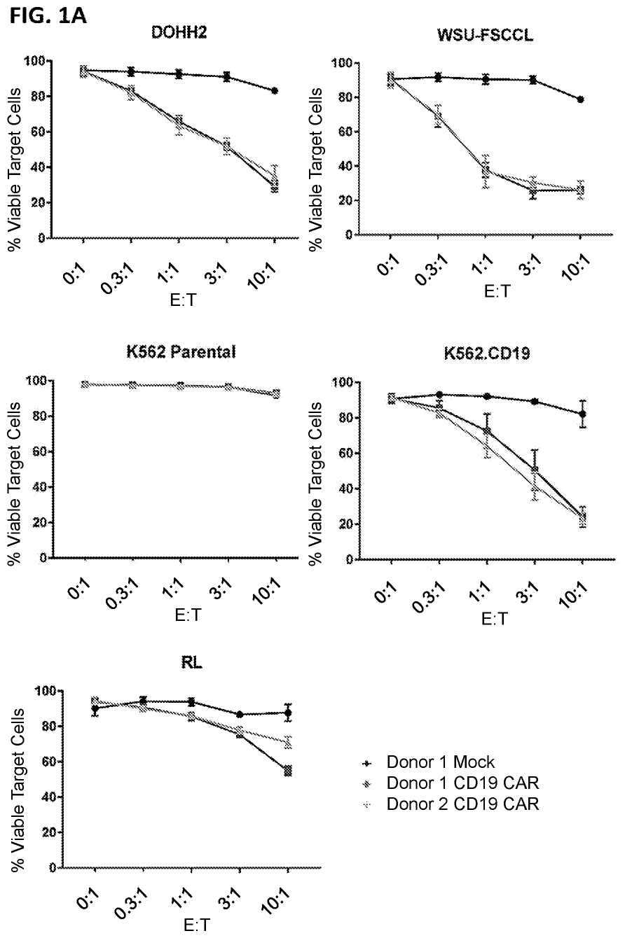

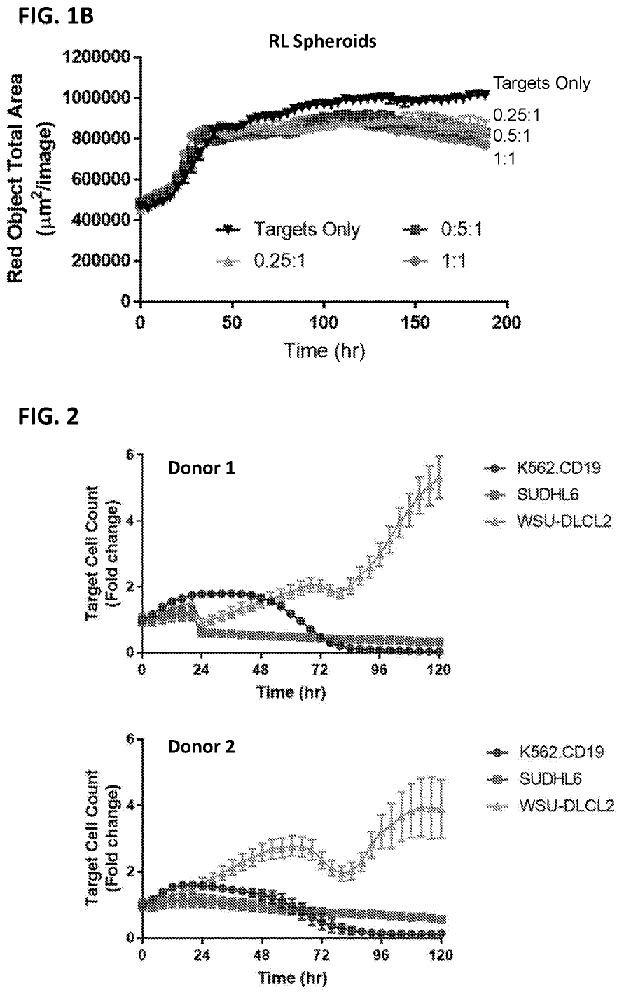

Anti-CD19 CAR T cell compositions were cultured with CD19-expressing target cells, including K562 cells transduced to express CD19 (K562.CD19) and the CD19-expressing lymphoma cell lines DOHH2, WSU-FSCLL, and RL. As a control, anti-CD19 CAR T cell compositions were co-cultured with K562 cells not expressing CD19 (K562 parental). Co-cultures with T cells not expressing the CAR (mock) were also used as controls. The CAR+ T cell compositions and cell lines were co-cultured for 24 hours at effector cell to target cell ratios (E:T) between 0:1 to 10:1 (0:1, 0.3:1, 1:1, 3:1, and 10:1). The target cells were labeled with CellTrace Violet (CTV). Following culture, target cells were harvested and stained with LIVE/DEAD Fixable Near-IR Dead Cell Stain and analyzed by flow cytometry. Viable target cells were identified as CTV positive, LIVE/DEAD negative. As shown in FIG. 1A, the percentage of viable target cells decreased with increasing E:T ratios in co-cultures of anti-CD19 CAR T cells with CD19-expressing DOHH2, WSU-FSCLL, and K562.CD19 target cell lines. By contrast, the RL cells were more resistant to cytolytic killing by anti-CD19 CAR T cells generated from both donors.

Separately, RL spheroids were generated by culturing CD19-expressing RL cells engineered with a lentiviral reagent to express NucLight Red in culture plates for 72 hours. An anti-CD19 CAR-expressing T cell composition was generated from a human adult donor, substantially as described above. The anti-CD19 CAR T cells were co-cultured with the RL spheroids for up to 196 hours at E:T ratios of 0.25:1, 0.5:1, and 1:1. Spheroid size was measured over time by the red object total area (m²/image). The size of spheroids co-cultured with anti-CD19 CAR T cells over time is shown in FIG. 1B.

The cytolytic activity of anti-CD19 CAR T cells against two additional CD19-expressing lymphoma target cells lines, SUDHL6 and WSU-DLCL2, was assessed and compared to cytolytic activity against K562.CD19 target cells. Target cells were engineered with a lentiviral reagent to express NucLight Red (NLR) to permit tracking of target cells by microscopy. Anti-CD19 CAR-expressing T cell compositions were generated from two human adult donors, substantially as described above. Target cells were co-cultured with the donor-derived CAR T cell compositions for up to 120 hours at an E:T ratio of 2.5:1. Red target cell counts were tracked over time by live cell imaging and counts were normalized to the number of red target cells at baseline to determine the fold-change in target cells over time. As shown in FIG. 2, anti-CD19 CAR T cells exhibited substantial cytolytic activity against K562.CD19 and SUDHL6 lymphoma target cells. However, the fold-change in cell count of WSU-DLCL2 lymphoma target cells increased over the course of the assay, indicating that these cells were less susceptible to killing by anti-CD19 CAR T cells. These results indicate certain CD19-expressing cells are resistant to CD19-directed CAR-mediated cell killing and supports a hypothesis that some CD19-expressing tumors may be less sensitive to CD19-directed CAR-mediated cell killing.

Effect of a BH3 Family Member (BCL-2) Inhibitor on Anti-CD19 CAR T Cells

Anti-CD19 CAR-expressing T cell compositions were generated from three human adult donors, substantially as described in Example 1. Anti-CD19 CAR T cells were stimulated for 96 hours with an anti-idiotypic antibody in the presence of an exemplary BCL2 inhibitor venetoclax solubilized in dimethylsulfoxide (DMSO). Control anti-CD19 CAR T cells were stimulated for 96 hours with the anti-idiopathic antibody in the presence of an equal amount of DMSO. Specifically, anti-CD19 CAR-expressing cells were added to the wells of a 96-well plate that had been pre-coated with an anti-idiotypic antibody specific to the scFv of the anti-CD19 CAR-expressing T cells described in Example 1 at a concentration of 30 μg/ml. The cells were cultured in the presence of 5% human serum at 37° C. and 5% CO2. After 96 hours, cells were lysed and ATP from viable cells was detected via a luciferin reporter assay and reported as relative luminescence units. Venetoclax-treated CAR T cell counts were assessed and normalized to DMSO control CAR T cell counts.

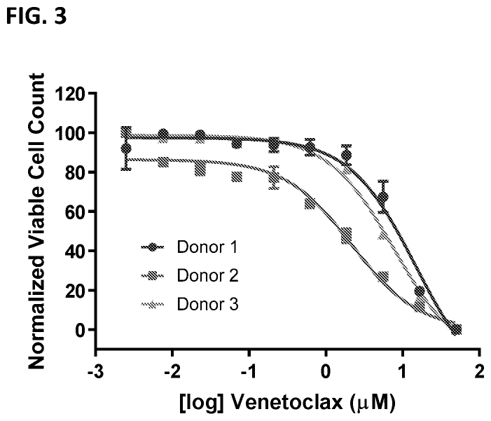

As shown in FIG. 3, the percentage of viable anti-CD 19 CAR-expressing T cells decreased with increasing doses of venetoclax. Among the three donors, the average IC50 of venetoclax was 7.9 μM, and the average IC₁₀ of venetoclax was 0.88 04. In some cases, clinical observations have revealed that a daily 400 milligram dose of venetoclax yields a maximum serum concentration (Cₘₐₓ) of 2.4±1.3 04. These results are consistent with an observation that potentiation of anti-CD19 CAR-T cell activity can be seen at lower concentrations than those that may have a detrimental effect on CAR T-cell viability, under certain conditions.

### Example 2: Effect of a BH3 Family Member (BCL-2) Inhibitor on CD19-Directed Tumor Cell Resistance

Anti-CD19 CAR-expressing T cell compositions were generated as described in Example 1 and were cultured with a CD19-expressing lymphoma target cell lines, either SUDHL6, WSU-DLCL2, or Granta-519 at an E:T ratio of 2.5:1. The co-cultures were carried out in the presence of 20 nM or 200 nM of the exemplary BCL2 inhibitor, venetoclax. As controls, target tumor cell lines were incubated only in the presence of the inhibitor or the anti-CD19 CAR T cells. Target cells were labeled with NucLight Red (NLR) to permit tracking of target cells by microscopy and target cell killing was monitored by the loss of fluorescent signal over time, substantially as described in Example 1.

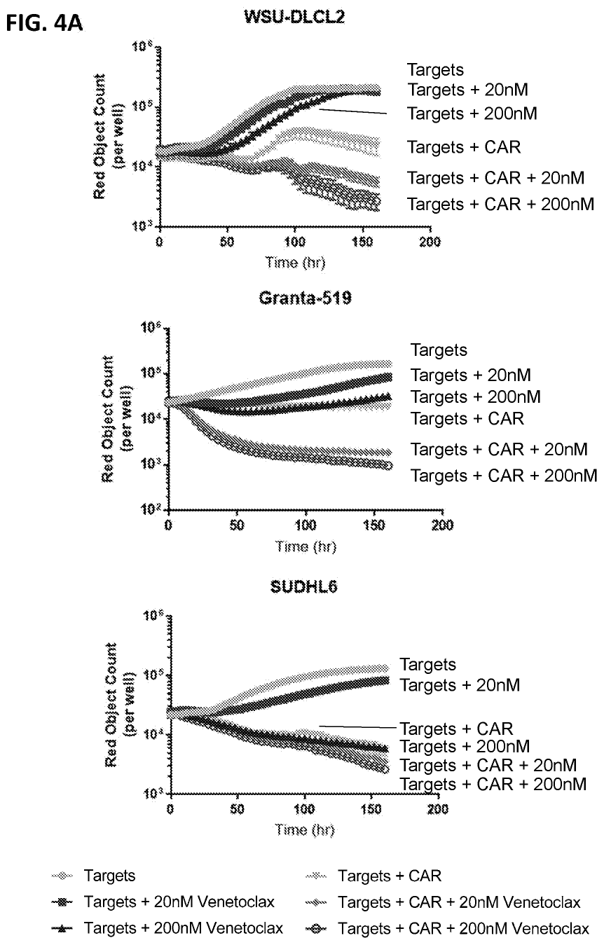

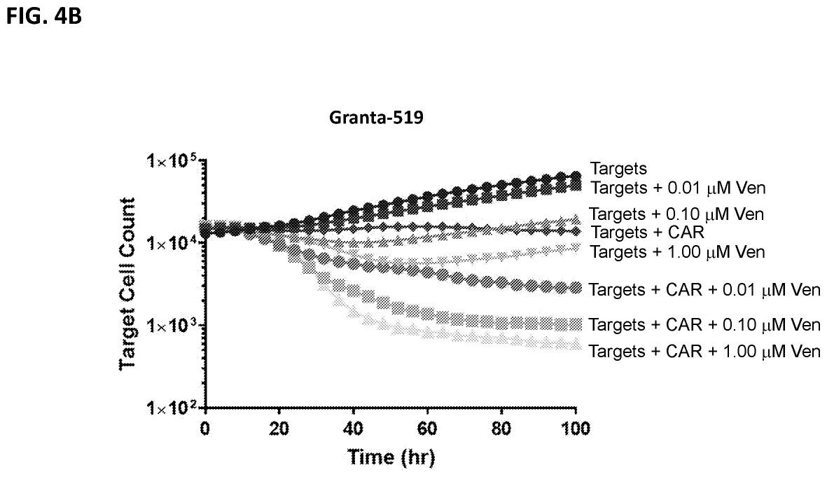

As shown in FIG. 4A, anti-CD19 CAR T cells exhibited cytolytic activity against SUDHL6 cells but WSU-DLCL2 and Granta-519 cell lines were more resistant to CD19-directed CAR T cell killing. The presence of venetoclax alone at either 20 nM or 200 nM did not substantially impact tumor cell killing in this assay for the resistant tumor cell lines, although killing of the sensitive tumor cell line SUDHL6 was achieved in cultures incubated with 200 nM venetoclax. CD19-directed CAR T cell killing of tumor resistant cell lines was enhanced in co-cultures that were incubated with 20 or 200 nM venetoclax. These results suggest that even low-dose venetoclax, e.g. 20 nM, can sensitize tumor cells to CD19-directed CAR T cell-mediated cytotoxicity that are otherwise resistant.

In a further experiment, anti-CD19 CAR-expressing T cell compositions, generated as described above, were cultured with the Granta-519 CD19-expressing lymphoma target cell line. Granta-519 cells were engineered with a lentiviral reagent to express NucLight Red (NLR) to permit tracking of target cells by microscopy. As described above, the Granta-519 cell line was determined to be relatively resistant to CD19-directed CAR T cell-mediated cytotoxicity, such that co-culture of the CAR T cells and the target cells at an E:T ratio of 1:1 represented a suboptimal dose of CAR T cells. The anti-CD19 CAR T cells were provided in the co-cultures at the suboptimal dose, and the co-cultures were carried out in the presence of 0, 0.01, 0.1, or 1 μM of the exemplary BCL2 inhibitor venetoclax. For comparison, target tumor cell lines were incubated only in the presence of 0, 0.01, 0.1, or 1 μM of the inhibitor. Red target cell counts were enumerated by live cell imaging and tracked over time.

As shown in FIG. 4B, the lymphoma target cells were relatively resistant to CD19-directed CAR T cell killing at the suboptimal dose of CAR T cells, as indicated by a modest decrease of target cell number following co-culture with the anti-CD19 CAR T cells in the absence of venetoclax. The presence of venetoclax alone at 0.01 μM did not substantially impact target cell number in this assay, whereas the presence of venetoclax alone at 0.1 μM and at 1.0 μM had a modest effect on target cell number. However, co-cultures containing target lymphoma cells and anti-CD19 CAR T cells in the presence of even 0.01 μM of venetoclax demonstrated a decrease in the number of target cells, compared to target cells treated with only 0.01 μM of the inhibitor, consistent with a potentially synergistic effect of the combination. Co-cultures containing target lymphoma cells and anti-CD19 CAR T cells in the presence of 0.1 μM and 1.0 μM venetoclax demonstrated an even greater CD19-directed CAR T cell killing.

These results are consistent with an observation that low doses of venetoclax may sensitize cells that are otherwise relatively resistant to a suboptimal dose of anti-CD19 CAR T cells.

### Example 3: Effect of a BH3 Family Member (BCL-2) Inhibitor on RL Cell Resistance to CD19-Targeting CAR T Cells

Anti-CD19 CAR-expressing T cell compositions, generated as described in Example 1, were cultured with the CD19-expressing lymphoma target cell line RL. Target cells were engineered with a lentiviral reagent to express NucLight Red (NLR) to permit tracking of the cells by microscopy. The RL cell line was determined to be relatively resistant to CD19-directed CAR T cell-mediated cytotoxicity, such that co-culture of the CAR T cells and the target cells at an E:T ratio of 1:1 represented a suboptimal dose of CART cells. The anti-CD19 CART cells were co-cultured with target RL cells at an E:T ratio of 1:1, and the co-cultures were carried out in the presence of 0.1 μM of the exemplary BCL2 inhibitor venetoclax. For comparison, target cells were incubated only in the presence of the CAR T cells or 0.1 μM of the inhibitor.

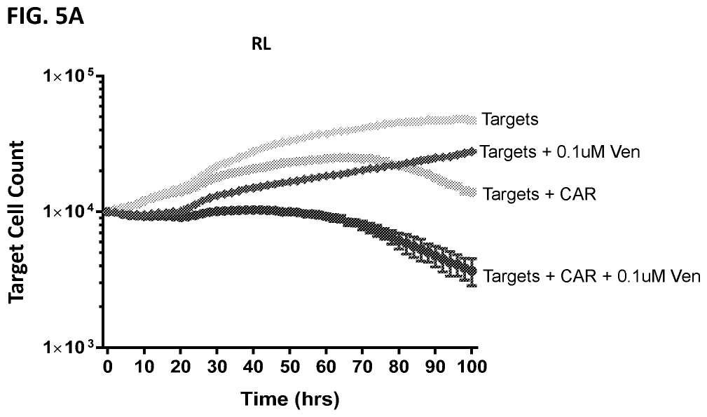

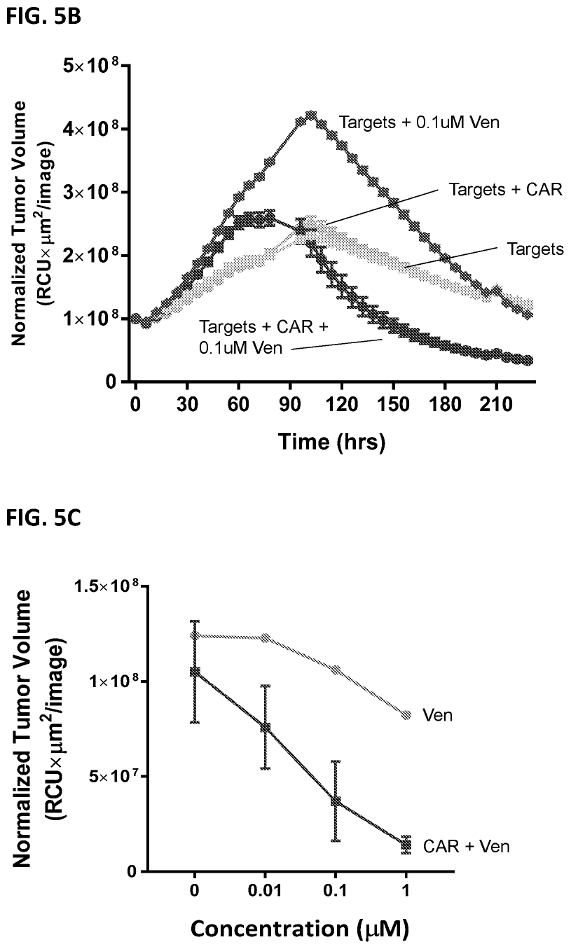

As shown in FIG. 5A, the RL cells were relatively resistant to CD19-directed CAR T cells alone, but were sensitized to the CD19-directed CAR T cell killing in the presence of 0.1 venetoclax.

The resistance of the RL cell line to CD19-directed CAR T cells was further assessed in a 3D tumor model. An anti-CD19 CAR-expressing T cell composition was generated from a human adult donor, substantially as described in Example 1. RL tumor cells engineered to express NLR were allowed to form spheroids for 72 hours, then co-cultured for 9 days with the anti-CD19 CAR T cells at an E:T ratio of 1:1. A subtherapeutic concentration of the exemplary BCL2 inhibitor venetoclax (0.1 μM) was added at the beginning of co-culture, and tumor volume was measured over time (FIG. 5B). While initial tumor volume was not reduced in the presence of the anti-CD19 CAR T cells alone, the addition of 0.1 μM venetoclax resulted in decreased tumor volume.

In a related experiment, spheroids were generated from CD19-expressing RL cells as described, and co-cultured for 9 days with anti-CD19 CAR T cells and increasing concentrations of venetoclax (0.01 μM, 0.1 μM, or 1.0 μM), or with only corresponding concentrations of venetoclax. As shown in FIG. 5C, at day 9 of the co-culture, larger concentrations of venetoclax resulted in larger decreases in tumor size. However, the size of the RL spheroids cultured with both anti-CD19 CAR T cells and venetoclax decreased substantially more compared to that of RL spheroids cultured with just venetoclax.

In both RL spheroid experiments, the combination of venetoclax and anti-CD19 CAR T cells was observed to induce apoptosis of tumor cells (data not shown), despite such effects not being observed in the presence of the CAR T cells alone. These results indicated that venetoclax may sensitize tumor cells to anti-CD19 CAR T cell-mediated cell death, such that the combination exhibits more potent antitumor activity than venetoclax or the anti-CD19 CAR T cells alone.

### Example 4: Effect of a BCL-2 Inhibitor on Chronically Stimulated Anti-CD19 CAR T Cells Against CD19-Directed Tumor Cell Resistance

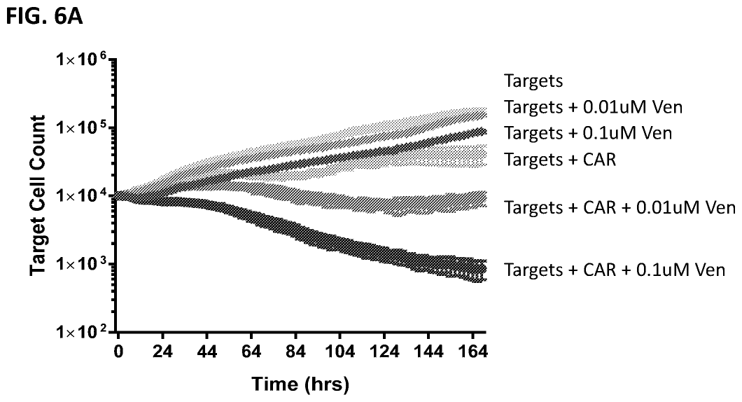

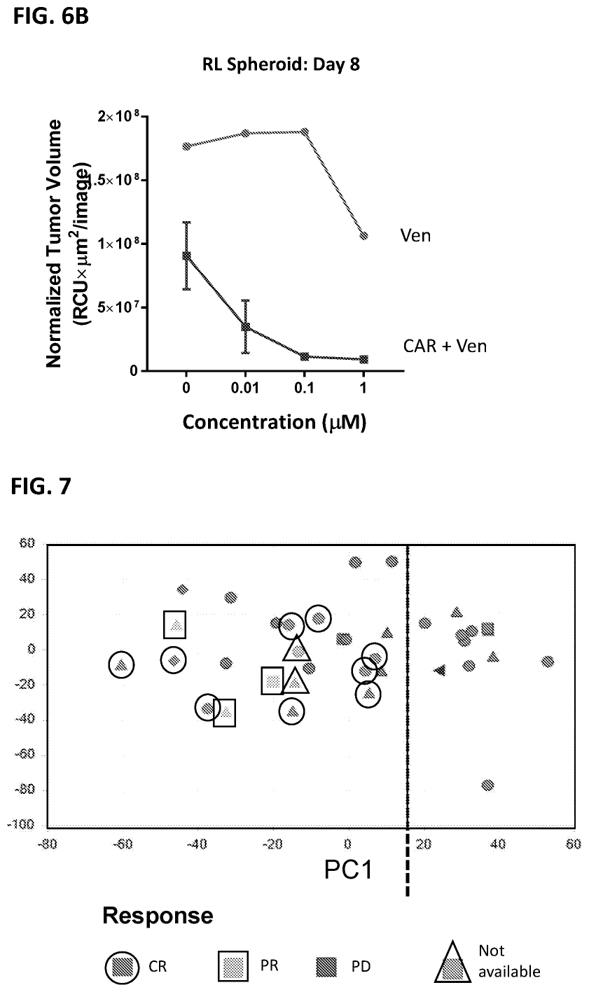

Anti-CD19 CAR-expressing T cells, generated from healthy donors as described above, were chronically stimulated under conditions to exhaust T cell activity by incubation for 8 days with beads coated in anti-idiotypic (anti-ID) antibody directed against the CAR. After the 8 days of chronic stimulation, the anti-CD19 CAR-expressing T cells were harvested and subsequently co-cultured with RL cells engineered to express NLR for 8 days at an E:T ratio of 1:1. As a control, RL cells were cultured in the absence of anti-CD19 CAR T cells. Subtherapeutic concentrations of the exemplary BCL2 inhibitor venetoclax (0.01 μM or 0.1 μM) were added to the cultures, and the number of cells was assessed over time (FIG. 6A).

In another experiment, spheroids were generated from CD19-expressing RL cells as previously described, and co-cultured for 8 days with anti-CD19 CAR T cells at an E:T ratio of 1:1 and increasing concentrations of venetoclax (0.01 μM, 0.1 μM, or 1.0 μM), or with only corresponding concentrations of venetoclax. As before, the anti-CD19 CAR T cells were chronically stimulated with anti-ID antibody-coated beads prior to co-culture with the RL spheroids. As shown in FIG. 6B, decreases in tumor size were only observed with 1 μM venetoclax when target cells were cultured in the absence of CD19-targeting CAR T cells. By contrast, greater decreases in tumor size were seen with increasing concentrations of venetoclax when target cells were cultured in the presence of CD19-targeting CAR T cells. Overall, the size of the RL spheroids cultured with both anti-CD19 CAR T cells and venetoclax decreased more, and with lower concentrations of venetoclax, compared to that of RL spheroids cultured with just venetoclax.

These data indicate that treatment with a BCL2 inhibitor enhanced the potency of chronically stimulated anti-CD19 CAR T cells. Without wishing to be bound by theory, the results indicate that addition of a BCL2 inhibitor may restore or reverse an exhausted or chronically stimulated state in CAR T cells, such as may be found in patients with terminally differentiated CAR T cells and/or high disease burden.

### Example 5: Gene Expression Data from Subjects with Diffuse Large B-Cell Lymphoma

Therapeutic CAR T cell compositions containing autologous T cells expressing a chimeric antigen-receptor (CAR) specific for CD19 were administered to subjects with B cell malignancies, and expression of genes in pre-treatment tumor biopsies that correlated to response in subjects administered the CAR T cell compositions was determined for a subset of subjects.

Specifically, autologous anti-CD19 directed therapeutic T cell compositions were generated from and used to treat adult human subjects with relapsed or refractory (R/R) aggressive non-Hodgkin's lymphoma (NHL), including diffuse large B-cell lymphoma (DLBCL), de novo or transformed from indolent lymphoma (NOS), high-grade B-cell lymphoma, with MYC and BCL2 and/or BCL6 rearrangements with DLBCL histology (double/triple hit), DLBCL transformed from chronic lymphocytic leukemia (CLL) or marginal zone lymphomas (MZL), primary mediastinal large b-cell lymphoma (PMBCL), and follicular lymphoma grade 3b (FL3B) after failure of 2 lines of therapy. Among the subjects treated were those having Eastern Cooperative Oncology Group (ECOG) scores of between 0 and 2 (median follow-up 3.2 months). No subjects were excluded based on prior allogeneic stem cell transplantation (SCT), secondary central nervous system (CNS) involvement or an ECOG score of 2, and there was no minimum absolute lymphocyte count (ALC) for apheresis required.

The therapeutic T cell compositions administered had been generated by a process including immunoaffinity-based (e.g., immunomagnetic selection) enrichment of CD4⁺ and CD8⁺ cells from leukapheresis samples from the individual subjects to be treated. Isolated CD4⁺ and CD8⁺ T cells were separately activated and independently transduced with a viral vector (e.g., lentiviral vector) encoding an anti-CD19 CAR, followed by separate expansion and cryopreservation of the engineered cell populations. The CAR contained an anti-CD19 scFv derived from a murine antibody (variable region derived from FMC63, V_{L}-linker-V_{H} orientation), an immunoglobulin-derived spacer, a transmembrane domain derived from CD28, a costimulatory region derived from 4-1BB, and a CD3-zeta intracellular signaling domain. The viral vector further contained sequences encoding a truncated receptor, which served as a surrogate marker for CAR expression and was separated from the CAR sequence by a T2A ribosome skip sequence.

The cryopreserved cell compositions were thawed prior to intravenous administration. The therapeutic T cell dose was administered as a defined cell composition by administering a formulated CD4⁺ CAR⁺ cell population and a formulated CD8⁺ CAR⁺ population administered at a target ratio of approximately 1:1. Subjects were administered a single or double dose of CAR-expressing T cells (each single dose via separate infusions of CD4⁺ CAR-expressing T cells and CD8⁺ CAR-expressing T cells, respectively) as follows: a single dose of dose level 1 (DL-1) containing 5×10⁷ total CAR-expressing T cells, a double dose of DL1 in which each dose was administered approximately fourteen (14) days apart (administered on day 1 and day 14), or a single dose of dose level 2 (DL-2) containing 1×10⁸ total CAR-expressing T cells. The target dose level and the numbers of T cell subsets for the administered compositions are set forth in Table E1.

TABLE E1
Target dose levels and number of T cell subsets for
cell compositions containing anti-CD19 CAR T cells

| | Helper T cell | Cytotoxic T | Total T |
|---|---|---|---|
| Dose | (T_{H}) Dose | Cell (T_{C}) Dose | Cell Dose |
| level | (CD8⁺CAR⁺) | (CD8⁺CAR⁺) | (CD3⁺ CAR⁺) |
| 1 | 25 × 10⁶ | 25 × 10⁶ | 50 × 10⁶ |
| 2 | 50 × 10⁶ | 50 × 10⁶ | 100 × 10⁶ |

Beginning at prior to CARP⁺ T cell infusion, subjects received a lymphodepleting chemotherapy with fludarabine (flu, 30 mg/m²) and cyclophosphamide (Cy, 300 mg/m²) for three (3) days. The subjects received CAR-expressing T cells 2-7 days after lymphodepletion.

After administration of the CAR T cell composition, subjects were monitored for clinical response, including at 3 months after administration, and response to the CAR T cell composition was determined by assessing whether the subject had progressive disease (PD), partial response (PR), or complete response (CR).

Tumor biopsies from a subset of patients (n=36) were collected prior to administration of the CAR T cells and analyzed by RNA sequencing (RNA-seq) for gene expression on the complementary DNA (cDNA) samples prepared from the RNA isolated from the tumor biopsies Principal component analysis (PCA) was performed for the RNA-seq data sets, generated from DESeq2-normalized counts. Expression of genes by RNA-Seq from the pretreatment tumor biopsies were correlated, post facto, to response following administration of the autologous therapeutic CAR-T cell composition.

Results in FIG. 7 are shown for 36 tumor biopsy samples that were analyzed among the subset of subjects in an ongoing clinical trial. A hypothetical threshold was set assuming, at 3 months following administration of the CD19-directed CAR T cell composition, 30% (11/36) of subjects will not respond to CAR-T cell treatment and 70% (25/36) of subjects will be able to respond; actual response results are shown. The results show a separation of gene expression between subjects who did not respond to treatment (subjects exhibited progressive disease (PD); designated resistant tumors) and subjects that were more responsive to treatment (designated sensitive tumors). Among samples from subjects with resistant tumors, an increased expression of genes associated with dysregulated cell cycle and cell proliferation pathways was observed. For example, higher expression of genes related to Myc targets, p53 signaling and EZH2 upregulated genes was observed. These results are consistent with a finding that upregulation of survival machinery, such as anti-apoptotic factors, in tumor cells may lead to tumor resistance and poor response to tumor-targeted immunotherapies.

### Example 6: Effects of Chronic BCL2 Inhibition on Anti-CD19 CAR T Cells Co-Cultured with Target Cells

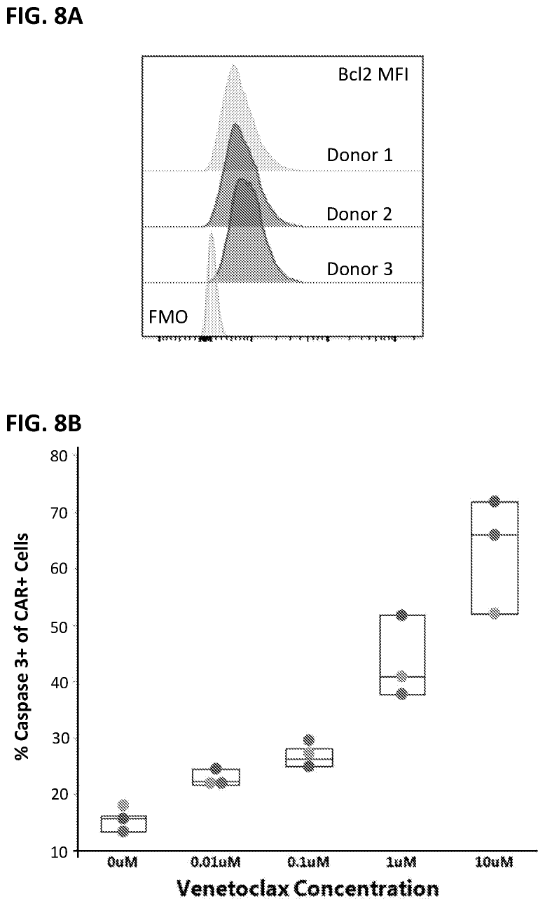

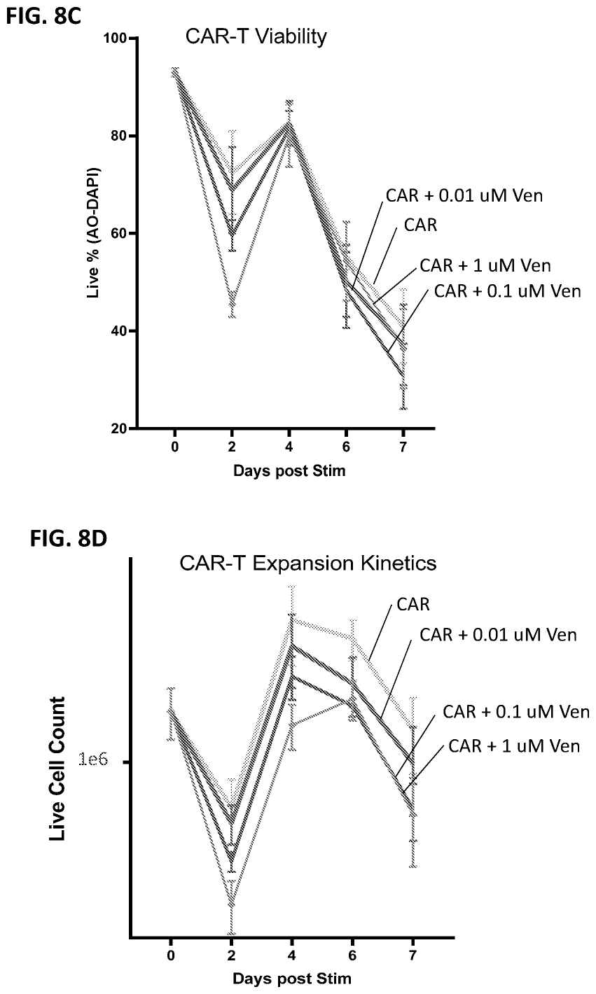

Anti-CD19 CAR-expressing T cell compositions were generated from three healthy human donors, substantially as described in Example 1. Expression of BCL2 by the CAR T cells was confirmed by flow cytometry and compared to a fluorescence minor one (FMO) control, as shown in FIG. 8A (mean fluorescence intensity; MFI).

Anti-CD19 CAR-expressing T cell compositions from healthy donors were incubated with anti-ID antibody-coated beads at 37° C. in the presence of increasing concentrations of venetoclax (0.01 μM, 0.1 μM, 1 μM, or 10 μM) for 48 hours. As a control, CART cells were incubated under the same conditions, but without venetoclax. Caspase 3 expression, viability, and expansion kinetics of the CAR T cells were subsequently assessed. As shown in FIG. 8B (each dot represents an individual donor), at 48 hours post-stimulation, increasing concentrations of venetoclax resulted in larger percentages of CAR+ T cells expressing caspase 3, indicative of cell death.

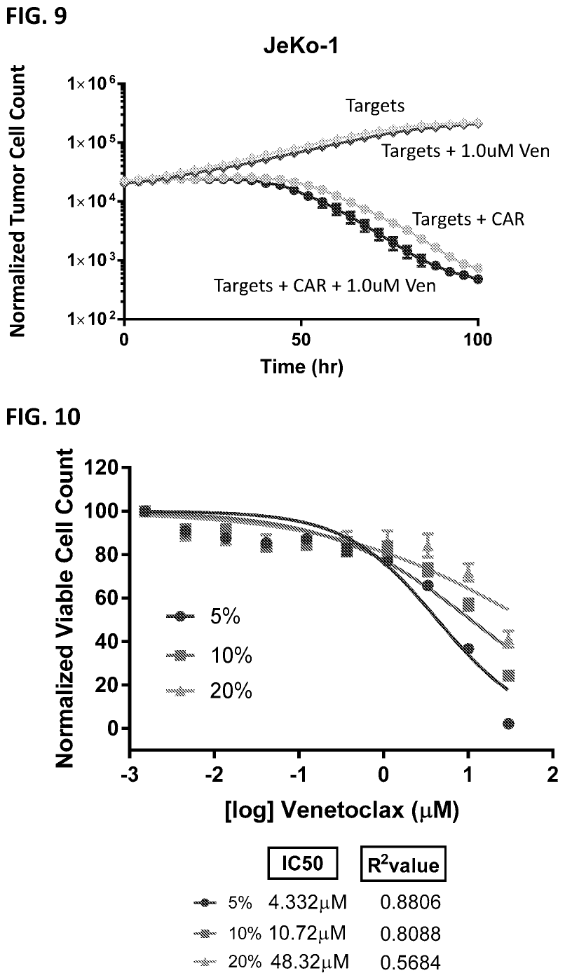

Following culture with the anti-ID beads, and optionally venetoclax, similar expansion profiles of CAR+ T cells were observed, regardless of venetoclax treatment or dose (FIGS. 8C and 8D). Further, the cytolytic activity of anti-CD19 CAR T cells was not affected when co-cultured with JeKo-1 target cells for 100 hours in the presence of 1.0 μM venetoclax. As shown in FIG. 9, the number of JeKo-1 target cells was not reduced by venetoclax treatment alone, but was substantially decreased when co-cultured with CD19-targeting CAR T cells in the presence or absence of venetoclax. Without wishing to be bound by theory, the data indicate that venetoclax may affect the cell viability of anti-CD19 CAR T cells, but that the viability, expansion kinetics, and cytolytic activity of viable CAR T cells are unaffected.

Venetoclax can bind human plasma proteins. Thus, to determine whether serum concentration may modulate the effects of venetoclax on CAR T cells, anti-CD19 CAR T cells were cultured in the presence of 5%, 10%, or 20% serum and treated with increasing concentrations of venetoclax. The IC50 of venetoclax against anti-CD19 CAR T cells was found to increase with increasing concentrations of serum (FIG. 10), indicating that the effects of venetoclax on CAR T cell viability may depend on serum concentration.

### Example 7: Effects of a BCL-2 Inhibitor on Anti-CD19 CAR T Cells in a Murine Model of Mantle Cell Lymphoma

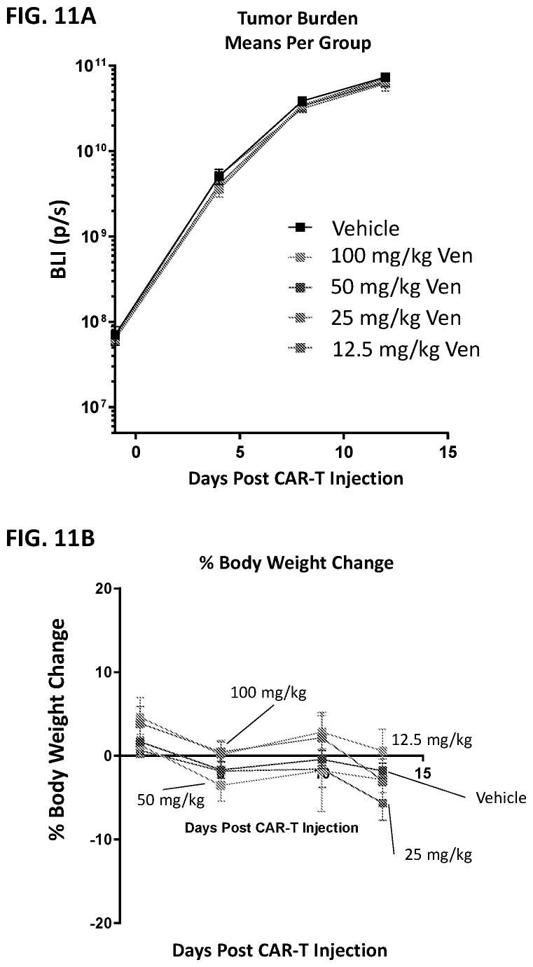

NOD scid gamma (NSG) mice were injected intravenously with 2×10⁶ JeKo-1 mantle cell lymphoma cells (Day −7) and treated daily with 12.5, 25, 50, or 100 mg/kg of the exemplary BCL2 inhibitor venetoclax for 21 days (Day 0 through Day 21). Tumor burden (FIG. 11A) and body weight (FIG. 11B) were assessed over the course of treatment.

Consistent with the results in Example 7, the JeKo-1 lymphoma cell line was observed to be resistant to treatment with venetoclax alone, regardless of dose level (FIG. 11A showing tumor burden, as assessed by bioluminescence imaging; BLI).

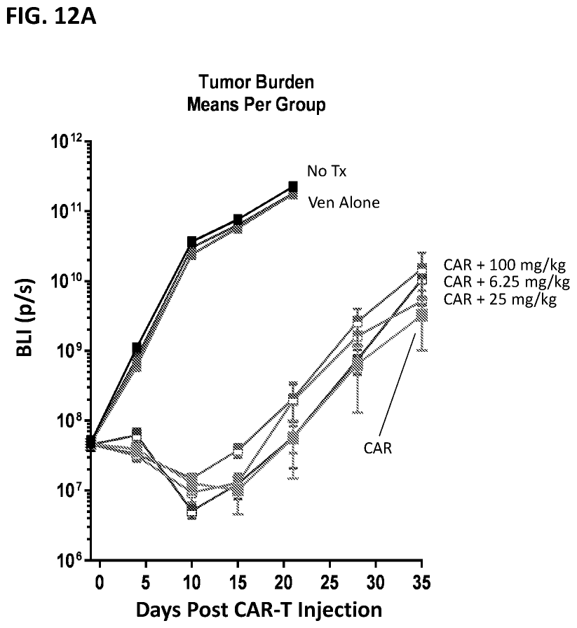

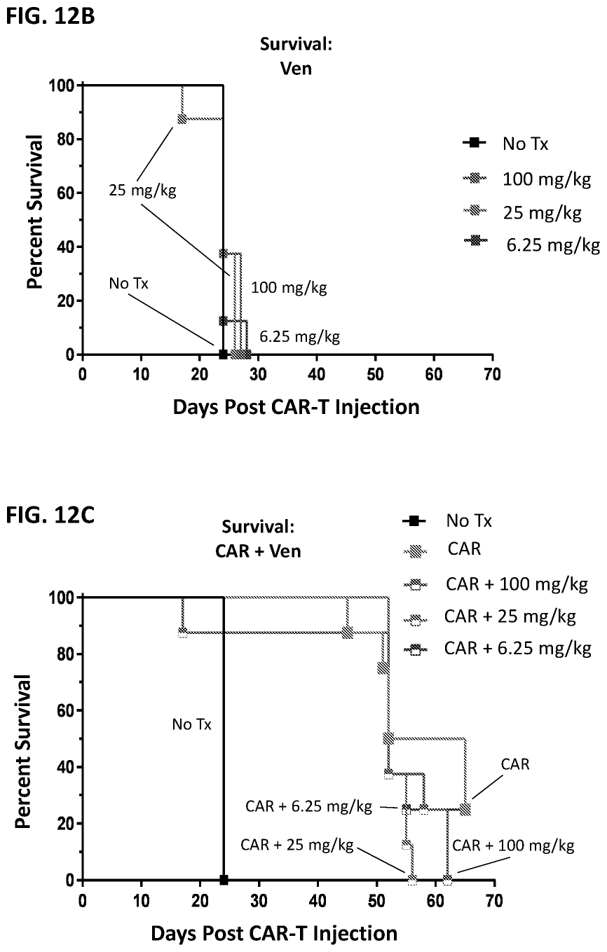

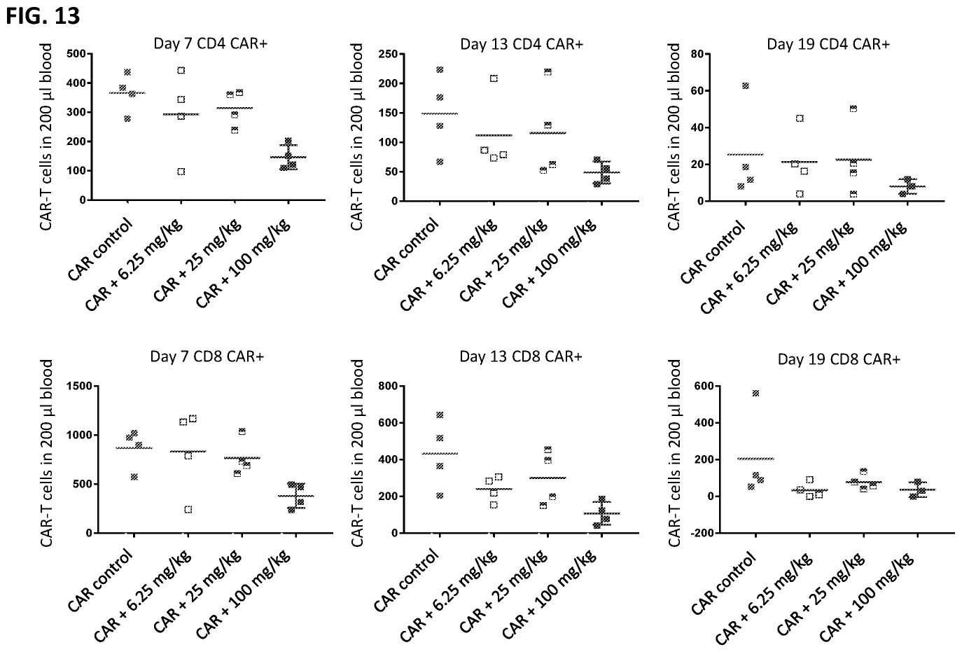

To determine the effects of venetoclax on anti-CD19 CAR T cells in a venetoclax-resistant murine model of MCL, NSG mice were injected intravenously with 2×10⁶ JeKo-1 cells on Day −7, and then treated with intravenous infusions of 1×10⁶ anti-CD19 CAR T cells, generated substantially as described in previous Example, on Day −1 and Day 0. Venetoclax was administered daily for 21 days (Day 0 to Day 21) at 6.25, 25, or 100 mg/kg. As a control, a subset of mice were only treated with venetoclax. Tumor burden (FIG. 12A), survival (FIGS. 12B and 12C), and CAR T cell number (FIG. 13) were assessed.

Tumor burden increased similarly in untreated mice and mice treated with venetoclax alone. However, treatment with CD19-targeting CAR T cells, alone or in combination with venetoclax, resulted in reduced tumor volume at all measured time points, as compared to untreated and venetoclax-only treated mice (FIG. 12A). Similar effects on survival were observed, as shown in FIGS. 12B and 12C.

The number of CD4+ and CD8+ CAR T cells was assessed in mice treated with CD19-targeting CAR T cells, alone or in combination with venetoclax, on days 7, 13, and 19. Dose-wise decreases in both CD4+ and CD8+ CAR T cell subsets were observed with increasing concentrations of venetoclax (FIG. 13).

Together, these data indicate that the cytolytic ability of the CD19-targeting CAR T may not be compromised by the presence of the exemplary BCL2 inhibitor venetoclax, despite losses in CAR T cell viability observed with higher doses of the inhibitor.

### Example 8: Delayed Administration of a BCL2 Inhibitor Following Treatment with Anti-CD19 CAR T Cells

RL target cells were co-cultured with CD19-targeting CAR T cells, generated substantially as described in Example 1, for 6 days in the absence or presence of the exemplary BCL2 inhibitor venetoclax (0.01 μM, 0.1 μM, or 1.0 μM). Venetoclax was added concurrently with co-culture or 24, 48, or 72 hours after the start of co-culture. As a control, RL target cells were cultured in the presence of venetoclax, but without CAR T cells.

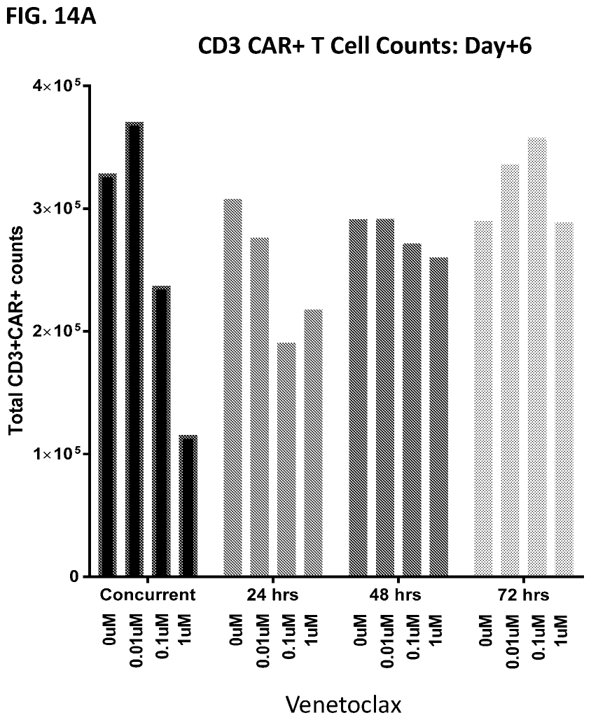

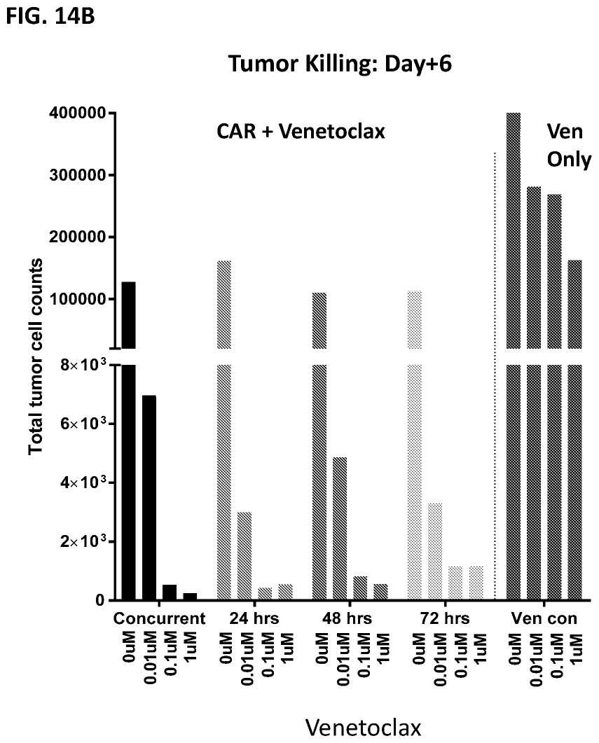

After 6 days of co-culture, the number of CD3+ CAR T cells and target cells was assessed. As shown in FIG. 14A, the number of CAR T cells was reduced when 0.1 μM or 1.0 μM of venetoclax was added concurrently with co-culture or 24 hours later. By contrast, a reduction in CD3+ CAR T cells was not observed when venetoclax was added 48 or 72 hours following co-culture, regardless of the concentration of venetoclax. For all time points assessed, 0.01 μM of venetoclax was not observed to reduce CD3+ CAR T cell number. As shown in FIG. 14B, similar decreases in target cell number were observed for all conditions, except for target cells cultured only in the presence of venetoclax.

These results indicate that delaying treatment with a BCL2 inhibitor may reduce effects of the inhibitor on CAR T cell viability, while preserving CAR T cell cytolytic activity.

### Example 9: Treatment of Chronic Lymphocytic Leukemia and Small Lymphocytic Lymphoma with CD19-Targeting CAR T Cells and Venetoclax

Therapeutic CAR T cell compositions containing autologous T cells expressing a chimeric antigen-receptor (CAR) specific for CD19 and venetoclax are administered to subjects with B cell malignancies as a combination therapy.

Specifically, autologous anti-CD19 directed therapeutic T cell compositions are generated from and used to treat adult human subjects with chronic lymphocytic leukemia (CLL) and small lymphocytic lymphoma (SLL) who have failed two or more prior lines of therapy. Treated subjects have been previously treated with an inhibitor of BTK (e.g. ibrutinib), and either have not been previously treated with venetoclax (venetoclax naïve) or have been previously treated with venetoclax. For subjects previously treated with venetoclax, the subjects are not venetoclax-intolerant and have not exhibited progressive disease (PD) while being treated with venetoclax within the previous 6 months. Eligible subjects must exhibit measurable disease (lymph nodes >1.5 cm in greater transverse diameter; GTD) or evaluable or measurable disease in peripheral blood (PB), bone marrow (BM), liver, or spleen, and must have minimum residual disease (MRD) in PB of greater than or equal to 10⁻⁴. Subjects are screened at baseline for expression of BCL-2 family proteins, and subjects who have been previously treated with venetoclax are additionally screened for BCL2 mutations. Subjects are not excluded on the basis of BCL2 mutation status.

T cell compositions enriched for CD4⁺ and/or CD8⁺ cells from leukapheresis samples from individual subjects to be treated are administered to the subjects. The compositions enriched for CD4+ and/or CD8+ T cells are activated and transduced with a viral vector (e.g., lentiviral vector) encoding an anti-CD19 CAR, followed by expansion and cryopreservation of the engineered cell populations. The CAR contains an anti-CD19 scFv (e.g. containing a variable region derived from the murine antibody FMC63), a spacer linking the antigen-binding domain to a transmembrane domain, the transmembrane domain (e.g. derived from CD28), a costimulatory region (e.g. derived from 4-1BB), and a CD3-zeta intracellular signaling domain.

The cryopreserved cell compositions are thawed prior to intravenous administration. Subjects are administered a dose of 1×10⁸ total CAR-expressing T cells (e.g., via separate infusions of CD4⁺ CAR-expressing T cells and CD8⁺ CAR-expressing T cells, optionally provided at a 1:1 ratio of CD4+ to CD8+ cells).

Prior to CAR⁺ T cell infusion, leukapheresis samples are obtained from subjects. Subjects are then administered venetoclax bridging therapy for three weeks prior to lymphodepleting therapy. As bridging therapy, subjects are administered 20 milligrams (mg) of venetoclax daily for the first week, 50 mg of venetoclax daily for the second week, and 100 mg of venetoclax daily for the third week. Following venetoclax washout (e.g. 1-7 days), subjects receive a lymphodepleting chemotherapy with fludarabine (Flu, 30 mg/m²) and cyclophosphamide (Cy, 300 mg/m²) for 3 days. The subjects receive the dose of CAR-expressing T cells (e.g. 1×10⁸ total CAR-expressing T cells) 2-7 days after lymphodepletion, on Day 0. On Day 7, venetoclax administration is initiated. Venetoclax is dosed at 200 mg daily until about 3 months after initiation of administration of the CAR+ T cells, e.g. venetoclax is administered starting at Day 7 and once daily for a total of eleven weeks of treatment with the inhibitor.

Subjects are monitored for clinical response, and response to the treatment is determined by assessing whether the subject exhibits progressive disease (PD), partial response (PR), or complete response (CR). Subjects who have minimum residual disease (MRD) (≥10⁴ in PB) or who do not exhibit complete response (CR) by the end of the treatment, e.g. by Day 84, may continue treatment with venetoclax per standard of care, such as for a total treatment duration of 12 or 24 months.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| # | SEQUENCE | ANNOTATION |
|---|---|---|
| 1 | ESKYGPPCPPCP | spacer |
| | | (IgG4hinge) (aa) |
| 2 | GAATCTAAGTACGGACCGCCCTGCCCCCCTTGCCCT | spacer |
| | | (IgG4hinge) (nt) |
| 3 | ESKYGPPCPPCPGQPREPQVYTLPPSQEEMTKNQVSLTCLVKGFYPSDIAV | Hinge-CH3 |
| | EWESNGQPENNYKTTPPVLDSDGSFFLYSRLTVDKSRWQEGNVFSCSVMHE | spacer |
| | ALHNHYTQKSLSLSLGK | |
| 4 | ESKYGPPCPPCPAPEFLGGPSVFLFPPKPKDTLMISRTPEVTCVVVDVSQE | Hinge-CH2-CH3 |
| | DPEVQFNWYVDGVEVHNAKTKPREEQFNSTYRVVSVLTVLHQDWLNGKEYK | spacer |
| | CKVSNKGLPSSIEKTISKAKGQPREPQVYTLPPSQEEMTKNQVSLTCLVKG | |
| | FYPSDIAVEWESNGQPENNYKTTPPVLDSDGSFFLYSRLTVDKSRWQEGNV | |
| | FSCSVMHEALHNHYTQKSLSLSLGK | |
| 5 | RWPESPKAQASSVPTAQPQAEGSLAKATTAPATTRNTGRGGEEKKKEKEKE | IgD-hinge-Fc |
| | EQEERETKTPECPSHTQPLGVYLLTPAVQDLWLRDKATFTCFVVGSDLKDA | |
| | HLTWEVAGKVPTGGVEEGLLERHSNGSQSQHSRLTLPRSLWNAGTSVTCTL | |
| | NHPSLPPQRLMALREPAAQAPVKLSLNLLASSDPPEAASWLLCEVSGFSPP | |
| | NILLMWLEDQREVNTSGFAPARPPPQPGSTTFWAWSVLRVPAPPSPQPATY | |
| | TCVVSHEDSRTLLNASRSLEVSYVTDH | |
| 6 | LEGGGEGRGSLLTCGDVEENPGPR | T2A |
| 7 | MLLLVTSLLLCELPHPAFLLIPRKVCNGIGIGEFKDSLSINATNIKHFKNC | tEGFR |
| | TSISGDLHILPVAFRGDSFTHTPPLDPQELDILKTVKEITGFLLIQAWPEN | |
| | RTDLHAFENLEIIRGRTKQHGQFSLAVVSLNITSLGLRSLKEISDGDVIIS | |
| | GNKNLCYANTINWKKLFGTSGQKTKIISNRGENSCKATGQVCHALCSPEGC | |
| | WGPEPRDCVSCRNVSRGRECVDKCNLLEGEPREFVENSECIQCHPECLPQA | |
| | MNITCTGRGPDNCIQCAHYIDGPHCVKTCPAGVMGENNTLVWKYADAGHVC | |
| | HLCHPNCTYGCTGPGLEGCPTNGPKIPSIATGMVGALLLLLVVALGIGLFM | |
| 8 | FWVLVVVGGVLACYSLLVTVAFIIFWV | CD28 (amino |
| | | acids 153-179 of |
| | | Accession No. |
| | | P10747) |
| 9 | IEVMYPPPYLDNEKSNGTIIHVKGKHLCPSPLFPGPSKP | CD28 (amino |
| | FWVLVVVGGVLACYSLLVTVAFIIFWV | acids 114-179 of |
| | | Accession No. |
| | | P10747) |
| 10 | RSKRSRLLHSDYMNMTPRRPGPTRKHYQPYAPPRDFAAYRS | CD28 (amino |
| | | acids 180-220 of |
| | | P10747) |
| 11 | RSKRSRGGHSDYMNMTPRRPGPTRKHYQPYAPPRDFAAYRS | CD28 (LL to GG) |
| 12 | KRGRKKLLYIFKQPFMRPVQTTQEEDGCSCRFPEEEEGGCEL | 4-1BB (amino |
| | | acids 214-255 of |
| | | Q07011.1) |
| 13 | RVKFSRSADAPAYQQGQNQLYNELNLGRREEYDVLDKRRGRDPEMGGKPRR | CD3 zeta |
| | KNPQEGLYNELQKDKMAEAYSEIGMKGERRRGKGHDGLYQGLSTATKDTYD | |
| | ALHMQALPPR | |
| 14 | RVKFSRSAEPPAYQQGQNQLYNELNLGRREEYDVLDKRRGRDPEMGGKPRR | CD3 zeta |
| | KNPQEGLYNELQKDKMAEAYSEIGMKGERRRGKGHDGLYQGLSTATKDTYD | |
| | ALHMQALPPR | |
| 15 | RVKFSRSADAPAYKQGQNQLYNELNLGRREEYDVLDKRRGRDPEMGGKPRR | CD3 zeta |
| | KNPQEGLYNELQKDKMAEAYSEIGMKGERRRGKGHDGLYQGLSTATKDTYD | |
| | ALHMQALPPR | |
| 16 | RKVCNGIGIGEFKDSLSINATNIKHFKNCTSISGDLHILPVAFRGDSFTHT | tEGFR |
| | PPLDPQELDILKTVKEITGFLLIQAWPENRTDLHAFENLEIIRGRTKQHGQ | |
| | FSLAVVSLNITSLGLRSLKEISDGDVIISGNKNLCYANTINWKKLFGTSGQ | |
| | KTKIISNRGENSCKATGQVCHALCSPEGCWGPEPRDCVSCRNVSRGRECVD | |
| | KCNLLEGEPREFVENSECIQCHPECLPQAMNITCTGRGPDNCIQCAHYIDG | |
| | PHCVKTCPAGVMGENNTLVWKYADAGHVCHLCHPNCTYGCTGPGLEGCPTN | |
| | GPKIPSIATGMVGALLLLLVVALGIGLFM | |
| 17 | EGRGSLLTCGDVEENPGP | T2A |
| 18 | GSGATNFSLLKQAGDVEENPGP | P2A |
| 19 | ATNFSLLKQAGDVEENPGP | P2A |
| 20 | QCTNYALLKLAGDVESNPGP | E2A |
| 21 | VKQTLNFDLLKLAGDVESNPGP | F2A |
| 22 | —PGGG—(SGGGG)₅—P— wherein P is proline, G is | Linker |
| | glycine and S is serine | |
| 23 | GSADDAKKDAAKKDGKS | Linker |
| 24 | atgcttctcctggtgacaagccttctgctctgtgagttaccacacccagca | GMCSFR alpha |
| | ttcctcctgatccca | chain signal |
| | | sequence |
| 25 | MLLLVTSLLLCELPHPAFLLIP | GMCSFR alpha |
| | | chain signal |
| | | sequence |
| 26 | MALPVTALLLPLALLLHA | CD8 alpha signal |
| | | peptide |
| 27 | Glu Pro Lys Ser Cys Asp Lys Thr His Thr Cys Pro Pro | Hinge |
| | Cys Pro | |
| 28 | Glu Arg Lys Cys Cys Val Glu Cys Pro Pro Cys Pro | Hinge |
| 29 | ELKTPLGDTHTCPRCPEPKSCDTPPPCPRCPEPKSCDTPPPCPRCPEPKSC | Hinge |
| | DTPPPCPRCP | |
| 30 | Glu Ser Lys Tyr Gly Pro Pro Cys Pro Ser Cys Pro | Hinge |
| 31 | Glu Ser Lys Tyr Gly Pro Pro Cys Pro Pro Cys Pro | Hinge |
| 32 | Tyr Gly Pro Pro Cys Pro Pro Cys Pro | Hinge |
| 33 | Lys Tyr Gly Pro Pro Cys Pro Pro Cys Pro | Hinge |
| 34 | Glu Val Val Val Lys Tyr Gly Pro Pro Cys Pro Pro Cys | Hinge |
| | Pro | |
| 35 | RASQDISKYLN | CDR L1 |
| 36 | SRLHSGV | CDR L2 |
| 37 | GNTLPYTFG | CDR L3 |
| 38 | DYGVS | CDR H1 |
| 39 | VIWGSETTYYNSALKS | CDR H2 |
| 40 | YAMDYWG | CDR H3 |
| 41 | EVKLQESGPGLVAPSQSLSVTCTVSGVSLPDYGVSWIRQPPRKGLEWLGVI | VH |
| | WGSETTYYNSALKSRLTIIKDNSKSQVFLKMNSLQTDDTAIYYCAKHYYYG | |
| | GSYAMDYWGQGTSVTVSS | |
| 42 | DIQMTQTTSSLSASLGDRVTISCRASQDISKYLNWYQQKPDGTVKLLIYHT | VL |
| | SRLHSGVPSRFSGSGSGTDYSLTISNLEQEDIATYFCQQGNTLPYTFGGGT | |
| | KLEIT | |
| 43 | DIQMTQTTSSLSASLGDRVTISCRASQDISKYLNWYQQKPDGTVKLLIYHT | scFv |
| | SRLHSGVPSRFSGSGSGTDYSLTISNLEQEDIATYFCQQGNTLPYTFGGGT | |
| | KLEITGSTSGSGKPGSGEGSTKGEVKLQESGPGLVAPSQSLSVTCTVSGVS | |
| | LPDYGVSWIRQPPRKGLEWLGVIWGSETTYYNSALKSRLTIIKDNSKSQVF | |
| | LKMNSLQTDDTAIYYCAKHYYYGGSYAMDYWGQGTSVTVSS | |
| 44 | KASQNVGTNVA | CDR L1 |
| 45 | SATYRNS | CDR L2 |
| 46 | QQYNRYPYT | CDR L3 |
| 47 | SYWMN | CDR H1 |
| 48 | QIYPGDGDTNYNGKFKG | CDR H2 |
| 49 | KTISSVVDFYFDY | CDR H3 |
| 50 | EVKLQQSGAELVRPGSSVKISCKASGYAFSSYWMNWVKQRPGQGLEWIGQI | VH |
| | YPGDGDTNYNGKFKGQATLTADKSSSTAYMQLSGLTSEDSAVYFCARKTIS | |
| | SVVDFYFDYWGQGTTVTVSS | |
| 51 | DIELTQSPKFMSTSVGDRVSVTCKASQNVGTNVAWYQQKPGQSPKPLIYSA | VL |
| | TYRNSGVPDRFTGSGSGTDFTLTITNVQSKDLADYFCQQYNRYPYTSGGGT | |
| | KLEIKR | |
| 52 | GGGGSGGGGSGGGGS | Linker |
| 53 | EVKLQQSGAELVRPGSSVKISCKASGYAFSSYWMNWVKQRPGQGLEWIGQI | scFv |
| | YPGDGDTNYNGKFKGQATLTADKSSSTAYMQLSGLTSEDSAVYFCARKTIS | |
| | SVVDFYFDYWGQGTTVTVSSGGGGSGGGGSGGGGSDIELTQSPKFMSTSVG | |
| | DRVSVTCKASQNVGTNVAWYQQKPGQSPKPLIYSATYRNSGVPDRFTGSGS | |
| | GTDFTLTITNVQSKDLADYFCQQYNRYPYTSGGGTKLEIKR | |
| 54 | HYYYGGSYAMDY | HC-CDR3 |
| 55 | HTSRLHS | LC-CDR2 |
| 56 | QQGNTLPYT | LC-CDR3 |
| 57 | gacatccagatgacccagaccacctccagcctgagcgccagcctgggcgac | Sequence |
| | cgggtgaccatcagctgccgggccagccaggacatcagcaagtacctgaac | encoding scFv |
| | tggtatcagcagaagcccgacggcaccgtcaagctgctgatctaccacacc | |
| | agccggctgcacagcggcgtgcccagccggtttagcggcagcggctccggc | |
| | accgactacagcctgaccatctccaacctggaacaggaagatatcgccacc | |
| | tacttttgccagcagggcaacacactgccctacacctttggcggcggaaca | |
| | aagctggaaatcaccggcagcacctccggcagcggcaagcctggcagcggc | |
| | gagggcagcaccaagggcgaggtgaagctgcaggaaagcggccctggcctg | |
| | gtggcccccagccagagcctgagcgtgacctgcaccgtgagcggcgtgagc | |
| | ctgcccgactacggcgtgagctggatccggcagccccccaggaagggcctg | |
| | gaatggctgggcgtgatctggggcagcgagaccacctactacaacagcgcc | |
| | ctgaagagccggctgaccatcatcaaggacaacagcaagagccaggtgttc | |
| | ctgaagatgaacagcctgcagaccgacgacaccgccatctactactgcgcc | |
| | aagcactactactacggcggcagctacgccatggactactggggccagggc | |
| | accagcgtgaccgtgagcagc | |
| 58 | X₁PPX₂P | Hinge |
| | X₁ is glycine, cysteine or arginine | |
| | X₂ is cysteine or threonine | |
| 59 | GSTSGSGKPGSGEGSTKG | Linker |

What is claimed:

1. A method of treating cancer, the method comprising:
(1) administering a cytotoxic therapy to a subject having a cancer, wherein the cytotoxic therapy is a T cell therapy comprising or enriched in T cells and specifically binds to an antigen associated with, expressed by, or present on cells of the cancer; and
(2) administering to the subject an inhibitor of a prosurvival BCL2 family protein in a dosing regimen comprising initiation of administration of the inhibitor at a time between at or about 1 day prior and at or about 8 days after initiation of administration of the cytotoxic therapy.

2. A method of treating cancer, the method comprising administering to a subject having a cancer an inhibitor of a prosurvival BCL2 family protein, wherein the inhibitor is administered in a dosing regimen comprising initiation of administration of the inhibitor at a time between at or about 1 day prior to and at or about 8 days after initiation of administration of a cytotoxic therapy, wherein the cytotoxic therapy is a T cell therapy comprising or enriched in T cells and specifically binds to an antigen associated with, expressed by, or present on cells of the cancer.

3. A method of treating a cancer in a subject, the method comprising administering a cytotoxic therapy to a subject having a cancer, wherein the cytotoxic therapy is a T cell therapy comprising or enriched in T cells and specifically binds to an antigen associated with, expressed by, or present on cells of the cancer, wherein the subject is administered or is to be administered an inhibitor of a prosurvival BCL2 family protein for a period of time in a dosing regimen comprising initiation of administration of the inhibitor at a time between at or about 1 day prior to and at or about 8 days after initiation of administration of the cytotoxic therapy.

4. The method of any of claims 1-3, wherein the dosing regimen of the inhibitor comprises initiation of administration of the inhibitor after administration of the cytotoxic therapy.

5. The method of any of claims 1-4, wherein the dosing regimen of the inhibitor comprises initiation of administration of the inhibitor within 7 days after initiation of administration of the cytotoxic therapy.

6. The method of any of claims 1-4, wherein the dosing regimen of the inhibitor comprises initiation of administration of the inhibitor within 3 days after initiation of administration of the cytotoxic therapy.

7. The method of any of claims 1-6, wherein the initiation of administration of the inhibitor is no more than 2 days after initiation of administration of the cytotoxic therapy, optionally wherein the initiation of administration of the inhibitor is within 1 day after the initiation of administration of the cytotoxic therapy.

8. The method of any of claims 1-7, wherein the dosing regimen of the inhibitor comprises initiation of administration of the inhibitor at or after activation-induced cell death (AICD) of the cells of the cell therapy has peaked following initiation of administration of the cell therapy.

9. The method of any of claims 1-3, wherein at least one dose of the inhibitor in the dosing regimen is administered concurrently with the cytotoxic therapy and/or on the same day as the cytotoxic therapy.

10. The method of any of claims 1-9, wherein the subject is not administered or has not received administration of rituximab and/or ibrutinib within 7 days prior to the initiation of administration of the cytotoxic therapy.

11. The method of any of claims 1-10, wherein the cytotoxic therapy is capable of or results in cell-mediated cytotoxicity of one of more of cells of the cancer.

12. The method of any of claims 1-11, wherein the cytotoxic therapy is capable of or mediates perforin- and/or granzyme-mediated apoptosis of one or more cells of the cancer.

13. The method of any of claims 1-12, wherein the cell therapy comprises cells that are autologous to the subject.

14. The method of any of claims 1-13, wherein the cell therapy is selected from among the group consisting of a tumor infiltrating lymphocytic (TIL) therapy, a transgenic TCR therapy, and a chimeric antigen receptor (CAR)-expressing cell therapy.

15. The method of any of claims 1-14, wherein the cell therapy comprises a dose of cells expressing a recombinant receptor that specifically binds to the antigen.

16. The method of any of claims 1-15, wherein the administration of the cytotoxic therapy comprises administration of between at or about 1×10⁵ and at or about 5×10⁸ total recombinant receptor-expressing T cells or total T cells, between at or about 1×10⁵ and at or about 1×10⁸ total recombinant receptor-expressing T cells or total T cells, between at or about 5×10⁵ and at or about 1×10⁷ total recombinant receptor-expressing T cells or total T cells, or between at or about 1×10⁶ and at or about 1×10⁷ total recombinant receptor-expressing T cells or total T cells, each inclusive.

17. The method of any of claims 1-16, wherein the cell therapy comprises or is enriched in CD3+, CD4+, CD8+, or CD4+ and CD8+ T cells.

18. The method of any of claims 1-17, wherein the cell therapy comprises or is enriched in CD4+ and CD8+ T cells.

19. The method of claim 19, wherein the CD4+ and CD8+ T cells of the cell therapy comprises a defined ratio of CD4+ recombinant receptor-expressing T cells to CD8+ recombinant receptor-expressing T cells that is or is approximately 1:1 or is between approximately 1:3 and approximately 3:1.

20. The method of any of claims 1-19, wherein the cell therapy comprises administering CD4⁺ and CD8⁺ T cells, wherein T cells of each dose comprises a recombinant receptor, optionally a CAR, that specifically binds to the antigen, wherein the administration comprises administering a plurality of separate compositions, the plurality of separate compositions comprising a first composition comprising or enriched in the CD8⁺ T cells and a second composition comprising or enriched in the CD4⁺ T cells.

21. The method of claim 20, wherein:
the CD4+ T cells comprising the recombinant receptor in the one of the first and second compositions and the CD8+ T cells comprising the recombinant receptor in the other of the first and second compositions are present at a defined ratio that is or is approximately 1:1 or is between approximately 1:3 and approximately 3:1; and/or
the CD4+ T cells comprising the recombinant receptor and the CD8+ T cells comprising the recombinant receptor administered in the first and second compositions are present at a defined ratio, which ratio is or is approximately 1:1 or is between approximately 1:3 and approximately 3:1.

22. The method of any of claims 15-21, wherein the recombinant receptor is a T cell receptor (TCR) or a functional non-T cell receptor.

23. The method of any of claims 15-22, wherein the recombinant receptor is a chimeric antigen receptor (CAR).

24. The method of claim 23, wherein the cell therapy comprises administration of from or from about 1×10⁵ to 5×10⁸ total CAR-expressing T cells, 1×10⁶ to 2.5×10⁸ total CAR-expressing T cells, 5×10⁶ to 1×10⁸ total CAR-expressing T cells, 1×10⁷ to 2.5×10⁸ total CAR-expressing T cells, 5×10⁷ to 1×10⁸ total CAR-expressing T cells, each inclusive.

25. The method of claim 23 or claim 24, wherein the cell therapy comprises administration of at or about 1×10⁸ CAR-expressing cells.

26. The method of any of claims 1-25, wherein the antigen is a tumor antigen.

27. The method of any of claims 1-26, wherein the antigen is selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha(IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1).

28. The method of any of claims 1-26, wherein the antigen is associated with a B cell malignancy, optionally wherein the antigen is expressed on human B cells, optionally wherein the antigen is CD20, CD19, CD22, ROR1, CD45, CD21, CD5, CD33, Igkappa, Iglambda, CD79a, CD79b or CD30.

29. The method of any of claims 1-29, wherein the antigen is CD19.

30. The method of any of claims 23-29, wherein the CAR comprises an extracelluar antigen binding domain that binds to the antigen, a transmembrane domain, and an intracellular signaling region comprising an intracellular signaling domain of a CD3-zeta (CD3) chain and a costimulatory signaling domain.

31. The method of claim 30, wherein the costimulatory signaling region comprises a signaling domain of 4-1BB.

32. The method of claim 31, wherein the costimulatory signaling region comprises a signaling domain of CD28.

33. The method of any of claims 1-32, wherein the method comprises, prior to administration of the cytotoxic therapy, administering a lymphodepleting agent or therapy to the subject.

34. The method of claim 33, wherein the lymphodepleting therapy is completed between 2 and 7 days before the initiation of administration of the cytotoxic therapy.

35. The method of claim 33 or claim 34, wherein the lymphodepleting therapy comprises the administration of fludarabine and/or cyclophosphamide.

36. The method of any of claims 33-35, wherein the lymphodepleting therapy comprises administration of cyclophosphamide at about 200-400 mg/m², optionally at or about 300 mg/m², inclusive, and/or fludarabine at about 20-40 mg/m², optionally 30 mg/m², daily for 2-4 days, optionally for 3 days; or wherein the lymphodepleting therapy comprises administration of cyclophosphamide at about 500 mg/m².

37. The method of any one of claims 32-36, wherein:
the lymphodepleting therapy comprises administration of cyclophosphamide at or about 300 mg/m² and fludarabine at about 30 mg/m² daily for 3 days; and/or
the lymphodepleting therapy comprises administration of cyclophosphamide at or about 500 mg/m² and fludarabine at about 30 mg/m² daily for 3 days.

38. The method of any of claims 1-537 wherein:
the dosing regimen of the inhibitor comprises a subtherapeutic amount of the inhibitor;
the dosing regimen of the inhibitor is not sufficient to reduce tumor burden in the subject or reduces tumor burden by less than 10% when administered as a monotherapy in the absence of the combined administration with the cytotoxic therapy; and/or
the dosing regimen of the inhibitor does not result in a complete or partial response in a group of similarly treated subjects, or results in such a response in no more than 10% of such subjects, when administered as a monotherapy in the absence of the combined administration with the cytotoxic therapy.

39. The method of any of claims 1-38, wherein the dosing regimen of the inhibitor comprises once daily dosing.

40. The method of claim 39, wherein the once daily dose is between at or about 20 mg and at or 400 mg, inclusive.

41. The method of claim 39 or claim 40, wherein the once daily dose is between at or about 20 mg and at or about 200 mg, inclusive.

42. The method of any of claims 39-41, wherein the once daily dose is an amount of the inhibitor of between at or about 50 mg and at or about 100 mg, inclusive.

43. The method of any of claims 39-42, wherein the once daily dose is at or about 50 mg.

44. The method of any of claims 39-43, wherein the once daily dose is at or about 100 mg.

45. The method of any of claims 39-41, wherein the once daily dose is at or about 200 mg.

46. The method of claim 39 or claim 45, wherein the once daily dose is at or about 400 mg.

47. The method of any of claims 1-46, wherein, prior to administration of the dosing regimen of the inhibitor, the subject has been previously treated with an inhibitor of a prosurvival Bcl-2 family protein, optionally wherein the subject has been previously treated with venetoclax.

48. The method of claim 47, wherein the previous treatment with the inhibitor is administered at a time between the collecting of the autologous cells from the subject and prior to administering a lymphodepleting therapy to the subject, as a bridging therapy prior to the administration of the cytotoxic therapy, optionally wherein the collecting is by apheresis or leukapheresis.

49. The method of claim 48, wherein the previous treatment of the inhibitor is administered in a dose-ramp up schedule, wherein the dose-ramp up schedule comprises administration of escalating doses of the inhibitor.

50. The method of claim 48 or claim 49, wherein the inhibitor is administered to the subject in escalating doses until a maximum dose of 100 mg daily is reached.

51. The method of any of claims 48-50, wherein the escalating doses comprise a first dose that is at about 20 mg per day, a second dose that is at about 50 mg per day, and a third dose that is at about 100 mg per day.

52. The method of any of claims 48-51, wherein each escalating dose is administered once daily for a week and/or the last escalating dose is administered once daily for a week or until the end of the bridging therapy.

53. The method of any of claims 48-52, wherein the previous treatment with the inhibitor is ceased at least 1 day prior to administration of the lymphodepleting therapy.

54. The method of any of claims 48-53, wherein the previous treatment with the inhibitor is ceased: for at least at or about 3 days or for at least at or about 4 days prior to administration of the lymphodepleting therapy; for at least an amount of time until the concentration of the inhibitor in the subject's bloodstream is reduced by about three half-lives or about four half-lives; or for at least an amount of time until the inhibitor is eliminated from the bloodstream of the subject.

55. The method of any of claims 1-54, wherein the inhibitor inhibits one or more prosurvival BCL2 family protein selected from among the group consisting of BCL2, BCLXL, BCLW, BCLB, MCL1, and combinations thereof.

56. The method of any of claims 1-55, wherein the one or more prosurvival BCL2 family protein is BCL2, BCLXL, and/or BCLW.

57. The method of any of claims 1-56, wherein the inhibitor is selected from among the group consisting of venetoclax, navitoclax, ABT737, maritoclax, obatoclax, and clitocine.

58. The method of any of claims 1-56, wherein the inhibitor is venetoclax.

59. The method of any of claims 1-58, wherein the cancer is a hematological malignancy.

60. The method of any of claims 1-59, wherein the cancer is a B cell malignancy.

61. The method of any of claims 1-60, wherein the cancer is a myeloma, leukemia or lymphoma.

62. The method of any of claims 1-61, wherein the cancer is an acute lymphoblastic leukemia (ALL), adult ALL, chronic lymphoblastic leukemia (CLL), a small lymphocytic lymphoma (SLL), non-Hodgkin lymphoma (NHL), a large B cell lymphoma.

63. The method of any of claims 1-62, wherein the cancer is a chronic lymphocytic leukemia (CLL).

64. The method of any of claims 1-62, wherein the cancer is a small lymphocytic lymphoma (SLL).

65. The method of any of claims 1-62, wherein the cancer is a non-Hodgkin lymphoma (NHL), optionally wherein the NHL is a diffuse large B cell lymphoma.

66. The method of any of claims 1-65, wherein the subject has relapsed following remission after treatment with, or become refractory to, failed and/or was intolerant to treatment with the one or more prior therapies for treating the cancer.

67. The method of any of claims 1-66, wherein the cancer exhibits overexpression of a prosurvival BCL2 family protein that is targeted by the inhibitor.

68. The method of any of claims 1-67, wherein the dosing regimen of the inhibitor comprises administration of the inhibitor, optionally once daily, for a period of time of at least 3 months after the initiation of the administration of the cytotoxic therapy.

69. The method of claim 68, wherein the method further comprises continued administration of the dosing regimen of the inhibitor if at the end of the period of time, optionally at or about at 3 months, the subject does not exhibit a clinical remission, optionally wherein the subject exhibits a partial response (PR) or stable disease (SD), or has minimal residual disease (MRD) greater than or equal to 10⁻⁴.

70. The method of claim 68, wherein administration of the inhibitor in the dosing regimen is discontinued at the end of the period of time, optionally at or about at 3 months, if the subject exhibits clinical remission.

71. The method of any of claims 1-70, wherein:
the method increases the cytotoxic activity of the cytotoxic therapy compared to a method that does not involve the administration of the inhibitor; and/or
the method increases cytolytic killing, optionally via perforin- and/or granzyme-mediated apoptosis, of one or more of the cancer cells compared to a method that does not involve the administration of the inhibitor.

72. The method of any of claims 1-71, wherein:
at least 35%, at least 40% or at least 50% of subjects treated according to the method achieve a complete response (CR) that is durable, or is durable in at least 60, 70, 80, 90, or 95% of subjects achieving the CR, for at or greater than 6 months or at or greater than 9 months; and/or
wherein at least 60, 70, 80, 90, or 95% of subjects achieving a CR by six months remain in response, remain in CR, and/or survive or survive without progression, for greater at or greater than 3 months and/or at or greater than 6 months and/or at greater than nine months; and/or
at least 50%, at least 60% or at least 70% of the subjects treated according to the method achieve objective response (OR) optionally wherein the OR is durable, or is durable in at least 60, 70, 80, 90, or 95% of subjects achieving the OR, for at or greater than 6 months or at or greater than 9 months; and/or
wherein at least 60, 70, 80, 90, or 95% of subjects achieving an OR by six months remain in response or surviving for greater at or greater than 3 months and/or at or greater than 6 months.

73. The method of any one of claims 1-72, wherein the subject is a human.

74. A method of treatment with a cytotoxic therapy, the method comprising:
(a) assessing the level or amount of one or more prosurvival gene in a biological sample from a subject having or suspected of having a cancer, wherein the level or amount of the one or more prosurvival gene is the level or amount of a protein or a polynucleotide encoded by the one or more prosurvival gene;
(b) selecting the subject for treatment with a cytotoxic therapy if the level or amount of the one or more prosurvival gene is below a gene reference value, wherein the cytotoxic therapy is a T cell therapy comprising or enriched in T cells and specifically binds to an antigen associated with, expressed by, or present on cells of the cancer; and
(c) administering to the selected patient the cytotoxic therapy.

75. The method of claim 74, wherein an inhibitor of a prosurvival gene is not administered to the subject at or after initiation of the administration of the cytotoxic therapy, optionally is not administered within 7 days, 14 days or 28 days after the administration of the cytotoxic therapy.

76. A method of selecting a subject having a cancer for administering an inhibitor of a prosurvival BCL2 family protein, the method comprising:
(a) assessing the level or amount of one or more prosurvival gene in a biological sample from a subject having or suspected of having a cancer,
wherein the level or amount of the one or more prosurvival gene is the level or amount of a protein or a polynucleotide encoded by the one or more prosurvival gene, wherein the subject is to receive administration of a cytotoxic therapy, that is a T cell therapy comprising or enriched in T cells and that specifically binds to an antigen associated with, expressed by, or present on cells of the cancer, and wherein the biological sample is obtained from the subject prior to the administration of the cytotoxic therapy; and
(b) selecting the subject for treatment with an inhibitor of a prosurvival BCL2 family protein if the level or amount of the one or more prosurvival gene is above a gene reference value.

77. The method of claim 76, further comprising administering to the selected subject the inhibitor in combination with the cytotoxic therapy.

78. A method of identifying a subject having a cancer that is predicted to be resistant to treatment with a cytotoxic therapy, the method comprising:
(a) assessing the level or amount of one or more prosurvival gene in a biological sample from the subject,
wherein the level or amount of the one or more prosurvival gene is the level or amount of a protein and a polynucleotide encoded by the one or more prosurvival gene, wherein the subject is a candidate for administration of a dose of a cytotoxic therapy, wherein the cytotoxic therapy is a T cell therapy comprising or enriched in T cells and that specifically binds to an antigen associated with, expressed by, or present on cells of the cancer, and wherein the biological sample is obtained from the subject prior to the administration of the cytotoxic therapy; and
(b) identifying the subject as having a cancer that is predicted to be resistant to treatment with the cytotoxic therapy if the level or amount of the one or more prosurvival gene is above a gene reference value.

79. The method of claim 78, wherein if the subject is identified as having a cancer that is predicted to be resistant to treatment with the cytotoxic therapy, further comprising administering an alternative treatment to the identified subject, wherein the alternative treatment is selected from among the following: a combination treatment comprising the cytotoxic therapy and an additional agent that modulates or increases the activity of the cytotoxic therapy; an increased dose of the cytotoxic therapy; and/or a chemotherapeutic agent.

80. The method of claim 79, wherein the alternative treatment is a combination treatment comprising the cytotoxic therapy and an additional agent that modulates or increases the activity of the T cell therapy, optionally wherein the additional agent is an immune checkpoint inhibitor, a modulator of a metabolic pathway, an adenosine receptor antagonist, a kinase inhibitor, an anti-TGFβ antibody or an anti-TGFβR antibody, a cytokine, or a prosurvival BCL2 family protein inhibitor.

81. The method of claim 79 or claim 80, wherein the alternative treatment is a combination treatment comprising the cytotoxic therapy and a prosurvival BCL2 family protein inhibitor.

82. The method of claim 81, further comprising administering to the selected subject the inhibitor in combination with the cytotoxic therapy.

83. The method of any of claims 74-82, wherein the gene reference value is within 25%, within 20%, within 15%, within 10%, or within 5% of an average level or amount of the one or more prosurvuval gene in (a) a population of subjects not having the cancer or (b) a population of subjects having the cancer and administered the cytotoxic therapy, who went on to exhibit a partial response (PR) or complete response (CR) following administration of the therapy.

84. The method of claim 83, wherein the population of subjects having the cancer went on to exhibit the PR or CR at least 1 month, 2 months, 3 months, 4 months, 5 months, 6 months, 7 months, 8 months, 9 months, or more following administration of the cytotoxic therapy.

85. The method of any of claims 74-84, wherein the level or amount of the one or more prosurvival genes is assessed in the biological sample before a lymphodepleting therapy is administered to the subject, optionally within 7 days before, 6 days before, 5 days before, 4 days before, 3 days before, 2 days before, 1 day before, 16 hours before, 12 hours before, 6 hours before, 2 hours before, or 1 hour before the lymphodepleting therapy is administered to the subject.

86. A method of determining responsiveness of a subject having a cancer to a cytotoxic therapy, wherein the cytotoxic therapy is a T cell therapy comprising or enriched in T cells and that specifically binds to an antigen associated with, expressed by, or present on cells of the cancer, the method comprising:
(a) assessing the level or amount of expression of one or more prosurvival gene in a biological sample from the subject,
wherein the level or amount of the one or more prosurvival gene is the level or amount of a protein or a polynucleotide encoded by the one or more prosurvival gene, wherein the biological sample is obtained from the subject at a first time prior to the subject being administered the cytotoxic therapy, and wherein the subject is to receive treatment with the cytotoxic therapy;
(b) assessing the level or amount of expression of the one or more prosurvival gene in a biological sample from the subject at a second time after administration of the cytotoxic therapy to the subject,
wherein the level or amount of the one or more prosurvival gene is the level or amount of a protein and/or a polynucleotide encoded by the one or more prosurvival gene, wherein the biological sample is obtained at a second time after the administration of the cytotoxic therapy to the subject, and wherein the subject has been administered the cytotoxic therapy prior to the assessing in (b); and
(c) determining that the subject is responsive to the therapy if the level or amount of the one or more prosurvival gene at the second time is lower than the level or amount of the one or more prosurvival gene at the first time.

87. The method of claim 86, further comprising prior to the assessing in (b), administering to the subject the cytotoxic therapy.

88. The method of claim 86 or claim 87, wherein the biological sample is obtained from the subject at a time before a lymphodepleting therapy is administered to the subject, optionally within 7 days before, 6 days before, 5 days before, 4 days before, 3 days before, 2 days before, 1 day before, 16 hours before, 12 hours before, 6 hours before, 2 hours before, or 1 hour before the lymphodepleting therapy is administered to the subject.

89. The method of any of claims 74-88, wherein the one or more pro-survival gene is selected from among the following: a myc family gene, p53, and enhancer of zeste homolog 2 (EZH2).

90. The method of claim 89, wherein the one or more pro-survival gene is or comprises a myc family gene.

91. The method of claim 90, wherein a myc family gene comprises one or more of c-myc, l-myc, and n-myc.

92. The method of claim 89, wherein the one or more pro-survival gene is or comprises p53.

93. The method of claim 89, wherein the one or more pro-survival gene is or comprises EZH2.

94. The method of any of claims 74-93, wherein the cytotoxic therapy comprises cells that are autologous to the subject.

95. The method of any of claims 74-94, wherein the cytotoxic therapy is selected from among the group consisting of a tumor infiltrating lymphocytic (TIL) therapy, a transgenic TCR therapy, and a chimeric antigen receptor (CAR)-expressing cell therapy.

96. The method of any of claims 74-95, wherein the cytotoxic therapy comprises a dose of cells expressing a recombinant receptor that specifically binds to the antigen.

97. The method of any of claims 74-96, wherein the cytotoxic therapy comprises or is enriched in CD3+, CD4+, CD8+, or CD4+ and CD8+ T cells.

98. The method of any of claims 74-97, wherein the cytotoxic therapy comprises or is enriched in CD4+ and CD8+ T cells.

99. The method of claim 98, wherein the CD4+ and CD8+ T cells of the cytotoxic therapy comprises a defined ratio of CD4+ recombinant receptor-expressing T cells to CD8+ CAR-expressing T cells and/or of CD4+ recombinant-expressing T cells to CD8+ CAR-expressing T cells, that is or is approximately 1:1 or is between approximately 1:3 and approximately 3:1.

100. The method of any of claims 96-99, wherein the recombinant receptor is a T cell receptor (TCR) or a functional non-T cell receptor.

101. The method of any of claims 96-100, wherein the recombinant receptor is a chimeric antigen receptor (CAR).

102. The method of claim 101, wherein the CAR comprises an extracelluar antigen binding domain that binds to the antigen, a transmembrane domain, and an intracellular signaling region comprising an intracellular signaling domain of a CD3-zeta (CD3ζ) chain and a costimulatory signaling domain.

103. The method of claim 102, wherein the costimulatory signaling region comprises a signaling domain of 4-1BB.

104. The method of claim 103, wherein the costimulatory region comprises a signaling domain of CD28.

105. The method of any of claims 77, 82-85 and 89-104, wherein the inhibitor is administered in combination with the cytotoxic therapy in accord with any of methods 1-73.

106. The method of any of claims 75-105, wherein the inhibitor of a prosurvival gene is a BCL2 family protein inhibitor, wherein the inhibitor inhibits one or more prosurvival BCL2 family protein selected from among the group consisting of BCL2, BCLXL, BCLW, BCLB, MCL1, and combinations thereof.

107. The method of claim 106, wherein the one or more prosurvival BCL2 family protein is BCL2, BCLXL, and/or BCLW.

108. The method of claim 106 or claim 107, wherein the inhibitor is selected from among the group consisting of venetoclax, navitoclax, ABT737, maritoclax, obatoclax, and clitocine.

109. The method of any of claims 75-108, wherein the inhibitor is venetoclax.

110. The method of any of claims 74-109, wherein the cancer is a hematological malignancy.

111. The method of any of claims 74-110, wherein the cancer is a B cell malignancy.

112. The method of any of claims 74-111, wherein the cancer is a myeloma, leukemia or lymphoma.

113. The method of any of claims 74-112, wherein the cancer is an acute lymphoblastic leukemia (ALL), adult ALL, chronic lymphoblastic leukemia (CLL), a small lymphocytic lymphoma (SLL), non-Hodgkin lymphoma (NHL), a large B cell lymphoma.

114. The method of any of claims 74-113, wherein the cancer is a chronic lymphocytic leukemia (CLL).

115. The method of any of claims 74-113, wherein the cancer is a small lymphocytic lymphoma (SLL).

116. The method of any of claims 74-113, wherein the cancer is a non-Hodgkin lymphoma (NHL), optionally wherein the NHL is a diffuse large B-cell lymphoma (DLBCL).

117. The method of any of claims 74-116, wherein the subject has relapsed following remission after treatment with, or become refractory to, failed and/or was intolerant to treatment with the one or more prior therapies for treating the cancer.

118. The method of any of claims 74-117, wherein the biological sample is a tumor biopsy, optionally a lymph node biopsy.

119. The method of any one of claims 74-118, wherein the subject is a human.
